SEQUENCE LISTING (195 sequences)

SEQ ID NO: 1-2 - skipped (2 entries)

SEQ ID NO: 3 - RNA, 10 nt, synthetic construct
  source 1..10: mol_type = other RNA; note = Description of Artificial Sequence: Synthetic oligonucleotide
tttttttttt

SEQ ID NO: 4 - DNA, 10 bp, synthetic construct
  source 1..10: mol_type = other DNA; note = Description of Artificial Sequence: Synthetic oligonucleotide; note = Description of Combined DNA/RNA Molecule: Synthetic oligonucleotide
  misc_feature 1..4: note = DNA
  misc_feature 5..6: note = RNA
  misc_feature 7..10: note = DNA
tttttttttt

SEQ ID NO: 5-11 - skipped (7 entries)

SEQ ID NO: 12 - DNA, 10 bp, synthetic construct
  source 1..10: mol_type = other DNA; note = Description of Artificial Sequence: Synthetic oligonucleotide
tttttttttt

SEQ ID NO: 13 - DNA, 12 bp, synthetic construct
  source 1..12: mol_type = other DNA; note = Description of Artificial Sequence: Synthetic oligonucleotide
tttttttttttt

SEQ ID NO: 14 - DNA, 14 bp, synthetic construct
  source 1..14: mol_type = other DNA; note = Description of Artificial Sequence: Synthetic oligonucleotide
tttttttttttttt

SEQ ID NO: 15-17 - skipped (3 entries)

SEQ ID NO: 18 - protein, 1228 aa, unidentified
  source 1..1228: note = Description of Unknown: Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 19 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 20 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 21 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 22 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 23 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 24 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 25 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 26 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 27 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 28 - protein, 1224 aa, synthetic construct
  source 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 29 - protein, 1297 aa, synthetic construct
  source 1..1297: note = Description of Artificial Sequence: Synthetic polypeptide
MATLVSFTKQYQVQKTLRFELIPQGKTQANIDAKGFINDDLKRDENYMKVKGVIDELHKNFIEQTLVNVDYDWRSLATAIKNYRKDRSDTNKKNLEKTQEAARKEIIAWFEGKRGNSAFKNNQKSFYGKLFKKELFSEILRSDDLEYDEETQDAIACFDKFTTYFVGFHENRKNMYSTEAKSTSVAYRVVNENFSKFLSNCEAFSVLEAVCPNVLVEAEQELHLHKAFSDLKLSDVFKVEAYNKYLSQTGIDYYNQIIGGISSAEGVRKIRGVNEVVNNAIQQNDELKVALRNKQFTMVQLFKQILSDRSTLSFVSEQFTSDQEVITVVKQFNDDIVNNKVLAVVKTLFENFNSYDLEKIYINSKELASVSNALLKDWSKIRNAVLENKIIELGANPPKTKISAVEKEVKNKDFSIAELASYNDKYLDKEGNDKEICSIANVVLEAVGALEIMLAESLPADLKTLENKNKVKGILDAYENLLHLLNYFKVSAVNDVDLAFYGAFEKVYVDISGVMPLYNKVRNYATKKPYSVEKFKLNFAMPTLADGWDKNKERDNGSIILLKDGQYYLGVMNPQNKPVIDNAVCNDAKGYQKMVYKMFPEISKMVTKCSTQLNAVKAHFEDNTNDFVLDDTDKFISDLTITKEIYDLNNVLYDGKKKFQIDYLRNTGDFAGYHKALETWIDFVKEFLSKYRSTAIYDLTTLLPTNYYEKLDVFYSDVNNLCYKIDYENISVEQVNEWVEEGNLYLFKIYNKDFATGSTGKPNLHTMYWNAVFAEENLHDVVVKLNGGAELFYRPKSNMPKVEHRVGEKLVNRKNVNGEPIADSVHKEIYAYANGKISKSELSENAQEELPLAIIKDVKHNITKDKRYLSDKYFFHVPITLNYKANGNPSAFNTKVQAFLKNNPDVNIIGIDRGERNLLYVVVIDQQGNIIDKKQVSYNKVNGYDYYEKLNQREKERIEARQSWGAVGKIKELKEGYLSLVVREIADMMVKYNAIVVMENLNAGFKRVRGGIAEKAVYQKFEKMLIDKLNYLVFKDVEAKEAGGVLNAYQLTDKFDSFEKMGNQSGFLFYVPAAYTSKIDPVTGFANVFSTKHITNTEAKKEFICSFNSLRYDEAKDKFVLECDLNKFKIVANSHIKNWKFIIGGKRIVYNSKNKTYMEKYPCEDLKATLNASGIDFSSSEIINLLKNVPANREYGKLFDETYWAIMNTLQMRNSNALTGEDYIISAVADDNEKVFDSRTCGAELPKDADANGAYHIALKGLYLLQRIDISEEGEKVDLSIKNEEWFKFVQQKEYAR

SEQ ID NO: 30 - protein, 1229 aa, synthetic construct
  source 1..1229: note = Description of Artificial Sequence: Synthetic polypeptide
MKEQFINRYPLSKTLRFSLIPVGETENNFNKNLLLKKDKQRAENYEKVKCYIDRFHKEYIESVLSKARIEKVNEYANLYWKSNKDDSDIKAMESLENDMRKQISKQLTSTEIYKKRLFGKELICEDLPSFLTDKDERETVECFRSFTTYFKGFNTNRENMYSSDGKSTAIAYRCINDNLPRFLDNVKSFQKVFDNLSDETITKLNTDLYNIFGRNIEDIFSVDYFEFVLTQSGIEIYNSMIGGYTCSDKTKIQGLNECINLYNQQVAKNEKSKKLPLMKPLYKQILSEKDSVSFIPEKFNSDNEVLHAIDDYYTGHIGDFDLLTELLQSLNTYNANGIFVKSGVAITDISNGAFNSWNVLRSAWNEKYEALHPVTSKTKIDKYIEKQDKIYKAIKSFSLFELQSLGNENGNEITDWYISSINESNSKIKEAYLQAQKLLNSDYEKSYNKRLYKNEKATELVKNLLDAIKEFQKLIKPLNGTGKEENKDELFYGKFTSYYDSIADIDRLYDKVRNYITQKPYSKDKIKLNFDNPQLLGGWDKNKESDYRTVLLHKDGLYYLAVMDKSHSKAFVDAPEITSDDKDYYEKMEYKLLPGPNKMLPKVFFASKNIDTFQPSDRILDIRKRESFKKGATFNKAECHEFIDYFKDSIKKHDDWSQFGFKFSPTESYNDISEFYREISDQGYSVRFNKISKNYIDGLVNNGYIYLFQIYNKDFSKYSKGTPNLHTLYFKMLFDERNLSNVVYKLNGEAEMFYREASIGDKEKITHYANQPIKNKNPDNEKKESVFEYDIVKDKRFTKRQFSLHLPITINFKAHGQEFLNYDVRKAVKYKDDNYVIGIDRGERNLIYISVINSNGEIVEQMSLNEIISDNGHKVDYQKLLDTKEKERDKARKNWTSVENIKELKEGYISQVVHKICELVIKYDAVIAMEDLNFGFKRGRFPVEKQVYQKFENMLISKLNLLIDKKAEPTEDGGLLRAYQLTNKFDGVNKAKQNGIIFYVPAWDTSKIDPATGFVNLLKPKCNTSVPEAKKLFETIDDIKYNANTDMFEFYIDYSKFPRCNSDFKKSWTVCTNSSRILTFRNKEKNNKWDNKQIVLTDEFKSLFNEFGIDYKGNLKDSILSISNADFYRRLIKLLSLTLQMRNSITGSTLPEDDYLISPVANKSGEFYDSRNYKGTNAALPCDADANGAYNIARKALWAINVLKDTPDDMLNKAKLSITNAEWLEYTQK

SEQ ID NO: 31 - protein, 1470 aa, synthetic construct
  source 1..1470: note = Description of Artificial Sequence: Synthetic polypeptide
MNNPRGAFGGFTNLYSLSKTLRFELKPYLEIPEGEKGKLFGDDKEYYKNCKTYTEYYLKKANKEYYDNEKVKNTDLQLVNFLHDERIEDAYQVLKPVFDTLHEEFITDSLESAEAKKIDFGNYYGLYEKQKSEQNKDEKKKIDKPLETERGKLRKAFTPIYEAEGKNLKNKAGKEKKDKDILKESGFKVLIEAGILKYIKNNIDEFADKKLKNNEGKEITKKDIETALGAENIEGIFDGFFTYFSGFNQNRENYYSTEEKATAVASRIVDENLSKFCDNILLYRKNENDYLKIFNFLKNKGKDLKLKNSKFGKENEPEFIPAYDMKNDEKSFSVADFVNCLSQGEIEKYNAKIANANYLINLYNQNKDGNSSKLSMFKILYKQIGCGEKKDFIKTIKDNAELKQILEKACEAGKKYFIRGKSEDGGVSNIFDFTDYIQSHENYKGVYWSDKAINTISGKYFANWDTLKNKLGDAKVFNKNTGEDKADVKYKVPQAVMLSELFAVLDDNAGEDWREKGIFFKASLFEGDQNKSEIIKNANRPSQALLKMICDDMESLAKNFIDSGDKILKISDRDYQKDENKQKIKNWLDNALWINQILKYFKVKANKIKGDSIDARIDSGLDMLVFSSDNPAEDYDMIRNYLTQKPQDEINKLKLNFENSSLAGGWDENKEKDNSCIILKDEQDKQYLAVMKYENTKVFEQKNSQLYIADNAAWKKMIYKLVPGASKTLPKVFFSKKWTANRPTPSDIVEIYQKGSFKKENVDFNDKKEKDESRKEKNREKIIAELQKTCWMDIRYNIDGKIESAKYVNKEKLAKLIDFYKENLKKYPSEEESWDRLFAFGFSDTKSYKSIDQFYIEVDKQGYKLEFVTINKARLDEYVRDGKIYLFEIRSRDNNLVNGEEKTSAKNLQTIYWNAAFGGDDNKPKLNGEAEIFYRPAIAENKLNKKKDKNGKEIIDGYRFSKEKFIFHCPITLNFCLKETKINDKLNAALAKPENGQGVYFLGIDRGEKHLAYYSLVNQKGEILEQGTLNLPFLDKNGKSRSIKVEKKSFEKDSNGGIIKDKDGNDKIKIEFVECWNYNDLLDARAGDRDYARKNWTTIGTIKELKDGYISQVVRKIVDLSIYKNTETKEFREMPAFIVLEDLNIGFKRGRQKIEKQVYQKLELALAKKLNFLVDKKADIGEIGSVTKAIQLTPPVNNFGDMENRKQFGNMLYIRADYTSQTDPATGWRKSIYLKSGSESNVKEQIEKSFFDIRYESGDYCFEYRDRHGKMWQLYSSKNGVSLDRFHGERNNSKNVWESEKQPLNEMLDILFDEKRFDKSKSLYEQMFKGGVALTRLPKEINKKDKPAWESLRFVIILIQQIRNTGKNGDDRNGDFIQSPVRDEKTGEHFDSRIYLDKEQKGEKADLPTSGDANGAYNIARKGIVVAEHIKRGFDKLYISDEEWDTWLAGDEIWDKWLKENRESLTKTRK

SEQ ID NO: 32 - protein, 1282 aa, synthetic construct
  source 1..1282: note = Description of Artificial Sequence: Synthetic polypeptide
MNGNRIIVYREFVGVTPVAKTLRNELRPIGHTQEHIIHNGLIQEDELRQEKSTELKNIMDDYYREYIDKSLSGVTDLDFTLLFELMNLVQSSPSKDNKKALEKEQSKMREQICTHMQSDSNYKNIFNAKFLKEILPDFIKNYNQYDAKDKAGKLETLALFNGFSTYFTDFFEKRKNVFTKEAVSTSIAYRIVHENSLTFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDEHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYNDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKENDLSEAAKEYLDKVEVRTAQKDIVKDYRYTVDKYFIHTPITINYKVTARNNVNDMAVKYIAQNDDIHVIGIDRGERNLIYISVIDSHGNIVKQKSYNILNNYDYKKKLVEKEKTREYARKNWKSIGNIKELKEGYISGVVHEIAMLMVEYNAIIAMEDLNYGFKRGRFKVERQVYQKFESMLINKLNYFASKGKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMSKTQWTVYTNGERLQSEFNNARRTGKTKSINLTETIKLLLEDNEINYADGHDVRIDMEKMDEDKNSEFFAQLLSLYKLTVQMRNSYTEAEEQEKGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 33 - protein, 1225 aa, synthetic construct
  source 1..1225: note = Description of Artificial Sequence: Synthetic polypeptide
MKKIDSFVNYYPLSKTLRFSLIPVGKTEDNFNAKLLLEEDEKRAIEYEKVKRYIDRYHKHFIETVLANFHLDDVNEYAELYYKAGKDDKDLKYMEKLEGKMRKSISAAFTKDKKYKEIFGQEIIKNILPEFLENEDEKESVKMFQGFFTYFTGFNDNRKNMYTHEAQTTAISYRCINENLPKFLDNVQSFAKIKESISSDIMNKLDEVCMDLYGVYAQDMFCTDYFSFVLSQSGIDRYNNIIGGYVDDKGVKIQGINEYINLYNQQVDEKNKRLPLMKKLYKQILIEKESISFIPEKFESDNIVINAISDYYHNNVENLFDDFNKLFNEFSEYDDNGIFVTSGLAVTDISNAVFGSWNIISDSWNEEYKDSHPMKKTTNAEKYYEDMKKEYKKNLSFTIAELQRLGEAGCNDECKGDIKEYYKTTVAEKIENIKNAYEISKDLLASDYEKSNDKKLCKNDSAISLLKNLLDSIKDLEKTIKPLLGTGKEENKDDVFYGKFTNLYEMISEIDRLYDKVRNYVTQKPYSKDKIKLNFENPQHLGGWDKNKERDYRSVLLKKEDKYYLAIMDKSNNKAFIDFPDDGECYEKIEYKLLPGPNKMLPKVFFASSNIEYFAPSKKILEIRSRESFKKGDMFNLKDCHEFIDFFKESIKKHEDWSQFGFEFSPTEKYNDISEFYNEVKIQGYSLKYKNVSKKYIDELIECGQLYLFQIYNKDFSVYAKGNPNLHTMYFKMLFDERNLANVVYQLNGGAEMFYRKASIKDSEKIVHHANQPIKNKNADNVKKESVFEYDIIKDKRFTKRQFSIHIPITLNFKAKGQNFINNDVRMALKKADENYVIGIDRGERNLLYICVINSKGEIVEQKSLNEIIGDNGYRVDYHKLLDKKEAERDEARKSWGTIENIKELKEGYLSQIVHEISKLVIKYDAVIAIEDLNSGFKKGRFKVEKQVYQKFENMLCTKLNYLVDKNADANECGGLLKAYQLTNKEDGANRGRQNGIIFSVPAWLTSKIDPVTGFADLLRPKYKSVSESVEFISKIDNIRYNSKEDYFEFDIDYSKFPNSTASYKKKWTVCTYGERIINVRNKEKNNMWDNKTIVLTDEFKKLFADFGVDVSKNIKESVLAIDSKDFYYRFINLLANTLQLRNSEVGNVDVDYLISPVKGVDGSFYDSRLVKEKTLPENADANGAYNIARKALWAIDVLKQTKDEELKNANLSIKNAEWLEYVQK

SEQ ID NO: 34 - protein, 1293 aa, synthetic construct
  source 1..1293: note = Description of Artificial Sequence: Synthetic polypeptide
MRTMVTFEDFTKQYQVSKTLRFELIPQGKTLENMKRDGIISVDRQRNEDYQKAKGILDKLYKYILDFTMETVVIDWEALATATEEFRKSKDKKTYEKVQSKIRTALLEHVKKQKVGTEDLFKGMFSSKIITGEVLAAFPEIRLSDEENLILEKFKDFTTYFTGFFENRKNVFTDEALSTSFTYRLVNDNFIKFFDNCIVFKNVVNISPHMAKSLETCASDLGIFPGVSLEEVFSISFYNRLLTQTGIDQFNQLLGGISGKEGEHKQQGLNEIINLAMQQNLEVKEVLKNKAHRFTPLFKQILSDRSTMSFIPDAFADDDEVLSAVDAYRKYLSEKNIGDRAFQLISDMEAYSPELMRIGGKYVSVLSQLLFYSWSEIRDGVKAYKESLITGKKTKKELENIDKEIKYGVTLQEIKEALPKKDIYEEVKKYAMSVVKDYHAGLAEPLPEKIETDDERASIKHIMDSMLGLYRFLEYFSHDSIEDTDPVFGECLDTILDDMNETVPLYNKVRNFSTRKVYSTEKFKLNFNNSSLANGWDKNKEQANGAILLRKEGEYFLGIFNSKNKPKLVSDGGAGIGYEKMIYKQFPDFKKMLPKCTISLKDTKAHFQKSDEDFTLQTDKFEKSIVITKQIYDLGTQTVNGKKKFQVDYPRLTGDMEGYRAALKEWIDFGKEFIQAYTSTAIYDTSLFRDSSDYPDLPSFYKDVDNICYKLTFEWIPDAVIDDCIDDGSLYLFKLHNKDFSSGSIGKPNLHTLYWKALFEEENLSDVVVKLNGQAELFYRPKSLTRPVVHEEGEVIINKTTSTGLPVPDDVYVELSKFVRNGKKGNLTDKAKNWLDKVTVRKMPHAITKDRRFTVDKFFFHVPITLNYKADSSPYRFNDFVRQYIKDCSDVKIIGIDRGERNLIYAVVIDGKGNIIEQRSFNTVGTYNYQEKLEQKEKERQTARQDWATVTKIKDLKKGYLSAVVHELSKMIVKYKAIVALENLNVGFKRMRGGIAERSVYQQFEKALIDKLNYLVFKDEEQSGYGGVLNAYQLTDKFESFSKMGQQTGFLFYVPAAYTSKIDPLTGFINPFSWKHVKNREDRRNFLNLFSKLYYDVNTHDFVLAYHHSNKDSKYTIKGNWEIADWDILIQENKEVFGKTGTPYCVGKRIVYMDDSTTGHNRMCAYYPHTELKKLLSEYGIEYTSGQDLLKIIQEFDDDKLVKGLFYIIKAALQMRNSNSETGEDYISSPIEGRPGICFDSRAEADTLPYDADANGAFHIAMKGLLLTERIRNDDKLAISNEEWLNYIQEMRG

SEQ ID NO: 35 - protein, 1399 aa, synthetic construct
  source 1..1399: note = Description of Artificial Sequence: Synthetic polypeptide
MNKDIRKNFTDFVGISEIQKTLRFILIPIGKTAQNIDKYNMFEDDEIRHEYYPILKEACDDFYRNHIDQQFENLELDWSKLDEALASEDRDLINETRATYRQVLFNRLKNSVDIKGDSKKNKTLSLESSDKNLGKKKTKNTFQYNFNDLFKAKLIKAILPLYIEYIYEGEKLENAKKALKMYNRFTSRLSNFWQARANIFTDDEISTGSPYRLVNDNFTIFRINNSIYTKNKPFIEEDILEFEKKLKSKKIIKDFESVDDYFTVNAFNKLCTQNGIDKYNSILGGFTTKEREKVKGLNELFNLAQQSINKGKKGEYRKNIRLGKLTKLKKQILAISDSTSFLIEQIEDDQDLYNKIKDFFELLLKEEIENENIFTQYANLQKLIEQADLSKIYINAKHLNKISHQVTGKWDSLNKGIALLLENININEESKEKSEVISNGQTKDISSEAYKRYLQIQSEEKDIERLRTQIYFSLEDLEKALDLVLIDENMDRSDKSILSYVQSPDLNVNFERDLTDLYSRIMKLEENNEKLLANHSAIDLIKEFLDLIMLRYSRWQILFCDSNYELDQTFYPIYDAVMEILSNIIRLYNLARNYLSRKPDRMKKKKINFNNPTLADGWSESKIPDNSSMLFIKDGMYYLGIIKNRAAYSELLEAESLQSSEKKKSENSSYERMNYHFLPDAFRSIPKSSIAMKAVKEHFEINQKTADLLLDTDKFSKPLRITKEIFDMQYVDLHKNKKKYQVDYLRDTGDKKGYRKALNTWLNFCKDFISKYKGRNLFDYSKIKDADHYETVNEFYNDVDKYSYHIFFTSVAETTVEKFISEGKLYLFQLYNKDFSPHSTGKPNLHTIYWRALFSEENLTSKNIKLNGQAEIFFRPKQIETPFTHKKGSILVNRFDVNGNPIPINVYQEIKGFKNNVIKWDDLNKTTQEGLENDQYLYFESEFEIIKDRRYTEDQLFFHVPISFNWDIGSNPKINDLATQYIVNSNDIHIIGIDRGENHLIYYSVIDLQGAIVEQGSLNTITEYTENKFLNNKTNNLRKIPYKDILQQREDERADARIKWHAIDKIKDLKDGYLGQIVHFLAKLIIKYNAIVILEDLNYGFKRGRFKVERQVYQKFEMALMKKLNVLVFKDYDIDEIGGPLKPWQLTRPIDSYERMGRQNGILFYVPAAYTSAVDPVTGFANLFYLNNVKNSEKFHFFSKFESIKYHSDQDMFSFAFDYNNFGTTTRINDLSKSKWQVFTNHERSVWNNKEKNYVTQNLTDLIKKLLRTYNIEFKNNQNVLDSILKIENNTDKENFARELFRLFRLTIQLRNTTVNENNTEITENELDYIISPVKDKNGNFFDSRDELKNLPDNGDANGAYNIARKGLLYIEQLQESIKTGKLPTLSISTLDWFNYIMK

SEQ ID NO: 36 - protein, 1272 aa, synthetic construct
  source 1..1272: note = Description of Artificial Sequence: Synthetic polypeptide
MTPIFCNFVVYQIMLFNNNININVKTMNKKHLSDFTNLFPVSKTLRFRLEPQGKTMENIVKAQTIETDEERSHDYEKTKEYIDDYHRQFIDDTLDKFAFKVESTGNNDSLQDYLDAYLSANDNRTKQTEEIQTNLRKAIVSAFKMQPQFNLLFKKEMVKHLLPQFVDTDDKKRIVAKFNDFTTYFTGFFTNRENMYSDEAKSTSIAYRIVNQNLIKFVENMLTFKSHILPILPQEQLATLYDDFKEYLNVASIAEMFELDHFSIVLTQRQIEVYNSVIGGRKDENNKQIKPGLNQYINQHNQAVKDKSARLPLLKPLFNQILSEKAGVSFLPKQFKSASEVVKSLNEAYAELSPVLAAIQDVVTNITDYDCNGIFIKNDLGLTDIAQRFYGNYDAVKRGLRNQYELETPMHNGQKAEKYEEQVAKHLKSIESVSLAQINQVVTDGGDICDYFKAFGATDDGDIQRENLLASINNAHTAISPVLNKENANDNELRKNTMLIKDLLDAIKRLQWFAKPLLGAGDETNKDQVFYGKFEPLYNQLDETISPLYDKVRSYLTKKPYSLDKFKINFEKSNLLGGWDPGADRKYQYNAVILRKDNDFYLGIMRDEATSKRKCIQVLDCNDEGLDENFEKVEYKQIKPSQNMPRCAFAKKECEENADIMELKRKKNAKSYNTNKDDKNALIRHYQRYLDRTYPEFGFVYKDADEYDTVKAFTDSMDSQDYKLSFLQVSETGLNKLVDEGDLYLFKITNKDFSSYAKGRPNLHTIYWRMLFDPKNLANVVYKLEGKAEVFFRRKSLASTTTHKAKQAIKNKSRYNEAVKPQSTFDYDIIKDRRFTADKFEFHVPIKMNFKAAGWNSTRLTNEVREFIKSQGVRHIIGIDRGERHLLYLTMIDMDGNIVKQCSLNAPAQDNARASEVDYHQLLDSKEADRLAARRNWGTIENIKELKQGYLSQVVHLLATMMVDNDAILVLENLNAGFMRGRQKVEKSVYQKFEKMLIDKLNYIVDKGQSPDKPTGALHAVQLTGLYSDFNKSNMKRANVRQCGFVFYIPAWNTSKIDPVTGFVNLFDTHLSSMGEIKAFFSKFDSIRYNQDKGWFEFKFDYSRFTTRAEGCRTQWTVCTYGERIWTHRSKNQNNQFVNDTVNVTQQMLQLLQDCGIDPNGNLKEAIANIDSKKSLETLLHLFKLTVQMRNSVTGSEVDYMISPVADERGHFFDSRESDEHLPANADANGAFNIARKGLMVVRQIMATDDVSKIKFAVSNKDWLRFAQHIDD

SEQ ID NO: 37 - protein, 1313 aa, synthetic construct
  source 1..1313: note = Description of Artificial Sequence: Synthetic polypeptide
MNKGGYVIMEKMTEKNRWENQFRITKTIKEEIIPTGYTKVNLQRVNMLKREMERNEDLKKMKEICDEYYRNMIDVSLRLEQVRTLGWESLIHKYRMLNKDEKEIKALEKEQEDLRKKISKGFGEKKAWTGEQFIKKILPQYLMDHYTGEELEEKLRIVKKFKGCTMFLSTFFKNRENIFTDKPIHTAVGHRITSENAMLFAANINTYEKMESNVTLEIERLQREFWRRGINISEIFTDAYYVNVLTQKQIEAYNKICGDINQHMNEYCQKQKLKFSEFRMRELKKQILAVVGEHFEIPEKIESTKEVYRELNEYYESLKELHGQFEELKSVQLKYSQIYVQKKGYDRISRYIGGQWDLIQECMKKDCASGMKGTKKNHDAKIEEEVAKVKYQSIEHIQKLVCTYEEDRGHKVTDYVDEFIVSVCDLLGADHIITRDGERIELPLQYEPGTDLLKNDTINQRRLSDIKTILDWHMDMLEWLKTFLVNDLVIKDEEFYMAIEELNERMQCVISVYNRIRNYVTQKGYEPEKIRICFDKGTILTGWTTGDNYQYSGFLLMRNDKYYLGIINTNEKSVRKILDGNEECKDENDYIRVGYHLINDASKQLPRIFVMPKAGKKSEILMKDEQCDYIWDGYCHNKHNESKEFMRELIDYYKRSIMNYDKWEGYCFKFSSTESYDNMQDFYKEVREQSYNISFSYINENVLEQLDKDGKIYLFQVYNKDFAAGSTGTPNLHTMYLQNLFSSQNLELKRLRLGGNAELFYRPGTEKDVTHRKGSILVDRTYVREEKDGIEVRDTVPEKEYLEIYRYLNGKQKGDLSESAKQWLDKVHYREAPCDIIKDKRYAQEKYFLHFSVEINPNAEGQTALNDNVRRWLSEEEDIHVIGIDRGERNLIYVSLMDGKGRIKDQKSYNIVNSGNKEPVDYLAKLKVREKERDEARRNWKAIGKIKDIKTGYLSYVVHEIVEMAVREKAIIVMEDLNYGFKRGRFKVERQVYQKFEEMLINKLNYVVDKQLSVDEPGGLLRGYQLAFIPKDKKSSMRQNGIVFYVPAGYTSKIDPTTGFVNIFKFPQFGKGDDDGNGKDYDKIRAFFGKFDEIRYECDEKVTADNTREVKERYRFDFDYSKFETHLVHMKKTKWTVYAEGERIKRKKVGNYWTSEVISDIALRMSNTLNIAGIEYKDGHNLVNEICALRGKQAGIILNELLEIVRLTVQLRNSTTEGDVDERDEIISPVLNEKYGCFYHSTEYKQQNGDVLPKDADANGAYCIGLKGIYEIRQIKNKWKEDMTKGEGKALNEGMRISHDQWFEFIQNMNKGE

SEQ ID NO: 38 - protein, 1134 aa, synthetic construct
  source 1..1134: note = Description of Artificial Sequence: Synthetic polypeptide
MNELVKNRCKQTKTICQKLIPIGKTRETIEKYNLMEIDRKIAANKELMNKLFSLIAGKHINDTLSKCTDLDFEPLLTSLSSLNNAKENDRDNLREYYDSVFEEKKTLAEEISSRLTAVKFAGKDFFTKNIPDFLETYEGDDKNEMSELVSLVIENTVTAGYVKKLEKIDRSMEYRLVSGTVVKRVLTDNADIYEKNIEKAKDFDYGVLNIDEASQFTTLVAKDYANYLTADGIAIYNVGIGKINLALNEYCQKNKEYSYNKLALLPLQKMLYGEKLSLFEKLEDFTSDEELINSYNKFAKTVNESGLAEIIKKAVPSYDEIVIKPNKISNYSNSITGHWSLVNRIMKDYLENNGIKNADKYMEKGLTLSEIGDALENKNIKHSDFISNLINDLGHTYTEIKENKESLKKDESVNALIIKKELDMLLSILQNLKVFDIDNEMFDTGFGIEVSKAIEILGYGVPLYNKIRNYITKKPDPKKKFMTKFGSATIGTGITTSVEGSKKATFLKDGDAVFLLLYNTAGCKANNVSVSNLADLINSSLEIENSGKCYQKMIYQTPGDIKKQIPRVFVYKSEDDDLIKDFKAGLHKTDLSFLNGRLIPYLKEAFATHETYKNYTFSYRNSYESYDEFCEHMSEQAYILEWKWIDKKLIDDLVEDGSLLMFRVWNRFMKKKEGKISKHAKIVNELFSDENASNAAIKLLSVFDIFYRDKQIDNPIVHKAGTTLYNKRTKDGEVIVDYTTMVKNKEKRPNVYTTTKKYDIIKDRRYTEEQFEIHLHVNIGKEENKEKLETSKVINEKKNTLVVTRSNEHLLYVVIFDENDNILLKKSLNTVKGMNFKSKLEVVEIQKKENMQSWKTVGSNQALMEGYLSFAIKEIADLVKEYDAILVLEQNSVGKNILNERVYTRFKEMLITNLSLDVDYENKDFYSYTELGGKVASWRDCVTNGICIQVPSAYKYKDPTTSFSTISMYAKTTAEKSKKLKQIKSFKYNRERGLFELVIAKGVGLENNIVCDSFGSRSIIENDISKEVSCTLKIEKYLIDAGIEYNDEKEVLKDLDTAAKTDAVHKAVTLLLKCFNESPDGRYYISPCGEHFTLCDAPEVLSAINYYIRSRYIREQIVEGVKKMEYKKTILLAK

SEQ ID NO: 39 - protein, 1259 aa, synthetic construct
  source 1..1259: note = Description of Artificial Sequence: Synthetic polypeptide
MNYKTGLEDFIGKESLSKTLRNALIPTESTKIHMEEMGVIRDDELRAEKQQELKEIMDDYYRTFIEEKLGQIQGIQWNSLFQKMEETMEDISVRKDLDKIQNEKRKEICCYFTSDKRFKDLFNAKLITDILPNFIKDNKEYTEEEKAEKEQTRVLFQRFATAFTNYFNQRRNNFSEDNISTAISFRIVNENSEIHLQNMRAFQRIEQQYPEEVCGMEEEYKDMLQEWQMKHIYSVDFYDRELTQPGIEYYNGICGKINEHMNQFCQKNRINKNDFRMKKLHKQILCKKSSYYEIPFRFESDQEVYDALNEFIKTMKKKEIIRRCVHLGQECDDYDLGKIYISSNKYEQISNALYGSWDTIRKCIKEEYMDALPGKGEKKEEKAEAAAKKEEYRSIADIDKIISLYGSEMDRTISAKKCITEICDMAGQISIDPLVCNSDIKLLQNKEKTTEIKTILDSFLHVYQWGQTFIVSDIIEKDSYFYSELEDVLEDFEGITTLYNHVRSYVTQKPYSTVKFKLHFGSPTLANGWSQSKEYDNNAILLMRDQKFYLGIFNVRNKPDKQIIKGHEKEEKGDYKKMIYNLLPGPSKMLPKVFITSRSGQETYKPSKHILDGYNEKRHIKSSPKFDLGYCWDLIDYYKECIHKHPDWKNYDFHFSDTKDYEDISGFYREVEMQGYQIKWTYISADEIQKLDEKGQIFLFQIYNKDFSVHSTGKDNLHTMYLKNLFSEENLKDIVLKLNGEAELFFRKASIKTPIVHKKGSVLVNRSYTQTVGNKEIRVSIPEEYYTEIYNYLNHIGKGKLSSEAQRYLDEGKIKSFTATKDIVKNYRYCCDHYFLHLPITINFKAKSDVAVNERTLAYIAKKEDIHIIGIDRGERNLLYISVVDVHGNIREQRSFNIVNGYDYQQKLKDREKSRDAARKNWEEIEKIKELKEGYLSMVIHYIAQLVVKYNAVVAMEDLNYGFKTGRFKVERQVYQKFETMLIEKLHYLVFKDREVCEEGGVLRGYQLTYIPESLKKVGKQCGFIFYVPAGYTSKIDPTTGFVNLFSFKNLTNRESRQDFVGKFDEIRYDRDKKMFEFSFDYNNYIKKGTILASTKWKVYTNGTRLKKIVVNGKYTSQSMEVELTDAMEKMLQRAGIEYHDGKDLKGQIVEKGIEAEIIDIFRLTVQMRNSRSESEDREYDRLISPVLNDKGEFFDTATADKTLPQDADANGAYCIALKGLYEVKQIKENWKENEQFPRNKLVQDNKTWFDFMQKKRYL

SEQ ID NO: 40 - protein, 1271 aa, synthetic construct
  source 1..1271: note = Description of Artificial Sequence: Synthetic polypeptide
MEDKQFLERYKEFIGLNSLSKTLRNSLIPVGSTLKHIQEYGILEEDSLRAQKREELKGIMDDYYRNYIEMHLRDVHDIDWNELFEALTEVKKNQTDDAKKRLEKIQEKKRKEIYQYLSDDAVFSEMFKEKMISGILPDFIRCNEGYSEEEKEEKLKTVALFHRFTSSFNDFFLNRKNVFTKEAIVTAIGYRVVHENAEIFLENMVAFQNIQKSAESQISIIERKNEHYFMEWKLSHIFTADYYMMLMTQKAIEHYNEMCGVVNQQMREYCQKEKKNWNLYRMKRLHKQILSNASTSFKIPEKYENDAEVYESVNSFLQNVMEKTVMERIAVLKNSTDNFDLSKIYITAPYYEKISNYLCGSWNTITDCLTHYYEQQIAGKGARKDQKVKAAVKADKWKSLSEIEQLLKEYARAEEVKRKPEEYIAEIENIVSLKEAHLLEYHPEVNLIENEKYATEIKDVLDNYMELFHWMKWFYIEEAVEKEVNFYGELDDLYEEIKDIVPLYNKVRNYVTQKPYSDTKIKLNFGTPTLANGWSKSKEYDYNAILLQKDGKYYMGIFNPIQKPEKEIIEGHSQPLEGNEYKKMVYYYLPSANKMLPKVLLSKKGMEIYQPSEYIINGYKERRHIKSEEKFDLQFCHDLIDYFKSGIERNSDWKVFGFDFSDTDTYQDISGFYREVEDQGYKIDWTYIKEADIDRLNEEGKLYLFQIYNKDFSEKSTGRENLHTMYLKNLFSEENVREQVLKLNGEAEIFFRKSSVKKPIIHKKGTMLVNRTYMEEVNGNSVRRNIPEKEYQEIYNYKNHRLKGELSTEAKKYLEKAVCHETKKDIVKDYRYSVDKFFIHLPITINYRASGKETLNSVAQRYIAHQNDMHVIGIDRGERNLIYVSVINMQGEIKEQKSFNIINEFNYKEKLKEREQSRGAARRNWKEIGQIKDLKEGYLSGVIHEIAKMMIKYHAIIAMEDLNYGFKRGRFKVERQVYQKFENMLIQKLNYLVFKDRPADEDGGVLRGYQLAYIPDSVKKMGRQCGMIFYVPAAFTSKIDPTTGFVDIFKHKVYTTEQAKREFILSFDEICYDVERQLFRFTFDYANFVTQNVTLARNNWTIYTNGTRAQKEFGNGRMRDKEDYNPKDKMVELLESEGIEFKSGKNLLPALKKVSNAKVFEELQKIVRFTVQLRNSKSEENDVDYDHVISPVLNEEGNFFDSSKYKNKEEKKESLLPVDADANGAYCIALKGLYIMQAIQKNWSEEKALSPDVLRLNNNDWFDYIQNKRYR

SEQ ID NO: 41 - protein, 1367 aa, synthetic construct
  source 1..1367: note = Description of Artificial Sequence: Synthetic polypeptide
MEKSLNDFIGLYSVSKTLRFELKPVSETLENIKKFHFLEEDKKKANDYKDVKKIIDNYHKYFIDDVLKNASFNWKKLEEAIREYNKNKSDDSALVAEQKKLGDAILKLFTSDKRYKALTAATPKELFESILPDWFGEQCNQDLNKAALKTFQKFTSYFTGFQENRKNVYSAEAIPTAVPYRIVNDNFPKFLQNVLIFKTIQEKCPQIIDEVEKELSSYLGKEKLAGIFTLESFNKYLGQGGKENQRGIDFYNQIIGGVVEKEGGINLRGVNQFLNLYWQQHPDFTKEDRRIKMVPLYKQILSDRSSLSFKIESIENDEELKNALLECADKLELKNDEKKSIFEEVCDLFSSVKNLDLSGIYINRKDINSVSRILTGDWSWLQSRMNVYAEEKFTTKAEKARWQKSLDDEGENKSKGFYSLTDLNEVLEYSSENVAETDIRITDYFEHRCRYYVDKETEMFVQGSELVALSLQEMCDDILKKRKAMNTVLENLSSENKLREKTDDVAVIKEYLDAVQELLHRIKPLKVNGVGDSTFYSVYDSIYSALSEVISVYNKTRNYITKKAASPEKYKLNFDNPTLADGWDLNKEQANTSVILRKDGMFYLGIMNPKNKPKFAEKYDCGNESCYEKMIYKQFDATKQIPKCSTQKKEVQKYFLSGATEPYILNDKKSFKSELIITKDIWFMNNHVWDGEKFVPKRDNETRPKKFQIGYFKQTGDFDGYKNALSNWISFCKNFLQSYLSATVYDYNFKNSEEYEGLDEFYNYLNATCYKLNFINIPETEINKMVSEGKLYLFQIYNKDFASGSTGMPNMHTLYWKNLFSDENLKNVCLKLNGEAELFYRPAGIKEPVIHKEGSYLVNRTTEDGESIPEKIYFEIYKNANGKLEKLSDEAQNYISNHEVVIKKAGHEIIKDRHYTEPKFLFHVPLTINFKASGNSYSINENVRKFLKNNPDVNIIGLDRGERHLIYLSLINQKGEIIKQFTFNEVERNKNGRTIKVNYHEKLDQREKERDAARKSWQAIGKIAELKEGYLSAVIHQLTKLMVEYNAVVVMEDLNFGFKRGRFHVEKQVYQKFEHILIDKSNYLVFKDRGLNEPGGVLNGYQIAGQFESFQKLGKQSGMLFYVPAGYTSKIDPKTGFVSMMNFKDLTNVHKKRDFFSKFDNIHYDEANGSFVFTFDYKKFDGKAKEEMKLTKWSVYSRDKRIVYFAKTKSYEDVLPTEKLQKIFESNGIDYKSGNNIQDSVMAIGADLKEGAKPSKEISDFWDGLLSNFKLILQMRNSNARTGEDYIISPVMADDGTFFDSREEFKKGEDAKLPLDADANGAYHIALKGLSLINKINLSKDEELKKFDMKISNADWFKFAQEKNYAK

SEQ ID NO: 42 - protein, 1229 aa, synthetic construct
  source 1..1229: note = Description of Artificial Sequence: Synthetic polypeptide
MEEKKMSKIEKFIGKYKISKTLRFRAVPVGKTQDNIEKKGILEKDKKRSEDYEKVKAYLDSLHRDFIENTLKKVKLNELNEYACLFFSGTKDDGDKKKMEKLEEKMRKTISNEFCNDEMYKKIFSEKILSENNEEDVSDIVSSYKGFFTSLNGYVNNRKNLYVSDAKPTSIAYRCINENLPKFLRNVECYKKVVQVIPKEQIEYMSNNLNLSPYRIEDCFNIDFFEFCLSQGGIDLYNTFIGGYSKKDGTKVQGINEIVNLYNQKNKKDKEKYKLPQFTPLFKQILSDRDTKSFSIEKLENIYEVVELVKKSYSDEMFDDIETVFSNLNYYDASGIYVKNGPAITHISMNLTKDWATIRNNWNYEYDEKHSTKKNKNIEKYEDTRNTMYKKIDSFTLEYISRLVGKDIDELVKYFENEVANFVMDIKKTYSKLTPLFDRCQKENFDISEDEVNDIKGYLDNVKLLESFMKSFTINGKENNIDYVFYGKFTDDYDKLHEFDHIYNKVRNYITTSRKPYKLDKYKLYFDNPQLLGGWDINKEKDYRTVMLTKDGKYYFAIIDKGEHPFDNIPKDYFDNNGYYKKIIYRQIPNAAKYLSSKQIVPQNPPEEVKRILDKKKADSKSLTEEEKNIFIDYIKSDFLKNYKLLFDKNNNPYFNFAFRESSTYESLNEFFEDVERQAYSVRYENLPADYIDNLVNEGKIYLFEIYSKDFSEYSKGTNNLHTMYFKALFDNDNLKNTVFKLSGNAELFIRPASIKKDELVIHPKNQLLQNKNPLNPKKQSIFDYDLVKDKRFFENQYMLHISIEINKNERDAKKIKNINEMVRKELKDSDDNYIIGIDRGERNLLYVCVINSAGKIVEQMSLNEIINEYNGIKHTVDYQGLLDKCEKERNAQRQSWKSIENIKELKDGYISQVVHKLCQLVEKYDAIIAMENLNGGFKRGRTKFEKQVYQKFENKLINKMEYMADKKRKTTENGGILRGYQLTNGCINNSYQNGFIFYVPAWLTSKIDPTTGFVDLLKPKYTNVEEAHLWINKFNSITYDKKLDMFAFNINYSQFPRADIDYRKIWTFYTNGYRIETFRNSEKNNEFDWKEVHLTSVIKKLLEEYQINYISGKNIIDDLIQIKDKPFWNSFIKYIRLTLQMRNSITGRTDVDYIISPVINNEGTFYDSRKDLDEITLPQDADANGAYNIARKALWIIEKLKESPDEELNKVKLAITQREWLEYAQINI

SEQ ID NO: 43 - protein, 1261 aa, synthetic construct
  source 1..1261: note = Description of Artificial Sequence: Synthetic polypeptide
MIIHNCYIGGSFMKKIDSFTNCYSLSKTLRFKLIPIGATQSNFDLNKMLDEDKKRAENYSKAKSIIDKYHRFFIDKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQPTAISYRCINDNLPKFLDNVRTFKNSDVASILNDNLKILNEDFDGIYGTSAEDVFNVDYFPFVLSQKGIEAYNSILGGYTNSDGSKIKGLNEYINLYNQKNENIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSIESFSLADIQKYADVSESSETNAEVTEWLRNEIKEKCNLAVQGYESSKDLISKPYTESKKLFNNDNAVELIKNALDSVKELENVLRLLLGTGKEESKDENFYGEFLPCYERICEVDSLYDKVRNYMTQKLYKTDKIKLNFQNPQFLGGWDRNKEADYSAVLLRRNSLYYIAIMPSGYKRVFEKIPAPKADETVYEKVIYKLLPGPNKMLPKVFFSKKGIETFNPPKEILEKYELGTHKTGDGFNLDDCHALIDYFKSALDVHSDWSNFGFRFSDTSTYKNIADFYNEVKNQGYKITFCDVPQSYINELVDEGKLYLFQLYNKDFSEHSKGTPNLHTLYFKMLFDERNLENVVFKLNGEAEMFYREASISKDDMIVHPKNQPIKNKNEQNSRKQSTFEYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNLGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 44 - protein, 1242 aa, synthetic construct
  source 1..1242: note = Description of Artificial Sequence: Synthetic polypeptide
MKEQFVNQYPISKTLRFSLIPIGKTEENFNKNLLLKEDEKKAEEYQKVKGYIDRYHKFFIETALCNINFEGFEEYSLLYYKCSKDDNDLKTMEDIEIKLRKQISKTMTSHKLYKDLFGENMIKTILPNFLDSDEEKNSLEMFRGFYTYFSGFNTNRKNMYTEEAKSTSIAYRCINDNLPKFLDNSKSFEKIKCALNKEELKAKNEEFYEIFQIYATDIFNIDFFNFVLTQPGIDKYNGIIGGYTCSDGTKVQGLNEIINLYNQQIAKDDKSKRLPLLKMLYKQILSDRETVSFIPEKFSSDNEVLESINNYFSKNVSNAIKSLKELFQGFEAYNMNGIFISSGVAITDLSNAVFGDWNAISTAWEKAYFETNPPKKNKSQEKYEEELKANYKKIKSFSLDEIQRLGSIAKSPDSIGSVAEYYKITVTEKIDNITELYDGSKELLNCNYSESYDKKLIKNDTVIEKVKTLLDAVKSLEKLIKPLVGTGKEDKDELFYGTFLPLYTSLSAVDRLYDKVRNYATQKPYSKDKIKLNFNCSSFLSGWATDYSSNGGLIFEKDGLYYLGIVNKKFTTEEIDYLQQNADENPAQRIVYDFQKPDNKNTPRLFIRSKGTNYSPSVKEYNLPVEEIVELYDKRYFTTEYRNKNPELYKASLVKLIDYFKLGFTRHESYRHYDFKWKKSEEYNDISEFYKDVEISCYSLKQEKINYNTLLNFVAENRIYLFQIYNKDFSKYSKGTPNLHTRYFKALFDENNLSDVVFKLNGGSEMFFRKASIKDNEKVVHPANQPIDNKNPDNSKKQSTFDYELIKDKRFTKHQFSIHIPITMNFKARGRDFINNDIRKAIKSEYKPYVIGIDRGERNLIYISVINNNGEIVEQMSLNDIISDNGYKVDYQRLLDRKEKERDNARKSWGTIENIKELKEGYISQVIHKICELVIKYDAVIAMEDLNFGFKRGRFNVEKQVYQKFENMLISKLNYLCDKKSEANSEGGLLKAYQLTNKFDGVNKGKQNGIIFYVPAWLTSKIDPVTGFVDLLHPKYISVEETHSLFEKLDDIRYNFEKDMFEFDIDYSKLPKCNADFKQKWTVCTNADRIMTFRNSEKNNEWDNKRILLSDEFKRLFEEFGIDYCHNLKNKILSISNKDFCYRFIKLFALTMQMRNSITGSTNPEDDYLISPVRDENGVFYDSRNFIGSKAGLPIDADANGAYNIARKGLWAINAIKSTADDMLDKVDLSISNAKWLEYVQK

SEQ ID NO: 45 - protein, 1307 aa, synthetic construct
  source 1..1307: note = Description of Artificial Sequence: Synthetic polypeptide
MADLSQFTHKYQVPKTLRFELIPQGKTLENLSAYGMVADDKQRSENYKKLKPVIDRIYKYFIEESLKNTNLDWNPLYEAIREYRKEKTTATITNLKEQQDICRRAIASRFEGKVPDKGDKSVKDFNKKQSKLFKELFGKELFTDSVLEQLPGVSLSDEDKALLKSFDKFTTYFVGFYDNRKNVFSSDDISTGIPHRLVQENFPKFIDNCDDYKRLVLVAPELKEKLEKAAEATKIFEDVSLDEIFSIKFYNRLLQQNQIDQFNQLLGGIAGAPGTPKIQGLNETLNLSMQQDKTLEQKLKSVPHRFSPLYKQILSDRSSLSFIPESFSCDAEVLLAVQEYLDNLKTEHVIEDLKEVFNRLTTLDLKHIYVNSTKVTAFSQALFGDWNLCREQLRVYKMSNGNEKITKKALGELESWLKNSDIAFTELQEALADEALPAKVNLKVQEAISGLNEQMAKSLPKELKIPEEKEELKALLDAIQEVYHTLEWFIVSDDVETDTDFYVPLKETLQIIQPIIPLYNKVRNFATQKPYSVEKFKLNFANPTLADGWDENKEQQNCAVLFQKGNNYYLGILNPKNKPDFDNVDTEKQGNCYQKMVYKQFPDFSKMMPKCTTQLKEVKQHFEGKDSDYILNNKNFIKPLTITREVYDLNNVLYDGKKKFQIDYLRKTKDEDGYYTALHTWIDFAKKFVASYKSTSIYDTSTILPPEKYEKLNEFYGALDNLFYQIKFENIPEEIIDTYVEDGKLFLFQIYNKDFAAGATGAPNLHTIYWKAVFDPENVKDVVVKLNGQAELFYRPKSNMDVIRHKVGEKLVNRTLKDGSILTDELHKELYLYANGSLKKGLSEDAKIILDKNLAVIYDVHHEIVKDRRFTTDKFFFHVPLTLNYKCDKNPVKFNAEVQEYLKENPDTYVIGIDRGERNLIYAVVIDPKGRIVEQKSFNVINGFDYHGKLDQREKERVKARQAWTAVGKIKELKQGYLSLVVHEISKMMVRYQAVVVLENLNVGFKRVRSGIAEKAVYQQFEKMLINKLNYLMFKDAGGTEPGSVLNAYQLTDRFESFAKMGLQTGFLFYIPAAFTSKIDPATGFVDPFRWGAIKTLADKREFLSGFESLKFDSTTGNFILHFDVSKNKNFQKKLEGFVPDWDIIIEANKMKTGKGATYIAGKRIEFVRDNNSQGHYEDYLPCNALAETLRQCDIPYEEGKDILPLILEKNDSKLLHSVFKVVRLTLQMRNSNAETGEDYISSPVEDVSGSCFDSRMENEKLPKDADANGAYHIALKGMLALERLRKDEKMAISNNDWLNYIQEKRA

SEQ ID NO: 46 - protein, 1286 aa, synthetic construct
  source 1..1286: note = Description of Artificial Sequence: Synthetic polypeptide
MTNFDNFTKKYVNSKTIRLEAIPVGKTLKNIEKMGFIAADRQRDEDYQKAKSVIDHIYKAFMDDCLKDLFLDWDPLYEAVVACWRERSPEGRQALQIMQADYRKKIADRFRNHELYGSLFTKKIFDGSVAQRLPDLEQSAEEKSLLSNFNKFTSYFRDFFDKRKRLFSDDEKHSAIAYRLINENFLKFVANCEAFRRMTERVPELREKLQNTGSLQVYNGLALDEVFSADFYNQLIVQKQIDLYNQLIGGIAGEPGTPNIQGLNATINLALQGDSSLHEKLAGIPHRFNPLYKQILSDVSTLSFVPSAFQSDGEMLAAVRGFKVQLESGRVLQNVRRLFNGLETEADLSRVYVNNSKLAAFSSMFFGRWNLCSDALFAWKKGKQKKITNKKLTEIKKWLKNSDIAIAEIQEAFGEDFPRGKINEKIQAQADALHSQLALPIPENLKALCAKDGLKSMLDTVLGLYRMLQWFIVGDDNEKDSDFYFGLGKILGSLDPVLVLYNRVRNYITKKPYSLTKFRLNFDNSQLLNGWDENNLDTNCASIFIKDGKYYLGISNKNNRPQFDTVATSGKSGYQRMVYKQFANWGRDLPHSTTQMKKVKKHFSASDADYVLDGDKFIRPLIITKEIFDLNNVKFNGKKKLQVDYLRNTGDREGYTHALHTWINFAKDFCACYKSTSIYDISCLRPTDQYDNLMDFYADLGNLSHRIVWQTIPEEAIDNYVEQGQLFLFQLYNKDFAPGADGKPNLHTLYWKAVFNPENLEDVVVKLNGKAELFYRPRSNMDVVRHKVGEKLVNRKLKNGLTLPSRLHEEIYRYVNGTLNKDLSADARSVLPLAVVRDVQHEIIKDRRFTADKFFFHASLTFNFKSSDKPVGFNEDVREYLREHPDTYVVGVDRGERNLIYIVVIDPQGNIVEQRSFNMINGIDYWSLLDQKEKERVEAKQAWETVGKIKDLKCGYLSFLIHEITKIIIKYHAVVILENLSLGFKRVRTGIAEKAVYQQFERMLVTKLGYVVFKDRAGKAPGGVLNAYQLTDNTRTAENTGIQNGFLFYVPAAFTSRVDPATGFFDFYDWGKIKTATDKKNFIAGFNSVRYERSTGDFIVHVGAKNLAVRRVAEDVRTEWDIVIEANVRKMGIDGNSYISGKRIRYRSGEQGHGQYENHLPCQELIRALQQYGIQYETGKDILPAILQQDDAKLTDTVFDVFRLALQMRNTSAETGEDYFNSVVRDRSGRCFDTRRAEAAMPKEADANDAYHIALKGLFVLEKLRKGESIGIKNTEWLRYVQQRHS

SEQ ID NO: 47 - protein, 1264 aa, synthetic construct
  source 1..1264: note = Description of Artificial Sequence: Synthetic polypeptide
MENYGGFTGLYPLQKTLKFELRPQGRTMEHLVSSNFFEEDRDRAEKYKIVKKVIDNYHKDFINECLSKRSFDWTPLMKTSEKYYASKEKNGKKKQDLDQKIIPTIENLSEKDRKELELEQKRMRKEIVSVFKEDKRFKYLFSEKLFSILLKDEDYSKEKLTEKEILALKSFNKFSGYFIGLHKNRANFYSEGDESTAIAYRIVNENFPKFLSNLKKYREVCEKYPEIIQDAEQSLAGLNIKMDDIFPMENFNKVMTQDGIDLYNLAIGGKAQALGEKQKGLNEFLNEVNQSYKKGNDRIRMTPLFKQILSERTSYSYILDAFDDNSQLITSINGFFTEVEKDKEGNTFDRAVGLIASYMKYDLSRVYIRKADLNKVSMEIFGSWERLGGLLRIFKSELYGDVNAEKTSKKVDKWLNSGEFSLSDVINAIAGSKSAETFDEYILKMRVARGEIDNALEKIKCINGNFSEDENSKMIIKAILDSVQRLFHLFSSFQVRADFSQDGDFYAEYNEIYEKLFAIVPLYNRVRNYLTKNNLSMKKIKLNFKNPALANGWDLNKEYDNTAVIFLREGKYYLGIMNPSKKKNIKFEEGSGTGPFYKKMAYKLLPDPNKMLPKVFFAKKNINYYNPSDEIVKGYKAGKYKKGENFDIDFCHKLIDFFKESIQKNEDWRAFNYLFSATESYKDISDFYSEVEDQGYRMYFLNVPVANIDEYVEKGDLFLFQIYNKDFASGAKGNKDMHTIYWNAAFSDENLRNVVVKLNGEAELFYRDKSIIEPICHKKGEMLVNRTCFDKTPVPDKIHKELFDYHNGRAKTLSIEAKGYLDRVGVFQASYEIIKDRRYSENKMYFHVPLKLNFKADGKKNLNKMVIEKFLSDKDVHIIGIDRGERNLLYYSVIDRRGNIIDQDSLNIIDGFDYQKKLGQREIERREARQSWNSIGKIKDLKEGYLSKAVHKVSKMVLEYNAIVVLEDLNFGFKRGRFKVEKQVYQKFEKMLIDKLNYLVFKEVLDSRDAGGVLNAYQLTTQLESFNKLGKQSGILFYVPAAYTSKIDPTTGFVSLFNTSRIESDSEKKDFLSGFDSIVYSAKDGGIFAFKFDYRNRNFQREKTDHKNIWTVYTNGDRIKYKGRMKGYEITSPTKRIKDVLSSSGIRYDDGQELRDSIIQSGNKVLINEVYNSFIDTLQMRNSDGEQDYIISPVKNRNGEFFRTDPDRRELPVDADANGAYHIALRGELLMQKIAEDFDPKSDKFTMPKMEHKDWFEFMQTRGD

SEQ ID NO: 48 - protein, 1232 aa, synthetic construct
  source 1..1232: note = Description of Artificial Sequence: Synthetic polypeptide
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 49 - protein, 1067 aa, synthetic construct
  source 1..1067: note = Description of Artificial Sequence: Synthetic polypeptide
MKNGINLFKTKTTKTKGVDMEKYQITKTIRFKLLPDNAHEIVEKVKSLKTSNVDELMDEVKNVHLKGLELLFALKKYFYFDGNQCKSFKSTLEIKARWLRLYTPDQYYLKKSSKNSYQLKSLSYFKDVFNDWLFNWEESVSELAIIYEKYKICQHQRDSRADIALLIKKLSMKEYFPFISDLIDCVNDKNSNKTFLMKLSEELSVLLEKCNSRALPYQSNGIVVGKASLNYYTVSKSEKMLQNEYEDVCQSLDKNYDITEMKVILYKEKLDNLNFKDVTIANAYNLLKENKALQKRLFSEYVSQGKVLSLIKTELPLFSNINDNDFEKYKEWSNEIKKLADKKNTFCKKTQQDKIKDIQNKISELKKKRGALFQYKFTSFQKHCDNYKKVAVQYGKLKARKKAIEKDEIEANLLRYWSVILEQEDKHSLVLIPKNNAKDAKQYIETINTKGGKYIIHHLDSLTLRALNKLCFNAVDIEKGQMVRENTFYQGIKEEFERNKINCDNQGVLKIQGLYSFKTEGGQINEKEAVEFFKEVLKSNYAREVLNLPYDLESNIFQKEYTNLDQFRQDLEKCCYALHSKIGKDDLDEFTRRFEAQVFDITSIDLKSKKEKTKTTGEMKKHTQLWLEFWKGAIEQNFATRVNPELSIFWRAPKSSREKKYGKGSDLYDPNKNNRYLYEQYTLALTITENAGSHFKDIAFKDTSKIKEAIKEFNMSLSQSKYCFGIDRGNAELVSLCLIKNEKDFPFEKFPVYRLRDLTYQGDFKDKHDQMRYGVAIKNISYFIDQEDLFEKNNLSAIDMTTAKLIKNKIVLNGDVLTYLKLKEETAKHKLTQFFQGSSINKNSRVYFDEDENVFKITTNRNHNPEEIIYFYRGEYGAIKNKNDLEDILNEYLCKMETGESEIVLLNRVNHLRDAISANIVGILSYLIDLFPETIVALENLAKGTIDRHVSQSYENITRRFEWALYRKLLNKQLAPPELKENILLREGDDKIDQFGIIHFVEEKNTSKDCPNCRKTTQQTNDNKFKEKKFVCKSCGFDTSKDRKGMDSLNSPDTVAAYNVARKKFES

SEQ ID NO: 50 - protein, 1309 aa, synthetic construct
  source 1..1309: note = Description of Artificial Sequence: Synthetic polypeptide
MAKETKEFKTFDDFTNLYEVQKTLRFELEAVPETEIVLENRGIWYKRDKKRADEKPIVKFYMDILHREFTDEALEKIKESGVLNLSGYFKLFEELRRLQNHGANTKEEKKLKLEEIRAKKREISNELSQIRRVFSVRGFDVVDSDWKKKYTIEGKKIKNDKSKTYLILSENILNFLENRFTSKEVERLRSIDKKHVEDYGNVVNSGGENIFATFKGFFGYFDSLIKNRENFYETDGKAGRVATRSVDENLNFFAENLHIFSTDLPKALKDDLSDTQKAIFERSYYKNCLLQKDIKSYNLIIGDINKEINKHRQQRDTKIKFLNTLFKQILSIEEKEQYKHIEINNDEDLIRAIRDFISLNESKISEGTKIFNQFIQRCLQKEDLGQIYLPKDSVNTIAHRIFKPWDEIMALFDRKYFVSLEEIKDLTESSVWKERVLEESKTKSLIFKDTHIHTIISGQEIFSNFILILEKEYKNQFSGFISETRRGKAAFVGYDESLKNLRATIKWFEGKNLKLSETEKVEWIKAIKDYADAALRIFQMTKYLWLPVVGDEEDKDYLRIKAEIDQLTKDNDFYNKINAFIDGYKPEPFIYRSSFQEYLTRRPFSTDKFKINFENSRLLDGWDKDMIDDRMGILLQRDGDYFLGILNKEDRHCLDNLVDVKSEDKNSYALMQFKQLTGLYRQLPRMAFPKKKQPVLEANAEIKKIKEDFDFLQKQKKEREVNVNVVFDNKKLNLLINHYAEFLKENYKDEKCYDFSLLNKEKVYESLSDFYADVDKITYSLSFIQVSIDQLIKTGKILLFRLKNKDLLKGSLGQNKNLHTYYFHALFERENLSQGRIRLGAQAEIFFRPASIEKEKDKNRSNALKKSPKTRYVKEILKNKRYSEDKVFLHLPIQLNADAYDLPSINQNVFEFIKNRQEKVKIIGIDRGEKNLAYYSVISQNSNGKIKIEEPPRDLNLGYLEPLDELENKRQDERKAWQSISEIKSKRDGYISYAVSKIVELMLKYQAIIVLEDLSGKFKRSRMKFEKAPYQQLELALIKKLNYLVKKNSKSGKPGHYLSAYQLTEPVGSYKEMGKQTGIIFYTQAGYTSRTCPTCGWRKRVQGLYYKDRTSAQRRFDPKTGVKIFYDSVNDRFVFQYHPVYEQKELKEWDKEIYSDVTRIRWNNEEKKNNEYRKGDITLKIKRLFRDRGIDLSRNINEQLVNVGDASFWEELINLLRLITEIRNIDNENNRDFIECPHCHFQSENGFHGVAWNGDANGAYNIARKGLLITKAVCDPEKNVGDITWSDLKVDMKDWDAATDEWAKKNPEK

SEQ ID NO: 51 - protein, 1456 aa, synthetic construct
  source 1..1456: note = Description of Artificial Sequence: Synthetic polypeptide
MENEKIFSDLTNRYQVVKTLPFELKPVPRTRVLLGLDNPNKGEIFSKDRERAENFTIIKKYIDRLHSLFINESLKKADIDFSNFYKQYGKNINTKNNKNIDDDNDINDDEKEDSENDNLKKYRQEIANLFNKSKYKSWVNVGKDGDKISGMLFEKGLIDLLRTHFSDNLNEDIEIPELFSNKKIKDTRKLKEIINSFGKDGKDGQNFTTYFSVSFHNNRKNYYKSDGKMGRVSTRIVDENLERFCKNIYLYKEIIGKNEIKEIFSGNWDIYLQKKPNFSNDKTYKKLDEFKNDKYDWEMIFRDVNSYNKYFLQSDIEFYNYIRGKLNQDINEYNGKKRDSKEKINSQFENLRNQVHGEKKNYDDDFEIDEDNIIQFINEIFVRHNQNKMRFSEKLFSDFIDLLMVDNGDKLDKVYFSQKAVENAIARYYFVEETTNEGREPLLISLLLQNAGKDRKKLSNKPIKLGDIKFVLDQANNKPAEDIFKNRYVLSESNNDGIINANDKNHWANLLRLIKKDFYFHKDNLIKSQDKLALETKYNKGSDEGERQIETIKNFAESAKAILRMTKYFDLRKNGVIQNVIGGKDPIHEEVDKYFDGDVLSGEESCRISKYYDALRNFITKKAWSADKIILNFDCSEFLGGWDRSQEQKKRGIILRHRDGDEERYYLAVLGKNGKQYFENRTLFKGCESSDWQKIEYNVIQKPHMSLPKNLITPFFKKDKITNERFIDRSKKGAKALIEIDINPSDEFLNNYNLGKHTKENLDKSFLCDYFKYLMDAIAKYYKGEFNFNFPDVSNFDNTQPFYSFIEKNAYSIKYFGISSKEIEKLIADCYYKEDVYLFQIYCKDFEIDPKIGKAKYGNEFRTKAEIRKSKGEEAGNENLNTKYFKLLFDEKNLKNQNGIVYKLNGGAKMFYRPSSIKKDEKIDGKWRYKEDKYSLNITITCNFSSKKDDLSIDKDINKKIAEVNANSDFRIISIDRGEKNLAYCCVMDENANILDIKSLNRITRYDKNGKAIKEKNMFHEVKDGKLCYGEPVYDFYKDYQNLLDEREIKRLVNRRSWNVIEDIKNLKKGYVALLINYICKAVVIAINEGKYPIIVLESLDKGMLHNRVKIEKQIYRGVEEGLVRKLNYFVDKKTDNVLNAWQLLAKFETVGSSLDRKKQLGIIFYVDPGYTSITCPCCGFRQRKYIKAERAEENFKEIKIKFDGKRYSFAYDYRCIDDNGKEKSKEDIIYSNVKRLLRSGRNGRAVQIEDVTDELTNLFKKHNINIEQDINEQLAGKDNKFWKQLLWWFNAIEQIRNTQSLRRKFNTEENKLEILENNDCDFILCPHCYFDSNKDKFQNKIWNGDANGAFNIGRKGIIDIFEIKKHQRMLSDFMEQWGIDKLPKANGGNQAVIEIVKNDKKYNLCILNNKKIPYYCLRIGKEKIDSIADDRKCNQLPDLMVNWKKWDMWLDKWGK

SEQ ID NO: 52 - protein, 1486 aa, synthetic construct
  source 1..1486: note = Description of Artificial Sequence: Synthetic polypeptide
MPEVKNVFQDFTNLYELSKTLRFELKPVPETEKILELNAAKTKKFPKDLYRAENFEIIKKYTDELHRTYIRETLNNVNIDYLKFLEIFRINGKKKNEMTDENEESDENNEKDDIQKIKKELRSKIGNLFNKWNNDKDNKFKDWVKIDVGKKEKEVSGDLFGKELITILKNYFKNKLDSKVNVPMLFFNEQEIKNGEAKKQRKLEAVFENFDKFTTYFTDSFYNNRKNYYKTEGRVGQVATRIIDENLPRFCSNLIAFNEVVSLYSTLLNNFDLGWKEYLNEKKINQTWVEKFELSNYDWKALFNDVNYYNQCLLQEGIDKYNYIIKKLNKDINEYTQNKYKSVEKGNNNNPDINFFQKLHKQIHGERDFKLIEIDIDENNIFTKILPEFILHSDMKLMTKIDEEVGVEEIVGAERIIKIFIKQELKDLEKIYLSRRAIETISAKWFHSWETLKDLILGYLNKDLLESKKRKKVPDFVDFNIIKIVLENNKDDYKDLFKRKYFEADKNEFVDWIDSSGGTKKLEFGGENWINFLNVFEYEFGTLLTEYKKNKNALLYLIDKKIDYDKNNEVGQTAAIKNFADSALGIFRMVSYFALRKKGVMVEPKNGKDEIFYAFVDRYLDGDDNDREEQNKIVQYYNTLRNFVTQKAWSIDKVRLCFDCGEFLKGWDKDKIHERLGIILRNNNKFYLGILNKNHKQIFIKIKSHDNNNFYYVIYDYKQLNNVYRQIPRLAFPSRSVKKGDAYMLRAIQERKKKFFLEDEEFIELQEIKNEYDKIGNDLSKEKLTKLIEYYKKVVISNYSSLYNVSNLNNKKFNSINEFNQYVENLMYSLIPTRISPDFIKEKISKGELYLFQIYNKDFELDESIGKEKFGEDFAPVIMDGKNNLHTEYFKLLFNDSNLKNPNGVVFKLSGGAKMFYRPATENLPIKKDRDGNIIKNKKGENVIVGQRYKEDKYFLHLPIILNFVNKGKNYSINDMVNKAITNASDDQDKFRIIGLDRGEKHLVYYSVINERQEIIEIGSLNNISRKDNKGEIIEEKNWYHDKFGNIEKEPTKEYHKDYHNLLDQREIERLKSRQSWEKIENIKELKEGYISAVINKICNLVIKAIKENKIPIVALENLNSGMKRGRIKIDKQIYQKLELKLAKKLNFLVDKKEKNYLSAWQFTPKIETFSGDIEKKNQVGIIFYVDPAFTSATCPNCGFRKRIKMDPQNAKKKIKDMEITYENGIYKFDYPIENGENDVVYSDVERLKWDNEKKKVIKTKNVSDDFGKLFEDIKDKNNLKKELLSIGEENKEFWKEFSRCFNLLLRIRNSKLIKRKLNDDTGKVEIIADDDLADRDRDFIYCPQCHFHSEGGDVFGEFVKKKYLGKDNFEFNGDANGAYNIARKTIIAVNKIKDYQLGLNHFIEKYRISELPNNGKDKKNIFYNNNSYILSFFEVQDEKFRKVKVYGLKKDGDRQIIQKKEMWYRRYPDIFVNNKEWDKFVQNKS

SEQ ID NO: 53 - protein, 1403 aa, synthetic construct
  source 1..1403: note = Description of Artificial Sequence: Synthetic polypeptide
MLFFMSTDITNKPREKGVFDNFTNLYEFSKTLTFGLIPLKWDDNKKMIVEDEDFSVLRKYGVIEEDKRIAESIKIAKFYLNILHRELIGKVLGSLKFEKKNLENYDRLLGEIEKNNKNENISEDKKKEIRKNFKKELSIAQDILLKKVGEVFESNGSGILSSKNCLDELTKRFTRQEVDKLRRENKDIGVEYPDVAYREKDGKEETKSFFAMDVGYLDDFHKNRKQLYSVKGKKNSLGRRILDNFEIFCKNKKLYEKYKNLDIDFSEIERNFNLTLEKVFDFDNYNERLTQEGLDEYAKILGGESNKQERTANIHGLNQIINLYIQKKQSEQKAEQKETGKKKIKFNKKDYPTFTCLQKQILSQVFRKEIIIESDRDLIRELKFFVEESKEKVDKARGIIEFLLNHEENDIDLAMVYLPKSKINSFVYKVFKEPQDFLSVFQDGASNLDFVSFDKIKTHLENNKLTYKIFFKTLIKENHDFESFLILLQQEIDLLIDGGETVTLGGKKESITSLDEKKNRLKEKLGWFEGKVRENEKMKDEEEGEFCSTVLAYSQAVLNITKRAEIFWLNEKQDAKVGEDNKDMIFYKKFDEFADDGFAPFFYFDKFGNYLKRRSRNTTKEIKLHFGNDDLLEGWDMNKEPEYWSFILRDRNQYYLGIGKKDGEIFHKKLGNSVEAVKEAYELENEADFYEKIDYKQLNIDRFEGIAFPKKTKTEEAFRQVCKKRADEFLGGDTYEFKILLAIKKEYDDFKARRQKEKDWDSKFSKEKMSKLIEYYITCLGKRDDWKRFNLNFRQPKEYEDRSDFVRHIQRQAYWIDPRKVSKDYVDKKVAEGEMFLFKVHNKDFYDFERKSEDKKNHTANLFTQYLLELFSCENIKNIKSKDLIESIFELDGKAEIRFRPKTDDVKLKIYQKKGKDVTYADKRDGNKEKEVIQHRRFAKDALTLHLKIRLNFGKHVNLFDFNKLVNTELFAKVPVKILGMDRGENNLIYYCFLDEHGEIENGKCGSLNRVGEQIITLEDDKKVKEPVDYFQLLVDREGQRDWEQKNWQKMTRIKDLKKAYLGNVVSWISKEMLSGIKEGVVTIGVLEDLNSNFKRTRFFRERQVYQGFEKALVNKLGYLVDKKYDNYRNVYQFAPIVDSVEEMEKNKQIGTLVYVPASYTSKICPHPKCGWRERLYMKNSASKEKIVGLLKSDGIKISYDQKNDRFYFEYQWEQEHKSDGKKKKYSGVDKVFSNVSRMRWDVEQKKSIDFVDGTDGSITNKLKSLLKGKGIELDNINQQIVNQQKELGVEFFQSIIFYFNLIMQIRNYDKEKSGSEADYIQCPSCLFDSRKPEMNGKLSAITNGDANGAYNIARKGFMQLCRIRENPQEPMKLITNREWDEAVREWDIYSAAQKIPVLSEEN

SEQ ID NO: 54 - protein, 1356 aa, synthetic construct
  source 1..1356: note = Description of Artificial Sequence: Synthetic polypeptide
MTIKKHKPFTNFECLTPVQKTLRFRLIPVGRTTEFVKCRNIIEADRKRSEMYPLLKELADRFYREFMTDQLSNLLFDWSPLVEALLLARNNTDPRENQRIASLVRDEQKKYRTLLLKRLSGQVDRNGTPLPKNTASVNKKYYDDLFKARFVTETLPAYLEHLKNKPDGRISDELFDAYKDALDSYQKFTSRLTNFWQARKNIFTDEDIATGFAYRIVHEIVPDYLFNRRVYEQHKLDFPEPLDLLETELKKKNLIANDESLDALFTIPAINRLLTQKGVDLHNAVIGGFFTDDHTKVQGFNELANLKNQTLKNVSDNSEIKPVGKMTRLKKHILSISESTSFLFEQIESDDDLLARIIEFNNTLSEPDIDGLSIADINDQLYNIMTGVDPSTILVHARNLNKLSHEASLSWNRLRDGLYQMATESPYREDERFKRYIDASEEERDLSKLKNDIYFSLQELQFALDQSIDLEEEATPTEDIFLPFEFPGMDLKSELTVLFRSIEQLISSETKLIGNPDAIATIKKYLDAIMARYSIWNLLSCEAVELQDDLFYPEYDRVMGSLSNIILLYNLARNYLSRKPSSKEKFRLNFDKPTLADGWSESKVPDNFSVLLRKDDLFYLGILKDRKAYRVLSYENCDETAKNIKGYYERMIYHFSPDAYRMIPKCSTARKDVKKHFGEQGETTGYTLYPGASNFVKPFTIPYEIYRLQTELVNDKKRYQADYLKQTEDEEGYRQAVTAWIDFCKSYLESYEGTSTFDYSHLLKSEDYEDVNQFYADVDRASYSIYFEKVSVDLIHTMVDRGDLYLFQLYNKDFSPHSTGKPNLHTMYWRALFSNDNLQNNTIKLNGQAELFYRPKQVEQPTVHLQGSYLLNRFDKHGDVIPAGLYCEIYNHINERHPEGYTLSEEATQGLLDGRFVYREAPFELVKDKRYTEDQLFLHVPLEFNWTASANVPFENLANEYIKKDSDLHIIGIDRGERNLLYYSVINLQGDIVKQGSLNTLIQQTTLKGETVERQIPYQSMLKQREDERAEARQNWQSIDRIKDLKEGYLSHVIYKLSRLIIKYHAIVVMENLNVGFKRGRFKVERQVYQKFEVALINKLNALSFKEYEPNELGGVMRPWQLARRVVSPEDTRSQNGIVFYVPASYTSIVDPVTGFANLFYLNRIRNKDLNSFYGHFQEIRYDHEFDRFIFRFNYADFGVFCRIKNVPSRTWNLVSGERKAFNPKRRMIEKRDTTDEIKKALEAHGIAYQNEQNLLPLLLENENLLARIHRSFRLVLQLRNSDSDRDDIVSPALDKENNTFDSGQQPYESSLPINADANGAYNIARKGLLLVDKVKNDKRAVLSNREWFEYLMAEE

SEQ ID NO: 55 - protein, 1296 aa, synthetic construct
  source 1..1296: note = Description of Artificial Sequence: Synthetic polypeptide
MENKDYSLSRFTKQYQNSKTVRFALTPIGRTEEYIIQNQYIEAARRKNQAYKIVKPIIDEKFRSMIDDVLTHCEKQDWVTLDKLILQYQNNKCRENMDALAEQQEEIRKNISEEFTKSDEYKNFFGKEDSKKLFKIFLPEYLNQINASESDKEAVNEFQKFKTYFSNFLIVRADIFKADNKHNTIPYRIVNENFMIFAGNKRTFSNIIRLIPNALEEIAKDGMKKEEWSFYNIQNVDSWFEPDSFQMCMSQKGIQKYNFIIGLVNSYINLYTQQNPQATEVKRSRLKLRMLHKQILSDRVNPSWLPEQFKEGEEGEKQIYEAILALENDLIKNCFDKKYDLWIQSIDIQNPRIYIAASEMARVSSALHMGWNGLNDVRKTILLKSDKKQAKVEKILKQDVSLKDLSDTLNRYADIYKEEQIPSLYQYIEYGSELLQDCAITRKEYHDLLNGNSNTLSLNQNEKLIEGLKAYLDSYQAIVHFLNVFIVGDELDKDTDFYAELDGLVESLSEIVPLYNKVRNYITRKVYSLDKMRIMFERSDFLGGWGQSFDTKEALLFQKDNLYYIGIIEKKYTNMDVEYLHEGIKEGNRAIRFIYNFQKADNKNIPRTFIRSKGTNYAPAVRKYNLPIESIIDIYDVGKFKTNYKKINEKEYYESLEKLIDYFKDGILKNENYKKFHFNWKPSNEYENINEFYNDTNNACFLLEKEEINYDHLKEQANQGKIYLFQISSKDFNEGSKGTPNLQTMYWRELFSNQNCKDGVIKLCGGASIYMRDASIKQPVVHRKNAWLINKWYKVNGQNVVIPDNTYVKFTKIAQERMNEDELTPQERQLWNSGLIQKKKATHDIMKDRRFTKKQYMLHAPLTINYKQQDSPRYFNEKVRSFLKDNPDINIIGIDRGEKNLIYITIIDQKGNILKGMQKSFNQIEEKGKEGRTIDYYSKLESVEARHDAARKNWKQIGTIRELKEGYLSQVVHEITQLMIQYNAVIVMENLNMGFKKGRMKVEKSVYQKFEKMLIDKMNYLAFKRDMQGNAIDPYEVGGVMNGYQLTDRFTSFADMGSQNGFIFYVPAAYTSVIDPVTGFVNVFQKTEFKTNDFLHRFDSISWNDKEQSFVFTFDYQNFKCNGTCYQNKWSLYADVDRIETIIKNNQVDRIEPCNPNQKLIDFFDKKGIIYRDGHNIVDDLEKYDSKTISEIIHNFKLILQLRNSMRNPDTGEIIDYIASPVMHNEERFDSRKRNPELPQDADANGAYHIALKGLMFLQKINEYADSDGNMDNRKLKITNEEWFKYMQTRKEHTYF

SEQ ID NO: 56 - protein, 1250 aa, synthetic construct
  source 1..1250: note = Description of Artificial Sequence: Synthetic polypeptide
MSNKTSSITTTNKLSYTGFHNNGKQSKTLMFELKPIGRTTEHLDRKGYLADDIDRAESYKTFKEIADNFHKNLIEESLATFTFSDTLKDYFDLWLSPVRTNEDTPKLRKMEAKLRKELSSALKQHPSFAATSSGKRLIDEALYPNASDKERQCLDRFKGRSSYLDSYTEVRSFIYTDLCKHNTIAYRVVNENLKIYLENILAYEKLMQTAVNGKLETVKEMFHDLYPTFSMDISIFFTSYGFDYCLSQNAITRYNILLGGWSDDNGIHHKGLNNYINEYNQTVPRNKRLPKLNKLQKMILSEENSMSFIIDKFENDVDLANAIRYWLKNCQFDALNLLIWTLDVHYNLDEIHFKNDNQGKNISDLSQALFKNHHVIRDAWDYDYDIVNAKAKSRQKPERYAEKRDKAFKKINSFSLSYLANILSQYDNQYANFVAQFKTRISVHIQNVQQMIADKTLDMRLDPLMLLKSISSDTKLVEDIKRVLDSLKDMQRMLTPLLGEGTEPNRDAMFYSDFEPLMNYVDTLTPLYNKVRNYITKKPYSTKKTSLYFGASNFGSGFDVTKLPVSHTIIMRDKGCYYLAVIDNNKLIDKLYDHNDNDGYEYMVYKQIPSPIKYFSLKNILPQDPPDDIRQLLEDRKNGAKWSHDDETRFIDYIVNEFLPTYPPIHDKNGNPYFSWKFKNPDEYESLNEFFDDVSKQAYQTSFRFVSRDFVDDAVENGDIFFFQIYNQDFSPASHGKPSPHTLWFRALFSDVNLETKDIRLKGNATAYFRPASIFYTDEKWRKGHHYEQLKNKFKFPIIKDKRYALDKFFFHITLEINCNATVEKYFNNRVNEEIRKADRYNILAINRGERNLLYAVVMDQDGTILEQKSFNIIKSELPNKTVKETDYWKKLHAREKERDTARKSWKSIECIKDLKKGYLSYVVKTITDMMFEYNAVLVMENLDIEMKRSRQKIEKNVYAQFQNAIIQKLSMYVNKDIDLHIARTAPGGTLNPYQLTYIPASRTKTPKQNGFVFFLNPWNITEIDPTTGFVDLFQTCFRTKNEYKDFFAKFKDIRYNEAQGWFEFDTDYTYFRDKEKAGKRTRWNICSYGTRLRRFRNPDKNYAEDAMTVYPTQMLKDLFDEYNIPYAPASAKSTSISIKDDIIQIDKLDFYKKLLYILKLIVQLRNTSPSSTEQEDDYIISPVINEDTNWFYDSRDYNEESLLPCNTDANGAYSLALKCNMVIDRIKNTIPGEPVDMYISNADWLDARQ

SEQ ID NO: 57 - protein, 1328 aa, synthetic construct
  source 1..1328: note = Description of Artificial Sequence: Synthetic polypeptide
MNSKTSIFDFSNIFGRDITLRFKLTPVTINSKGEVKDANGADPYRPYLSADEELQEQYELLKTAIDAYHQMYIDKKLKHILCLPLTEKGKDGVEHDTAKSKFVKSCLAYIKDYGEKDKKRQTADLRTFISRVFADDNISSLPPYKVKSDFITKTLRQWLEQPDTKVEKKEAILDLIEKNGSKLYANCQGLLEARQRLYEKDGKSTSVPYRCIDRNLPRFSKDYHLFEKILGDCSDVFDFEQLDKDFSEELKGIARLSGIRVESVREVFQPLLYLAYLNQEGIQYLNTIIGTKKEKGTSALGLNEYINQYNQKQGIKKKKDGIPMLNKLNNQILFGDEVFIETLAEHKEAIPVIKKVVSSLGKLGAFDGECHENKLYQFLLSLSSYAGNIYVNTKVVAQISSSLWGDYSILYDAVKHDKNGRLIQKSVTLGELNEKIERLKLEDNRDAFEYFRRSQVKDVVHGSSNVGVFEQLKNCYNDFVEKKILKCSFFSEDQVLVIQRLFDSILSLQRIFKVFCPSLYEVDSDGLFVAKFSDYWNVLRGFDKDYDLLRNLFKRKPYSTDKIRVHFGLSNLMDGFVDSWTDKKDKGTQYNGYILRQAHSFVDENTSKELQEFQRYNYYLVISGNVRLFREKGNALVCEKKKEKLVASDEFSGFERFDYYQSSINNFNREFKRLTGRDRKSFTDEILQNEGKKELKSTYIENLIKVAKSMKRLTALQNLVSDEKVRKYSENLDYETLSAEIGQILATGRERKYVPVSTNEMKNLLKSSKNNKGEEVRTFMFRISNKDLSYAETMQKGERKSHGAENMHTMYFRALLDTLQNTFDIGTGTVYFRKASDKRKMKYDEKNPTHRKGDELAFKNPYNKGKKKSVFGYDLIKDRRYTKDSYLFHLSITQNYQKKGNAEDLNAMVRDYIRTQEDLRVIGIDRGERNLLYATMIDGEGHILAQKSFNVIGYQGTTASGESFQVETDYHQLLNEKAEKMRSLQREWKEMDKIQDMKDGYLSVVVHELAKMVVENNAIIVMEDLNMGFMESRQSQLANVYQKFEEKLRNKLQFYVDKRKRNDEPSGLYHALQLAGTETKDNQNGFIFYIPAWNTSKIDSVTGFVNLFNLKYTNIKDAKAFFSTFEKIEKNVETGHYDFTFSYSSMARKKMAKRMDGTRDSWTISTHGSRIVREQKGNYWEYREIESLTSEFDALFEKYSIDTRCRLKEAIDKCGEAEFFKELIRLMKWTLQLRNYDDRGNDYIVSPVCYRGNEYYCSLDYDNEEGMCISKIPCQMPKDADANGAFNIARKGLMLCERLKKGEKIGVIKGTEWLQYVQNMSERYVGMV

SEQ ID NO: 58 - protein, 1434 aa, synthetic construct
  source 1..1434: note = Description of Artificial Sequence: Synthetic polypeptide
MINTMEQPKKSIWDEFTNLYSLQKTLRFELKPQGKTKELVRTLFINPEEHHHKLISDDLELSKNYKKVKKLIDCMHRNIINNVLSKHQFTGEELKKLDKNSNAEDNDTETDNADKKDPFAKIRERLTKALNEESKIMFDNKLLNPKKGKNKGECELKKWMDKAEDKYFELGNNEKIDKEAVKADMERLEGFFTYFGGFNKNRENVYSSKKIATAIPFRIIHDNFPIFKKNIENYKKITEKHPELAKLLNEKGANEIFQLEHFNKCLTQDGIDVYNNEKLGIIAKEQGKEQDKGINQLINEYAQKKNKEIKENAKGGEKPKKIKIAVFDKLKKQILSISKTKSFQFEVFEDTSDIINGINKRYTFLTEAKEGMSIVDEIKKIIGSVGDEKYSLDEIYLKEKFISTLSKKLFNYSRYIEVALEKWYDDRYDDKINKSGTDKRKFISAKQFSITSIQDAINYYLEKYEKDEELSKKYTGKNIIVDYFKNPTITIEHKQKEEVISEEKDLFKELEVRRNVIQHILNGDYKKDLKEEKQQDGDSEKVKAFLDALLEFNYILNPFIIKDKNLRKEQEKDEEFYNEIKKLQESIFEAEILDLYNQTRNYITKKPYKLDKFKLTFGSGYFLSGWSNDMEEREGSILIKYNEDRSKNYYLIIMAKPLTDDDKKQLFSDNGTHSKICIYEFQKMDMKNFPRMFINSKGSNPAPAIEKYNLPIKTIWADYQKYKNLNQKGKDKFLEENPDFRHNLIGYFKICAEKHESLAPFKHQFSSIWKPTKEYENLAQFYKDTLEACYNLKFENVNFDNISQLVSSGKLHLFKIHNKDFNPGSTGKKNLHTLYWEMLFDEKNLQDVIFKLSGGAELFYREASILKNKIIHKIGEKVLKKFFKLPDGKLEPVPAESIKNLSAYFRKELPEHELTEIDRKYIDNYSIIGKKDDKLGIMKDERFTVDKIQFHCPITINFKSKNKNFINDDVLEYLHKRDDVHIIGLDRGERHLIYLTMINKDGKIVDNMQFSLNELQRRYKINGNEEIQKINYQKLLDTREVSRTEARRNWQTIENIKNLKEGYLSLIVHQLAKLMIEKNAIVVMENLNYGFKDSRARVEKQIYQKFESILIKKLQYLVMDKNNLYDSGGVLSAYQLTNQEVPAYKYISKQNGFLFYVPPDYTSKIDPETGFINLLDTRYYSRKNAVALLNKFDKIYYDRDNKYFRFDFDYNSTDSNGNKNFDKLRVDISELTRTKWSVCSHPAKRSITVQINNKWVRQPINDVTDKLIKLFEDKQIGYESGKCLKDEILKVEDAKFFEDLLRYLSVLLALRHTYTENGVEYDLIISSVEKAPGSNEFFVSGKDNNLPANADANGAYNIARKGLWLLRKLDEIDNQELAIKKFNELKHAKEIKKNGEESKEDKGDRKRKKKWVSQWCPNKEWLAFAQSMQDVSEK

SEQ ID NO: 59 - protein, 1263 aa, synthetic construct
  source 1..1263: note = Description of Artificial Sequence: Synthetic polypeptide
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 60 - protein, 1283 aa, synthetic construct
  source 1..1283: note = Description of Artificial Sequence: Synthetic polypeptide
MSNLNTFISPEFTGKIKMTKSLKVSMIPIGETEHWIAKHKVFEKDRELFDKNLKARPILDEFIKYTVSRALPNLLFDFEAYYLVKKDRTKARAFEKELAKTVTDLILKEMDELKSASLIDSADFVKTTLKKFAGTHDIPGLSRIEAIESLEAASKLTALNGKFNTSRIAIINTLIPKRIIENFDIYLSNMEKIRNVYESGEFGFLFERYPDTLLFMEPANYRTVCSPEAIEDYNRFISGYGDSTESWIKGFNQELSEASNSSKSSNGGVRRYSLIKPLHKQHLFETKKFFTFASISSDDDVRELINSVKGSTEDACLNALAFFSSSDPKTLFVKGSYLHTLSAFLYGSANSYILPERIKEGEKARLTAEYDSVAKKTKAVTTRYNVAMNNISKKINEKIFSLADIDAYCCDISKRRSVREILLGIMQEMYAAVYGENGKWSNIEAEAVLDSKTKIWKAKNGAVAKAVNDYLTAILEIRKFIRPFALRMEELEELGLDTSSALDAGEITNTLFEAVRAQKLVHAYLTRNDADIALSTQVYFGGTQKAAASWWNYETGDIQNRQIALAKKDGMYYFIGTFDERGSYSIEPASPGEDYYEMLDVKKGQDANKQIKKVLFSNKAIREHFADSSNDYVITTKVNSPITVRREIFDKYQAGEFKLTSQKIRKGDLVGEKEMTYYREYMDLLFQMAKGYTEYSRFNMDTLLPIEEYDTENDLLDDVNTNTIDYRWVRISAACIDDGVRNGDIFVFRAQTSSMYGKRENKKGYTGLFLELVSDENLLVTRGMSLNSAMSIYYRAKVHDAITVHKKGDVLVNKFTNARERIPENSYKAICAFYNSGKSIEELTIEDRDWLAKATTRICSGEIIKDRRYTKNQYSISISYNINRSVNNRKRVDLATIVDDTASAGRIISVTRGTKDLVYYTVIDDGGSVIEARSLNVINGINYAKMLAQISEERHDSNANFDIPKRVETIKEAYCAFAVHEIISAALKHNALIVVELISDAIKDKYSLLDNQVFLKFENVLKNCLMSVKVKGARGMEPGSISNPLQLCNADDKSFRNGILYQIPSSYINICPVTGYADIIDYYNIVSAGDIRNFFVRFENIVYNKEKARFEFSFDLKNIPIKLEKCPDRTKWTVLGRGEITTYDPLTKSNHYVFDAAQMLAETVSKEGLDPCANIVEHIDELSAATLKKMFNTFRNIAKGIVSECDEVPVSYYKSPVIDEADIKNKSLDNKSISEIKCYNLDEKARYYLALAKSSSDGENKNRYVSSTAIEWLNYIQEKRTHE

SEQ ID NO: 61 - protein, 500 aa, synthetic construct
  source 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 62 - protein, 507 aa, synthetic construct
  source 1..507: note = Description of Artificial Sequence: Synthetic polypeptide
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 63 - protein, 529 aa, synthetic construct
  source 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 64 - protein, 726 aa, synthetic construct
  source 1..726: note = Description of Artificial Sequence: Synthetic polypeptide
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 65 - protein, 517 aa, synthetic construct
  source 1..517: note = Description of Artificial Sequence: Synthetic polypeptide
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 66 - protein, 481 aa, synthetic construct
  source 1..481: note = Description of Artificial Sequence: Synthetic polypeptide
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 67 - protein, 358 aa, synthetic construct
  source 1..358: note = Description of Artificial Sequence: Synthetic polypeptide
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 68 - protein, 507 aa, synthetic construct
  source 1..507: note = Description of Artificial Sequence: Synthetic polypeptide
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 69 - protein, 534 aa, synthetic construct
  source 1..534: note = Description of Artificial Sequence: Synthetic polypeptide
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 70 - protein, 537 aa, synthetic construct
  source 1..537: note = Description of Artificial Sequence: Synthetic polypeptide
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 71 - protein, 540 aa, synthetic construct
  source 1..540: note = Description of Artificial Sequence: Synthetic polypeptide
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 72 - protein, 542 aa, synthetic construct
  source 1..542: note = Description of Artificial Sequence: Synthetic polypeptide
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 73 - protein, 564 aa, synthetic construct
  source 1..564: note = Description of Artificial Sequence: Synthetic polypeptide
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 74 - protein, 610 aa, synthetic construct
  source 1..610: note = Description of Artificial Sequence: Synthetic polypeptide
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 75 - protein, 369 aa, synthetic construct
  source 1..369: note = Description of Artificial Sequence: Synthetic polypeptide
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 76 - protein, 552 aa, synthetic construct
  source 1..552: note = Description of Artificial Sequence: Synthetic polypeptide
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 77 - protein, 534 aa, synthetic construct
  source 1..534: note = Description of Artificial Sequence: Synthetic polypeptide
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 78 - protein, 577 aa, synthetic construct
  source 1..577: note = Description of Artificial Sequence: Synthetic polypeptide
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 79 - protein, 613 aa, synthetic construct
  source 1..613: note = Description of Artificial Sequence: Synthetic polypeptide
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 80 - protein, 615 aa, synthetic construct
  source 1..615: note = Description of Artificial Sequence: Synthetic polypeptide
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 81 - protein, 775 aa, synthetic construct
  source 1..775: note = Description of Artificial Sequence: Synthetic polypeptide
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 82 - protein, 777 aa, synthetic construct
  source 1..777: note = Description of Artificial Sequence: Synthetic polypeptide
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 83 - protein, 610 aa, synthetic construct
  source 1..610: note = Description of Artificial Sequence: Synthetic polypeptide
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 84 - protein, 632 aa, synthetic construct
  source 1..632: note = Description of Artificial Sequence: Synthetic polypeptide
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 85 - protein, 625 aa, synthetic construct
  source 1..625: note = Description of Artificial Sequence: Synthetic polypeptide
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 86 - protein, 517 aa, synthetic construct
  source 1..517: note = Description of Artificial Sequence: Synthetic polypeptide
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 87 - protein, 410 aa, synthetic construct
  source 1..410: note = Description of Artificial Sequence: Synthetic polypeptide
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 88 - protein, 486 aa, synthetic construct
  source 1..486: note = Description of Artificial Sequence: Synthetic polypeptide
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 89 - protein, 602 aa, synthetic construct
  source 1..602: note = Description of Artificial Sequence: Synthetic polypeptide
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 90 - protein, 494 aa, synthetic construct
  source 1..494: note = Description of Artificial Sequence: Synthetic polypeptide
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 91 - protein, 526 aa, synthetic construct
  source 1..526: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 92 - protein, 649 aa, synthetic construct
  source 1..649: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 93 - protein, 414 aa, synthetic construct
  source 1..414: note = Description of Artificial Sequence: Synthetic polypeptide
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 94 - protein, 413 aa, synthetic construct
  source 1..413: note = Description of Artificial Sequence: Synthetic polypeptide
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 95 - protein, 449 aa, synthetic construct
  source 1..449: note = Description of Artificial Sequence: Synthetic polypeptide
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 96 - protein, 711 aa, synthetic construct
  source 1..711: note = Description of Artificial Sequence: Synthetic polypeptide
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 97 - protein, 574 aa, synthetic construct
  source 1..574: note = Description of Artificial Sequence: Synthetic polypeptide
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 98 - protein, 400 aa, synthetic construct
  source 1..400: note = Description of Artificial Sequence: Synthetic polypeptide
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 99 - protein, 743 aa, synthetic construct
  source 1..743: note = Description of Artificial Sequence: Synthetic polypeptide
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 100 - protein, 769 aa, synthetic construct
  source 1..769: note = Description of Artificial Sequence: Synthetic polypeptide
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 101 - protein, 666 aa, synthetic construct
  source 1..666: note = Description of Artificial Sequence: Synthetic polypeptide
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 102 - protein, 564 aa, synthetic construct
  source 1..564: note = Description of Artificial Sequence: Synthetic polypeptide
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 103 - protein, 565 aa, synthetic construct
  source 1..565: note = Description of Artificial Sequence: Synthetic polypeptide
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 104 - protein, 499 aa, synthetic construct
  source 1..499: note = Description of Artificial Sequence: Synthetic polypeptide
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 105 - protein, 560 aa, synthetic construct
  source 1..560: note = Description of Artificial Sequence: Synthetic polypeptide
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 106 - protein, 404 aa, synthetic construct
  source 1..404: note = Description of Artificial Sequence: Synthetic polypeptide
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 107 - protein, 392 aa, synthetic construct
  source 1..392: note = Description of Artificial Sequence: Synthetic polypeptide
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 108 - protein, 500 aa, synthetic construct
  source 1..500: note = Description of Artificial Sequence: Synthetic polypeptide
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 109 - protein, 507 aa, synthetic construct
  source 1..507: note = Description of Artificial Sequence: Synthetic polypeptide
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 110 - protein, 529 aa, synthetic construct
  source 1..529: note = Description of Artificial Sequence: Synthetic polypeptide
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 111 - protein, 726 aa, synthetic construct
  source 1..726: note = Description of Artificial Sequence: Synthetic polypeptide
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 112 - protein, 517 aa, synthetic construct
  source 1..517: note = Description of Artificial Sequence: Synthetic polypeptide
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 113 - protein, 481 aa, synthetic construct
  source 1..481: note = Description of Artificial Sequence: Synthetic polypeptide
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 114 - protein, 534 aa, synthetic construct
  source 1..534: note = Description of Artificial Sequence: Synthetic polypeptide
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 115 - protein, 537 aa, synthetic construct
  source 1..537: note = Description of Artificial Sequence: Synthetic polypeptide
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 116 - protein, 541 aa, synthetic construct
  source 1..541: note = Description of Artificial Sequence: Synthetic polypeptide
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 117 - protein, 542 aa, synthetic construct
  source 1..542: note = Description of Artificial Sequence: Synthetic polypeptide
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 118 - protein, 564 aa, synthetic construct
  source 1..564: note = Description of Artificial Sequence: Synthetic polypeptide
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 119 - protein, 610 aa, synthetic construct
  source 1..610: note = Description of Artificial Sequence: Synthetic polypeptide
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 120 - protein, 552 aa, synthetic construct
  source 1..552: note = Description of Artificial Sequence: Synthetic polypeptide
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 121 - protein, 534 aa, synthetic construct
  source 1..534: note = Description of Artificial Sequence: Synthetic polypeptide
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 122 - protein, 577 aa, synthetic construct
  source 1..577: note = Description of Artificial Sequence: Synthetic polypeptide
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 123 - protein, 613 aa, synthetic construct
  source 1..613: note = Description of Artificial Sequence: Synthetic polypeptide
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 124 - protein, 615 aa, synthetic construct
  source 1..615: note = Description of Artificial Sequence: Synthetic polypeptide
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 125 - protein, 775 aa, synthetic construct
  source 1..775: note = Description of Artificial Sequence: Synthetic polypeptide
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 126 - protein, 777 aa, synthetic construct
  source 1..777: note = Description of Artificial Sequence: Synthetic polypeptide
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 127 - protein, 610 aa, synthetic construct
  source 1..610: note = Description of Artificial Sequence: Synthetic polypeptide
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 128 - protein, 632 aa, synthetic construct
  source 1..632: note = Description of Artificial Sequence: Synthetic polypeptide
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 129 - protein, 625 aa, synthetic construct
  source 1..625: note = Description of Artificial Sequence: Synthetic polypeptide
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 130 - protein, 517 aa, synthetic construct
  source 1..517: note = Description of Artificial Sequence: Synthetic polypeptide
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 131 - protein, 410 aa, synthetic construct
  source 1..410: note = Description of Artificial Sequence: Synthetic polypeptide
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 132 - protein, 602 aa, synthetic construct
  source 1..602: note = Description of Artificial Sequence: Synthetic polypeptide
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 133 - protein, 494 aa, synthetic construct
  source 1..494: note = Description of Artificial Sequence: Synthetic polypeptide
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 134 - protein, 649 aa, synthetic construct
  source 1..649: note = Description of Artificial Sequence: Synthetic polypeptide
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 135 - protein, 414 aa, synthetic construct
  source 1..414: note = Description of Artificial Sequence: Synthetic polypeptide
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 136 - protein, 413 aa, synthetic construct
  source 1..413: note = Description of Artificial Sequence: Synthetic polypeptide
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 137 - protein, 449 aa, synthetic construct
  source 1..449: note = Description of Artificial Sequence: Synthetic polypeptide
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 138 - protein, 711 aa, synthetic construct
  source 1..711: note = Description of Artificial Sequence: Synthetic polypeptide
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 139 - protein, 574 aa, synthetic construct
  source 1..574: note = Description of Artificial Sequence: Synthetic polypeptide
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 140 - protein, 743 aa, synthetic construct
  source 1..743: note = Description of Artificial Sequence: Synthetic polypeptide
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 141 - protein, 769 aa, synthetic construct
  source 1..769: note = Description of Artificial Sequence: Synthetic polypeptide
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 142 - protein, 564 aa, synthetic construct
  source 1..564: note = Description of Artificial Sequence: Synthetic polypeptide
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 143 - protein, 565 aa, synthetic construct
  source 1..565: note = Description of Artificial Sequence: Synthetic polypeptide
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 144 - protein, 499 aa, synthetic construct
  source 1..499: note = Description of Artificial Sequence: Synthetic polypeptide
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 145 - protein, 358 aa, synthetic construct
  source 1..358: note = Description of Artificial Sequence: Synthetic polypeptide
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 146 - protein, 369 aa, synthetic construct
  source 1..369: note = Description of Artificial Sequence: Synthetic polypeptide
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 147 - protein, 486 aa, synthetic construct
  source 1..486: note = Description of Artificial Sequence: Synthetic polypeptide
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 148 - protein, 400 aa, synthetic construct
  source 1..400: note = Description of Artificial Sequence: Synthetic polypeptide
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 149 - protein, 666 aa, synthetic construct
  source 1..666: note = Description of Artificial Sequence: Synthetic polypeptide
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 150 - protein, 560 aa, synthetic construct
  source 1..560: note = Description of Artificial Sequence: Synthetic polypeptide
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 151 - protein, 404 aa, synthetic construct
  source 1..404: note = Description of Artificial Sequence: Synthetic polypeptide
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 152 - protein, 392 aa, synthetic construct
  source 1..392: note = Description of Artificial Sequence: Synthetic polypeptide
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 153 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 154 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 155 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 156 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 157 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 158 - protein, 1285 aa, unidentified
  source 1..1285: note = Description of Unknown: Herbinix hemicellulosilytica sequence
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 159 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 160 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 161 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 162 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 163 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 164 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 165 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 166 - protein, 668 aa, unidentified
  source 1..668: note = Description of Unknown: Cas13c sequence
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 167 - protein, 796 aa, unidentified
  source 1..796: note = Description of Unknown: Cas13c sequence
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 168 - protein, 897 aa, unidentified
  source 1..897: note = Description of Unknown: Cas13c sequence
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 169 - protein, 919 aa, unidentified
  source 1..919: note = Description of Unknown: Cas13c sequence
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 170 - protein, 1110 aa, unidentified
  source 1..1110: note = Description of Unknown: Cas13c sequence
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY

SEQ ID NO: 171 - RNA, 41 nt, synthetic construct
  source 1..41: mol_type = other RNA; note = Description of Artificial Sequence: Synthetic oligonucleotide
taatttctactaagtgtagatccagaacacatagcgacatg

SEQ ID NO: 172 - RNA, 41 nt, synthetic construct
  source 1..41: mol_type = other RNA; note = Description of Artificial Sequence: Synthetic oligonucleotide
taatttctactaagtgtagattattggtctccttaaacctg

SEQ ID NO: 173 - DNA, 132 bp, Homo sapiens
  source 1..132: mol_type = unassigned DNA
cgtggccccactgatgagcttccctccgccctatgggaaaaagtggtctcatacagaacttataagattcccaaatccaaagacatttcacgtttatggtgatttcccagaacacatagcgacatgcaaata

SEQ ID NO: 174 - DNA, 126 bp, Homo sapiens
  source 1..126: mol_type = unassigned DNA
cctatcagaaacccaagagtcttctctgtctccacatgcccagtttctattggtctccttaaacctgtcttgtaaccttgataccaacctgcccagggcctcaccaccaacttcatccacgttcac

SEQ ID NO: 175 - RNA, 41 nt, synthetic construct
  source 1..41: mol_type = other RNA; note = Description of Artificial Sequence: Synthetic oligonucleotide
taatttctactaagtgtagataagcttcgcttgtagatgag

SEQ ID NO: 176 - RNA, 41 nt, synthetic construct
  source 1..41: mol_type = other RNA; note = Description of Artificial Sequence: Synthetic oligonucleotide
taatttctactaagtgtagatacttgcatgcttaagacgca

SEQ ID NO: 177 - RNA, 41 nt, synthetic construct
  source 1..41: mol_type = other RNA; note = Description of Artificial Sequence: Synthetic oligonucleotide
taatttctactaagtgtagatatcctggcttagaactaacg

SEQ ID NO: 178 - RNA, 41 nt, synthetic construct
  source 1..41: mol_type = other RNA; note = Description of Artificial Sequence: Synthetic oligonucleotide
taatttctactaagtgtagatattcgatgatacgcgaggaa

SEQ ID NO: 179 - RNA, 41 nt, synthetic construct
  source 1..41: mol_type = other RNA; note = Description of Artificial Sequence: Synthetic oligonucleotide
taatttctactaagtgtagatcaacataggtccacagttga

SEQ ID NO: 180 - RNA, 41 nt, synthetic construct
  source 1..41: mol_type = other RNA; note = Description of Artificial Sequence: Synthetic oligonucleotide
taatttctactaagtgtagatcaacatagttccacagttga

SEQ ID NO: 181 - RNA, 41 nt, synthetic construct
  source 1..41: mol_type = other RNA; note = Description of Artificial Sequence: Synthetic oligonucleotide
taatttctactaagtgtagatcagcatagttccacagttga

SEQ ID NO: 182 - RNA, 41 nt, synthetic construct
  source 1..41: mol_type = other RNA; note = Description of Artificial Sequence: Synthetic oligonucleotide
taatttctactaagtgtagatcagcgtacactaccagggta

SEQ ID NO: 183 - RNA, 41 nt, synthetic construct
  source 1..41: mol_type = other RNA; note = Description of Artificial Sequence: Synthetic oligonucleotide
taatttctactaagtgtagatccctaccaactagctaataa

SEQ ID NO: 184 - RNA, 41 nt, synthetic construct
  source 1..41: mol_type = other RNA; note = Description of Artificial Sequence: Synthetic oligonucleotide
taatttctactaagtgtagatctacaaagcttattcctcat

SEQ ID NO: 185 - RNA, 41 nt, synthetic construct
  source 1..41: mol_type = other RNA; note = Description of Artificial Sequence: Synthetic oligonucleotide
taatttctactaagtgtagatgggtctatatacaggtgctg

SEQ ID NO: 186 - RNA, 41 nt, synthetic construct
  source 1..41: mol_type = other RNA; note = Description of Artificial Sequence: Synthetic oligonucleotide
taatttctactaagtgtagatgggtctgtatacaggtgctg

SEQ ID NO: 187 - RNA, 41 nt, synthetic construct
  source 1..41: mol_type = other RNA; note = Description of Artificial Sequence: Synthetic oligonucleotide
taatttctactaagtgtagatgtgactcagcgtcagtcttg

SEQ ID NO: 188 - RNA, 41 nt, synthetic construct
  source 1..41: mol_type = other RNA; note = Description of Artificial Sequence: Synthetic oligonucleotide
taatttctactaagtgtagatgttaacaccaagtgtgcatc

SEQ ID NO: 189 - RNA, 41 nt, synthetic construct
  source 1..41: mol_type = other RNA; note = Description of Artificial Sequence: Synthetic oligonucleotide
taatttctactaagtgtagattaggaaatgacaaagcgatg

SEQ ID NO: 190 - RNA, 41 nt, synthetic construct
  source 1..41: mol_type = other RNA; note = Description of Artificial Sequence: Synthetic oligonucleotide
taatttctactaagtgtagattcatttcctacaaagcttat

SEQ ID NO: 191 - RNA, 41 nt, synthetic construct
  source 1..41: mol_type = other RNA; note = Description of Artificial Sequence: Synthetic oligonucleotide
taatttctactaagtgtagattgcatagacttatatatccg

SEQ ID NO: 192 - RNA, 41 nt, synthetic construct
  source 1..41: mol_type = other RNA; note = Description of Artificial Sequence: Synthetic oligonucleotide
taatttctactaagtgtagataggtatgtttagtgaggggg

SEQ ID NO: 193 - RNA, 41 nt, synthetic construct
  source 1..41: mol_type = other RNA; note = Description of Artificial Sequence: Synthetic oligonucleotide
taatttctactaagtgtagatgtgaggggggtgaagtcgta

SEQ ID NO: 194 - RNA, 41 nt, synthetic construct
  source 1..41: mol_type = other RNA; note = Description of Artificial Sequence: Synthetic oligonucleotide
taatttctactaagtgtagatgatttgggaatcttataagt

SEQ ID NO: 195 - DNA, 131 bp, Homo sapiens
  source 1..131: mol_type = unassigned DNA
atgggaaaaagtggtctcatacagaacttataagattcccaaatccaaagacatttcacgtttatggtgatttcccagaacacatagcgacatgcaaatattgcagggcgccactcccctgtccctcacag

(19) United States
(12) Patent Application Publication
(10) Pub. No.: US 2024/0084401 A1
(43) Pub. Date: Mar. 14, 2024

(54) HIGH-PLEX GUIDE POOLING FOR NUCLEIC ACID DETECTION

(71) Applicant: Mammoth Biosciences, Inc., Brisbane, CA (US)

(72) Inventors: Matthew VEROSLOFF, South San Francisco, CA (US); Clare FASCHING, Redwood City, CA (US); Carley Gelenter HENDRIKS, Burlingame, CA (US); Xin MIAO, Mountain View, CA (US); James Paul BROUGHTON, South San Francisco, CA (US); Lucas Benjamin HARRINGTON, San Francisco, CA (US); Janice Sha CHEN, San Francisco, CA (US)

(21) Appl. No.: 17/938,640

(22) Filed: Oct. 6, 2022

Related U.S. Application Data
(63) Continuation of application No. PCT/US21/26719, filed on Apr. 9, 2021.
(60) Provisional application No. 63/008,500, filed on Apr. 10, 2020.

(51) Int. Cl. C12Q 1/689; C12N 9/22; C12Q 1/6806; C12Q 1/6816; C12Q 1/6844

(52) U.S. Cl. CPC C12Q 1/689; C12N 9/22; C12Q 1/6806; C12Q 1/6816; C12Q 1/6844; C12N 2310/20

(57) ABSTRACT

Provided herein, in certain embodiments, are various methods, reagents, and devices for detection of multiple target nucleic acids in a sample, or multiple segments of a target nucleic acid in a sample, using a programmable nuclease. In certain embodiments, the present disclosure provides compositions of pools of guide nucleic acids, programmable nucleases, and detector nucleic acids and methods of using said compositions for detection of different segments of one target nucleic acid or different target nucleic acids in a sample.

### CROSS-REFERENCE

This application claims the benefit of U.S. Provisional Patent Application No. 63/008,500 filed on Apr. 10, 2020, which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 10, 2022, is named 203477-731601_SL.xml and is 324,549 bytes in size.

### BACKGROUND

Various communicable diseases can easily spread from an individual or environment to an individual. The detection of the ailments, especially at the early stages of infection, may provide guidance on treatment or intervention to reduce the progression or transmission of the ailment. Increased sensitivity of disease detection assays may provide earlier detection, leading to reduced transmission.

### SUMMARY

In various aspects, the present disclosure provides a composition comprising a programmable nuclease and a pool of guide nucleic acids comprising greater than 20 distinct guide nucleic acid sequences, wherein at least one guide nucleic acid of the pool hybridizes to a segment of a target nucleic acid.

In some aspects, the pool of guide nucleic acids comprises at least 50 distinct guide nucleic acid sequences, at least 100 distinct guide nucleic acid sequences, at least 500 distinct guide nucleic acid sequences, or at least 1000 distinct guide nucleic acid sequences. In some aspects, the pool of guide nucleic acids comprises at least two guide nucleic acids that hybridize to a different segment of the target nucleic acid. In some aspects, a guide nucleic acid of the pool of guide nucleic acids has a sequence selected from a group of tiled guide nucleic acids that correspond to nucleic acids of the target nucleic acid.

In some aspects, a) the tiled guide nucleic acids are sequential along the target nucleic acid upon hybridization to the target nucleic acid; b) the tiled guide nucleic acids are non-sequential along the target nucleic acid upon hybridization to the target nucleic acid; c) the tiled guide nucleic acids are overlapping along the target nucleic acid upon hybridization to the target nucleic acid; or d) any combination thereof.

In some aspects, the target nucleic acid is from a pathogen. In some aspects, at least two guide nucleic acids of the pool of guide nucleic acids hybridize to segments of distinct target nucleic acids. In some aspects, at least two target nucleic acids of the distinct target nucleic acids are from different pathogens. In further aspects, the pathogen is a virus, a bacterium, a fungus, a protozoan, or a worm. In some aspects, a guide nucleic acid of the pool of guide nucleic acids hybridize to a segment from Staphylococcus aureus, methicillin resistant Staphylococcus aureus, Candida albicans, Pseudomonas aeruginosa, Acinetobacter baumannii, Stenotrophomonas maltophilia, Clostridium difficile, Escherichia coli, Mycobacterium tuberculosis, or Legionella sp.

In some aspects, at least two guide nucleic acids of the pool of guide nucleic acids differs from one another by at least one base. In some aspects, a total concentration of the pool of guide nucleic acids is about 400 nM, about 1000 nM (1 μM), or about 2000 nM (2 μM). In some aspects, each guide nucleic acid of the pool of guide nucleic acids comprises from 20 to 50 bases. In some aspects, each guide nucleic acid comprises from 30 to 50 bases.

In some aspects, the programmable nuclease is a Type V CRISPR-Cas enzyme. In further aspects, the programmable nuclease comprises three partial RuvC domains. In still further aspects, the programmable nuclease comprises a RuvC-I subdomain, a RuvC-II subdomain, and a RuvC-III subdomain. In some aspects, the programmable nuclease is a Cas12 enzyme. In further aspects, the Cas12 enzyme is Cas12a, Cas12b, Cas12c, CasY, or Cas12e. In still further aspects, the Cas 12 enzyme has at least 60% sequence identity to SEQ ID NO: 28.

In some aspects, the programmable nuclease is a Cas14 enzyme. In further aspects, the Cas14 enzyme is Cas14a, Cas14b, Cas14c, Cas14d, Cas14e, Cas14f, Cas14g, or Cas14h.

In other aspects, the programmable nuclease comprises at least two HEPN domains. In further aspects, the programmable nuclease is a Type VI Cas enzyme. In still further aspects, the programmable nuclease is a Cas13 enzyme. In still further aspects, the Cas13 enzyme is Cas13a, Cas13b, Cas13c, Cas13d, or Cas13e.

In some aspects, the target nucleic acid is DNA. In other aspects, the target nucleic acid is RNA. In some aspects, the composition further comprises the target nucleic acid. In some aspects, the target nucleic acid comprises distinct target nucleic acids.

In various aspects, the present disclosure provides a method of assaying for a segment of a target nucleic acid in a sample, the method comprising: contacting the sample to the composition of any one of claims 1-30; and assaying for a signal produce by cleavage of a detector nucleic acid.

In some aspects, the method further comprises reverse transcribing the target nucleic acid, amplifying the target nucleic acid, in vitro transcribing the target nucleic acid, or any combination thereof. In some aspects, the amplifying is isothermal amplification.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts a pool of different guide nucleic acids complexed 1:1 with programmable nucleases.

FIG. 2 shows raw fluorescence over time of multiplexed DETECTR reactions using an LbCas12a programmable nuclease (SEQ ID NO: 18). Each multiplexed DETECTR reaction was performed with two distinct guide RNA sequences. In each reaction, a first guide nucleic acid sequence was present at either 19-fold, 49-fold, or 99-fold higher concentration than the second guide nucleic acid sequence to simulate 20-plex, 50-plex, or 100-plex high-plex DETECTR reactions, respectively. An 8-nucleotide single-stranded DNA detector nucleic acid labeled at the 5′ end with FAM and labeled at the 3′ end with Iowa Black FQ with a sequence of SEQ ID NO: 9 was used in each reaction.

FIG. 2A shows a first set of multiplexed DETECTR reactions in which a guide RNA targeting a human β-globin gene (SEQ ID NO: 172) was present in 19-fold (“20plex”), 49-fold (“50plex”), or 99-fold (“100plex”) higher concentration than a guide RNA targeting a human RNAase P gene (SEQ ID NO: 171). The pooled guide RNAs were used to detect the presence or absence of a double-stranded DNA target nucleic acid corresponding an amplified segment of the human RNase P gene (SEQ ID NO: 173, top row) or an amplified segment of the human β-globin gene (SEQ ID NO: 174, bottom row). Each multiplexed DETECTR reaction was performed in the presence of 0 pM, 10 pM, 100 pM, or 1000 pM of the target nucleic acid.

FIG. 2B shows a second set of multiplexed DETECTR reactions in which a guide RNA targeting a human RNAase P gene (SEQ ID NO: 171) was present in 19-fold (“20plex”), 49-fold (“50plex”), or 99-fold (“100plex”) higher concentration than a guide RNA targeting a human β-globin gene (SEQ ID NO: 172). The pooled guide RNAs were used to detect the presence or absence of a double-stranded DNA target nucleic acid corresponding an amplified segment of the human RNase P gene (SEQ ID NO: 173, top row) or an amplified segment of the human β-globin gene (SEQ ID NO: 174, bottom row). Each multiplexed DETECTR reaction was performed in the presence of 0 pM, 10 pM, 100 pM, or 1000 pM of the target nucleic acid.

FIG. 3 shows the raw fluorescence over time data from FIG. 2. Each spectrum is the result of a separate DETECTR reaction, with time (spanning approximately 90 minutes) as the x-axis and raw fluorescence yield on the y-axis. All spectra are shown with the same scales. A blank spectrum indicates that a reaction was not run.

FIG. 4 shows raw fluorescence over time of high-plex DETECTR reactions using an LbCas12a programmable nuclease (SEQ ID NO: 18, dashed lines) and a Cas12 variant programmable nuclease (SEQ ID NO: 28, solid lines). A guide RNA pool of 20 distinct guide nucleic acid sequences was used to detect the presence or absence of target nucleic acids in Borrelia culture diluted 10-fold (“Dilution-1”), 10²-fold (“Dilution-2”), 10³-fold (“Dilution-3”), 10⁴-fold (“Dilution-4”), 10⁵-fold (“Dilution-5”), 10⁶-fold (“Dilution-6”), or 10⁷-fold (“Dilution-7”) in a negative matrix and PCR-amplified. Diluted Borrelia cultures were PCR-amplified prior to detection to amplify the 16S gene. Negative plasma (“NegPlasma”), Zymo standard with Pseudomonas aeruginosa, Escherichia coli, Salmonella enterica, Lactobacillus subtilis, Saccharomyces cerevisiae, and Cryptococcus neoformans (“Zymo”), and water (“H2O’) were tested as negative controls.

FIG. 5 shows the maximum fluorescence rates of the high-plex DETECTR reactions shown in FIG. 4. Left columns in each condition correspond to reactions using the Cas12 variant programmable nuclease (SEQ ID NO: 28), and right columns correspond to reactions using the LbCas12a programmable nuclease (SEQ ID NO: 18).

FIG. 6 shows the time to result of the high-plex DETECTR reactions shown in FIG. 4 and FIG. 5. Left columns in each condition correspond to reactions using the Cas12 variant programmable nuclease (SEQ ID NO: 28), and right columns correspond to reactions using the LbCas12a programmable nuclease (SEQ ID NO: 18). A low time to result is indicative of a positive DETECTR reaction.

FIG. 7 shows raw fluorescence over time of multiplexed DETECTR reactions using a Cas12 programmable nuclease (SEQ ID NO: 18). The multiplexed DETECTR reactions were performed with four distinct guide RNA sequences, a first guide nucleic acid directed to a segment of a target nucleic acid comprising a human RNase P gene (SEQ ID NO: 172), and three off target guide sequences. The pool of off-target guide nucleic acid sequences were present at either 499-fold or 999-fold the first guide nucleic acid sequence to simulate 500-plex and 1000-plex DETECTR reactions, respectively. Additionally, single plex assays were also performed with no off-target guide nucleic acids present. An 8-nucleotide single-stranded DNA detector nucleic acid labeled at the 5′ end with FAM and labeled at the 3′ end with Iowa Black FQ with a sequence of SEQ ID NO: 9 was used in each reaction.

FIG. 8 shows the raw fluorescence over time data from FIG. 7. Each spectrum is the result of a separate DETECTR reaction, with time (spanning approximately 90 minutes) as the x-axis and raw fluorescence yield on the y-axis. All spectra are shown with the same scales. A blank spectrum indicates that a reaction was not run.

FIG. 9 shows raw fluorescence over time of multiplexed DETECTR reactions using a Cas12 programmable nuclease (SEQ ID NO: 28). Each multiplexed DETECTR reaction was performed with two distinct guide RNA sequences. In each reaction, a first guide nucleic acid sequence was present at either 19-fold, 49-fold, 99-fold, or 199-fold higher concentration than the second guide nucleic acid sequence to simulate 20-plex, 50-plex, 100-plex, or 199-plex high-plex DETECTR reactions, respectively. An 8-nucleotide single-stranded DNA detector nucleic acid labeled at the 5′ end with FAM and labeled at the 3′ end with Iowa Black FQ with a sequence of SEQ ID NO: 9 was used in each reaction.

FIG. 9A shows raw fluorescence data for simulated 20-plex, 50-plex, 100-plex, and 200-plex DETECTR reactions with target nucleic acid corresponding to an amplified segment of the human RNase P gene (top, SEQ ID NO: 195) and with an amplified segment of the human β-globin gene (bottom, SEQ ID NO: 174) at concentrations of 1 nM, 100 pM, or 0 pM (left, middle, and right columns, respectively). Guide RNA targeting a human RNase P gene (SEQ ID NO: 171) was present in 19-fold (“20plex”), 49-fold (“50plex”) 99-fold (“100plex”), or 199-fold (“200plex) higher concentration than a guide RNA sequence targeting a human RNase P gene (SEQ ID NO: 171).

FIG. 9B shows raw fluorescence data for simulated 20-plex, 50-plex, 100-plex, and 200-plex DETECTR reactions with target nucleic acid corresponding to an amplified segment of the human RNase P gene (top, SEQ ID NO: 195) and with an amplified segment of the human β-globin gene (bottom, SEQ ID NO: 174) at concentrations of 1 nM, 100 pM, or 0 pM (left, middle, and right columns, respectively). Guide RNA targeting a human β-globin gene (SEQ ID NO: 172) was present in 19-fold (“20plex”), 49-fold (“50plex”) 99-fold (“100plex”), or 199-fold (“200plex) higher concentration than a guide RNA sequence targeting a β-globin gene (SEQ ID NO: 171).

FIG. 10 shows the maximum fluorescence rate of the high-plex DETECTR reactions shown in FIG. 9. The left column corresponds to DETECTR reactions with 1000 pM (1 nM) target nucleic acid. The middle column corresponds to DETECTR reactions with 100 pM target nucleic acid. The right column corresponds to DETECTR reactions with 0 pM target nucleic acid. Bottom rows in each condition correspond to reactions using the human RNAase P gene (SEQ ID NO: 173) as the target nucleic acid, and top rows correspond to reactions the human β-globin gene (SEQ ID NO: 172) as the target nucleic acid.

FIG. 11 depicts an assay procedure in which bacterial DNA encoding the 16S ribosomal subunit is amplified and then subject to interrogation by a high-plex DETECTR reaction.

FIG. 12 depicts a high-plex DETECTR reaction designed to detect single nucleotide polymorphisms (SNP) in a DNA sample.

FIG. 13 depicts results of a DETECTR assay showing enhanced Cas12a-based detection of the GF184 target using a pooled-guide (pooled-gRNA) format compared to DETECTR Cas12a-based assay using an individual gRNA format.

FIG. 14 depicts results of a DETECTR assay showing enhanced sensitivity of the Cas13a-based detection of the SC2 target using a pooled-guide format compared to the Cas13a-based assays using an individual guide format.

FIG. 15 shows that relative quantification performed by counting the number of positive droplets showed that the Cas13a-DETECTR assay samples containing the pooled guide RNAs generated more crystals containing the amplified products per starting copy of the target RNA than the Cas13a-DETECTR assay samples containing the guide RNAs in individual format.

FIG. 16 shows that measurement of signal intensity following amplification showed that the Cas13a-DETECTR assay samples containing the pooled guide RNAs generated more signal intensity per starting copy of the target template RNA than the Cas13a-DETECTR assay samples containing the guide RNAs in individual format.

FIG. 17 shows that measurement of signal intensity following amplification showed that the Cas13a-DETECTR assay samples containing the pooled guide RNAs generated more signal intensity per starting copy of the target template RNA than the Cas13a-DETECTR assay samples containing the guide RNAs in individual format. FIG. 17 also shows that relative quantification performed by counting the number of positive droplets showed that the Cas13a-DETECTR assay samples containing the pooled guide RNAs generated more crystals containing the amplified products per starting copy of the target template RNA than the Cas13a-DETECTR assay samples containing the guide RNAs in individual format.

FIG. 18 shows that Cas13a DETECTR assay samples containing the pooled guides (R4637, R4638, R4667, R4676, R4684, R4689, R4691) did not exhibit higher target detection sensitivity per starting copy of the target than the Cas13a DETECTR samples containing the single guides R4684, R4667, or R4785 (RNAseP guide) in individual format.

### DETAILED DESCRIPTION

Disclosed herein are non-naturally occurring compositions and systems comprising at least one of an engineered Cas protein and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid comprises a non-natural nucleobase sequence. In some instances, the non-natural sequence is a nucleobase sequence that is not found in nature. The non-natural sequence may comprise a portion of a naturally-occurring sequence, wherein the portion of the naturally-occurring sequence is not present in nature absent the remainder of the naturally-occurring sequence. In some instances, the guide nucleic acid comprises two naturally-occurring sequences arranged in an order or proximity that is not observed in nature. In some instances, compositions and systems comprise a ribonucleotide complex comprising a CRISPR/Cas effector protein and a guide nucleic acid that do not occur together in nature. Engineered guide nucleic acids may comprise a first sequence and a second sequence that do not occur naturally together. For example, an engineered guide nucleic acid may comprise a sequence of a naturally-occurring repeat region and a spacer region that is complementary to a naturally-occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein comprise a multi-vector system encoding a Cas protein and a guide nucleic acid described herein, wherein the guide nucleic acid and the Cas protein are encoded by the same or different vectors. In some embodiments, the engineered guide and the engineered Cas protein are encoded by different vectors of the system.

The present disclosure provides various methods, reagents, and devices for high sensitivity detection of multiple target nucleic acids in a sample using a programmable nuclease. In particular, the various methods, reagents, and devices disclosed herein use programmable nucleases complexed with multiple guide nucleic acid sequences to detect multiple target nucleic acids in a sample. In some embodiments, the multiple target nucleic acids are associated with one or more diseases.

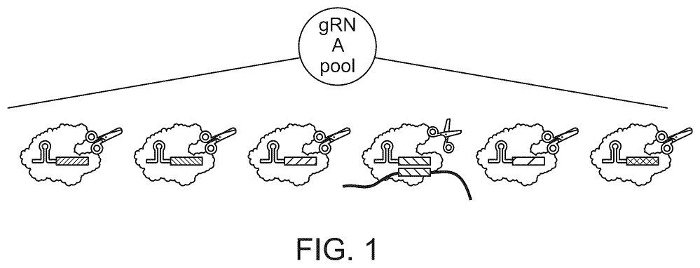

The compositions disclosed herein include high-plex pools of guide nucleic acids (e.g., guide RNAs) comprising multiple distinct guide nucleic acid sequences (e.g., guide RNA sequences), wherein at least one guide nucleic acid of the pool hybridizes to a segment of a target nucleic acid, as is depicted in FIG. 1. For example, the pool of guide nucleic acids comprises greater than 20 distinct guide nucleic acid sequences, such as 21 distinct guide nucleic acid sequences (referred to as a 21-plex). In some instances, the pool of guide nucleic acids can comprise at least 30 distinct nucleic acid sequences (30-plex), at least 50 nucleic acid sequences (50-plex), at least 100 nucleic acid sequences (100-plex), at least 500 nucleic acid sequences (500-plex), or at least 1000 nucleic acid sequences (1000-plex). The pools of guide nucleic acids can include multiple copies of the same guide nucleic acid. For example, a 21-plex guide pool of the present disclosure can have 21 distinct guide nucleic acid sequences and can have multiple copies of each of the 21 distinct guide nucleic acid sequences. Said compositions of pools of guide nucleic acids can be used with other reagents disclosed herein (e.g., programmable nucleases, detector nucleic acids) to detect a target nucleic acid in any sample described herein, for example, using the DETECTR methods described herein.

In some embodiments, the methods, reagents, and devices of this disclosure may be used for high sensitivity detection of a single target population of nucleic acids in a biological sample by pooling programmable nucleases complexed with multiple guide nucleic acids directed toward multiple target sequences within the single target population to be detected. Therefore, the present disclosure provides pools of guide nucleic acids having at least two guide nucleic acid sequences that are different from one another, thereby targeting different sequences of a target nucleic acid from one another. Pooling guide nucleic acids that align to multiple segments of the same target population (e.g., the same target genome) may enhance the sensitivity of the DETECTR assay disclosed herein. In some embodiments, the pools of guide nucleic acids disclosed herein, thus, comprise at least one guide nucleic acid that hybridizes to a segment of a target nucleic acid. In some cases, each guide nucleic acid sequence of the pool of guide nucleic acids hybridizes to distinct segments of the same target nucleic acid. For example, the distinct guide nucleic acid sequences of the pools of guide nucleic acids disclosed herein can have a sequence from a group of tiled guide nucleic acids that correspond to nucleic acids of the target nucleic acid. The tiled guide nucleic acids can be sequential along the target nucleic acid upon hybridization to the target nucleic acid. The tiled guide nucleic acids can be non-sequential along the target nucleic acid upon hybridization to the target nucleic acid, the tiled guide nucleic acid can be overlapping along the target nucleic acid upon hybridization to the target nucleic acid, or any combination hereof.

In some embodiments, the methods, reagents, and devices of this disclosure may be used for high sensitivity detection of multiple target populations in a biological sample by pooling programmable nucleases complexed with multiple guide nucleic acid directed toward target sequences in multiple target populations to be detected. Pooling guide nucleic acids that align to multiple target sequences within different target populations (e.g., different target genomes) may increase the sensitivity of the DETECTR assays disclosed herein for diseases associated with multiple pathogenic species (e.g., tick-bome pathogens). A target population may be, for example, a chromosome, a plasmid, a bacterial genome, a viral genome, a fungal genome, or an amoeboid genome. In some embodiments, the multiple guide nucleic acid sequences may comprise at least 20 distinct guide nucleic acid sequences. A method utilizing multiple guide nucleic acid (e.g., at least 20 distinct guide nucleic acid sequences) may be referred to as a “high plex” detection method. A high-plex composition for detection of a target nucleic acid, as disclosed herein, may comprise at least 20, at least 50, at least 100, at least 250, at least 500, at least 1000, or more guide nucleic acid sequences. Each guide nucleic acid sequence may be directed to a distinct target nucleic acid. The distinct target nucleic acids may be within a single target population. The distinct target nucleic acids may be within multiple target populations (e.g., 2, 3, 4, 5, 10, 15, 20, 30, 50, or more target populations). The distinct target nucleic acids may be different variants or alleles of one or more target sequences. Thus, in some cases, each guide nucleic acid sequence of the pool of guide nucleic acids hybridizes to segments from distinct target nucleic acids. For example, in a 21-plex guide pool, at least two of the guide nucleic acid sequences of the 21-plex guide pool can bind to segments of two different target nucleic acids. As another example, in a 21-plex guide pool, each of the 21 guide nucleic acid sequences can bind to segments of 21 different target nucleic acids. These different target nucleic acids can be from different pathogens or different strains of the same pathogen.

The compositions of pools of guide nucleic acids, programmable nucleases, and methods of use thereof disclosed herein can be used as a companion diagnostic with any of the diseases disclosed herein (e.g., bacterial, viral, fungal, or amoebic diseases), or can be used in reagent kits, point-of-care diagnostics, or over-the-counter diagnostics. The methods may be used as a point of care diagnostic or as a lab test for detection of a target nucleic acid and, thereby, detection of a condition in a subject from which the sample was taken. The methods may be used in various sites or locations, such as in laboratories, in hospitals, in physician offices/laboratories (POLs), in clinics, at remotes sites, or at home. Sometimes, the present disclosure provides various methods, reagents, and devices for consumer genetic use or for over the counter use.

Also described herein are methods, reagents, and devices for detecting the presence of a target nucleic acid in a sample. The methods, reagents, and devices for detecting the presence of a target nucleic acid in a sample can be used in a rapid lab tests for detection of a target nucleic acid of interest (e.g., target nucleic acids from a target population). In particular, provided herein are methods, reagents, and devices wherein the rapid lab tests can be performed in a single system. The target nucleic acid may be a portion of a nucleic acid from a virus or a bacterium or other agents responsible for a disease in the sample. The target nucleic acid may be a portion of an RNA or DNA from any organism in the sample. In some embodiments, programmable nucleases disclosed herein are activated by RNA or DNA to initiate trans cleavage activity of a detector nucleic acid. A detector nucleic acid can be an RNA or DNA with a detection moiety that emits a detectable signal upon trans cleavage of the RNA or DNA by the programmable nuclease. A programmable nuclease as disclosed herein is, in some cases, binds to a target RNA to initiate trans cleavage of a detector nucleic acid, and this programmable nuclease can be referred to as an RNA-activated programmable RNA nuclease. In some instances, a programmable nuclease as disclosed herein binds to a target DNA to initiate trans cleavage of a detector nucleic acid, and this programmable nuclease can be referred to as a DNA-activated programmable RNA nuclease. In some cases, a programmable nuclease as described herein is capable of being activated by a target RNA or a target DNA. For example, a Cas13 enzyme, such as Cas13a, disclosed herein is activated by a target RNA nucleic acid or a target DNA nucleic acid to transcollaterally cleave RNA detector nucleic acid. In some embodiments, the Cas13 binds to a target ssDNA which initiates trans cleavage of RNA detector nucleic acid. The detection of the target nucleic acid in the sample may indicate the presence of the disease in the sample and may provide information for taking action to reduce the transmission of the disease to individuals in the disease-affected environment or near the disease-carrying individual. The detection of the target nucleic acid in the sample may indicate the presence of a disease mutation, such as a single nucleotide polymorphism (SNP) that provides antibiotic resistance to a disease-causing bacteria. The detection of the target nucleic acid is facilitated by a programmable nuclease. The programmable nuclease can become activated after binding of a guide nucleic acid with a target nucleic, in which the activated programmable nuclease can cleave the target nucleic acid and can have trans cleavage activity, which can also be referred to as “collateral” or “transcollateral” cleavage. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety is released from the detector nucleic acid and generates a detectable signal that is immobilized to on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or level of the target nucleic acid associated with an ailment, such as a disease. The programmable nuclease can be a CRISPR-Cas (clustered regularly interspaced short palindromic repeats—CRISPR associated) nucleoprotein complex with trans cleavage activity, which can be activated by binding of a guide nucleic acid of the CRISPR-Cas nucleoprotein complex with a target nucleic acid. A reaction comprising production of a detectable signal upon cleavage of a detector nucleic acid by an activated programmable nuclease may be referred to herein as a DETECTR reaction. A DETECTR reaction comprising detection of a plurality of target nucleic acids using a pool of guide nucleic acids and may be referred to herein as a “multiplexed” or “high-plex” DETECTR reaction. A DETECTR reaction comprising detection of multiple target nucleic acids (e.g., at least 2 different segments of target nucleic acids) using multiple distinct guide nucleic acid sequences (e.g., greater than 20 guide nucleic acid sequences) may be referred to herein as a high-plex DETECTR reaction.

In one aspect, described herein is a method for detecting multiple target nucleic acids within a single target population. The method may comprising contacting programmable nucleases to a pool of guide nucleic acids comprising multiple guide nucleic acid sequences. The programmable nucleases are capable of being activated when complexed with a guide nucleic acid and a target sequence. Each guide nucleic acid of the pool of guide nucleic acids may be directed to a different segment within a single target nucleic acid to be detected (e.g., a target nucleic acid associated with a disease). The method may further comprising contacting the programmable nucleases complexed with the pool of guide nucleic acids to a biological sample and a detector nucleic acid, wherein the detector nucleic acid is capable of being cleaved by the activated programmable nuclease, thereby generating a detectable signal, to detect the presence or absence of the target nucleic acid in the biological sample.

In another aspect, described herein is a method for detecting multiple target nucleic acids within multiple target populations. The programmable nucleases are capable of being activated when complexed with a guide nucleic acid and a target sequence. The method may comprising contacting programmable nucleases to a pool of guide nucleic acids comprising multiple guide nucleic acid sequences. Each guide nucleic acid sequence of the pool of guide nucleic acids may be directed to a different target nucleic acids within a plurality of target nucleic acids to be detected (e.g., target nucleic acids associated with one or more diseases). The method may further comprising contacting the programmable nucleases complexed with the pool of guide nucleic acids to a biological sample and a detector nucleic acid, wherein the detector nucleic acid is capable of being cleaved by the activated programmable nuclease, thereby generating a detectable signal, to detect the presence or absence of one or more of the plurality of target nucleic acids in the biological sample.

In another aspect, described herein is a method for detecting multiple variations of a target nucleic acid within a single target population. The method may comprising contacting programmable nucleases to a plurality of guide nucleic acids. The programmable nucleases are capable of being activated when complexed with a guide nucleic acid and a target sequence. Each guide nucleic acid sequence of the pool of guide nucleic acids may be directed to a different variations (e.g., different alleles) of a target nucleic acid sequence within a single target nucleic acid to be detected (e.g., a target nucleic acid associated with a disease). The method may further comprising contacting the programmable nucleases complexed with the pool of guide nucleic acids to a biological sample and a detector nucleic acid, wherein the detector nucleic acid is capable of being cleaved by the activated programmable nuclease, thereby generating a detectable signal, to detect the presence or absence of the target nucleic acid in the biological sample.

Also described herein is a kit for detecting one or more target populations (e.g., one or more target populations associated with a disease). The kit may comprise a support medium; a pool of guide nucleic acid sequences targeted to different target nucleic acid sequences; a programmable nuclease capable of being activated when complexed with a guide nucleic acid and a target nucleic acid; and a single-stranded detector nucleic acid comprising a detection moiety, wherein the detector nucleic acid is capable of being cleaved by the activated nuclease, thereby generating a first detectable signal.

A biological sample from an individual or an environmental sample can be tested to determine whether the individual has a communicable disease. The biological sample can be tested to detect the presence or absence of at least one target nucleic acid from one or more target populations associated with the disease (e.g., a bacterial genome, a viral genome, a fungal genome, or an amoeboid genome). The at least one target nucleic acid from the one or more target populations associated with the disease that is detected can also indicate that one or more of the target populations is wild-type or comprises a mutation that confers resistance to treatment, such as antibiotic treatment. A sample from an individual or from an environment is applied to the reagents described herein. If the target nucleic acid is present in the sample, the target nucleic acid binds to the guide nucleic acid to activate the programmable nuclease. The activated programmable nuclease cleaves the detector nucleic acid and generates a detectable signal that can be visualized, for example on a support medium. If the target nucleic acid is absent in the sample or below the threshold of detection, the guide nucleic acid remains unbound, the programmable nuclease remains inactivated, and the detector nucleic acid remains uncleaved.

Such methods, reagents, and devices described herein may allow for detection of target nucleic acid, and in turn the disease associated with the target nucleic acids (e.g., a bacterial infection, a viral infection, a fungal infection, or an amoeboid infection), in remote regions or low resource settings without specialized equipment. Also, such methods, reagents, and devices described herein may allow for detection of target nucleic acid, and in turn the disease associated with the target nucleic acids, in healthcare clinics or doctor offices without specialized equipment. In some cases, this provides a point of care testing for users to quickly and easily test for a disease or infection with high sensitivity at home or in an office of a healthcare provider. Assays that deliver results in under an hour, for example, in 15 to 60 minutes, are particularly desirable for at home testing for many reasons. For example, antivirals can be most effective when administered within the first 48 hours after disease exposure. Thus, the methods disclosed herein, which are capable of delivering results in under an hour. may allow for the delivery of anti-viral therapy during the first 48 hours after infection. Additionally, the systems and assays provided herein, which are capable of delivering quick diagnoses and results, can help keep or send a patient at home, improve comprehensive disease surveillance, and prevent the spread of an infection. Assays that detect a target population (e.g., a target population associated with a disease) with high sensitivity may provide early and accurate detection of a disease. In some cases, early and accurate detection may improve antibiotic stewardship by enabling healthcare providers to selectively administer antibiotics based on the infecting target population. In other cases, this provides a test, which can be used in a lab to detect one or more nucleic acid populations or varieties of interest in a sample from a subject. In particular, provided herein are methods, reagents, and devices, wherein the high sensitivity lab tests can be performed in a single assay. In some cases, this may be valuable in detecting diseases in a developing country and as a global healthcare tool to detect the spread of a disease or efficacy of a treatment or provide early detection of a disease.

Some methods as described herein use an editing technique, such as a technique using an editing enzyme or a programmable nuclease and guide nucleic acid, to detect one or more target nucleic acid populations. An editing enzyme or a programmable nuclease in the editing technique can be activated by one or more target nucleic acids, after which the activated editing enzyme or activated programmable nuclease can cleave nearby single-stranded nucleic acids, such detector nucleic acids with a detection moiety. A target nucleic acid population (e.g., a target population from a chromosome, a plasmid, a bacterial genome, a viral genome, a fungal genome, or an amoeboid genome), can be amplified by isothermal amplification and then an editing technique can be used to detect the marker. In some instances, the editing technique can comprise an editing enzyme or programmable nuclease that, when activated, cleaves nearby RNA or DNA as the readout of the detection. The methods as described herein in some instances comprise obtaining a cell-free DNA sample, amplifying DNA from the sample, using an editing technique to cleave detector nucleic acids, and reading the output of the editing technique. In other instances, the method comprises obtaining a fluid sample from a patient, and without amplifying a nucleic acid of the fluid sample, using an editing technique to cleave detector nucleic acids, and detecting the nucleic acid. The method can also comprise using single-stranded detector DNA, cleaving the single-stranded detector DNA using an activated editing enzyme, wherein the editing enzyme cleaves at least 50% of a population of single-stranded detector DNA as measured by a change in color. A number of samples, guide nucleic acids, programmable nucleases or editing enzymes, support mediums, target nucleic acids, single-stranded detector nucleic acids, and reagents are consistent with the devices, systems, fluidic devices, kits, and methods disclosed herein.

Also disclosed herein are detector nucleic acids and methods detecting a target nucleic using the detector nucleic acids. Often, the detector nucleic acid is a protein-nucleic acid. For example, a method of assaying for one or more target nucleic acid populations in a sample comprises contacting the sample to a plurality of complexes comprising a guide nucleic acid, each guide nucleic acid sequence comprising a segment that is reverse complementary to a segment of a target nucleic acid sequence within a target nucleic acid population and programmable nucleases that exhibits sequence independent cleavage upon forming complexes comprising the segment of the guide nucleic acid binding to the segment of the target nucleic acid; and assaying for a signal indicating cleavage of at least some protein-nucleic acids of a population of protein-nucleic acids, wherein the signal indicates a presence of one or more of the target nucleic acid populations in the sample and wherein absence of the signal indicates an absence of the target nucleic acid population in the sample. Often, the protein-nucleic acid is an enzyme-nucleic acid or an enzyme substrate-nucleic acid. The nucleic acid can be DNA, RNA, or a DNA/RNA hybrid. The methods described herein use a programmable nuclease, such as a Cas enzyme, to detect one or more target nucleic acid populations. A method of assaying for one or more target nucleic acid populations in a sample, for example, comprises: a) contacting the sample to a plurality of complexes comprising a plurality of guide nucleic acids, each guide nucleic acid sequence comprising a segment that is reverse complementary to a segment of a nucleic acid target sequence within a target nucleic acid population, and programmable nucleases that exhibits sequence independent cleavage upon forming complexes comprising the segment of the guide nucleic acid binding to the segment of the target nucleic acid; b) contacting the complexes to a substrate; c) contacting the substrate to a reagent that differentially reacts with a cleaved substrate; and d) assaying for a signal indicating cleavage of the substrate, wherein the signal indicates a presence of one or more of the target nucleic acid populations in the sample and wherein absence of the signal indicates an absence of the target nucleic acid population in the sample. Often, the substrate is an enzyme-nucleic acid. Sometimes, the substrate is an enzyme substrate-nucleic acid.

Cleavage of the protein-nucleic acid produces a signal. For example, cleavage of the protein-nucleic acid produces a calorimetric signal, a potentiometric signal, an amperometric signal, an optical signal, or a piezo-electric signal. Various devices can be used to detect these different types signals, which indicate whether a target nucleic acid is present in the sample.

### Sample

A number of samples are consistent with the methods, reagents, and devices disclosed herein. These samples are, for example, consistent with the high-plex detection methods disclosed herein, wherein the high-plex detection methods comprise contacting a sample to programmable nucleases complexed with a pool of guide nucleic acids (e.g., guide RNAs), and a detector nucleic acid. The pool of guide nucleic acids, can have any number of distinct guide nucleic acid sequences (e.g., guide RNA sequences), as disclosed herein. For example, the pool of guide nucleic acids can have at least 21 distinct guide nucleic acid sequences (corresponding to a 21-plex), at least 50 distinct guide nucleic acid sequences (corresponding to a 50-plex), at least 100 distinct guide nucleic acid sequences (corresponding to a 100-plex), at least 500 distinct guide nucleic acid sequences (corresponding to a 500-plex), or at least 1000 distinct guide nucleic acid sequences (corresponding to a 1000-plex). Said distinct guide nucleic acid sequences in the pool of guide nucleic acids can hybridize to different segments of a target nucleic acid that may be present in any sample disclosed as follows. Additionally, and or alternatively, said distinct guide nucleic acid sequences in the pool of guide nucleic acids can hybridize to different segments of distinct target nucleic acids (e.g., target nucleic acids from different pathogens or different strains from the same pathogen) that may be present in any sample disclosed as follows.

These samples can comprise a target nucleic acid for detection of an ailment, such as a disease, pathogen, or virus, such as influenza. A pathogen can be a virus, a bacterium, a fungus, a protozoan, or a worm. Generally, a sample from an individual or an animal or an environmental sample can be obtained to test for presence of a disease, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquefied prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μL. The sample, in some cases, is contained in no more than 1, 5, 10, 15, 20, 25, 30, 35 40, 45, 50, 55, 60, 65, 70, 75, 80, 90, 100, 200, 300, 400, 500 μL, or any of value from 1 μL to 500 μL. Sometimes, the sample is contained in more than 500 μL.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample used for disease testing may comprise at least one target sequence that can bind to a guide nucleic acid of the reagents described herein. The sample used for disease testing may comprise multiple target sequences, corresponding to multiple target nucleic acids. In some cases, the target sequence is a portion of a nucleic acid population. The multiple target sequences may be located within a single nucleic acid population. They multiple target sequences may be located within multiple target nucleic acid populations. A portion of a nucleic acid can be from a genomic locus, a transcribed mRNA, or a reverse transcribed cDNA. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target sequence can be reverse complementary to a guide nucleic acid. Each target sequences of the multiple target sequences can be reverse complementary to a distinct guide nucleic acid.

In some cases, the target sequence is a portion of a nucleic acid population from a virus or a bacterium or other agents responsible for a disease in the sample (e.g., a bacterial genome, a viral genome, a fungal genome, or an amoeboid genome). The target sequence, in some cases, is a portion of a nucleic acid population from a sexually transmitted infection or a contagious disease, in the sample. In some examples, in the target nucleic acid is a portion of a nucleic acid from a human immunodeficiency virus (HIV), a human papillomavirus (HPV), a Chlamydia trachomatis bacterium, a Neisseria gonorrhoeae bacterium, or a Treponema pallidum bacterium. The target sequence, in some cases, is a portion of a nucleic acid population from an upper respiratory tract infection, a lower respiratory tract infection, or a contagious disease, in the sample. The target sequence, in some cases, is a portion of a nucleic acid population from a hospital acquired infection, healthcare-associated infection (HAI), or a contagious disease, in the sample. The target sequence, in some cases, is an ssRNA. These target sequences may be from a disease, and the disease may include but is not limited to influenza virus, including influenza A virus (IAV) or influenza B virus (IBV), rhinovirus, cold viruses, a respiratory virus, an upper respiratory virus, a lower respiratory virus, or respiratory syncytial virus. In some examples the disease may be severe acute respiratory syndrome (SARS), a coronavirus, SARS-CoV, or SARS-CoV-2. In some examples, the disease is SARS-CoV-2 (also known as 2019 novel coronavirus, or 2019-nCoV). The coronavirus may be a variant of SARS-CoV-2, particularly the variant known as 20B/501Y.V1, VOC 202012/01, or B.1.1.7 lineage, or the variant known as: 20C/501Y.V2 or B.1.351 lineage. In some examples, the disease is IAV. In some examples, the disease is IBV. Pathogens include viruses, fungi, helminths, protozoa, and parasites. Pathogens include, e.g., Mycobacterium tuberculosis, Streptococcus agalactiae, methicillin-resistant Staphylococcus aureus, Legionella pneumophila, Streptococcus pyogenes, Escherichia coli, Neisseria meningitidis, Pneumococcus, Hemophilus influenzae B, influenza virus, respiratory syncytial virus (RSV), M. pneumoniae, Streptococcus intermdius, Streptococcus pneumoniae, and Streptococcus pyogenes. In some examples, the pathogen is a Group A streptococcus bacterium. In some examples, the pathogen is a Neisseria gonorrhoeae bacterium. In some examples, the pathogen is a Mycoplasma genitalium bacterium. In some examples, the pathogen is a Trichomonas vaginalis parasite. In some examples, the pathogen is a Treponema pallidum bacterium. In some examples, the pathogen is a bacterium or fungus causing a urinary tract infection. In some examples, the bacterium is a Helicobacter pylori bacterium. In some examples, the pathogen is a species of candida. In some examples, the pathogen is a bacterium causing bacterial vaginosis. In some examples, the pathogen is a Clostridioides difficile bacterium. In some examples, the pathogen is a norovirus. In some examples, the pathogen is a hepatitis B virus. In some examples, the pathogen is a virus, fungus, bacterium, parasite or other pathogen causing meningitis. In some examples, the pathogen is a herpes simplex virus. In some examples, the pathogen is a lentivirus. In some examples, the pathogen is a hepatitis C virus. In some examples, the pathogen is a zika virus. In some examples, the pathogen is a human immunodeficiency virus 1 or a human immunodeficiency virus 2. Pathogens may comprise multiple pathogenic species. For example, tick-borne pathogens may comprise one or more infections genera or species (e.g., one or more species of Borrelia, Babesia, or Rickettsia). In another example, pathogens may include healthcare-associated infections (HAI), which may comprise one or more genera or species. Pathogens may comprise multiple species of a genus (e.g., one or more species of Borrelia, one or more species of Babesia, or one or more species of Rickettsia). Often the target nucleic acid comprises a sequence from a virus or a bacterium or other agents responsible for a disease that can be found in the sample. Pathogenic viruses include but are not limited to influenza virus; RSV; an ssRNA virus, a respiratory virus, an upper respiratory virus, a lower respiratory virus, or a rhinovirus. Pathogens include, e.g., Mycobacterium tuberculosis, Streptococcus agalactiae, Legionella pneumophila, Streptococcus pyogenes, Hemophilus influenzae B influenza virus, respiratory syncytial virus (RSV), or Mycobacterium tuberculosis

The sample can be used for identifying a disease status. For example, a sample is any sample described herein, and is obtained from a subject for use in identifying a disease status of a subject. Sometimes, a method comprises obtaining a serum sample from a subject; and identifying a disease status of the subject.

In some instances, the target nucleic acid is a single-stranded nucleic acid. Alternatively or in combination, the target nucleic acid is a double stranded nucleic acid and is prepared into single-stranded nucleic acids before or upon contacting the reagents. The target nucleic acid may be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acids are consistent with the methods and compositions disclosed herein. Some methods described herein can detect two or more target nucleic acid sequences present in the sample in various concentrations or amounts. In some cases, the sample has at least 2 target nucleic acid sequences. In some cases, the sample has at least 3, 4, 5, 6, 7, 8, 9, 10, 20, 30, 40, or 50 target nucleic acid sequences. Some methods described herein can detect at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, or more target nucleic acid sequences. The target nucleic acid populations may be from one or more target nucleic acid sequences. For example, the target nucleic acid sequences may be from at least 1, at least 2, at least 3, at least 4, at least, 5, at least 6, at least 7, at least 8, at least 9, at least 10, at least 20, at least 30, at least 40, at least 50, at least 60, at least 70, at least 80, at least 90, at least 100, or more target nucleic acid populations. In some cases, the method detects target nucleic acids sequences that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid sequences can be present at different concentrations or amounts in the sample.

Any of the above disclosed samples are consistent with the systems, assays, and programmable nucleases disclosed herein and can be used as a companion diagnostic with any of the diseases disclosed herein (e.g., tick-bome pathogens or healthcare-associated infections), or can be used in reagent kits, point-of-care diagnostics, or over-the-counter diagnostics.

### Reagents

A number of reagents are consistent with the methods, reagents, and devices disclosed herein. Reagents disclosed herein for detection of a target nucleic acid are compatible with the pools of guide nucleic acids (e.g., guide RNAs) disclosed herein (e.g., a 21-plex pool of guide nucleic acids, a 50-plex pool of guide nucleic acids, a 100-plex pool of guide nucleic acids, a 500-plex pool of guide nucleic acids, or a 1000-plex pool of guide nucleic acids). The pool of guide nucleic acids, can have any number of distinct guide nucleic acid sequences, as disclosed herein. For example, the pool of guide nucleic acids can have at least 21 distinct guide nucleic acid sequences (corresponding to a 21-plex), at least 50 distinct guide nucleic acid sequences (corresponding to a 50-plex), at least 100 distinct guide nucleic acid sequences (corresponding to a 100-plex), at least 500 distinct guide nucleic acid sequences (corresponding to a 500-plex), or at least 1000 distinct guide nucleic acid sequences (corresponding to a 1000-plex). Said distinct guide nucleic acid sequences in the pool of guide nucleic acids can hybridize to different segments of a target nucleic acid that may be detected using the reagents disclosed herein, as follows. Additionally, and or alternatively, said distinct guide nucleic acid sequences in the pool of guide nucleic acids can hybridize to different segments of distinct target nucleic acids (e.g., target nucleic acids from different pathogens or different strains from the same pathogen) that may be detected using the reagents disclosed herein, as follows.

These reagents are compatible with the samples, methods, and devices as described herein for detection of an ailment, such as a disease. The reagents described herein for detecting a disease comprise multiple guide nucleic acids, each guide nucleic acid targeting a target nucleic acid segment indicative of the disease. Each guide nucleic acid binds to the target nucleic acid comprising a segment of a nucleic acid sequence (e.g., a nucleic acid from a virus or a bacterium or other agents responsible for a disease) as described herein. Each guide nucleic acid can bind to the target nucleic acid comprising a portion of a nucleic acid (e.g., a target nucleic acid from a bacterium or other agents responsible for a disease) as described herein and further comprising a mutation, such as a single nucleotide polymorphism (SNP), that can confer resistance to a treatment, such as antibiotic treatment. Each guide nucleic acid binds to the target nucleic acid comprising a portion of a nucleic acid. Each guide nucleic acid is complementary to a target nucleic acid. Often the guide nucleic acid binds specifically to the target nucleic acid. The target nucleic acid may be a RNA, DNA, or synthetic nucleic acids.

Disclosed herein are methods of assaying for a plurality of target nucleic acids as described herein. For example, a method of assaying for a plurality of target nucleic acids in a sample comprises contacting the sample to a complex comprising a plurality of guide nucleic acid sequences, each guide nucleic acid sequence comprising a segment that is reverse complementary to a segment of the target nucleic acid, and programmable nucleases that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of one or more target nucleic acid of the plurality of target nucleic acids in the sample and wherein absence of the signal indicates an absence of the target nucleic acids in the sample. As another example, a method of assaying for a target nucleic acid in a sample, for example, comprises: a) contacting the sample to a plurality of complexes, each complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the plurality of complexes to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. A signal can be a calorimetric, potentiometric, amperometric, optical (e.g., fluorescent, colorometric, etc.), or piezo-electric signal. Often, the signal is present prior to detector nucleic acid cleavage and changes upon detector nucleic acid cleavage. Sometimes, the signal is absent prior to detector nucleic acid cleavage and is present upon detector nucleic acid cleavage. The detectable signal can be immobilized on a support medium for detection. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The CRISPR-Cas nucleoprotein complex can comprise a Cas enzyme complexed with a guide nucleic acid. The guide nucleic acid can be a guide RNA. The guide nucleic acid can comprise a CRISPR RNA (crRNA) and a trans-activating crRNA (tracrRNA). In some embodiments, the guide RNA comprises just the crRNA. The crRNA can complex with the tracrRNA to form the guide RNA. The crRNA can be made up of a repeat region and a spacer sequence. The entire spacer or a segment of the spacer of the crRNA can hybridize to a target nucleic acid.

The CRISPR-Cas nucleoprotein complex used to detect a modified target nucleic acids, wherein the CRISPR-Cas nucleoprotein complex can comprise CRISPR RNAs (crRNAs), trans-activating crRNAs (tracrRNAs), Cas enzymes, and detector nucleic acids.

A guide nucleic acid (e.g., guide RNA) can comprise a sequence that is reverse complementary to the sequence of a target nucleic acid. Said sequence that is reverse complementary to the sequence of the target nucleic acid in the guide nucleic acid can be a crRNA. Said sequence the is reverse complementary to the sequence of the target nucleic acid in the guide nucleic acid can be a or a portion of a crRNA. For example, either part or the entire sequence of the spacer region of the crRNA can be said sequence that is reverse complementary to the sequence of the target nucleic acid. Sometimes, a guide nucleic acid comprises a crRNA and tracrRNA. The guide nucleic acid can bind specifically to the target nucleic acid. In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. It is understood that the sequence of a polynucleotide need not be 100% complementary to that of its target nucleic acid to be specifically hybridizable or hybridizable or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid (e.g., guide RNA) can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a bacterial, viral, or fungal strain. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that correspond to nucleic acids of a target nucleic acid; and assaying for a signal produce by cleavage of at least some detector nucleic acids of a population of detector nucleic acids. Pooling of guide nucleic acids can ensure broad spectrum identification, or broad coverage, of a target species within a single reaction. This can be particularly helpful in diseases or indications, like sepsis, that may be caused by multiple organisms.

A “plurality of guide nucleic acids” and a “pool of guide nucleic acids” can be used interchangeably herein. The pool of guide nucleic acids (e.g., guide RNAs) disclosed herein may comprise at least 20 distinct guide nucleic acid sequences, at least 30 distinct guide nucleic acid sequences, at least 40 distinct guide nucleic acid sequences, at least 50 distinct guide nucleic acid sequences, at least 60 distinct guide nucleic acid sequences, at least 70 distinct guide nucleic acid sequences, at least 80 distinct guide nucleic acid sequences, at least 90 distinct guide nucleic acid sequences, at least 100 distinct guide nucleic acid sequences, at least 200 distinct guide nucleic acid sequences, at least 300 distinct guide nucleic acid sequences, at least 400 distinct guide nucleic acid sequences, at least 500 distinct guide nucleic acid sequences, at least 600 distinct guide nucleic acid sequences, at least 700 distinct guide nucleic acid sequences, at least 800 distinct guide nucleic acid sequences, at least 900 distinct guide nucleic acid sequences, at least 1000 distinct guide nucleic acid sequences, or more distinct guide nucleic acid sequences. In each pool of guide nucleic acids, multiple copies of each of the guide nucleic acid sequences can be present. The plurality, or pool, of guide nucleic acids can have multiple copies of each distinct guide nucleic acid sequence. Each guide nucleic acid sequence in the pool of guide nucleic acids may be directed to a distinct segment target nucleic acid. The distinct target nucleic acids may be from a single target nucleic acid population. The distinct target nucleic acids may be from multiple target nucleic acid populations. The distinct target nucleic acids may be different variants of a target sequence from a single target nucleic acid population or multiple target nucleic acid populations. Each guide nucleic acid sequence of the pool of guide nucleic acid sequences may be complexed with a programmable nuclease.

Described herein are reagents comprising a programmable nuclease capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and degrades non-specifically nucleic acid in its environment. The programmable nuclease has trans cleavage activity once activated. A programmable nuclease can be a Cas enzyme. A guide nucleic acid and a Cas enzyme can form a CRISPR-Cas nucleoprotein complex.

Several programmable nucleases are consistent with the methods and devices of the present disclosure. For example, Cas enzymes are programmable nucleases used in the methods and systems disclosed herein. Cas enzymes can include any of the known Classes and Types of CRISPR-Cas enzymes. For example, programmable nucleases disclosed herein include Class 1 CRISPR-Cas enzymes, such as the Type I, Type IV, or Type III CRISPR-Cas enzymes. Programmable nucleases disclosed herein also include the Class 2 CRISPR-Cas enzymes, such as the Type II, Type V, and Type VI CRISPR-Cas enzymes. Preferable programmable nucleases included in the several devices disclosed herein (e.g., a microfluidic device such as a pneumatic valve device or a sliding valve device or a lateral flow assay) and methods of use thereof include a Type V or Type VI CRISPR-Cas enzyme.

In some embodiments, the Type V CRISPR-Cas enzyme is a programmable Cas12 nuclease. Type V CRISPR-Cas enzymes (e.g., Cas12 or Cas14) lack an HNH domain. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 enzymes additionally include two domains for PAM recognition termed the PAM interacting (PI) domain and the wedge (WED) domain. (Murugan et al., Mol Cell. 2017 Oct. 5; 68(1): 15-25). A programmable Cas12 nuclease can be a Cas12a (also referred to as Cpf1) enzyme, a Cas12b enzyme, Cas12c enzyme, Cas12d enzyme, or a Cas12e enzyme. In some cases, a suitable Cas12 enzyme comprises an amino acid sequence having at least 80%, at least 85%, at least 90%, at least 95%, at least 98%, at least 99%, or 100%, amino acid sequence identity to any one of SEQ ID NO: 18-SEQ ID NO: 60.

TABLE 1
Cas12 Enzyme Sequences

| SEQ | | |
|---|---|---|
| ID | | |
| NO | Description | Sequence |
| SEQ | Lachnospiraceae | MSKLEKFTNCYSLSKTLRFKAIPVGKTQEN |
| ID | bacterium | IDNKRLLVEDEKRAEDYKGVKKLLDRYYLS |
| NO: | ND2006 | FINDVLHSIKLKNLNNYISLFRKKTRTEKE |
| 18 | (LbCas12a) | NKELENLEINLRKEIAKAFKGNEGYKSLFK |
| | | KDIIETILPEFLDDKDEIALVNSFNGFTTA |
| | | FTGFFDNRENMFSEEAKSTSIAFRCINENL |
| | | TRYISNMDIFEKVDAIFDKHEVQEIKEKIL |
| | | NSDYDVEDFFEGEFFNFVLTQEGIDVYNAI |
| | | IGGFVTESGEKIKGLNEYINLYNQKTKQKL |
| | | PKFKPLYKQVLSDRESLSFYGEGYTSDEEV |
| | | LEVFRNTLNKNSEIFSSIKKLEKLFKNFDE |
| | | YSSAGIFVKNGPAISTISKDIFGEWNVIRD |
| | | KWNAEYDDIHLKKKAVVTEKYEDDRRKSFK |
| | | KIGSFSLEQLQEYADADLSVVEKLKEIIIQ |
| | | KVDEIYKVYGSSEKLFDADFVLEKSLKKND |
| | | AVVAIMKDLLDSVKSFENYIKAFFGEGKET |
| | | NRDESFYGDFVLAYDILLKVDHIYDAIRNY |
| | | VTQKPYSKDKFKLYFQNPQFMGGWDKDKET |
| | | DYRATILRYGSKYYLAIMDKKYAKCLQKID |
| | | KDDVNGNYEKINYKLLPGPNKMLPKVFFSK |
| | | KWMAYYNPSEDIQKIYKNGTFKKGDMFNLN |
| | | DCHKLIDFFKDSISRYPKWSNAYDFNFSET |
| | | EKYKDIAGFYREVEEQGYKVSFESASKKEV |
| | | DKLVEEGKLYMFQIYNKDFSDKSHGTPNLH |
| | | TMYFKLLFDENNHGQIRLSGGAELFMRRAS |
| | | LKKEELVVHPANSPIANKNPDNPKKTTTLS |
| | | YDVYKDKRFSEDQYELHIPIAINKCPKNIF |
| | | KINTEVRVLLKHDDNPYVIGIDRGERNLLY |
| | | IVVVDGKGNIVEQYSLNEIINNFNGIRIKT |
| | | DYHSLLDKKEKERFEARQNWTSIENIKELK |
| | | AGYISQVVHKICELVEKYDAVIALEDLNSG |
| | | FKNSRVKVEKQVYQKFEKMLIDKLNYMVDK |
| | | KSNPCATGGALKGYQITNKFESFKSMSTQN |
| | | GFIFYIPAWLTSKIDPSTGFVNLLKTKYTS |
| | | IADSKKFISSFDRIMYVPEEDLFEFALDYK |
| | | NFSRTDADYIKKWKLYSYGNRIRIFRNPKK |
| | | NNVFDWEEVCLTSAYKELFNKYGINYQQGD |
| | | IRALLCEQSDKAFYSSFMALMSLMLQMRNS |
| | | ITGRTDVDFLISPVKNSDGIFYDSRNYEAQ |
| | | ENAILPKNADANGAYNIARKVLWAIGQFKK |
| | | AEDEKLDKVKIAISNKEWLEYAQTSVKH |
| SEQ | Acidaminococcus | MTQFEGFTNLYQVSKTLRFELIPQGKTLKH |
| ID | sp. | IQEQGFIEEDKARNDHYKELKPIIDRIYKT |
| NO: | BV316 | YADQCLQLVQLDWENLSAAIDSYRKEKTEE |
| 19 | (AsCas12a) | TRNALIEEQATYRNAIHDYFIGRTDNLTDA |
| | | INKRHAEIYKGLFKAELFNGKVLKQLGTVT |
| | | TTEHENALLRSFDKFTTYFSGFYENRKNVF |
| | | SAEDISTAIPHRIVQDNFPKFKENCHIFTR |
| | | LITAVPSLREHFENVKKAIGIFVSTSIEEV |
| | | FSFPFYNQLLTQTQIDLYNQLLGGISREAG |
| | | TEKIKGLNEVLNLAIQKNDETAHIIASLPH |
| | | RFIPLFKQILSDRNTLSFILEEFKSDEEVI |
| | | QSFCKYKTLLRNENVLETAEALFNELNSID |
| | | LTHIFISHKKLETISSALCDHWDTLRNALY |
| | | ERRISELTGKITKSAKEKVQRSLKHEDINL |
| | | QEIISAAGKELSEAFKQKTSEILSHAHAAL |
| | | DQPLPTTLKKQEEKEILKSQLDSLLGLYHL |
| | | LDWFAVDESNEVDPEFSARLTGIKLEMEPS |
| | | LSFYNKARNYATKKPYSVEKFKLNFQMPTL |
| | | ASGWDVNKEKNNGAILFVKNGLYYLGIMPK |
| | | QKGRYKALSFEPTEKTSEGFDKMYYDYFPD |
| | | AAKMIPKCSTQLKAVTAHFQTHTTPILLSN |
| | | NFIEPLEITKEIYDLNNPEKEPKKFQTAYA |
| | | KKTGDQKGYREALCKWIDFTRDFLSKYTKT |
| | | TSIDLSSLRPSSQYKDLGEYYAELNPLLYH |
| | | ISFQRIAEKEIMDAVETGKLYLFQIYNKDF |
| | | AKGHHGKPNLHTLYWTGLFSPENLAKTSIK |
| | | LNGQAELFYRPKSRMKRMAHRLGEKMLNKK |
| | | LKDQKTPIPDTLYQELYDYVNHRLSHDLSD |
| | | EARALLPNVITKEVSHEIIKDRRFTSDKFF |
| | | FHVPITLNYQAANSPSKFNQRVNAYLKEHP |
| | | ETPIIGIDRGERNLIYITVIDSTGKILEQR |
| | | SLNTIQQFDYQKKLDNREKERVAARQAWSV |
| | | VGTIKDLKQGYLSQVIHEIVDLMIHYQAVV |
| | | VLENLNFGFKSKRTGIAEKAVYQQFEKMLI |
| | | DKLNCLVLKDYPAEKVGGVLNPYQLTDQFT |
| | | SFAKMGTQSGFLFYVPAPYTSKIDPLTGFV |
| | | DPFVWKTIKNHESRKHFLEGFDFLHYDVKT |
| | | GDFILHFKMNRNLSFQRGLPGFMPAWDIVF |
| | | EKNETQFDAKGTPFIAGKRIVPVIENHRFT |
| | | GRYRDLYPANELIALLEEKGIVFRDGSNIL |
| | | PKLLENDDSHAIDTMVALIRSVLQMRNSNA |
| | | ATGEDYINSPVRDLNGVCFDSRFQNPEWPM |
| | | DADANGAYHIALKGQLLLNHLKESKDLKLQ |
| | | NGISNQDWLAYIQELRN |
| SEQ | Francisella | MSIYQEFVNKYSLSKTLRFELIPQGKTLEN |
| ID | novicida | IKARGLILDDEKRAKDYKKAKQIIDKYHQF |
| NO: | U112 | FIEEILSSVCISEDLLQNYSDVYFKLKKSD |
| 20 | (FnCas12a) | DDNLQKDFKSAKDTIKKQISEYIKDSEKFK |
| | | NLFNQNLIDAKKGQESDLILWLKQSKDNGI |
| | | ELFKANSDITDIDEALEIIKSFKGWTTYFK |
| | | GFHENRKNVYSSNDIPTSIIYRIVDDNLPK |
| | | FLENKAKYESLKDKAPEAINYEQIKKDLAE |
| | | ELTFDIDYKTSEVNQRVFSLDEVFEIANFN |
| | | NYLNQSGITKFNTIIGGKFVNGENTKRKGI |
| | | NEYINLYSQQINDKTLKKYKMSVLFKQILS |
| | | DTESKSFVIDKLEDDSDVVTTMQSFYEQIA |
| | | AFKTVEEKSIKETLSLLFDDLKAQKLDLSK |
| | | IYFKNDKSLTDLSQQVFDDYSVIGTAVLEY |
| | | ITQQIAPKNLDNPSKKEQELIAKKTEKAKY |
| | | LSLETIKLALEEFNKHRDIDKQCRFEEILA |
| | | NFAAIPMIFDEIAQNKDNLAQISIKYQNQG |
| | | KKDLLQASAEDDVKAIKDLLDQTNNLLHKL |
| | | KIFHISQSEDKANILDKDEHFYLVFEECYF |
| | | ELANIVPLYNKIRNYITQKPYSDEKFKLNF |
| | | ENSTLANGWDKNKEPDNTAILFIKDDKYYL |
| | | GVMNKKNNKIFDDKAIKENKGEGYKKIVYK |
| | | LLPGANKMLPKVFFSAKSIKFYNPSEDILR |
| | | IRNHSTHTKNGSPQKGYEKFEFNIEDCRKF |
| | | IDFYKQSISKHPEWKDFGFRFSDTQRYNSI |
| | | DEFYREVENQGYKLTFENISESYIDSVVNQ |
| | | GKLYLFQIYNKDFSAYSKGRPNLHTLYWKA |
| | | LFDERNLQDVVYKLNGEAELFYRKQSIPKK |
| | | ITHPAKEAIANKNKDNPKKESVFEYDLIKD |
| | | KRFTEDKFFFHCPITINFKSSGANKFNDEI |
| | | NLLLKEKANDVHILSIDRGERHLAYYTLVD |
| | | GKGNIIKQDTFNIIGNDRMKTNYHDKLAAI |
| | | EKDRDSARKDWKKINNIKEMKEGYLSQVVH |
| | | EIAKLVIEYNAIVVFEDLNFGFKRGRFKVE |
| | | KQVYQKLEKMLIEKLNYLVFKDNEFDKTGG |
| | | VLRAYQLTAPFETFKKMGKQTGIIYYVPAG |
| | | FTSKICPVTGFVNQLYPKYESVSKSQEFFS |
| | | KFDKICYNLDKGYFEFSFDYKNFGDKAAKG |
| | | KWTIASFGSRLINFRNSDKNHNWDTREVYP |
| | | TKELEKLLKDYSIEYGHGECIKAAICGESD |
| | | KKFFAKLTSVLNTILQMRNSKTGTELDYLI |
| | | SPVADVNGNFFDSRQAPKNMPQDADANGAY |
| | | HIGLKGLMLLGRIKNNQEGKKLNLVIKNEE |
| | | YFEFVQNRNN |
| SEQ | Porphyromonas | MKTQHFFEDFTSLYSLSKTIRFELKPIGKT |
| ID | macacae | LENIKKNGLIRRDEQRLDDYEKLKKVIDEY |
| NO: | (PmCas12a) | HEDFIANILSSFSFSEEILQSYIQNLSESE |
| 21 | | ARAKIEKTMRDTLAKAFSEDERYKSIFKKE |
| | | LVKKDIPVWCPAYKSLCKKFDNFTTSLVPF |
| | | HENRKNLYTSNEITASIPYRIVHVNLPKFI |
| | | QNIEALCELQKKMGADLYLEMMENLRNVWP |
| | | SFVKTPDDLCNLKTYNHLMVQSSISEYNRF |
| | | VGGYSTEDGTKHQGINEWINIYRQRNKEMR |
| | | LPGLVFLHKQILAKVDSSSFISDTLENDDQ |
| | | VFCVLRQFRKLFWNTVSSKEDDAASLKDLF |
| | | CGLSGYDPEAIYVSDAHLATISKNIFDRWN |
| | | YISDAIRRKTEVLMPRKKESVERYAEKISK |
| | | QIKKRQSYSLAELDDLLAHYSEESLPAGFS |
| | | LLSYFTSLGGQKYLVSDGEVILYEEGSNIW |
| | | DEVLIAFRDLQVILDKDFTEKKLGKDEEAV |
| | | SVIKKALDSALRLRKFFDLLSGTGAEIRRD |
| | | SSFYALYTDRMDKLKGLLKMYDKVRNYLTK |
| | | KPYSIEKFKLHFDNPSLLSGWDKNKELNNL |
| | | SVIFRQNGYYYLGIMTPKGKNLFKTLPKLG |
| | | AEEMFYEKMEYKQIAEPMLMLPKVFFPKKT |
| | | KPAFAPDQSVVDIYNKKTFKTGQKGFNKKD |
| | | LYRLIDFYKEALTVHEWKLFNFSFSPTEQY |
| | | RNIGEFFDEVREQAYKVSMVNVPASYIDEA |
| | | VENGKLYLFQIYNKDFSPYSKGIPNLHTLY |
| | | WKALFSEQNQSRVYKLCGGGELFYRKASLH |
| | | MQDTTVHPKGISIHKKNLNKKGETSLFNYD |
| | | LVKDKRFTEDKFFFHVPISINYKNKKITNV |
| | | NQMVRDYIAQNDDLQIIGIDRGERNLLYIS |
| | | RIDTRGNLLEQFSLNVIESDKGDLRTDYQK |
| | | ILGDREQERLRRRQEWKSIESIKDLKDGYM |
| | | SQVVHKICNMVVEHKAIVVLENLNLSFMKG |
| | | RKKVEKSVYEKFERMLVDKLNYLVVDKKNL |
| | | SNEPGGLYAAYQLTNPLFSFEELHRYPQSG |
| | | ILFFVDPWNTSLTDPSTGFVNLLGRINYTN |
| | | VGDARKFFDRFNAIRYDGKGNILFDLDLSR |
| | | FDVRVETQRKLWTLTTFGSRIAKSKKSGKW |
| | | MVERIENLSLCFLELFEQFNIGYRVEKDLK |
| | | KAILSQDRKEFYVRLIYLFNLMMQIRNSDG |
| | | EEDYILSPALNEKNLQFDSRLIEAKDLPVD |
| | | ADANGAYNVARKGLMVVQRIKRGDHESIHR |
| | | IGRAQWLRYVQEGIVE |
| SEQ | Moraxella | MLFQDFTHLYPLSKTVRFELKPIDRTLEHI |
| ID | bovoculi | HAKNFLSQDETMADMHQKVKVILDDYHRDF |
| NO: | 237 | IADMMGEVKLTKLAEFYDVYLKFRKNPKDD |
| 22 | (MbCas12a) | ELQKQLKDLQAVLRKEIVKPIGNGGKYKAG |
| | | YDRLFGAKLFKDGKELGDLAKFVIAQEGES |
| | | SPKLAHLAHFEKFSTYFTGFHDNRKNMYSD |
| | | EDKHTAIAYRLIHENLPRFIDNLQILTTIK |
| | | QKHSALYDQIINELTASGLDVSLASHLDGY |
| | | HKLLTQEGITAYNTLLGGISGEAGSPKIQG |
| | | INELINSHHNQHCHKSERIAKLRPLHKQIL |
| | | SDGMSVSFLPSKFADDSEMCQAVNEFYRHY |
| | | ADVFAKVQSLFDGFDDHQKDGIYVEHKNLN |
| | | ELSKQAFGDFALLGRVLDGYYVDVVNPEFN |
| | | ERFAKAKTDNAKAKLTKEKDKFIKGVHSLA |
| | | SLEQAIEHYTARHDDESVQAGKLGQYFKHG |
| | | LAGVDNPIQKIHNNHSTIKGFLERERPAGE |
| | | RALPKIKSGKNPEMTQLRQLKELLDNALNV |
| | | AHFAKLLTTKTTLDNQDGNFYGEFGVLYDE |
| | | LAKIPTLYNKVRDYLSQKPFSTEKYKLNFG |
| | | NPTLLNGWDLNKEKDNFGVILQKDGCYYLA |
| | | LLDKAHKKVFDNAPNTGKSIYQKMIYKYLE |
| | | VRKQFPKVFFSKEAIAINYHPSKELVEIKD |
| | | KGRQRSDDERLKLYRFILECLKIHPKYDKK |
| | | FEGAIGDIQLFKKDKKGREVPISEKDLFDK |
| | | INGIFSSKPKLEMEDFFIGEFKRYNPSQDL |
| | | VDQYNIYKKIDSNDNRKKENFYNNHPKFKK |
| | | DLVRYYYESMCKHEEWEESFEFSKKLQDIG |
| | | CYVDVNELFTEIETRRLNYKISFCNINADY |
| | | IDELVEQGQLYLFQIYNKDFSPKAHGKPNL |
| | | HTLYFKALFSEDNLADPIYKLNGEAQIFYR |
| | | KASLDMNETTIHRAGEVLENKNPDNPKKRQ |
| | | FVYDIIKDKRYTQDKFMLHVPITMNFGVQG |
| | | MTIKEFNKKVNQSIQQYDEVNVIGIDRGER |
| | | HLLYLTVINSKGEILEQCSLNDITTASANG |
| | | TQMTTPYHKILDKREIERLNARVGWGEIET |
| | | IKELKSGYLSHVVHQISQLMLKYNAIVVLE |
| | | DLNFGFKRGRFKVEKQIYQNFENALIKKLN |
| | | HLVLKDKADDEIGSYKNALQLTNNFTDLKS |
| | | IGKQTGFLFYVPAWNTSKIDPETGFVDLLK |
| | | PRYENIAQSQAFFGKFDKICYNADKDYFEF |
| | | HIDYAKFTDKAKNSRQIWTICSHGDKRYVY |
| | | DKTANQNKGAAKGINVNDELKSLFARHHIN |
| | | EKQPNLVMDICQNNDKEFHKSLMYLLKTLL |
| | | ALRYSNASSDEDFILSPVANDEGVFFNSAL |
| | | ADDTQPQNADANGAYHIALKGLWLLNELKN |
| | | SDDLNKVKLAIDNQTWLNFAQNR |
| SEQ | Moraxella | MGIHGVPAALFQDFTHLYPLSKTVRFELKP |
| ID | bovoculi | IGRTLEHIHAKNFLSQDETMADMYQKVKVI |
| NO: | AAX08_ | LDDYHRDFIADMMGEVKLTKLAEFYDVYLK |
| 23 | 00205 | FRKNPKDDGLQKQLKDLQAVLRKESVKPIG |
| | (Mb2Cas12a) | SGGKYKTGYDRLFGAKLFKDGKELGDLAKF |
| | | VIAQEGESSPKLAHLAHFEKFSTYFTGFHD |
| | | NRKNMYSDEDKHTAIAYRLIHENLPRFIDN |
| | | LQILTTIKQKHSALYDQIINELTASGLDVS |
| | | LASHLDGYHKLLTQEGITAYNRIIGEVNGY |
| | | TNKHNQICHKSERIAKLRPLHKQILSDGMG |
| | | VSFLPSKFADDSEMCQAVNEFYRHYTDVFA |
| | | KVQSLFDGFDDHQKDGIYVEHKNLNELSKQ |
| | | AFGDFALLGRVLDGYYVDVVNPEFNERFAK |
| | | AKTDNAKAKLTKEKDKFIKGVHSLASLEQA |
| | | IEHHTARHDDESVQAGKLGQYFKHGLAGVD |
| | | NPIQKIHNNHSTIKGFLERERPAGERALPK |
| | | IKSGKNPEMTQLRQLKELLDNALNVAHFAK |
| | | LLTTKTTLDNQDGNFYGEFGVLYDELAKIP |
| | | TLYNKVRDYLSQKPFSTEKYKLNFGNPTLL |
| | | NGWDLNKEKDNFGVILQKDGCYYLALLDKA |
| | | HKKVFDNAPNTGKNVYQKMVYKLLPGPNKM |
| | | LPKVFFAKSNLDYYNPSAELLDKYAKGTHK |
| | | KGDNFNLKDCHALIDFFKAGINKHPEWQHF |
| | | GFKFSPTSSYRDLSDFYREVEPQGYQVKFV |
| | | DINADYIDELVEQGKLYLFQIYNKDFSPKA |
| | | HGKPNLHTLYFKALFSEDNLADPIYKLNGE |
| | | AQIFYRKASLDMNETTIHRAGEVLENKNPD |
| | | NPKKRQFVYDIIKDKRYTQDKFMLHVPITM |
| | | NFGVQGMTIKEFNKKVNQSIQQYDEVNVIG |
| | | IDRGERHLLYLTVINSKGEILEQRSLNDIT |
| | | TASANGTQVTTPYHKILDKREIERLNARVG |
| | | WGEIETIKELKSGYLSHVVHQINQLMLKYN |
| | | AIVVLEDLNFGFKRGRFKVEKQIYQNFENA |
| | | LIKKLNHLVLKDKADDEIGSYKNALQLTNN |
| | | FTDLKSIGKQTGFLFYVPAWNTSKIDPETG |
| | | FVDLLKPRYENIAQSQAFFGKFDKICYNTD |
| | | KGYFEFHIDYAKFTDKAKNSRQKWAICSHG |
| | | DKRYVYDKTANQNKGAAKGINVNDELKSLF |
| | | ARYHINDKQPNLVMDICQNNDKEFHKSLMC |
| | | LLKTLLALRYSNASSDEDFILSPVANDEGV |
| | | FFNSALADDTQPQNADANGAYHIALKGLWL |
| | | LNELKNSDDLNKVKLAIDNQTWLNFAQNR |
| SEQ | Moraxella | MGIHGVPAALFQDFTHLYPLSKTVRFELKP |
| ID | bovoculi | IGKTLEHIHAKNFLNQDETMADMYQKVKAI |
| NO: | AAX11_ | LDDYHRDFIADMMGEVKLTKLAEFYDVYLK |
| 24 | 00205 | FRKNPKDDGLQKQLKDLQAVLRKEIVKPIG |
| | (Mb3Cas12a) | NGGKYKAGYDRLFGAKLFKDGKELGDLAKF |
| | | VIAQEGESSPKLAHLAHFEKFSTYFTGFHD |
| | | NRKNMYSDEDKHTAIAYRLIHENLPRFIDN |
| | | LQILATIKQKHSALYDQIINELTASGLDVS |
| | | LASHLDGYHKLLTQEGITAYNTLLGGISGE |
| | | AGSRKIQGINELINSHHNQHCHKSERIAKL |
| | | RPLHKQILSDGMGVSFLPSKFADDSEVCQA |
| | | VNEFYRHYADVFAKVQSLFDGFDDYQKDGI |
| | | YVEYKNLNELSKQAFGDFALLGRVLDGYYV |
| | | DVVNPEFNERFAKAKTDNAKAKLTKEKDKF |
| | | IKGVHSLASLEQAIEHYTARHDDESVQAGK |
| | | LGQYFKHGLAGVDNPIQKIHNNHSTIKGFL |
| | | ERERPAGERALPKIKSDKSPEIRQLKELLD |
| | | NALNVAHFAKLLTTKTTLHNQDGNFYGEFG |
| | | ALYDELAKIATLYNKVRDYLSQKPFSTEKY |
| | | KLNFGNPTLLNGWDLNKEKDNFGVILQKDG |
| | | CYYLALLDKAHKKVFDNAPNTGKSVYQKMI |
| | | YKLLPGPNKMLPKVFFAKSNLDYYNPSAEL |
| | | LDKYAQGTHKKGDNFNLKDCHALIDFFKAG |
| | | INKHPEWQHFGFKFSPTSSYQDLSDFYREV |
| | | EPQGYQVKFVDINADYINELVEQGQLYLFQ |
| | | IYNKDFSPKAHGKPNLHTLYFKALFSEDNL |
| | | VNPIYKLNGEAEIFYRKASLDMNETTIHRA |
| | | GEVLENKNPDNPKKRQFVYDIIKDKRYTQD |
| | | KFMLHVPITMNFGVQGMTIKEFNKKVNQSI |
| | | QQYDEVNVIGIDRGERHLLYLTVINSKGEI |
| | | LEQRSLNDITTASANGTQMTTPYHKILDKR |
| | | EIERLNARVGWGEIETIKELKSGYLSHVVH |
| | | QISQLMLKYNAIVVLEDLNFGFKRGRFKVE |
| | | KQIYQNFENALIKKLNHLVLKDKADDEIGS |
| | | YKNALQLTNNFTDLKSIGKQTGFLFYVPAW |
| | | NTSKIDPETGFVDLLKPRYENIAQSQAFFG |
| | | KFDKICYNADRGYFEFHIDYAKFNDKAKNS |
| | | RQIWKICSHGDKRYVYDKTANQNKGATIGV |
| | | NVNDELKSLFTRYHINDKQPNLVMDICQNN |
| | | DKEFHKSLMYLLKTLLALRYSNASSDEDFI |
| | | LSPVANDEGVFFNSALADDTQPQNADANGA |
| | | YHIALKGLWLLNELKNSDDLNKVKLAIDNQ |
| | | TWLNFAQNR |
| SEQ | Thiomicrospira | MGIHGVPAATKTFDSEFFNLYSLQKTVRFE |
| ID | sp. XS5 | LKPVGETASFVEDFKNEGLKRVVSEDERRA |
| NO: | (TsCas12a) | VDYQKVKEIIDDYHRDFIEESLNYFPEQVS |
| 25 | | KDALEQAFHLYQKLKAAKVEEREKALKEWE |
| | | ALQKKLREKVVKCFSDSNKARFSRIDKKEL |
| | | IKEDLINWLVAQNREDDIPTVETFNNFTTY |
| | | FTGFHENRKNIYSKDDHATAISFRLIHENL |
| | | PKFFDNVISFNKLKEGFPELKFDKVKEDLE |
| | | VDYDLKHAFEIEYFVNFVTQAGIDQYNYLL |
| | | GGKTLEDGTKKQGMNEQINLFKQQQTRDKA |
| | | RQIPKLIPLFKQILSERTESQSFIPKQFES |
| | | DQELFDSLQKLHNNCQDKFTVLQQAILGLA |
| | | EADLKKVFIKTSDLNALSNTIFGNYSVFSD |
| | | ALNLYKESLKTKKAQEAFEKLPAHSIHDLI |
| | | QYLEQFNSSLDAEKQQSTDTVLNYFIKTDE |
| | | LYSRFIKSTSEAFTQVQPLFELEALSSKRR |
| | | PPESEDEGAKGQEGFEQIKRIKAYLDTLME |
| | | AVHFAKPLYLVKGRKMIEGLDKDQSFYEAF |
| | | EMAYQELESLIIPIYNKARSYLSRKPFKAD |
| | | KFKINFDNNTLLSGWDANKETANASILFKK |
| | | DGLYYLGIMPKGKTFLFDYFVSSEDSEKLK |
| | | QRRQKTAEEALAQDGESYFEKIRYKLLPGA |
| | | SKMLPKVFFSNKNIGFYNPSDDILRIRNTA |
| | | SHTKNGTPQKGHSKVEFNLNDCHKMIDFFK |
| | | SSIQKHPEWGSFGFTFSDTSDFEDMSAFYR |
| | | EVENQGYVISFDKIKETYIQSQVEQGNLYL |
| | | FQIYNKDFSPYSKGKPNLHTLYWKALFEEA |
| | | NLNNVVAKLNGEAEIFFRRHSIKASDKVVH |
| | | PANQAIDNKNPHTEKTQSTFEYDLVKDKRY |
| | | TQDKFFFHVPISLNFKAQGVSKFNDKVNGF |
| | | LKGNPDVNIIGIDRGERHLLYFTVVNQKGE |
| | | ILVQESLNTLMSDKGHVNDYQQKLDKKEQE |
| | | RDAARKSWTTVENIKELKEGYLSHVVHKLA |
| | | HLIIKYNAIVCLEDLNFGFKRGRFKVEKQV |
| | | YQKFEKALIDKLNYLVFKEKELGEVGHYLT |
| | | AYQLTAPFESFKKLGKQSGILFYVPADYTS |
| | | KIDPTTGFVNFLDLRYQSVEKAKQLLSDFN |
| | | AIRFNSVQNYFEFEIDYKKLTPKRKVGTQS |
| | | KWVICTYGDVRYQNRRNQKGHWETEEVNVT |
| | | EKLKALFASDSKTTTVIDYANDDNLIDVIL |
| | | EQDKASFFKELLWLLKLTMTLRHSKIKSED |
| | | DFILSPVKNEQGEFYDSRKAGEVWPKDADA |
| | | NGAYHIALKGLWNLQQINQWEKGKTLNLAI |
| | | KNQDWFSFIQEKPYQE |
| SEQ | Butyrivibrio | MGIHGVPAAYYQNLTKKYPVSKTIRNELIP |
| ID | sp. NC3005 | IGKTLENIRKNNILESDVKRKQDYEHVKGI |
| NO: | (BsCas12a) | MDEYHKQLINEALDNYMLPSLNQAAEIYLK |
| 26 | | KHVDVEDREEFKKTQDLLRREVTGRLKEHE |
| | | NYTKIGKKDILDLLEKLPSISEEDYNALES |
| | | FRNFYTYFTSYNKVRENLYSDEEKSSTVAY |
| | | RLINENLPKFLDNIKSYAFVKAAGVLADCI |
| | | EEEEQDALFMVETFNMTLTQEGIDMYNYQI |
| | | GKVNSAINLYNQKNHKVEEFKKIPKMKVLY |
| | | KQILSDREEVFIGEFKDDETLLSSIGAYGN |
| | | VLMTYLKSEKINIFFDALRESEGKNVYVKN |
| | | DLSKTTMSNIVFGSWSAFDELLNQEYDLAN |
| | | ENKKKDDKYFEKRQKELKKNKSYTLEQMSN |
| | | LSKEDISPIENYIERISEDIEKICIYNGEF |
| | | EKIVVNEHDSSRKLSKNIKAVKVIKDYLDS |
| | | IKELEHDIKLINGSGQELEKNLVVYVGQEE |
| | | ALEQLRPVDSLYNLTRNYLTKKPFSTEKVK |
| | | LNFNKSTLLNGWDKNKETDNLGILFFKDGK |
| | | YYLGIMNTTANKAFVNPPAAKTENVFKKVD |
| | | YKLLPGSNKMLPKVFFAKSNIGYYNPSTEL |
| | | YSNYKKGTHKKGPSFSIDDCHNLIDFFKES |
| | | IKKHEDWSKFGFEFSDTADYRDISEFYREV |
| | | EKQGYKLTFTDIDESYINDLIEKNELYLFQ |
| | | IYNKDFSEYSKGKLNLHTLYFMMLFDQRNL |
| | | DNVVYKLNGEAEVFYRPASIAENELVIHKA |
| | | GEGIKNKNPNRAKVKETSTFSYDIVKDKRY |
| | | SKYKFTLHIPITMNFGVDEVRRFNDVINNA |
| | | LRTDDNVNVIGIDRGERNLLYVVVINSEGK |
| | | ILEQISLNSIINKEYDIETNYHALLDERED |
| | | DRNKARKDWNTIENIKELKTGYLSQVVNVV |
| | | AKLVLKYNAIICLEDLNFGFKRGRQKVEKQ |
| | | VYQKFEKMLIEKLNYLVIDKSREQVSPEKM |
| | | GGALNALQLTSKFKSFAELGKQSGIIYYVP |
| | | AYLTSKIDPTTGFVNLFYIKYENIEKAKQF |
| | | FDGFDFIRFNKKDDMFEFSFDYKSFTQKAC |
| | | GIRSKWIVYTNGERIIKYPNPEKNNLFDEK |
| | | VINVTDEIKGLFKQYRIPYENGEDIKEIII |
| | | SKAEADFYKRLFRLLHQTLQMRNSTSDGTR |
| | | DYIISPVKNDRGEFFCSEFSEGTMPKDADA |
| | | NGAYNIARKGLWVLEQIRQKDEGEKVNLSM |
| | | TNAEWLKYAQLHLL |
| SEQ | AacCas12b | MAVKSIKVKLRLDDMPEIRAGLWKLHKEVN |
| ID | | AGVRYYTEWLSLLRQENLYRRSPNGDGEQE |
| NO: | | CDKTAEECKAELLERLRARQVENGHRGPAG |
| 27 | | SDDELLQLARQLYELLVPQAIGAKGDAQQI |
| | | ARKFLSPLADKDAVGGLGIAKAGNKPRWVR |
| | | MREAGEPGWEEEKEKAETRKSADRTADVLR |
| | | ALADFGLKPLMRVYTDSEMSSVEWKPLRKG |
| | | QAVRTWDRDMFQQAIERMMSWESWNQRVGQ |
| | | EYAKLVEQKNRFEQKNFVGQEHLVHLVNQL |
| | | QQDMKEASPGLESKEQTAHYVTGRALRGSD |
| | | KVFEKWGKLAPDAPFDLYDAEIKNVQRRNT |
| | | RRFGSHDLFAKLAEPEYQALWREDASFLTR |
| | | YAVYNSILRKLNHAKMFATFTLPDATAHPI |
| | | WTRFDKLGGNLHQYTFLFNEFGERRHAIRF |
| | | HKLLKVENGVAREVDDVTVPISMSEQLDNL |
| | | LPRDPNEPIALYFRDYGAEQHFTGEFGGAK |
| | | IQCRRDQLAHMHRRRGARDVYLNVSVRVQS |
| | | QSEARGERRPPYAAVFRLVGDNHRAFVHFD |
| | | KLSDYLAEHPDDGKLGSEGLLSGLRVMSVD |
| | | LGLRTSASISVFRVARKDELKPNSKGRVPF |
| | | FFPIKGNDNLVAVHERSQLLKLPGETESKD |
| | | LRAIREERQRTLRQLRTQLAYLRLLVRCGS |
| | | EDVGRRERSWAKLIEQPVDAANHMTPDWRE |
| | | AFENELQKLKSLHGICSDKEWMDAVYESVR |
| | | RVWRHMGKQVRDWRKDVRSGERPKIRGYAK |
| | | DVVGGNSIEQIEYLERQYKFLKSWSFFGKV |
| | | SGQVIRAEKGSRFAITLREHIDHAKEDRLK |
| | | KLADRIIMEALGYVYALDERGKGKWVAKYP |
| | | PCQLILLEELSEYQFNNDRPPSENNQLMQW |
| | | SHRGVFQELINQAQVHDLLVGTMYAAFSSR |
| | | FDARTGAPGIRCRRVPARCTQEHNPEPFPW |
| | | WLNKFVVEHTLDACPLRADDLIPTGEGEIF |
| | | VSPFSAEEGDFHQIHADLNAAQNLQQRLWS |
| | | DFDISQIRLRCDWGEVDGELVLIPRLTGKR |
| | | TADSYSNKVFYTNTGVTYYERERGKKRRKV |
| | | FAQEKLSEEEAELLVEADEAREKSVVLMRD |
| | | PSGIINRGNWTRQKEFWSMVNQRIEGYLVK |
| | | QIRSRVPLQDSACENTGDI |
| SEQ | Cas12 | MKKIDNFVGCYPVSKTLRFKAIPIGKTQEN |
| ID | Variant | IEKKRLVEEDEVRAKDYKAVKKLIDRYHRE |
| NO: | | FIEGVLDNVKLDGLEEYYMLFNKSDREESD |
| 28 | | NKKIEIMEERFRRVISKSFKNNEEYKKIFS |
| | | KKIIEEILPNYIKDEEEKELVKGFKGFYTA |
| | | FVGYAQNRENMYSDEKKSTAISYRIVNENM |
| | | PRFITNIKVFEKAKSILDVDKINEINEYIL |
| | | NNDYYVDDFFNIDFFNYVLNQKGIDIYNAI |
| | | IGGIVTGDGRKIQGLNECINLYNQENKKIR |
| | | LPQFKPLYKQILSESESMSFYIDEIESDDM |
| | | LIDMLKESLQIDSTINNAIDDLKVLFNNIF |
| | | DYDLSGIFINNGLPITTISNDVYGQWSTIS |
| | | DGWNERYDVLSNAKDKESEKYFEKRRKEYK |
| | | KVKSFSISDLQELGGKDLSICKKINEIISE |
| | | MIDDYKSKIEEIQYLFDIKELEKPLVTDLN |
| | | KIELIKNSLDGLKRIERYVIPFLGTGKEQN |
| | | RDEVFYGYFIKCIDAIKEIDGVYNKTRNYL |
| | | TKKPYSKDKFKLYFENPQLMGGWDRNKESD |
| | | YRSTLLRKNGKYYVAIIDKSSSNCMMNIEE |
| | | DENDNYEKINYKLLPGPNKMLPKVFFSKKN |
| | | REYFAPSKEIERIYSTGTFKKDTNFVKKDC |
| | | ENLITFYKDSLDRHEDWSKSFDFSFKESSA |
| | | YRDISEFYRDVEKQGYRVSFDLLSSNAVNT |
| | | LVEEGKLYLFQLYNKDFSEKSHGIPNLHTM |
| | | YFRSLFDDNNKGNIRLNGGAEMFMRRASLN |
| | | KQDVTVHKANQPIKNKNLLNPKKTTTLPYD |
| | | VYKDKRFTEDQYEVHIPITMNKVPNNPYKI |
| | | NHMVREQLVKDDNPYVIGIDRGERNLIYVV |
| | | VVDGQGHIVEQLSLNEIINENNGISIRTDY |
| | | HTLLDAKERERDESRKQWKQIENIKELKEG |
| | | YISQVVHKICELVEKYDAVIALEDLNSGFK |
| | | NSRVKVEKQVYQKFEKMLITKLNYMVDKKK |
| | | DYNKPGGVLNGYQLTTQFESFSKMGTQNGI |
| | | MFYIPAWLTSKMDPTTGFVDLLKPKYKNKA |
| | | DAQKFFSQFDSIRYDNQEDAFVFKVNYTKF |
| | | PRTDADYNKEWEIYTNGERIRVFRNPKKNN |
| | | EYDYETVNVSERMKELFDSYDLLYDKGELK |
| | | ETICEMEESKFFEELIKLFRLTLQMRNSIS |
| | | GRTDVDYLISPVKNSNGYFYNSNDYKKEGA |
| | | KYPKDADANGAYNIARKVLWAIEQFKMADE |
| | | DKLDKTKISIKNQEWLEYAQTHCE |
| SEQ | Cas12 | MATLVSFTKQYQVQKTLRFELIPQGKTQAN |
| ID | Variant | IDAKGFINDDLKRDENYMKVKGVIDELHKN |
| NO: | | FIEQTLVNVDYDWRSLATAIKNYRKDRSDT |
| 29 | | NKKNLEEYDEETQDAIACFDKFTTYFVGFH |
| | | ENRKNMYSTEAKSTSVAYRVVNENKTQEAA |
| | | RKEIIAWFEGKRGNSAFKNNQKSFYGKLFK |
| | | KELFSEILRSDDLFSKFLSNCEAFSVLEAV |
| | | CPNVLVEAEQELHLHKAFSDLKLSDVFKVE |
| | | AYNKYLSQTGIDYYNQIIGGISSAEGVRKI |
| | | RGVNEVVNNAIQQNDELKVALRNKQFTMVQ |
| | | LFKQILSDRSTLSFVSEQFTSDQEVITVVK |
| | | QFNDDIVNNKVLAVVKTLFENFNSYDLEKI |
| | | YINSKELASVSNALLKDWSKIRNAVLENKI |
| | | IELGANPPKTKISAVEKEVKNKDFSIAELA |
| | | SYNDKYLDKEGNDKEICSIANVVLEAVGAL |
| | | EIMLAESLPADLKTLENKNKVKGILDAYEN |
| | | LLHLLNYFKVSAVNDVDLAFYGAFEKVYVD |
| | | ISGVMPLYNKVRNYATKKPYSVEKFKLNFA |
| | | MPTLADGWDKNKERDNGSIILLKDGQYYLG |
| | | VMNPQNKPVIDNAVCNDAKGYQKMVYKMFP |
| | | EISKMVTKCSTQLNAVKAHFEDNTNDFVLD |
| | | DTDKFISDLTITKEIYDLNNVLYDGKKKFQ |
| | | IDYLRNTGDFAGYHKALETWIDFVKEFLSK |
| | | YRSTAIYDLTTLLPTNYYEKLDVFYSDVNN |
| | | LCYKIDYENISVEQVNEWVEEGNLYLFKIY |
| | | NKDFATGSTGKPNLHTMYWNAVFAEENLHD |
| | | VVVKLNGGAELFYRPKSNMPKVEHRVGEKL |
| | | VNRKNVNGEPIADSVHKEIYAYANGKISKS |
| | | ELSENAQEELPLAIIKDVKHNITKDKRYLS |
| | | DKYFFHVPITLNYKANGNPSAFNTKVQAFL |
| | | KNNPDVNIIGIDRGERNLLYVVVIDQQGNI |
| | | IDKKQVSYNKVNGYDYYEKLNQREKERIEA |
| | | RQSWGAVGKIKELKEGYLSLVVREIADMMV |
| | | KYNAIVVMENLNAGFKRVRGGIAEKAVYQK |
| | | FEKMLIDKLNYLVFKDVEAKEAGGVLNAYQ |
| | | LTDKFDSFEKMGNQSGFLFYVPAAYTSKID |
| | | PVTGFANVFSTKHITNTEAKKEFICSFNSL |
| | | RYDEAKDKFVLECDLNKFKIVANSHIKNWK |
| | | FIIGGKRIVYNSKNKTYMEKYPCEDLKATL |
| | | NASGIDFSSSEIINLLKNVPANREYGKLFD |
| | | ETYWAIMNTLQMRNSNALTGEDYIISAVAD |
| | | DNEKVFDSRTCGAELPKDADANGAYHIALK |
| | | GLYLLQRIDISEEGEKVDLSIKNEEWFKFV |
| | | QQKEYAR |
| SEQ | Cas12 | MKEQFINRYPLSKTLRFSLIPVGETENNFN |
| ID | Variant | KNLLLKKDKQRAENYEKVKCYIDRFHKEYI |
| NO: | | ESVLSKARIEKVNEYANLYWKSNKDDSDIK |
| 30 | | AMESLENDDETITKLNTDLYNIFGRNIEDI |
| | | FSVDYFEFVLTQSGIEIYNSMIGGYTCSDM |
| | | RKQISKQLTSTEIYKKRLFGKELICEDLPS |
| | | FLTDKDERETVECFRSFTTYFKGFNTNREN |
| | | MYSSDGKSTAIAYRCINDNLPRFLDNVKSF |
| | | QKVFDNLSKTKIQGLNECINLYNQQVAKNE |
| | | KSKKLPLMKPLYKQILSEKDSVSFIPEKFN |
| | | SDNEVLHAIDDYYTGHIGDFDLLTELLQSL |
| | | NTYNANGIFVKSGVAITDISNGAFNSWNVL |
| | | RSAWNEKYEALHPVTSKTKIDKYIEKQDKI |
| | | YKAIKSFSLFELQSLGNENGNEITDWYISS |
| | | INESNSKIKEAYLQAQKLLNSDYEKSYNKR |
| | | LYKNEKATELVKNLLDAIKEFQKLIKPLNG |
| | | TGKEENKDELFYGKFTSYYDSIADIDRLYD |
| | | KVRNYITQKPYSKDKIKLNFDNPQLLGGWD |
| | | KNKESDYRTVLLHKDGLYYLAVMDKSHSKA |
| | | FVDAPEITSDDKDYYEKMEYKLLPGPNKML |
| | | PKVFFASKNIDTFQPSDRILDIRKRESFKK |
| | | GATFNKAECHEFIDYFKDSIKKHDDWSQFG |
| | | FKFSPTESYNDISEFYREISDQGYSVRFNK |
| | | ISKNYIDGLVNNGYIYLFQIYNKDFSKYSK |
| | | GTPNLHTLYFKMLFDERNLSNVVYKLNGEA |
| | | EMFYREASIGDKEKITHYANQPIKNKNPDN |
| | | EKKESVFEYDIVKDKRFTKRQFSLHLPITI |
| | | NFKAHGQEFLNYDVRKAVKYKDDNYVIGID |
| | | RGERNLIYISVINSNGEIVEQMSLNEIISD |
| | | NGHKVDYQKLLDTKEKERDKARKNWTSVEN |
| | | IKELKEGYISQVVHKICELVIKYDAVIAME |
| | | DLNFGFKRGRFPVEKQVYQKFENMLISKLN |
| | | LLIDKKAEPTEDGGLLRAYQLTNKFDGVNK |
| | | AKQNGIIFYVPAWDTSKIDPATGFVNLLKP |
| | | KCNTSVPEAKKLFETIDDIKYNANTDMFEF |
| | | YIDYSKFPRCNSDFKKSWTVCTNSSRILTF |
| | | RNKEKNNKWDNKQIVLTDEFKSLFNEFGID |
| | | YKGNLKDSILSISNADFYRRLIKLLSLTLQ |
| | | MRNSITGSTLPEDDYLISPVANKSGEFYDS |
| | | RNYKGTNAALPCDADANGAYNIARKALWAI |
| | | NVLKDTPDDMLNKAKLSITNAEWLEYTQK |
| SEQ | Cas12 | MNNPRGAFGGFTNLYSLSKTLRFELKPYLE |
| ID | Variant | IPEGEKGKLFGDDKEYYKNCKTYTEYYLKK |
| NO: | | ANKEYYDNEKVKNTDLQLVNFLHDERIEDA |
| 31 | | YQVLKPLETERGKLRKAFTPIYEAEGKNLK |
| | | NKAGKEKKDKDILKESGFKVLIEAPVFDTL |
| | | HEEFITDSLESAEAKKIDFGNYYGLYEKQK |
| | | SEQNKDEKKKIDKGILKYIKNNIDEFADKK |
| | | LKNNEGKEITKKDIETALGAENIEGIFDGF |
| | | FTYFSGFNQNRENYYSTEEKATAVASRIVD |
| | | ENLSKFCDNILLYRKNENDYLKIFNFLKNK |
| | | GKDLKLKNSKFGKENEPEFIPAYDMKNDEK |
| | | SFSVADFVNCLSQGEIEKYNAKIANANYLI |
| | | NLYNQNKDGNSSKLSMFKILYKQIGCGEKK |
| | | DFIKTIKDNAELKQILEKACEAGKKYFIRG |
| | | KSEDGGVSNIFDFTDYIQSHENYKGVYWSD |
| | | KAINTISGKYFANWDTLKNKLGDAKVFNKN |
| | | TGEDKADVKYKVPQAVMLSELFAVLDDNAG |
| | | EDWREKGIFFKASLFEGDQNKSEIIKNANR |
| | | PSQALLKMICDDMESLAKNFIDSGDKILKI |
| | | SDRDYQKDENKQKIKNWLDNALWINQILKY |
| | | FKVKANKIKGDSIDARIDSGLDMLVFSSDN |
| | | PAEDYDMIRNYLTQKPQDEINKLKLNFENS |
| | | SLAGGWDENKEKDNSCIILKDEQDKQYLAV |
| | | MKYENTKVFEQKNSQLYIADNAAWKKMIYK |
| | | LVPGASKTLPKVFFSKKWTANRPTPSDIVE |
| | | IYQKGSFKKENVDFNDKKEKDESRKEKNRE |
| | | KIIAELQKTCWMDIRYNIDGKIESAKYVNK |
| | | EKLAKLIDFYKENLKKYPSEEESWDRLFAF |
| | | GFSDTKSYKSIDQFYIEVDKQGYKLEFVTI |
| | | NKARLDEYVRDGKIYLFEIRSRDNNLVNGE |
| | | EKTSAKNLQTIYWNAAFGGDDNKPKLNGEA |
| | | EIFYRPAIAENKLNKKKDKNGKEIIDGYRF |
| | | SKEKFIFHCPITLNFCLKETKINDKLNAAL |
| | | AKPENGQGVYFLGIDRGEKHLAYYSLVNQK |
| | | GEILEQGTLNLPFLDKNGKSRSIKVEKKSF |
| | | EKDSNGGIIKDKDGNDKIKIEFVECWNYND |
| | | LLDARAGDRDYARKNWTTIGTIKELKDGYI |
| | | SQVVRKIVDLSIYKNTETKEFREMPAFIVL |
| | | EDLNIGFKRGRQKIEKQVYQKLELALAKKL |
| | | NFLVDKKADIGEIGSVTKAIQLTPPVNNFG |
| | | DMENRKQFGNMLYIRADYTSQTDPATGWRK |
| | | SIYLKSGSESNVKEQIEKSFFDIRYESGDY |
| | | CFEYRDRHGKMWQLYSSKNGVSLDRFHGER |
| | | NNSKNVWESEKQPLNEMLDILFDEKRFDKS |
| | | KSLYEQMFKGGVALTRLPKEINKKDKPAWE |
| | | SLRFVIILIQQIRNTGKNGDDRNGDFIQSP |
| | | VRDEKTGEHFDSRIYLDKEQKGEKADLPTS |
| | | GDANGAYNIARKGIVVAEHIKRGFDKLYIS |
| | | DEEWDTWLAGDEIWDKWLKENRESLTKTRK |
| SEQ | Cas12 | MNGNRIIVYREFVGVTPVAKTLRNELRPIG |
| ID | Variant | HTQEHIIHNGLIQEDELRQEKSTELKNIMD |
| NO: | | DYYREYIDKSLSGVTDLDFTLLFELMNLVQ |
| 32 | | SSPSKDNKKDKAGKLETLALFNGFSTYFTD |
| | | FFEKRKNVFTKEAVSTSIAYRIVHENSLTF |
| | | LANMTSYKKISEKALDEIEVIEKNNQDKMG |
| | | DWELNQIFNPDFYNMVLIALEKEQSKMREQ |
| | | ICTHMQSDSNYKNIFNAKFLKEILPDFIKN |
| | | YNQYDAKQSGIDFYNEICGVVNAHMNLYCQ |
| | | QTKNNYNLFKMRKLHKQILAYTSTSFEVPK |
| | | MFEDDMSVYNAVNAFIDETEKGNIIGKLKD |
| | | IVNKYDELDEKRIYISKDFYETLSCFMSGN |
| | | WNLITGCVENFYDENIHAKGKSKEEKVKKA |
| | | VKEDKYKSINDVNDLVEKYIDEKERNEFKN |
| | | SNAKQYIREISNIITDTETAHLEYDEHISL |
| | | IESEEKADEMKKRLDMYMNMYHWAKAFIVD |
| | | EVLDRDEMFYSDIDDIYNILENIVPLYNRV |
| | | RNYVTQKPYNSKKIKLNFQSPTLANGWSQS |
| | | KEFDNNAIILIRDNKYYLAIFNAKNKPDKK |
| | | IIQGNSDKKNDNDYKKMVYNLLPGANKMLP |
| | | KVFLSKKGIETFKPSDYIISGYNAHKHIKT |
| | | SENFDISFCRDLIDYFKNSIEKHAEWRKYE |
| | | FKFSATDSYNDISEFYREVEMQGYRIDWTY |
| | | ISEADINKLDEEGKIYLFQIYNKDFAENST |
| | | GKENLHTMYFKNIFSEENLKDIIIKLNGQA |
| | | ELFYRRASVKNPVKHKKDSVLVNKTYKNQL |
| | | DNGDVVRIPIPDDIYNEIYKMYNGYIKEND |
| | | LSEAAKEYLDKVEVRTAQKDIVKDYRYTVD |
| | | KYFIHTPITINYKVTARNNVNDMAVKYIAQ |
| | | NDDIHVIGIDRGERNLIYISVIDSHGNIVK |
| | | QKSYNILNNYDYKKKLVEKEKTREYARKNW |
| | | KSIGNIKELKEGYISGVVHEIAMLMVEYNA |
| | | IIAMEDLNYGFKRGRFKVERQVYQKFESML |
| | | INKLNYFASKGKSVDEPGGLLKGYQLTYVP |
| | | DNIKNLGKQCGVIFYVPAAFTSKIDPSTGF |
| | | ISAFNFKSISTNASRKQFFMQFDEIRYCAE |
| | | KDMFSFGFDYNNFDTYNITMSKTQWTVYTN |
| | | GERLQSEFNNARRTGKTKSINLTETIKLLL |
| | | EDNEINYADGHDVRIDMEKMDEDKNSEFFA |
| | | QLLSLYKLTVQMRNSYTEAEEQEKGISYDK |
| | | IISPVINDEGEFFDSDNYKESDDKECKMPK |
| | | DADANGAYCIALKGLYEVLKIKSEWTEDGF |
| | | DRNCLKLPHAEWLDFIQNKRYE |
| SEQ | Cas12 | MKKIDSFVNYYPLSKTLRFSLIPVGKTEDN |
| ID | Variant | FNAKLLLEEDEKRAIEYEKVKRYIDRYHKH |
| NO: | | FIETVLANFHLDDVNEYAELYYKAGKDDKD |
| 33 | | LKYMEKLEGKMRKSISAAFTKDKKYKEIFG |
| | | QEIIKNILPEFLENEDEKESVKMFQGFFTY |
| | | FTGFNDNRKNMYTHEAQTTAISYRCINENL |
| | | PKFLDNVQSFAKIKESISSDIMNKLDEVCM |
| | | DLYGVYAQDMFCTDYFSFVLSQSGIDRYNN |
| | | IIGGYVDDKGVKIQGINEYINLYNQQVDEK |
| | | NKRLPLMKKLYKQILIEKESISFIPEKFES |
| | | DNIVINAISDYYHNNVENLFDDFNKLFNEF |
| | | SEYDDNGIFVTSGLAVTDISNAVFGSWNII |
| | | SDSWNEEYKDSHPMKKTTNAEKYYEDMKKE |
| | | YKKNLSFTIAELQRLGEAGCNDECKGDIKE |
| | | YYKTTVAEKIENIKNAYEISKDLLASDYEK |
| | | SNDKKLCKNDSAISLLKNLLDSIKDLEKTI |
| | | KPLLGTGKEENKDDVFYGKFTNLYEMISEI |
| | | DRLYDKVRNYVTQKPYSKDKIKLNFENPQH |
| | | LGGWDKNKERDYRSVLLKKEDKYYLAIMDK |
| | | SNNKAFIDFPDDGECYEKIEYKLLPGPNKM |
| | | LPKVFFASSNIEYFAPSKKILEIRSRESFK |
| | | KGDMFNLKDCHEFIDFFKESIKKHEDWSQF |
| | | GFEFSPTEKYNDISEFYNEVKIQGYSLKYK |
| | | NVSKKYIDELIECGQLYLFQIYNKDFSVYA |
| | | KGNPNLHTMYFKMLFDERNLANVVYQLNGG |
| | | AEMFYRKASIKDSEKIVHHANQPIKNKNAD |
| | | NVKKESVFEYDIIKDKRFTKRQFSIHIPIT |
| | | LNFKAKGQNFINNDVRMALKKADENYVIGI |
| | | DRGERNLLYICVINSKGEIVEQKSLNEIIG |
| | | DNGYRVDYHKLLDKKEAERDEARKSWGTIE |
| | | NIKELKEGYLSQIVHEISKLVIKYDAVIAI |
| | | EDLNSGFKKGRFKVEKQVYQKFENMLCTKL |
| | | NYLVDKNADANECGGLLKAYQLTNKEDGAN |
| | | RGRQNGIIFSVPAWLTSKIDPVTGFADLLR |
| | | PKYKSVSESVEFISKIDNIRYNSKEDYFEF |
| | | DIDYSKFPNSTASYKKKWTVCTYGERIINV |
| | | RNKEKNNMWDNKTIVLTDEFKKLFADFGVD |
| | | VSKNIKESVLAIDSKDFYYRFINLLANTLQ |
| | | LRNSEVGNVDVDYLISPVKGVDGSFYDSRL |
| | | VKEKTLPENADANGAYNIARKALWAIDVLK |
| | | QTKDEELKNANLSIKNAEWLEYVQK |
| SEQ | Cas12 | MRTMVTFEDFTKQYQVSKTLRFELIPQGKT |
| ID | Variant | LENMKRDGIISVDRQRNEDYQKAKGILDKL |
| NO: | | YKYILDFTMETVVIDWEALATATEEFRKSK |
| 34 | | DKKTYEKVQSKIRTALLEHVKKQKVGTEDL |
| | | FKGMFSSKIITGEVLAAFPEIRLSDEENLI |
| | | LEKFKDFTTYFTGFFENRKNVFTDEALSTS |
| | | FTYRLVNDNFIKFFDNCIVFKNVVNISPHM |
| | | AKSLETCASDLGIFPGVSLEEVFSISFYNR |
| | | LLTQTGIDQFNQLLGGISGKEGEHKQQGLN |
| | | EIINLAMQQNLEVKEVLKNKAHRFTPLFKQ |
| | | ILSDRSTMSFIPDAFADDDEVLSAVDAYRK |
| | | YLSEKNIGDRAFQLISDMEAYSPELMRIGG |
| | | KYVSVLSQLLFYSWSEIRDGVKAYKESLIT |
| | | GKKTKKELENIDKEIKYGVTLQEIKEALPK |
| | | KDIYEEVKKYAMSVVKDYHAGLAEPLPEKI |
| | | ETDDERASIKHIMDSMLGLYRFLEYFSHDS |
| | | IEDTDPVFGECLDTILDDMNETVPLYNKVR |
| | | NFSTRKVYSTEKFKLNFNNSSLANGWDKNK |
| | | EQANGAILLRKEGEYFLGIFNSKNKPKLVS |
| | | DGGAGIGYEKMIYKQFPDFKKMLPKCTISL |
| | | KDTKAHFQKSDEDFTLQTDKFEKSIVITKQ |
| | | IYDLGTQTVNGKKKFQVDYPRLTGDMEGYR |
| | | AALKEWIDFGKEFIQAYTSTAIYDTSLFRD |
| | | SSDYPDLPSFYKDVDNICYKLTFEWIPDAV |
| | | IDDCIDDGSLYLFKLHNKDFSSGSIGKPNL |
| | | HTLYWKALFEEENLSDVVVKLNGQAELFYR |
| | | PKSLTRPVVHEEGEVIINKTTSTGLPVPDD |
| | | VYVELSKFVRNGKKGNLTDKAKNWLDKVTV |
| | | RKMPHAITKDRRFTVDKFFFHVPITLNYKA |
| | | DSSPYRFNDFVRQYIKDCSDVKIIGIDRGE |
| | | RNLIYAVVIDGKGNIIEQRSFNTVGTYNYQ |
| | | EKLEQKEKERQTARQDWATVTKIKDLKKGY |
| | | LSAVVHELSKMIVKYKAIVALENLNVGFKR |
| | | MRGGIAERSVYQQFEKALIDKLNYLVFKDE |
| | | EQSGYGGVLNAYQLTDKFESFSKMGQQTGF |
| | | LFYVPAAYTSKIDPLTGFINPFSWKHVKNR |
| | | EDRRNFLNLFSKLYYDVNTHDFVLAYHHSN |
| | | KDSKYTIKGNWEIADWDILIQENKEVFGKT |
| | | GTPYCVGKRIVYMDDSTTGHNRMCAYYPHT |
| | | ELKKLLSEYGIEYTSGQDLLKIIQEFDDDK |
| | | LVKGLFYIIKAALQMRNSNSETGEDYISSP |
| | | IEGRPGICFDSRAEADTLPYDADANGAFHI |
| | | AMKGLLLTERIRNDDKLAISNEEWLNYIQE |
| | | MRG |
| SEQ | Cas12 | MNKDIRKNFTDFVGISEIQKTLRFILIPIG |
| ID | Variant | KTAQNIDKYNMFEDDEIRHEYYPILKEACD |
| NO: | | DFYRNHIDQQFENLELDWSKLDEALASEDR |
| 35 | | DLINETRATYNIFTDDEISTGSPYRLVNDN |
| | | FTIFRINNSIYTKNKPFIEEDILEFEKKLK |
| | | SKRQVLFNRLKNSVDIKGDSKKNKTLSLES |
| | | SDKNLGKKKTKNTFQYNFNDLFKAKLIKAI |
| | | LPLYIEYIYEGEKLENAKKALKMYNRFTSR |
| | | LSNFWQARAKIIKDFESVDDYFTVNAFNKL |
| | | CTQNGIDKYNSILGGFTTKEREKVKGLNEL |
| | | FNLAQQSINKGKKGEYRKNIRLGKLTKLKK |
| | | QILAISDSTSFLIEQIEDDQDLYNKIKDFF |
| | | ELLLKEEIENENIFTQYANLQKLIEQADLS |
| | | KIYINAKHLNKISHQVTGKWDSLNKGIALL |
| | | LENININEESKEKSEVISNGQTKDISSEAY |
| | | KRYLQIQSEEKDIERLRTQIYFSLEDLEKA |
| | | LDLVLIDENMDRSDKSILSYVQSPDLNVNF |
| | | ERDLTDLYSRIMKLEENNEKLLANHSAIDL |
| | | IKEFLDLIMLRYSRWQILFCDSNYELDQTF |
| | | YPIYDAVMEILSNIIRLYNLARNYLSRKPD |
| | | RMKKKKINFNNPTLADGWSESKIPDNSSML |
| | | FIKDGMYYLGIIKNRAAYSELLEAESLQSS |
| | | EKKKSENSSYERMNYHFLPDAFRSIPKSSI |
| | | AMKAVKEHFEINQKTADLLLDTDKFSKPLR |
| | | ITKEIFDMQYVDLHKNKKKYQVDYLRDTGD |
| | | KKGYRKALNTWLNFCKDFISKYKGRNLFDY |
| | | SKIKDADHYETVNEFYNDVDKYSYHIFFTS |
| | | VAETTVEKFISEGKLYLFQLYNKDFSPHST |
| | | GKPNLHTIYWRALFSEENLTSKNIKLNGQA |
| | | EIFFRPKQIETPFTHKKGSILVNRFDVNGN |
| | | PIPINVYQEIKGFKNNVIKWDDLNKTTQEG |
| | | LENDQYLYFESEFEIIKDRRYTEDQLFFHV |
| | | PISFNWDIGSNPKINDLATQYIVNSNDIHI |
| | | IGIDRGENHLIYYSVIDLQGAIVEQGSLNT |
| | | ITEYTENKFLNNKTNNLRKIPYKDILQQRE |
| | | DERADARIKWHAIDKIKDLKDGYLGQIVHF |
| | | LAKLIIKYNAIVILEDLNYGFKRGRFKVER |
| | | QVYQKFEMALMKKLNVLVFKDYDIDEIGGP |
| | | LKPWQLTRPIDSYERMGRQNGILFYVPAAY |
| | | TSAVDPVTGFANLFYLNNVKNSEKFHFFSK |
| | | FESIKYHSDQDMFSFAFDYNNFGTTTRIND |
| | | LSKSKWQVFTNHERSVWNNKEKNYVTQNLT |
| | | DLIKKLLRTYNIEFKNNQNVLDSILKIENN |
| | | TDKENFARELFRLFRLTIQLRNTTVNENNT |
| | | EITENELDYIISPVKDKNGNFFDSRDELKN |
| | | LPDNGDANGAYNIARKGLLYIEQLQESIKT |
| | | GKLPTLSISTLDWFNYIMK |
| SEQ | Cas12 | MTPIFCNFVVYQIMLFNNNININVKTMNKK |
| ID | Variant | HLSDFTNLFPVSKTLRFRLEPQGKTMENIV |
| NO: | | KAQTIETDEERSHDYEKTKEYIDDYHRQFI |
| 36 | | DDTLDKFAPQFNLLFKKEMVKHLLPQFVDT |
| | | DDKKRIVAKFNDFTTYFTGFFTNRENFKVE |
| | | STGNNDSLQDYLDAYLSANDNRTKQTEEIQ |
| | | TNLRKAIVSAFKMQMYSDEAKSTSIAYRIV |
| | | NQNLIKFVENMLTFKSHILPILPQEQLATL |
| | | YDDFKEYLNVASIAEMFELDHFSIVLTQRQ |
| | | IEVYNSVIGGRKDENNKQIKPGLNQYINQH |
| | | NQAVKDKSARLPLLKPLFNQILSEKAGVSF |
| | | LPKQFKSASEVVKSLNEAYAELSPVLAAIQ |
| | | DVVTNITDYDCNGIFIKNDLGLTDIAQRFY |
| | | GNYDAVKRGLRNQYELETPMHNGQKAEKYE |
| | | EQVAKHLKSIESVSLAQINQVVTDGGDICD |
| | | YFKAFGATDDGDIQRENLLASINNAHTAIS |
| | | PVLNKENANDNELRKNTMLIKDLLDAIKRL |
| | | QWFAKPLLGAGDETNKDQVFYGKFEPLYNQ |
| | | LDETISPLYDKVRSYLTKKPYSLDKFKINF |
| | | EKSNLLGGWDPGADRKYQYNAVILRKDNDF |
| | | YLGIMRDEATSKRKCIQVLDCNDEGLDENF |
| | | EKVEYKQIKPSQNMPRCAFAKKECEENADI |
| | | MELKRKKNAKSYNTNKDDKNALIRHYQRYL |
| | | DRTYPEFGFVYKDADEYDTVKAFTDSMDSQ |
| | | DYKLSFLQVSETGLNKLVDEGDLYLFKITN |
| | | KDFSSYAKGRPNLHTIYWRMLFDPKNLANV |
| | | VYKLEGKAEVFFRRKSLASTTTHKAKQAIK |
| | | NKSRYNEAVKPQSTFDYDIIKDRRFTADKF |
| | | EFHVPIKMNFKAAGWNSTRLTNEVREFIKS |
| | | QGVRHIIGIDRGERHLLYLTMIDMDGNIVK |
| | | QCSLNAPAQDNARASEVDYHQLLDSKEADR |
| | | LAARRNWGTIENIKELKQGYLSQVVHLLAT |
| | | MMVDNDAILVLENLNAGFMRGRQKVEKSVY |
| | | QKFEKMLIDKLNYIVDKGQSPDKPTGALHA |
| | | VQLTGLYSDFNKSNMKRANVRQCGFVFYIP |
| | | AWNTSKIDPVTGFVNLFDTHLSSMGEIKAF |
| | | FSKFDSIRYNQDKGWFEFKFDYSRFTTRAE |
| | | GCRTQWTVCTYGERIWTHRSKNQNNQFVND |
| | | TVNVTQQMLQLLQDCGIDPNGNLKEAIANI |
| | | DSKKSLETLLHLFKLTVQMRNSVTGSEVDY |
| | | MISPVADERGHFFDSRESDEHLPANADANG |
| | | AFNIARKGLMVVRQIMATDDVSKIKFAVSN |
| | | KDWLRFAQHIDD |
| SEQ | Cas12 | MNKGGYVIMEKMTEKNRWENQFRITKTIKE |
| ID | Variant | EIIPTGYTKVNLQRVNMLKREMERNEDLKK |
| NO: | | MKEICDEYYRNMIDVSLRLEQVRTLGWESL |
| 37 | | IHKYRMLNKDEKEIKALEKEQEDLRKKISK |
| | | GFGEKKAWTGEQFIKKILPQYLMDHYTGEE |
| | | LEEKLRIVKKFKGCTMFLSTFFKNRENIFT |
| | | DKPIHTAVGHRITSENAMLFAANINTYEKM |
| | | ESNVTLEIERLQREFWRRGINISEIFTDAY |
| | | YVNVLTQKQIEAYNKICGDINQHMNEYCQK |
| | | QKLKFSEFRMRELKKQILAVVGEHFEIPEK |
| | | IESTKEVYRELNEYYESLKELHGQFEELKS |
| | | VQLKYSQIYVQKKGYDRISRYIGGQWDLIQ |
| | | ECMKKDCASGMKGTKKNHDAKIEEEVAKVK |
| | | YQSIEHIQKLVCTYEEDRGHKVTDYVDEFI |
| | | VSVCDLLGADHIITRDGERIELPLQYEPGT |
| | | DLLKNDTINQRRLSDIKTILDWHMDMLEWL |
| | | KTFLVNDLVIKDEEFYMAIEELNERMQCVI |
| | | SVYNRIRNYVTQKGYEPEKIRICFDKGTIL |
| | | TGWTTGDNYQYSGFLLMRNDKYYLGIINTN |
| | | EKSVRKILDGNEECKDENDYIRVGYHLIND |
| | | ASKQLPRIFVMPKAGKKSEILMKDEQCDYI |
| | | WDGYCHNKHNESKEFMRELIDYYKRSIMNY |
| | | DKWEGYCFKFSSTESYDNMQDFYKEVREQS |
| | | YNISFSYINENVLEQLDKDGKIYLFQVYNK |
| | | DFAAGSTGTPNLHTMYLQNLFSSQNLELKR |
| | | LRLGGNAELFYRPGTEKDVTHRKGSILVDR |
| | | TYVREEKDGIEVRDTVPEKEYLEIYRYLNG |
| | | KQKGDLSESAKQWLDKVHYREAPCDIIKDK |
| | | RYAQEKYFLHFSVEINPNAEGQTALNDNVR |
| | | RWLSEEEDIHVIGIDRGERNLIYVSLMDGK |
| | | GRIKDQKSYNIVNSGNKEPVDYLAKLKVRE |
| | | KERDEARRNWKAIGKIKDIKTGYLSYVVHE |
| | | IVEMAVREKAIIVMEDLNYGFKRGRFKVER |
| | | QVYQKFEEMLINKLNYVVDKQLSVDEPGGL |
| | | LRGYQLAFIPKDKKSSMRQNGIVFYVPAGY |
| | | TSKIDPTTGFVNIFKFPQFGKGDDDGNGKD |
| | | YDKIRAFFGKFDEIRYECDEKVTADNTREV |
| | | KERYRFDFDYSKFETHLVHMKKTKWTVYAE |
| | | GERIKRKKVGNYWTSEVISDIALRMSNTLN |
| | | IAGIEYKDGHNLVNEICALRGKQAGIILNE |
| | | LLEIVRLTVQLRNSTTEGDVDERDEIISPV |
| | | LNEKYGCFYHSTEYKQQNGDVLPKDADANG |
| | | AYCIGLKGIYEIRQIKNKWKEDMTKGEGKA |
| | | LNEGMRISHDQWFEFIQNMNKGE |
| SEQ | Cas12 | MNELVKNRCKQTKTICQKLIPIGKTRETIE |
| ID | Variant | KYNLMEIDRKIAANKELMNKLFSLIAGKHI |
| NO: | | NDTLSKCTDLDFEPLLTSLSSLNNAKENDR |
| 38 | | DNLREYYDSVFEEKKTLAEEISSRLTAVKF |
| | | AGKDFFTKNIPDFLETYEGDDKNEMSELVS |
| | | LVIENTVTAGYVKKLEKIDRSMEYRLVSGT |
| | | VVKRVLTDNADIYEKNIEKAKDFDYGVLNI |
| | | DEASQFTTLVAKDYANYLTADGIAIYNVGI |
| | | GKINLALNEYCQKNKEYSYNKLALLPLQKM |
| | | LYGEKLSLFEKLEDFTSDEELINSYNKFAK |
| | | TVNESGLAEIIKKAVPSYDEIVIKPNKISN |
| | | YSNSITGHWSLVNRIMKDYLENNGIKNADK |
| | | YMEKGLTLSEIGDALENKNIKHSDFISNLI |
| | | NDLGHTYTEIKENKESLKKDESVNALIIKK |
| | | ELDMLLSILQNLKVFDIDNEMFDTGFGIEV |
| | | SKAIEILGYGVPLYNKIRNYITKKPDPKKK |
| | | FMTKFGSATIGTGITTSVEGSKKATFLKDG |
| | | DAVFLLLYNTAGCKANNVSVSNLADLINSS |
| | | LEIENSGKCYQKMIYQTPGDIKKQIPRVFV |
| | | YKSEDDDLIKDFKAGLHKTDLSFLNGRLIP |
| | | YLKEAFATHETYKNYTFSYRNSYESYDEFC |
| | | EHMSEQAYILEWKWIDKKLIDDLVEDGSLL |
| | | MFRVWNRFMKKKEGKISKHAKIVNELFSDE |
| | | NASNAAIKLLSVFDIFYRDKQIDNPIVHKA |
| | | GTTLYNKRTKDGEVIVDYTTMVKNKEKRPN |
| | | VYTTTKKYDIIKDRRYTEEQFEIHLHVNIG |
| | | KEENKEKLETSKVINEKKNTLVVTRSNEHL |
| | | LYVVIFDENDNILLKKSLNTVKGMNFKSKL |
| | | EVVEIQKKENMQSWKTVGSNQALMEGYLSF |
| | | AIKEIADLVKEYDAILVLEQNSVGKNILNE |
| | | RVYTRFKEMLITNLSLDVDYENKDFYSYTE |
| | | LGGKVASWRDCVTNGICIQVPSAYKYKDPT |
| | | TSFSTISMYAKTTAEKSKKLKQIKSFKYNR |
| | | ERGLFELVIAKGVGLENNIVCDSFGSRSII |
| | | ENDISKEVSCTLKIEKYLIDAGIEYNDEKE |
| | | VLKDLDTAAKTDAVHKAVTLLLKCFNESPD |
| | | GRYYISPCGEHFTLCDAPEVLSAINYYIRS |
| | | RYIREQIVEGVKKMEYKKTILLAK |
| SEQ | Cas12 | MNYKTGLEDFIGKESLSKTLRNALIPTEST |
| ID | Variant | KIHMEEMGVIRDDELRAEKQQELKEIMDDY |
| NO: | | YRTFIEEKLGQIQGIQWNSLFQKMEETMED |
| 39 | | ISVRKDLDKIQNEKRKEICCYFTSDKRFKD |
| | | LFNAKLITDILPNFIKDNKEYTEEEKAEKE |
| | | QTRVLFQRFATAFTNYFNQRRNNFSEDNIS |
| | | TAISFRIVNENSEIHLQNMRAFQRIEQQYP |
| | | EEVCGMEEEYKDMLQEWQMKHIYSVDFYDR |
| | | ELTQPGIEYYNGICGKINEHMNQFCQKNRI |
| | | NKNDFRMKKLHKQILCKKSSYYEIPFRFES |
| | | DQEVYDALNEFIKTMKKKEIIRRCVHLGQE |
| | | CDDYDLGKIYISSNKYEQISNALYGSWDTI |
| | | RKCIKEEYMDALPGKGEKKEEKAEAAAKKE |
| | | EYRSIADIDKIISLYGSEMDRTISAKKCIT |
| | | EICDMAGQISIDPLVCNSDIKLLQNKEKTT |
| | | EIKTILDSFLHVYQWGQTFIVSDIIEKDSY |
| | | FYSELEDVLEDFEGITTLYNHVRSYVTQKP |
| | | YSTVKFKLHFGSPTLANGWSQSKEYDNNAI |
| | | LLMRDQKFYLGIFNVRNKPDKQIIKGHEKE |
| | | EKGDYKKMIYNLLPGPSKMLPKVFITSRSG |
| | | QETYKPSKHILDGYNEKRHIKSSPKFDLGY |
| | | CWDLIDYYKECIHKHPDWKNYDFHFSDTKD |
| | | YEDISGFYREVEMQGYQIKWTYISADEIQK |
| | | LDEKGQIFLFQIYNKDFSVHSTGKDNLHTM |
| | | YLKNLFSEENLKDIVLKLNGEAELFFRKAS |
| | | IKTPIVHKKGSVLVNRSYTQTVGNKEIRVS |
| | | IPEEYYTEIYNYLNHIGKGKLSSEAQRYLD |
| | | EGKIKSFTATKDIVKNYRYCCDHYFLHLPI |
| | | TINFKAKSDVAVNERTLAYIAKKEDIHIIG |
| | | IDRGERNLLYISVVDVHGNIREQRSFNIVN |
| | | GYDYQQKLKDREKSRDAARKNWEEIEKIKE |
| | | LKEGYLSMVIHYIAQLVVKYNAVVAMEDLN |
| | | YGFKTGRFKVERQVYQKFETMLIEKLHYLV |
| | | FKDREVCEEGGVLRGYQLTYIPESLKKVGK |
| | | QCGFIFYVPAGYTSKIDPTTGFVNLFSFKN |
| | | LTNRESRQDFVGKFDEIRYDRDKKMFEFSF |
| | | DYNNYIKKGTILASTKWKVYTNGTRLKKIV |
| | | VNGKYTSQSMEVELTDAMEKMLQRAGIEYH |
| | | DGKDLKGQIVEKGIEAEIIDIFRLTVQMRN |
| | | SRSESEDREYDRLISPVLNDKGEFFDTATA |
| | | DKTLPQDADANGAYCIALKGLYEVKQIKEN |
| | | WKENEQFPRNKLVQDNKTWFDFMQKKRYL |
| SEQ | Cas12 | MEDKQFLERYKEFIGLNSLSKTLRNSLIPV |
| ID | Variant | GSTLKHIQEYGILEEDSLRAQKREELKGIM |
| NO: | | DDYYRNYIEMHLRDVHDIDWNELFEALTEV |
| 40 | | KKNQTDDAKKRLEKIQEKKRKEIYQYLSDD |
| | | AVFSEMFKEKMISGILPDFIRCNEGYSEEE |
| | | KEEKLKTVALFHRFTSSFNDFFLNRKNVFT |
| | | KEAIVTAIGYRVVHENAEIFLENMVAFQNI |
| | | QKSAESQISIIERKNEHYFMEWKLSHIFTA |
| | | DYYMMLMTQKAIEHYNEMCGVVNQQMREYC |
| | | QKEKKNWNLYRMKRLHKQILSNASTSFKIP |
| | | EKYENDAEVYESVNSFLQNVMEKTVMERIA |
| | | VLKNSTDNFDLSKIYITAPYYEKISNYLCG |
| | | SWNTITDCLTHYYEQQIAGKGARKDQKVKA |
| | | AVKADKWKSLSEIEQLLKEYARAEEVKRKP |
| | | EEYIAEIENIVSLKEAHLLEYHPEVNLIEN |
| | | EKYATEIKDVLDNYMELFHWMKWFYIEEAV |
| | | EKEVNFYGELDDLYEEIKDIVPLYNKVRNY |
| | | VTQKPYSDTKIKLNFGTPTLANGWSKSKEY |
| | | DYNAILLQKDGKYYMGIFNPIQKPEKEIIE |
| | | GHSQPLEGNEYKKMVYYYLPSANKMLPKVL |
| | | LSKKGMEIYQPSEYIINGYKERRHIKSEEK |
| | | FDLQFCHDLIDYFKSGIERNSDWKVFGFDF |
| | | SDTDTYQDISGFYREVEDQGYKIDWTYIKE |
| | | ADIDRLNEEGKLYLFQIYNKDFSEKSTGRE |
| | | NLHTMYLKNLFSEENVREQVLKLNGEAEIF |
| | | FRKSSVKKPIIHKKGTMLVNRTYMEEVNGN |
| | | SVRRNIPEKEYQEIYNYKNHRLKGELSTEA |
| | | KKYLEKAVCHETKKDIVKDYRYSVDKFFIH |
| | | LPITINYRASGKETLNSVAQRYIAHQNDMH |
| | | VIGIDRGERNLIYVSVINMQGEIKEQKSFN |
| | | IINEFNYKEKLKEREQSRGAARRNWKEIGQ |
| | | IKDLKEGYLSGVIHEIAKMMIKYHAIIAME |
| | | DLNYGFKRGRFKVERQVYQKFENMLIQKLN |
| | | YLVFKDRPADEDGGVLRGYQLAYIPDSVKK |
| | | MGRQCGMIFYVPAAFTSKIDPTTGFVDIFK |
| | | HKVYTTEQAKREFILSFDEICYDVERQLFR |
| | | FTFDYANFVTQNVTLARNNWTIYTNGTRAQ |
| | | KEFGNGRMRDKEDYNPKDKMVELLESEGIE |
| | | FKSGKNLLPALKKVSNAKVFEELQKIVRFT |
| | | VQLRNSKSEENDVDYDHVISPVLNEEGNFF |
| | | DSSKYKNKEEKKESLLPVDADANGAYCIAL |
| | | KGLYIMQAIQKNWSEEKALSPDVLRLNNND |
| | | WFDYIQNKRYR |
| SEQ | Cas12 | MEKSLNDFIGLYSVSKTLRFELKPVSETLE |
| ID | Variant | NIKKFHFLEEDKKKANDYKDVKKIIDNYHK |
| NO: | | YFIDDVLKNASFNWKKLEEAIREYNKNKSD |
| 41 | | DSALVAEAALKTFQKFTSYFTGFQENRKNV |
| | | YSAEAIPTAVPYRIVNDNFPKFLQNVQKKL |
| | | GDAILKLFTSDKRYKALTAATPKELFESIL |
| | | PDWFGEQCNQDLNKLIFKTIQEKCPQIIDE |
| | | VEKELSSYLGKEKLAGIFTLESFNKYLGQG |
| | | GKENQRGIDFYNQIIGGVVEKEGGINLRGV |
| | | NQFLNLYWQQHPDFTKEDRRIKMVPLYKQI |
| | | LSDRSSLSFKIESIENDEELKNALLECADK |
| | | LELKNDEKKSIFEEVCDLFSSVKNLDLSGI |
| | | YINRKDINSVSRILTGDWSWLQSRMNVYAE |
| | | EKFTTKAEKARWQKSLDDEGENKSKGFYSL |
| | | TDLNEVLEYSSENVAETDIRITDYFEHRCR |
| | | YYVDKETEMFVQGSELVALSLQEMCDDILK |
| | | KRKAMNTVLENLSSENKLREKTDDVAVIKE |
| | | YLDAVQELLHRIKPLKVNGVGDSTFYSVYD |
| | | SIYSALSEVISVYNKTRNYITKKAASPEKY |
| | | KLNFDNPTLADGWDLNKEQANTSVILRKDG |
| | | MFYLGIMNPKNKPKFAEKYDCGNESCYEKM |
| | | IYKQFDATKQIPKCSTQKKEVQKYFLSGAT |
| | | EPYILNDKKSFKSELIITKDIWFMNNHVWD |
| | | GEKFVPKRDNETRPKKFQIGYFKQTGDFDG |
| | | YKNALSNWISFCKNFLQSYLSATVYDYNFK |
| | | NSEEYEGLDEFYNYLNATCYKLNFINIPET |
| | | EINKMVSEGKLYLFQIYNKDFASGSTGMPN |
| | | MHTLYWKNLFSDENLKNVCLKLNGEAELFY |
| | | RPAGIKEPVIHKEGSYLVNRTTEDGESIPE |
| | | KIYFEIYKNANGKLEKLSDEAQNYISNHEV |
| | | VIKKAGHEIIKDRHYTEPKFLFHVPLTINF |
| | | KASGNSYSINENVRKFLKNNPDVNIIGLDR |
| | | GERHLIYLSLINQKGEIIKQFTFNEVERNK |
| | | NGRTIKVNYHEKLDQREKERDAARKSWQAI |
| | | GKIAELKEGYLSAVIHQLTKLMVEYNAVVV |
| | | MEDLNFGFKRGRFHVEKQVYQKFEHILIDK |
| | | SNYLVFKDRGLNEPGGVLNGYQIAGQFESF |
| | | QKLGKQSGMLFYVPAGYTSKIDPKTGFVSM |
| | | MNFKDLTNVHKKRDFFSKFDNIHYDEANGS |
| | | FVFTFDYKKFDGKAKEEMKLTKWSVYSRDK |
| | | RIVYFAKTKSYEDVLPTEKLQKIFESNGID |
| | | YKSGNNIQDSVMAIGADLKEGAKPSKEISD |
| | | FWDGLLSNFKLILQMRNSNARTGEDYIISP |
| | | VMADDGTFFDSREEFKKGEDAKLPLDADAN |
| | | GAYHIALKGLSLINKINLSKDEELKKFDMK |
| | | ISNADWFKFAQEKNYAK |
| SEQ | Cas12 | MEEKKMSKIEKFIGKYKISKTLRFRAVPVG |
| ID | Variant | KTQDNIEKKGILEKDKKRSEDYEKVKAYLD |
| NO: | | SLHRDFIENTLKKVKLNELNEYACLFFSGT |
| 42 | | KDDGDKKFFTSLNGYVNNRKNLYVSDAKPT |
| | | SIAYRCINENLPKFLRNVECYKKVVKMEKL |
| | | EEKMRKTISNEFCNDEMYKKIFSEKILSEN |
| | | NEEDVSDIVSSYKGQVIPKEQIEYMSNNLN |
| | | LSPYRIEDCFNIDFFEFCLSQGGIDLYNTF |
| | | IGGYSKKDGTKVQGINEIVNLYNQKNKKDK |
| | | EKYKLPQFTPLFKQILSDRDTKSFSIEKLE |
| | | NIYEVVELVKKSYSDEMFDDIETVFSNLNY |
| | | YDASGIYVKNGPAITHISMNLTKDWATIRN |
| | | NWNYEYDEKHSTKKNKNIEKYEDTRNTMYK |
| | | KIDSFTLEYISRLVGKDIDELVKYFENEVA |
| | | NFVMDIKKTYSKLTPLFDRCQKENFDISED |
| | | EVNDIKGYLDNVKLLESFMKSFTINGKENN |
| | | IDYVFYGKFTDDYDKLHEFDHIYNKVRNYI |
| | | TTSRKPYKLDKYKLYFDNPQLLGGWDINKE |
| | | KDYRTVMLTKDGKYYFAIIDKGEHPFDNIP |
| | | KDYFDNNGYYKKIIYRQIPNAAKYLSSKQI |
| | | VPQNPPEEVKRILDKKKADSKSLTEEEKNI |
| | | FIDYIKSDFLKNYKLLFDKNNNPYFNFAFR |
| | | ESSTYESLNEFFEDVERQAYSVRYENLPAD |
| | | YIDNLVNEGKIYLFEIYSKDFSEYSKGTNN |
| | | LHTMYFKALFDNDNLKNTVFKLSGNAELFI |
| | | RPASIKKDELVIHPKNQLLQNKNPLNPKKQ |
| | | SIFDYDLVKDKRFFENQYMLHISIEINKNE |
| | | RDAKKIKNINEMVRKELKDSDDNYIIGIDR |
| | | GERNLLYVCVINSAGKIVEQMSLNEIINEY |
| | | NGIKHTVDYQGLLDKCEKERNAQRQSWKSI |
| | | ENIKELKDGYISQVVHKLCQLVEKYDAIIA |
| | | MENLNGGFKRGRTKFEKQVYQKFENKLINK |
| | | MEYMADKKRKTTENGGILRGYQLTNGCINN |
| | | SYQNGFIFYVPAWLTSKIDPTTGFVDLLKP |
| | | KYTNVEEAHLWINKFNSITYDKKLDMFAFN |
| | | INYSQFPRADIDYRKIWTFYTNGYRIETFR |
| | | NSEKNNEFDWKEVHLTSVIKKLLEEYQINY |
| | | ISGKNIIDDLIQIKDKPFWNSFIKYIRLTL |
| | | QMRNSITGRTDVDYIISPVINNEGTFYDSR |
| | | KDLDEITLPQDADANGAYNIARKALWIIEK |
| | | LKESPDEELNKVKLAITQREWLEYAQINI |
| SEQ | Cas12 | MIIHNCYIGGSFMKKIDSFTNCYSLSKTLR |
| ID | Variant | FKLIPIGATQSNFDLNKMLDEDKKRAENYS |
| NO: | | KAKSIIDKYHRFFIDKVLSSVTENKAFDSF |
| 43 | | LEDVRAYAELYYRSNKDDSDKASMKTLESK |
| | | MRKFIALALQSDEGFKDLFGQNLIKKTLPE |
| | | FLESDTDKEIIAEFDGFSTYFTGFFNNRKN |
| | | MYSADDQPTAISYRCINDNLPKFLDNVRTF |
| | | KNSDVASILNDNLKILNEDFDGIYGTSAED |
| | | VFNVDYFPFVLSQKGIEAYNSILGGYTNSD |
| | | GSKIKGLNEYINLYNQKNENIHRIPKMKQL |
| | | FKQILSERESVSFIPEKFDSDDDVLSSIND |
| | | YYLERDGGKVLSIEKTVEKIEKLFSAVTDY |
| | | STDGIFVKNAAELTAVCSGAFGYWGTVQNA |
| | | WNNEYDALNGYKETEKYIDKRKKAYKSIES |
| | | FSLADIQKYADVSESSETNAEVTEWLRNEI |
| | | KEKCNLAVQGYESSKDLISKPYTESKKLFN |
| | | NDNAVELIKNALDSVKELENVLRLLLGTGK |
| | | EESKDENFYGEFLPCYERICEVDSLYDKVR |
| | | NYMTQKLYKTDKIKLNFQNPQFLGGWDRNK |
| | | EADYSAVLLRRNSLYYIAIMPSGYKRVFEK |
| | | IPAPKADETVYEKVIYKLLPGPNKMLPKVF |
| | | FSKKGIETFNPPKEILEKYELGTHKTGDGF |
| | | NLDDCHALIDYFKSALDVHSDWSNFGFRFS |
| | | DTSTYKNIADFYNEVKNQGYKITFCDVPQS |
| | | YINELVDEGKLYLFQLYNKDFSEHSKGTPN |
| | | LHTLYFKMLFDERNLENVVFKLNGEAEMFY |
| | | REASISKDDMIVHPKNQPIKNKNEQNSRKQ |
| | | STFEYDIVKDRRYTVDQFMLHIPITLNFTA |
| | | NGGTNINNEVRKALKDCDKNYVIGIDRGER |
| | | NLLYICVVDSEGRIIEQYSLNEIINEYNGN |
| | | TYSTDYHALLDKKEKERLESRKAWKTVENI |
| | | KELKEGYISQVVHKICELVEKYDAVIVMED |
| | | LNLGFKQGRSGKFEKSVYQKFEKMLIDKLN |
| | | YFADKKKSPEEIGSVLNAYQLTNAFESFEK |
| | | MGKQNGFIFYVPAYLTSKIDPTTGFADLLH |
| | | PSSKQSKESMRDFVGRFDSITENKTENYFE |
| | | FELDYNKFPRCNTDYRKKWTVCTYGSRIKT |
| | | FRNPEKNSEWDNKTVELTPAFMALFEKYSI |
| | | DVNGDIKAQIMSVDKKDFFVELIGLLRLTL |
| | | QMRNSETGKVDRDYLISPVKNSEGVFYNSD |
| | | DYKGIENASLPKDADANGAYNIARKGLWII |
| | | EQIKACENDAELNKIRLAISNAEWLEYAQK |
| | | K |
| SEQ | Cas12 | MKEQFVNQYPISKTLRFSLIPIGKTEENFN |
| ID | Variant | KNLLLKEDEKKAEEYQKVKGYIDRYHKFFI |
| NO: | | ETALCNINFEGFEEYSLLYYKCSKDDNDLK |
| 44 | | TMEDIEIKLRKQISKTMTSHKLYKDLFGEN |
| | | MIKTILPNFLDSDEEKNSLEMFRGFYTYFS |
| | | GFNTNRKNMYTEEAKSTSIAYRCINDNLPK |
| | | FLDNSKSFEKIKCALNKEELKAKNEEFYEI |
| | | FQIYATDIFNIDFFNFVLTQPGIDKYNGII |
| | | GGYTCSDGTKVQGLNEIINLYNQQIAKDDK |
| | | SKRLPLLKMLYKQILSDRETVSFIPEKFSS |
| | | DNEVLESINNYFSKNVSNAIKSLKELFQGF |
| | | EAYNMNGIFISSGVAITDLSNAVFGDWNAI |
| | | STAWEKAYFETNPPKKNKSQEKYEEELKAN |
| | | YKKIKSFSLDEIQRLGSIAKSPDSIGSVAE |
| | | YYKITVTEKIDNITELYDGSKELLNCNYSE |
| | | SYDKKLIKNDTVIEKVKTLLDAVKSLEKLI |
| | | KPLVGTGKEDKDELFYGTFLPLYTSLSAVD |
| | | RLYDKVRNYATQKPYSKDKIKLNFNCSSFL |
| | | SGWATDYSSNGGLIFEKDGLYYLGIVNKKF |
| | | TTEEIDYLQQNADENPAQRIVYDFQKPDNK |
| | | NTPRLFIRSKGTNYSPSVKEYNLPVEEIVE |
| | | LYDKRYFTTEYRNKNPELYKASLVKLIDYF |
| | | KLGFTRHESYRHYDFKWKKSEEYNDISEFY |
| | | KDVEISCYSLKQEKINYNTLLNFVAENRIY |
| | | LFQIYNKDFSKYSKGTPNLHTRYFKALFDE |
| | | NNLSDVVFKLNGGSEMFFRKASIKDNEKVV |
| | | HPANQPIDNKNPDNSKKQSTFDYELIKDKR |
| | | FTKHQFSIHIPITMNFKARGRDFINNDIRK |
| | | AIKSEYKPYVIGIDRGERNLIYISVINNNG |
| | | EIVEQMSLNDIISDNGYKVDYQRLLDRKEK |
| | | ERDNARKSWGTIENIKELKEGYISQVIHKI |
| | | CELVIKYDAVIAMEDLNFGFKRGRFNVEKQ |
| | | VYQKFENMLISKLNYLCDKKSEANSEGGLL |
| | | KAYQLTNKFDGVNKGKQNGIIFYVPAWLTS |
| | | KIDPVTGFVDLLHPKYISVEETHSLFEKLD |
| | | DIRYNFEKDMFEFDIDYSKLPKCNADFKQK |
| | | WTVCTNADRIMTFRNSEKNNEWDNKRILLS |
| | | DEFKRLFEEFGIDYCHNLKNKILSISNKDF |
| | | CYRFIKLFALTMQMRNSITGSTNPEDDYLI |
| | | SPVRDENGVFYDSRNFIGSKAGLPIDADAN |
| | | GAYNIARKGLWAINAIKSTADDMLDKVDLS |
| | | ISNAKWLEYVQK |
| SEQ | Cas12 | MADLSQFTHKYQVPKTLRFELIPQGKTLEN |
| ID | Variant | LSAYGMVADDKQRSENYKKLKPVIDRIYKY |
| NO: | | FIEESLKNTNLDWNPLYEAIREYRKEKTTA |
| 45 | | TITNLKEQQDICRRAIASRFEGKVPDKGDK |
| | | SVKDFNKKQSKLFKELFGKELFTDSVLEQL |
| | | PGVSLSDEDKALLKSFDKFTTYFVGFYDNR |
| | | KNVFSSDDISTGIPHRLVQENFPKFIDNCD |
| | | DYKRLVLVAPELKEKLEKAAEATKIFEDVS |
| | | LDEIFSIKFYNRLLQQNQIDQFNQLLGGIA |
| | | GAPGTPKIQGLNETLNLSMQQDKTLEQKLK |
| | | SVPHRFSPLYKQILSDRSSLSFIPESFSCD |
| | | AEVLLAVQEYLDNLKTEHVIEDLKEVFNRL |
| | | TTLDLKHIYVNSTKVTAFSQALFGDWNLCR |
| | | EQLRVYKMSNGNEKITKKALGELESWLKNS |
| | | DIAFTELQEALADEALPAKVNLKVQEAISG |
| | | LNEQMAKSLPKELKIPEEKEELKALLDAIQ |
| | | EVYHTLEWFIVSDDVETDTDFYVPLKETLQ |
| | | IIQPIIPLYNKVRNFATQKPYSVEKFKLNF |
| | | ANPTLADGWDENKEQQNCAVLFQKGNNYYL |
| | | GILNPKNKPDFDNVDTEKQGNCYQKMVYKQ |
| | | FPDFSKMMPKCTTQLKEVKQHFEGKDSDYI |
| | | LNNKNFIKPLTITREVYDLNNVLYDGKKKF |
| | | QIDYLRKTKDEDGYYTALHTWIDFAKKFVA |
| | | SYKSTSIYDTSTILPPEKYEKLNEFYGALD |
| | | NLFYQIKFENIPEEIIDTYVEDGKLFLFQI |
| | | YNKDFAAGATGAPNLHTIYWKAVFDPENVK |
| | | DVVVKLNGQAELFYRPKSNMDVIRHKVGEK |
| | | LVNRTLKDGSILTDELHKELYLYANGSLKK |
| | | GLSEDAKIILDKNLAVIYDVHHEIVKDRRF |
| | | TTDKFFFHVPLTLNYKCDKNPVKFNAEVQE |
| | | YLKENPDTYVIGIDRGERNLIYAVVIDPKG |
| | | RIVEQKSFNVINGFDYHGKLDQREKERVKA |
| | | RQAWTAVGKIKELKQGYLSLVVHEISKMMV |
| | | RYQAVVVLENLNVGFKRVRSGIAEKAVYQQ |
| | | FEKMLINKLNYLMFKDAGGTEPGSVLNAYQ |
| | | LTDRFESFAKMGLQTGFLFYIPAAFTSKID |
| | | PATGFVDPFRWGAIKTLADKREFLSGFESL |
| | | KFDSTTGNFILHFDVSKNKNFQKKLEGFVP |
| | | DWDIIIEANKMKTGKGATYIAGKRIEFVRD |
| | | NNSQGHYEDYLPCNALAETLRQCDIPYEEG |
| | | KDILPLILEKNDSKLLHSVFKVVRLTLQMR |
| | | NSNAETGEDYISSPVEDVSGSCFDSRMENE |
| | | KLPKDADANGAYHIALKGMLALERLRKDEK |
| | | MAISNNDWLNYIQEKRA |
| SEQ | Cas12 | MTNFDNFTKKYVNSKTIRLEAIPVGKTLKN |
| ID | Variant | IEKMGFIAADRQRDEDYQKAKSVIDHIYKA |
| NO: | | FMDDCLKDLFLDWDPLYEAVVACWRERSPE |
| 46 | | GRQALQIMQADYRKKIADRFRNHELYGSLF |
| | | TKKIFDGSVAQRLPDLEQSAEEKSLLSNFN |
| | | KFTSYFRDFFDKRKRLFSDDEKHSAIAYRL |
| | | INENFLKFVANCEAFRRMTERVPELREKLQ |
| | | NTGSLQVYNGLALDEVFSADFYNQLIVQKQ |
| | | IDLYNQLIGGIAGEPGTPNIQGLNATINLA |
| | | LQGDSSLHEKLAGIPHRFNPLYKQILSDVS |
| | | TLSFVPSAFQSDGEMLAAVRGFKVQLESGR |
| | | VLQNVRRLFNGLETEADLSRVYVNNSKLAA |
| | | FSSMFFGRWNLCSDALFAWKKGKQKKITNK |
| | | KLTEIKKWLKNSDIAIAEIQEAFGEDFPRG |
| | | KINEKIQAQADALHSQLALPIPENLKALCA |
| | | KDGLKSMLDTVLGLYRMLQWFIVGDDNEKD |
| | | SDFYFGLGKILGSLDPVLVLYNRVRNYITK |
| | | KPYSLTKFRLNFDNSQLLNGWDENNLDTNC |
| | | ASIFIKDGKYYLGISNKNNRPQFDTVATSG |
| | | KSGYQRMVYKQFANWGRDLPHSTTQMKKVK |
| | | KHFSASDADYVLDGDKFIRPLIITKEIFDL |
| | | NNVKFNGKKKLQVDYLRNTGDREGYTHALH |
| | | TWINFAKDFCACYKSTSIYDISCLRPTDQY |
| | | DNLMDFYADLGNLSHRIVWQTIPEEAIDNY |
| | | VEQGQLFLFQLYNKDFAPGADGKPNLHTLY |
| | | WKAVFNPENLEDVVVKLNGKAELFYRPRSN |
| | | MDVVRHKVGEKLVNRKLKNGLTLPSRLHEE |
| | | IYRYVNGTLNKDLSADARSVLPLAVVRDVQ |
| | | HEIIKDRRFTADKFFFHASLTFNFKSSDKP |
| | | VGFNEDVREYLREHPDTYVVGVDRGERNLI |
| | | YIVVIDPQGNIVEQRSFNMINGIDYWSLLD |
| | | QKEKERVEAKQAWETVGKIKDLKCGYLSFL |
| | | IHEITKIIIKYHAVVILENLSLGFKRVRTG |
| | | IAEKAVYQQFERMLVTKLGYVVFKDRAGKA |
| | | PGGVLNAYQLTDNTRTAENTGIQNGFLFYV |
| | | PAAFTSRVDPATGFFDFYDWGKIKTATDKK |
| | | NFIAGFNSVRYERSTGDFIVHVGAKNLAVR |
| | | RVAEDVRTEWDIVIEANVRKMGIDGNSYIS |
| | | GKRIRYRSGEQGHGQYENHLPCQELIRALQ |
| | | QYGIQYETGKDILPAILQQDDAKLTDTVFD |
| | | VFRLALQMRNTSAETGEDYFNSVVRDRSGR |
| | | CFDTRRAEAAMPKEADANDAYHIALKGLFV |
| | | LEKLRKGESIGIKNTEWLRYVQQRHS |
| SEQ | Cas12 | MENYGGFTGLYPLQKTLKFELRPQGRTMEH |
| ID | Variant | LVSSNFFEEDRDRAEKYKIVKKVIDNYHKD |
| NO: | | FINECLSKRSFDWTPLMKTSEKYYASKEKN |
| 47 | | GKKKQDLDQKIIPTIENLSEKDRKELELEQ |
| | | KRMRKEIVSVFKEDKRFKYLFSEKLFSILL |
| | | KDEDYSKEKLTEKEILALKSFNKFSGYFIG |
| | | LHKNRANFYSEGDESTAIAYRIVNENFPKF |
| | | LSNLKKYREVCEKYPEIIQDAEQSLAGLNI |
| | | KMDDIFPMENFNKVMTQDGIDLYNLAIGGK |
| | | AQALGEKQKGLNEFLNEVNQSYKKGNDRIR |
| | | MTPLFKQILSERTSYSYILDAFDDNSQLIT |
| | | SINGFFTEVEKDKEGNTFDRAVGLIASYMK |
| | | YDLSRVYIRKADLNKVSMEIFGSWERLGGL |
| | | LRIFKSELYGDVNAEKTSKKVDKWLNSGEF |
| | | SLSDVINAIAGSKSAETFDEYILKMRVARG |
| | | EIDNALEKIKCINGNFSEDENSKMIIKAIL |
| | | DSVQRLFHLFSSFQVRADFSQDGDFYAEYN |
| | | EIYEKLFAIVPLYNRVRNYLTKNNLSMKKI |
| | | KLNFKNPALANGWDLNKEYDNTAVIFLREG |
| | | KYYLGIMNPSKKKNIKFEEGSGTGPFYKKM |
| | | AYKLLPDPNKMLPKVFFAKKNINYYNPSDE |
| | | IVKGYKAGKYKKGENFDIDFCHKLIDFFKE |
| | | SIQKNEDWRAFNYLFSATESYKDISDFYSE |
| | | VEDQGYRMYFLNVPVANIDEYVEKGDLFLF |
| | | QIYNKDFASGAKGNKDMHTIYWNAAFSDEN |
| | | LRNVVVKLNGEAELFYRDKSIIEPICHKKG |
| | | EMLVNRTCFDKTPVPDKIHKELFDYHNGRA |
| | | KTLSIEAKGYLDRVGVFQASYEIIKDRRYS |
| | | ENKMYFHVPLKLNFKADGKKNLNKMVIEKF |
| | | LSDKDVHIIGIDRGERNLLYYSVIDRRGNI |
| | | IDQDSLNIIDGFDYQKKLGQREIERREARQ |
| | | SWNSIGKIKDLKEGYLSKAVHKVSKMVLEY |
| | | NAIVVLEDLNFGFKRGRFKVEKQVYQKFEK |
| | | MLIDKLNYLVFKEVLDSRDAGGVLNAYQLT |
| | | TQLESFNKLGKQSGILFYVPAAYTSKIDPT |
| | | TGFVSLFNTSRIESDSEKKDFLSGFDSIVY |
| | | SAKDGGIFAFKFDYRNRNFQREKTDHKNIW |
| | | TVYTNGDRIKYKGRMKGYEITSPTKRIKDV |
| | | LSSSGIRYDDGQELRDSIIQSGNKVLINEV |
| | | YNSFIDTLQMRNSDGEQDYIISPVKNRNGE |
| | | FFRTDPDRRELPVDADANGAYHIALRGELL |
| | | MQKIAEDFDPKSDKFTMPKMEHKDWFEFMQ |
| | | TRGD |
| SEQ | Cas12 | MLHAFTNQYQLSKTLRFGATLKEDEKKCKS |
| ID | Variant | HEELKGFVDISYENMKSSATIAESLNENEL |
| NO: | | VKKCERCYSEIVKFHNAWEKIYYRTDQIAV |
| 48 | | YKDFYRQLSRKARFDAGKQNSQLITLASLC |
| | | GMYQGAKLSRYITNYWKDNITRQKSFLKDF |
| | | SQQLHQYTRALEKSDKAHTKPNLINFNKTF |
| | | MVLANLVNEIVIPLSNGAISFPNISKLEDG |
| | | EESHLIEFALNDYSQLSELIGELKDAIATN |
| | | GGYTPFAKVTLNHYTAEQKPHVFKNDIDAK |
| | | IRELKLIGLVETLKGKSSEQIEEYFSNLDK |
| | | FSTYNDRNQSVIVRTQCFKYKPIPFLVKHQ |
| | | LAKYISEPNGWDEDAVAKVLDAVGAIRSPA |
| | | HDYANNQEGFDLNHYPIKVAFDYAWEQLAN |
| | | SLYTTVTFPQEMCEKYLNSIYGCEVSKEPV |
| | | FKFYADLLYIRKNLAVLEHKNNLPSNQEEF |
| | | ICKINNTFENIVLPYKISQFETYKKDILAW |
| | | INDGHDHKKYTDAKQQLGFIRGGLKGRIKA |
| | | EEVSQKDKYGKIKSYYENPYTKLTNEFKQI |
| | | SSTYGKTFAELRDKFKEKNEITKITHFGII |
| | | IEDKNRDRYLLASELKHEQINHVSTILNKL |
| | | DKSSEFITYQVKSLTSKTLIKLIKNHTTKK |
| | | GAISPYADFHTSKTGFNKNEIEKNWDNYKR |
| | | EQVLVEYVKDCLTDSTMAKNQNWAEFGWNF |
| | | EKCNSYEDIEHEIDQKSYLLQSDTISKQSI |
| | | ASLVEGGCLLLPIINQDITSKERKDKNQFS |
| | | KDWNHIFEGSKEFRLHPEFAVSYRTPIEGY |
| | | PVQKRYGRLQFVCAFNAHIVPQNGEFINLK |
| | | KQIENFNDEDVQKRNVTEFNKKVNHALSDK |
| | | EYVVIGIDRGLKQLATLCVLDKRGKILGDF |
| | | EIYKKEFVRAEKRSESHWEHTQAETRHILD |
| | | LSNLRVETTIEGKKVLVDQSLTLVKKNRDT |
| | | PDEEATEENKQKIKLKQLSYIRKLQHKMQT |
| | | NEQDVLDLINNEPSDEEFKKRIEGLISSFG |
| | | EGQKYADLPINTMREMISDLQGVIARGNNQ |
| | | TEKNKIIELDAADNLKQGIVANMIGIVNYI |
| | | FAKYSYKAYISLEDLSRAYGGAKSGYDGRY |
| | | LPSTSQDEDVDFKEQQNQMLAGLGTYQFFE |
| | | MQLLKKLQKIQSDNTVLRFVPAFRSADNYR |
| | | NILRLEETKYKSKPFGVVHFIDPKFTSKKC |
| | | PVCSKTNVYRDKDDILVCKECGFRSDSQLK |
| | | ERENNIHYIHNGDDNGAYHIALKSVENLIQ |
| | | MK |
| SEQ | Cas12 | MKNGINLFKTKTTKTKGVDMEKYQITKTIR |
| ID | Variant | FKLLPDNAHEIVEKVKSLKTSNVDELMDEV |
| NO: | | KNVHLKGLELLFALKKYFYFDGNQCKSFKS |
| 49 | | TLEIKARWLRLYTPDQYYLKKSSKNSYQLK |
| | | SLSYFKDVFNDWLFNWEESVSELAIIYEKY |
| | | KICQHQRDSRADIALLIKKLSMKEYFPFIS |
| | | DLIDCVNDKNSNKTFLMKLSEELSVLLEKC |
| | | NSRALPYQSNGIVVGKASLNYYTVSKSEKM |
| | | LQNEYEDVCQSLDKNYDITEMKVILYKEKL |
| | | DNLNFKDVTIANAYNLLKENKALQKRLFSE |
| | | YVSQGKVLSLIKTELPLFSNINDNDFEKYK |
| | | EWSNEIKKLADKKNTFCKKTQQDKIKDIQN |
| | | KISELKKKRGALFQYKFTSFQKHCDNYKKV |
| | | AVQYGKLKARKKAIEKDEIEANLLRYWSVI |
| | | LEQEDKHSLVLIPKNNAKDAKQYIETINTK |
| | | GGKYIIHHLDSLTLRALNKLCFNAVDIEKG |
| | | QMVRENTFYQGIKEEFERNKINCDNQGVLK |
| | | IQGLYSFKTEGGQINEKEAVEFFKEVLKSN |
| | | YAREVLNLPYDLESNIFQKEYTNLDQFRQD |
| | | LEKCCYALHSKIGKDDLDEFTRRFEAQVFD |
| | | ITSIDLKSKKEKTKTTGEMKKHTQLWLEFW |
| | | KGAIEQNFATRVNPELSIFWRAPKSSREKK |
| | | YGKGSDLYDPNKNNRYLYEQYTLALTITEN |
| | | AGSHFKDIAFKDTSKIKEAIKEFNMSLSQS |
| | | KYCFGIDRGNAELVSLCLIKNEKDFPFEKF |
| | | PVYRLRDLTYQGDFKDKHDQMRYGVAIKNI |
| | | SYFIDQEDLFEKNNLSAIDMTTAKLIKNKI |
| | | VLNGDVLTYLKLKEETAKHKLTQFFQGSSI |
| | | NKNSRVYFDEDENVFKITTNRNHNPEEIIY |
| | | FYRGEYGAIKNKNDLEDILNEYLCKMETGE |
| | | SEIVLLNRVNHLRDAISANIVGILSYLIDL |
| | | FPETIVALENLAKGTIDRHVSQSYENITRR |
| | | FEWALYRKLLNKQLAPPELKENILLREGDD |
| | | KIDQFGIIHFVEEKNTSKDCPNCRKTTQQT |
| | | NDNKFKEKKFVCKSCGFDTSKDRKGMDSLN |
| | | SPDTVAAYNVARKKFES |
| SEQ | Cas12 | MAKETKEFKTFDDFTNLYEVQKTLRFELEA |
| ID | Variant | VPETEIVLENRGIWYKRDKKRADEKPIVKF |
| NO: | | YMDILHREFTDEALEKIKESGVLNLSGYFK |
| 50 | | LFEELRRLQNHGANTKEEKKLKLEEIRAKK |
| | | REISNELSQIRRVFSVRGFDVVDSDWKKKY |
| | | TIEGKKIKNDKSKTYLILSENILNFLENRF |
| | | TSKEVERLRSIDKKHVEDYGNVVNSGGENI |
| | | FATFKGFFGYFDSLIKNRENFYETDGKAGR |
| | | VATRSVDENLNFFAENLHIFSTDLPKALKD |
| | | DLSDTQKAIFERSYYKNCLLQKDIKSYNLI |
| | | IGDINKEINKHRQQRDTKIKFLNTLFKQIL |
| | | SIEEKEQYKHIEINNDEDLIRAIRDFISLN |
| | | ESKISEGTKIFNQFIQRCLQKEDLGQIYLP |
| | | KDSVNTIAHRIFKPWDEIMALFDRKYFVSL |
| | | EEIKDLTESSVWKERVLEESKTKSLIFKDT |
| | | HIHTIISGQEIFSNFILILEKEYKNQFSGF |
| | | ISETRRGKAAFVGYDESLKNLRATIKWFEG |
| | | KNLKLSETEKVEWIKAIKDYADAALRIFQM |
| | | TKYLWLPVVGDEEDKDYLRIKAEIDQLTKD |
| | | NDFYNKINAFIDGYKPEPFIYRSSFQEYLT |
| | | RRPFSTDKFKINFENSRLLDGWDKDMIDDR |
| | | MGILLQRDGDYFLGILNKEDRHCLDNLVDV |
| | | KSEDKNSYALMQFKQLTGLYRQLPRMAFPK |
| | | KKQPVLEANAEIKKIKEDFDFLQKQKKERE |
| | | VNVNVVFDNKKLNLLINHYAEFLKENYKDE |
| | | KCYDFSLLNKEKVYESLSDFYADVDKITYS |
| | | LSFIQVSIDQLIKTGKILLFRLKNKDLLKG |
| | | SLGQNKNLHTYYFHALFERENLSQGRIRLG |
| | | AQAEIFFRPASIEKEKDKNRSNALKKSPKT |
| | | RYVKEILKNKRYSEDKVFLHLPIQLNADAY |
| | | DLPSINQNVFEFIKNRQEKVKIIGIDRGEK |
| | | NLAYYSVISQNSNGKIKIEEPPRDLNLGYL |
| | | EPLDELENKRQDERKAWQSISEIKSKRDGY |
| | | ISYAVSKIVELMLKYQAIIVLEDLSGKFKR |
| | | SRMKFEKAPYQQLELALIKKLNYLVKKNSK |
| | | SGKPGHYLSAYQLTEPVGSYKEMGKQTGII |
| | | FYTQAGYTSRTCPTCGWRKRVQGLYYKDRT |
| | | SAQRRFDPKTGVKIFYDSVNDRFVFQYHPV |
| | | YEQKELKEWDKEIYSDVTRIRWNNEEKKNN |
| | | EYRKGDITLKIKRLFRDRGIDLSRNINEQL |
| | | VNVGDASFWEELINLLRLITEIRNIDNENN |
| | | RDFIECPHCHFQSENGFHGVAWNGDANGAY |
| | | NIARKGLLITKAVCDPEKNVGDITWSDLKV |
| | | DMKDWDAATDEWAKKNPEK |
| SEQ | Cas12 | MENEKIFSDLTNRYQVVKTLPFELKPVPRT |
| ID | Variant | RVLLGLDNPNKGEIFSKDRERAENFTIIKK |
| NO: | | YIDRLHSLFINESLKKADIDFSNFYKQYGK |
| 51 | | NINTKNNKNIDDDNDINDDEKEDSENDNLK |
| | | KYRQEIANLFNKSKYKSWVNVGKDGDKISG |
| | | MLFEKGLIDLLRTHFSDNLNEDIEIPELFS |
| | | NKKIKDTRKLKEIINSFGKDGKDGQNFTTY |
| | | FSVSFHNNRKNYYKSDGKMGRVSTRIVDEN |
| | | LERFCKNIYLYKEIIGKNEIKEIFSGNWDI |
| | | YLQKKPNFSNDKTYKKLDEFKNDKYDWEMI |
| | | FRDVNSYNKYFLQSDIEFYNYIRGKLNQDI |
| | | NEYNGKKRDSKEKINSQFENLRNQVHGEKK |
| | | NYDDDFEIDEDNIIQFINEIFVRHNQNKMR |
| | | FSEKLFSDFIDLLMVDNGDKLDKVYFSQKA |
| | | VENAIARYYFVEETTNEGREPLLISLLLQN |
| | | AGKDRKKLSNKPIKLGDIKFVLDQANNKPA |
| | | EDIFKNRYVLSESNNDGIINANDKNHWANL |
| | | LRLIKKDFYFHKDNLIKSQDKLALETKYNK |
| | | GSDEGERQIETIKNFAESAKAILRMTKYFD |
| | | LRKNGVIQNVIGGKDPIHEEVDKYFDGDVL |
| | | SGEESCRISKYYDALRNFITKKAWSADKII |
| | | LNFDCSEFLGGWDRSQEQKKRGIILRHRDG |
| | | DEERYYLAVLGKNGKQYFENRTLFKGCESS |
| | | DWQKIEYNVIQKPHMSLPKNLITPFFKKDK |
| | | ITNERFIDRSKKGAKALIEIDINPSDEFLN |
| | | NYNLGKHTKENLDKSFLCDYFKYLMDAIAK |
| | | YYKGEFNFNFPDVSNFDNTQPFYSFIEKNA |
| | | YSIKYFGISSKEIEKLIADCYYKEDVYLFQ |
| | | IYCKDFEIDPKIGKAKYGNEFRTKAEIRKS |
| | | KGEEAGNENLNTKYFKLLFDEKNLKNQNGI |
| | | VYKLNGGAKMFYRPSSIKKDEKIDGKWRYK |
| | | EDKYSLNITITCNFSSKKDDLSIDKDINKK |
| | | IAEVNANSDFRIISIDRGEKNLAYCCVMDE |
| | | NANILDIKSLNRITRYDKNGKAIKEKNMFH |
| | | EVKDGKLCYGEPVYDFYKDYQNLLDEREIK |
| | | RLVNRRSWNVIEDIKNLKKGYVALLINYIC |
| | | KAVVIAINEGKYPIIVLESLDKGMLHNRVK |
| | | IEKQIYRGVEEGLVRKLNYFVDKKTDNVLN |
| | | AWQLLAKFETVGSSLDRKKQLGIIFYVDPG |
| | | YTSITCPCCGFRQRKYIKAERAEENFKEIK |
| | | IKFDGKRYSFAYDYRCIDDNGKEKSKEDII |
| | | YSNVKRLLRSGRNGRAVQIEDVTDELTNLF |
| | | KKHNINIEQDINEQLAGKDNKFWKQLLWWF |
| | | NAIEQIRNTQSLRRKFNTEENKLEILENND |
| | | CDFILCPHCYFDSNKDKFQNKIWNGDANGA |
| | | FNIGRKGIIDIFEIKKHQRMLSDFMEQWGI |
| | | DKLPKANGGNQAVIEIVKNDKKYNLCILNN |
| | | KKIPYYCLRIGKEKIDSIADDRKCNQLPDL |
| | | MVNWKKWDMWLDKWGK |
| SEQ | Cas12 | MPEVKNVFQDFTNLYELSKTLRFELKPVPE |
| ID | Variant | TEKILELNAAKTKKFPKDLYRAENFEIIKK |
| NO: | | YTDELHRTYIRETLNNVNIDYLKFLEIFRI |
| 52 | | NGKKKNEMTDENEESDENNEKDDIQKIKKE |
| | | LRSKIGNLFNKWNNDKDNKFKDWVKIDVGK |
| | | KEKEVSGDLFGKELITILKNYFKNKLDSKV |
| | | NVPMLFFNEQEIKNGEAKKQRKLEAVFENF |
| | | DKFTTYFTDSFYNNRKNYYKTEGRVGQVAT |
| | | RIIDENLPRFCSNLIAFNEVVSLYSTLLNN |
| | | FDLGWKEYLNEKKINQTWVEKFELSNYDWK |
| | | ALFNDVNYYNQCLLQEGIDKYNYIIKKLNK |
| | | DINEYTQNKYKSVEKGNNNNPDINFFQKLH |
| | | KQIHGERDFKLIEIDIDENNIFTKILPEFI |
| | | LHSDMKLMTKIDEEVGVEEIVGAERIIKIF |
| | | IKQELKDLEKIYLSRRAIETISAKWFHSWE |
| | | TLKDLILGYLNKDLLESKKRKKVPDFVDFN |
| | | IIKIVLENNKDDYKDLFKRKYFEADKNEFV |
| | | DWIDSSGGTKKLEFGGENWINFLNVFEYEF |
| | | GTLLTEYKKNKNALLYLIDKKIDYDKNNEV |
| | | GQTAAIKNFADSALGIFRMVSYFALRKKGV |
| | | MVEPKNGKDEIFYAFVDRYLDGDDNDREEQ |
| | | NKIVQYYNTLRNFVTQKAWSIDKVRLCFDC |
| | | GEFLKGWDKDKIHERLGIILRNNNKFYLGI |
| | | LNKNHKQIFIKIKSHDNNNFYYVIYDYKQL |
| | | NNVYRQIPRLAFPSRSVKKGDAYMLRAIQE |
| | | RKKKFFLEDEEFIELQEIKNEYDKIGNDLS |
| | | KEKLTKLIEYYKKVVISNYSSLYNVSNLNN |
| | | KKFNSINEFNQYVENLMYSLIPTRISPDFI |
| | | KEKISKGELYLFQIYNKDFELDESIGKEKF |
| | | GEDFAPVIMDGKNNLHTEYFKLLFNDSNLK |
| | | NPNGVVFKLSGGAKMFYRPATENLPIKKDR |
| | | DGNIIKNKKGENVIVGQRYKEDKYFLHLPI |
| | | ILNFVNKGKNYSINDMVNKAITNASDDQDK |
| | | FRIIGLDRGEKHLVYYSVINERQEIIEIGS |
| | | LNNISRKDNKGEIIEEKNWYHDKFGNIEKE |
| | | PTKEYHKDYHNLLDQREIERLKSRQSWEKI |
| | | ENIKELKEGYISAVINKICNLVIKAIKENK |
| | | IPIVALENLNSGMKRGRIKIDKQIYQKLEL |
| | | KLAKKLNFLVDKKEKNYLSAWQFTPKIETF |
| | | SGDIEKKNQVGIIFYVDPAFTSATCPNCGF |
| | | RKRIKMDPQNAKKKIKDMEITYENGIYKFD |
| | | YPIENGENDVVYSDVERLKWDNEKKKVIKT |
| | | KNVSDDFGKLFEDIKDKNNLKKELLSIGEE |
| | | NKEFWKEFSRCFNLLLRIRNSKLIKRKLND |
| | | DTGKVEIIADDDLADRDRDFIYCPQCHFHS |
| | | EGGDVFGEFVKKKYLGKDNFEFNGDANGAY |
| | | NIARKTIIAVNKIKDYQLGLNHFIEKYRIS |
| | | ELPNNGKDKKNIFYNNNSYILSFFEVQDEK |
| | | FRKVKVYGLKKDGDRQIIQKKEMWYRRYPD |
| | | IFVNNKEWDKFVQNKS |
| SEQ | Cas12 | MLFFMSTDITNKPREKGVFDNFTNLYEFSK |
| ID | Variant | TLTFGLIPLKWDDNKKMIVEDEDFSVLRKY |
| NO: | | GVIEEDKRIAESIKIAKFYLNILHRELIGK |
| 53 | | VLGSLKFEKKNLENYDRLLGEIEKNNKNEN |
| | | ISEDKKKEIRKNFKKELSIAQDILLKKVGE |
| | | VFESNGSGILSSKNCLDELTKRFTRQEVDK |
| | | LRRENKDIGVEYPDVAYREKDGKEETKSFF |
| | | AMDVGYLDDFHKNRKQLYSVKGKKNSLGRR |
| | | ILDNFEIFCKNKKLYEKYKNLDIDFSEIER |
| | | NFNLTLEKVFDFDNYNERLTQEGLDEYAKI |
| | | LGGESNKQERTANIHGLNQIINLYIQKKQS |
| | | EQKAEQKETGKKKIKFNKKDYPTFTCLQKQ |
| | | ILSQVFRKEIIIESDRDLIRELKFFVEESK |
| | | EKVDKARGIIEFLLNHEENDIDLAMVYLPK |
| | | SKINSFVYKVFKEPQDFLSVFQDGASNLDF |
| | | VSFDKIKTHLENNKLTYKIFFKTLIKENHD |
| | | FESFLILLQQEIDLLIDGGETVTLGGKKES |
| | | ITSLDEKKNRLKEKLGWFEGKVRENEKMKD |
| | | EEEGEFCSTVLAYSQAVLNITKRAEIFWLN |
| | | EKQDAKVGEDNKDMIFYKKFDEFADDGFAP |
| | | FFYFDKFGNYLKRRSRNTTKEIKLHFGNDD |
| | | LLEGWDMNKEPEYWSFILRDRNQYYLGIGK |
| | | KDGEIFHKKLGNSVEAVKEAYELENEADFY |
| | | EKIDYKQLNIDRFEGIAFPKKTKTEEAFRQ |
| | | VCKKRADEFLGGDTYEFKILLAIKKEYDDF |
| | | KARRQKEKDWDSKFSKEKMSKLIEYYITCL |
| | | GKRDDWKRFNLNFRQPKEYEDRSDFVRHIQ |
| | | RQAYWIDPRKVSKDYVDKKVAEGEMFLFKV |
| | | HNKDFYDFERKSEDKKNHTANLFTQYLLEL |
| | | FSCENIKNIKSKDLIESIFELDGKAEIRFR |
| | | PKTDDVKLKIYQKKGKDVTYADKRDGNKEK |
| | | EVIQHRRFAKDALTLHLKIRLNFGKHVNLF |
| | | DFNKLVNTELFAKVPVKILGMDRGENNLIY |
| | | YCFLDEHGEIENGKCGSLNRVGEQIITLED |
| | | DKKVKEPVDYFQLLVDREGQRDWEQKNWQK |
| | | MTRIKDLKKAYLGNVVSWISKEMLSGIKEG |
| | | VVTIGVLEDLNSNFKRTRFFRERQVYQGFE |
| | | KALVNKLGYLVDKKYDNYRNVYQFAPIVDS |
| | | VEEMEKNKQIGTLVYVPASYTSKICPHPKC |
| | | GWRERLYMKNSASKEKIVGLLKSDGIKISY |
| | | DQKNDRFYFEYQWEQEHKSDGKKKKYSGVD |
| | | KVFSNVSRMRWDVEQKKSIDFVDGTDGSIT |
| | | NKLKSLLKGKGIELDNINQQIVNQQKELGV |
| | | EFFQSIIFYFNLIMQIRNYDKEKSGSEADY |
| | | IQCPSCLFDSRKPEMNGKLSAITNGDANGA |
| | | YNIARKGFMQLCRIRENPQEPMKLITNREW |
| | | DEAVREWDIYSAAQKIPVLSEEN |
| SEQ | Cas12 | MTIKKHKPFTNFECLTPVQKTLRFRLIPVG |
| ID | Variant | RTTEFVKCRNIIEADRKRSEMYPLLKELAD |
| NO: | | RFYREFMTDQLSNLLFDWSPLVEALLLARN |
| 54 | | NTDPRENQRIASLVRDEQKKYRTLLLKRLS |
| | | GQVDRNGTPLPKNTASVNKKYYDDLFWQAR |
| | | KNIFTDEDIATGFAYRIVHEIVPDYLFNRR |
| | | VYEQHKLDFPEPLDLKARFVTETLPAYLEH |
| | | LKNKPDGRISDELFDAYKDALDSYQKFTSR |
| | | LTNFLETELKKKNLIANDESLDALFTIPAI |
| | | NRLLTQKGVDLHNAVIGGFFTDDHTKVQGF |
| | | NELANLKNQTLKNVSDNSEIKPVGKMTRLK |
| | | KHILSISESTSFLFEQIESDDDLLARIIEF |
| | | NNTLSEPDIDGLSIADINDQLYNIMTGVDP |
| | | STILVHARNLNKLSHEASLSWNRLRDGLYQ |
| | | MATESPYREDERFKRYIDASEEERDLSKLK |
| | | NDIYFSLQELQFALDQSIDLEEEATPTEDI |
| | | FLPFEFPGMDLKSELTVLFRSIEQLISSET |
| | | KLIGNPDAIATIKKYLDAIMARYSIWNLLS |
| | | CEAVELQDDLFYPEYDRVMGSLSNIILLYN |
| | | LARNYLSRKPSSKEKFRLNFDKPTLADGWS |
| | | ESKVPDNFSVLLRKDDLFYLGILKDRKAYR |
| | | VLSYENCDETAKNIKGYYERMIYHFSPDAY |
| | | RMIPKCSTARKDVKKHFGEQGETTGYTLYP |
| | | GASNFVKPFTIPYEIYRLQTELVNDKKRYQ |
| | | ADYLKQTEDEEGYRQAVTAWIDFCKSYLES |
| | | YEGTSTFDYSHLLKSEDYEDVNQFYADVDR |
| | | ASYSIYFEKVSVDLIHTMVDRGDLYLFQLY |
| | | NKDFSPHSTGKPNLHTMYWRALFSNDNLQN |
| | | NTIKLNGQAELFYRPKQVEQPTVHLQGSYL |
| | | LNRFDKHGDVIPAGLYCEIYNHINERHPEG |
| | | YTLSEEATQGLLDGRFVYREAPFELVKDKR |
| | | YTEDQLFLHVPLEFNWTASANVPFENLANE |
| | | YIKKDSDLHIIGIDRGERNLLYYSVINLQG |
| | | DIVKQGSLNTLIQQTTLKGETVERQIPYQS |
| | | MLKQREDERAEARQNWQSIDRIKDLKEGYL |
| | | SHVIYKLSRLIIKYHAIVVMENLNVGFKRG |
| | | RFKVERQVYQKFEVALINKLNALSFKEYEP |
| | | NELGGVMRPWQLARRVVSPEDTRSQNGIVF |
| | | YVPASYTSIVDPVTGFANLFYLNRIRNKDL |
| | | NSFYGHFQEIRYDHEFDRFIFRFNYADFGV |
| | | FCRIKNVPSRTWNLVSGERKAFNPKRRMIE |
| | | KRDTTDEIKKALEAHGIAYQNEQNLLPLLL |
| | | ENENLLARIHRSFRLVLQLRNSDSDRDDIV |
| | | SPALDKENNTFDSGQQPYESSLPINADANG |
| | | AYNIARKGLLLVDKVKNDKRAVLSNREWFE |
| | | YLMAEE |
| SEQ | Cas12 | MENKDYSLSRFTKQYQNSKTVRFALTPIGR |
| ID | Variant | TEEYIIQNQYIEAARRKNQAYKIVKPIIDE |
| NO: | | KFRSMIDDVLTHCEKQDWVTLDKLILQYQN |
| 55 | | NKCRENMDALAEQQEEIRKNISEEFTKSDE |
| | | YKNFFGKEDSKKLFKIFLPEYLNQINASES |
| | | DKEAVNEFQKFKTYFSNFLIVRADIFKADN |
| | | KHNTIPYRIVNENFMIFAGNKRTFSNIIRL |
| | | IPNALEEIAKDGMKKEEWSFYNIQNVDSWF |
| | | EPDSFQMCMSQKGIQKYNFIIGLVNSYINL |
| | | YTQQNPQATEVKRSRLKLRMLHKQILSDRV |
| | | NPSWLPEQFKEGEEGEKQIYEAILALENDL |
| | | IKNCFDKKYDLWIQSIDIQNPRIYIAASEM |
| | | ARVSSALHMGWNGLNDVRKTILLKSDKKQA |
| | | KVEKILKQDVSLKDLSDTLNRYADIYKEEQ |
| | | IPSLYQYIEYGSELLQDCAITRKEYHDLLN |
| | | GNSNTLSLNQNEKLIEGLKAYLDSYQAIVH |
| | | FLNVFIVGDELDKDTDFYAELDGLVESLSE |
| | | IVPLYNKVRNYITRKVYSLDKMRIMFERSD |
| | | FLGGWGQSFDTKEALLFQKDNLYYIGIIEK |
| | | KYTNMDVEYLHEGIKEGNRAIRFIYNFQKA |
| | | DNKNIPRTFIRSKGTNYAPAVRKYNLPIES |
| | | IIDIYDVGKFKTNYKKINEKEYYESLEKLI |
| | | DYFKDGILKNENYKKFHFNWKPSNEYENIN |
| | | EFYNDTNNACFLLEKEEINYDHLKEQANQG |
| | | KIYLFQISSKDFNEGSKGTPNLQTMYWREL |
| | | FSNQNCKDGVIKLCGGASIYMRDASIKQPV |
| | | VHRKNAWLINKWYKVNGQNVVIPDNTYVKF |
| | | TKIAQERMNEDELTPQERQLWNSGLIQKKK |
| | | ATHDIMKDRRFTKKQYMLHAPLTINYKQQD |
| | | SPRYFNEKVRSFLKDNPDINIIGIDRGEKN |
| | | LIYITIIDQKGNILKGMQKSFNQIEEKGKE |
| | | GRTIDYYSKLESVEARHDAARKNWKQIGTI |
| | | RELKEGYLSQVVHEITQLMIQYNAVIVMEN |
| | | LNMGFKKGRMKVEKSVYQKFEKMLIDKMNY |
| | | LAFKRDMQGNAIDPYEVGGVMNGYQLTDRF |
| | | TSFADMGSQNGFIFYVPAAYTSVIDPVTGF |
| | | VNVFQKTEFKTNDFLHRFDSISWNDKEQSF |
| | | VFTFDYQNFKCNGTCYQNKWSLYADVDRIE |
| | | TIIKNNQVDRIEPCNPNQKLIDFFDKKGII |
| | | YRDGHNIVDDLEKYDSKTISEIIHNFKLIL |
| | | QLRNSMRNPDTGEIIDYIASPVMHNEERFD |
| | | SRKRNPELPQDADANGAYHIALKGLMFLQK |
| | | INEYADSDGNMDNRKLKITNEEWFKYMQTR |
| | | KEHTYF |
| SEQ | Cas12 | MSNKTSSITTTNKLSYTGFHNNGKQSKTLM |
| ID | Variant | FELKPIGRTTEHLDRKGYLADDIDRAESYK |
| NO: | | TFKEIADNFHKNLIEESLATFTFSDTLKDY |
| 56 | | FDLWLSPVRTNEDTPKLRKMEAKLRKELSS |
| | | ALKQHPSFAATSSGKRLIDEALYPNASDKE |
| | | RQCLDRFKGRSSYLDSYTEVRSFIYTDLCK |
| | | HNTIAYRVVNENLKIYLENILAYEKLMQTA |
| | | VNGKLETVKEMFHDLYPTFSMDISIFFTSY |
| | | GFDYCLSQNAITRYNILLGGWSDDNGIHHK |
| | | GLNNYINEYNQTVPRNKRLPKLNKLQKMIL |
| | | SEENSMSFIIDKFENDVDLANAIRYWLKNC |
| | | QFDALNLLIWTLDVHYNLDEIHFKNDNQGK |
| | | NISDLSQALFKNHHVIRDAWDYDYDIVNAK |
| | | AKSRQKPERYAEKRDKAFKKINSFSLSYLA |
| | | NILSQYDNQYANFVAQFKTRISVHIQNVQQ |
| | | MIADKTLDMRLDPLMLLKSISSDTKLVEDI |
| | | KRVLDSLKDMQRMLTPLLGEGTEPNRDAMF |
| | | YSDFEPLMNYVDTLTPLYNKVRNYITKKPY |
| | | STKKTSLYFGASNFGSGFDVTKLPVSHTII |
| | | MRDKGCYYLAVIDNNKLIDKLYDHNDNDGY |
| | | EYMVYKQIPSPIKYFSLKNILPQDPPDDIR |
| | | QLLEDRKNGAKWSHDDETRFIDYIVNEFLP |
| | | TYPPIHDKNGNPYFSWKFKNPDEYESLNEF |
| | | FDDVSKQAYQTSFRFVSRDFVDDAVENGDI |
| | | FFFQIYNQDFSPASHGKPSPHTLWFRALFS |
| | | DVNLETKDIRLKGNATAYFRPASIFYTDEK |
| | | WRKGHHYEQLKNKFKFPIIKDKRYALDKFF |
| | | FHITLEINCNATVEKYFNNRVNEEIRKADR |
| | | YNILAINRGERNLLYAVVMDQDGTILEQKS |
| | | FNIIKSELPNKTVKETDYWKKLHAREKERD |
| | | TARKSWKSIECIKDLKKGYLSYVVKTITDM |
| | | MFEYNAVLVMENLDIEMKRSRQKIEKNVYA |
| | | QFQNAIIQKLSMYVNKDIDLHIARTAPGGT |
| | | LNPYQLTYIPASRTKTPKQNGFVFFLNPWN |
| | | ITEIDPTTGFVDLFQTCFRTKNEYKDFFAK |
| | | FKDIRYNEAQGWFEFDTDYTYFRDKEKAGK |
| | | RTRWNICSYGTRLRRFRNPDKNYAEDAMTV |
| | | YPTQMLKDLFDEYNIPYAPASAKSTSISIK |
| | | DDIIQIDKLDFYKKLLYILKLIVQLRNTSP |
| | | SSTEQEDDYIISPVINEDTNWFYDSRDYNE |
| | | ESLLPCNTDANGAYSLALKCNMVIDRIKNT |
| | | IPGEPVDMYISNADWLDARQ |
| SEQ | Cas12 | MNSKTSIFDFSNIFGRDITLRFKLTPVTIN |
| ID | Variant | SKGEVKDANGADPYRPYLSADEELQEQYEL |
| NO: | | LKTAIDAYHQMYIDKKLKHILCLPLTEKGK |
| 57 | | DGVEHDTALYEKDGKSTSVPYRCIDRNLPR |
| | | FSKDYHLFEKILGDCSDVFDFEQLDKDKSK |
| | | FVKSCLAYIKDYGEKDKKRQTADLRTFISR |
| | | VFADDNISSLPPYKVKSDFITKTLRQWLEQ |
| | | PDTKVEKKEAILDLIEKNGSKLYANCQGLL |
| | | EARQRFSEELKGIARLSGIRVESVREVFQP |
| | | LLYLAYLNQEGIQYLNTIIGTKKEKGTSAL |
| | | GLNEYINQYNQKQGIKKKKDGIPMLNKLNN |
| | | QILFGDEVFIETLAEHKEAIPVIKKVVSSL |
| | | GKLGAFDGECHENKLYQFLLSLSSYAGNIY |
| | | VNTKVVAQISSSLWGDYSILYDAVKHDKNG |
| | | RLIQKSVTLGELNEKIERLKLEDNRDAFEY |
| | | FRRSQVKDVVHGSSNVGVFEQLKNCYNDFV |
| | | EKKILKCSFFSEDQVLVIQRLFDSILSLQR |
| | | IFKVFCPSLYEVDSDGLFVAKFSDYWNVLR |
| | | GFDKDYDLLRNLFKRKPYSTDKIRVHFGLS |
| | | NLMDGFVDSWTDKKDKGTQYNGYILRQAHS |
| | | FVDENTSKELQEFQRYNYYLVISGNVRLFR |
| | | EKGNALVCEKKKEKLVASDEFSGFERFDYY |
| | | QSSINNFNREFKRLTGRDRKSFTDEILQNE |
| | | GKKELKSTYIENLIKVAKSMKRLTALQNLV |
| | | SDEKVRKYSENLDYETLSAEIGQILATGRE |
| | | RKYVPVSTNEMKNLLKSSKNNKGEEVRTFM |
| | | FRISNKDLSYAETMQKGERKSHGAENMHTM |
| | | YFRALLDTLQNTFDIGTGTVYFRKASDKRK |
| | | MKYDEKNPTHRKGDELAFKNPYNKGKKKSV |
| | | FGYDLIKDRRYTKDSYLFHLSITQNYQKKG |
| | | NAEDLNAMVRDYIRTQEDLRVIGIDRGERN |
| | | LLYATMIDGEGHILAQKSFNVIGYQGTTAS |
| | | GESFQVETDYHQLLNEKAEKMRSLQREWKE |
| | | MDKIQDMKDGYLSVVVHELAKMVVENNAII |
| | | VMEDLNMGFMESRQSQLANVYQKFEEKLRN |
| | | KLQFYVDKRKRNDEPSGLYHALQLAGTETK |
| | | DNQNGFIFYIPAWNTSKIDSVTGFVNLFNL |
| | | KYTNIKDAKAFFSTFEKIEKNVETGHYDFT |
| | | FSYSSMARKKMAKRMDGTRDSWTISTHGSR |
| | | IVREQKGNYWEYREIESLTSEFDALFEKYS |
| | | IDTRCRLKEAIDKCGEAEFFKELIRLMKWT |
| | | LQLRNYDDRGNDYIVSPVCYRGNEYYCSLD |
| | | YDNEEGMCISKIPCQMPKDADANGAFNIAR |
| | | KGLMLCERLKKGEKIGVIKGTEWLQYVQNM |
| | | SERYVGMV |
| SEQ | Cas12 | MINTMEQPKKSIWDEFTNLYSLQKTLRFEL |
| ID | Variant | KPQGKTKELVRTLFINPEEHHHKLISDDLE |
| NO: | | LSKNYKKVKKLIDCMHRNIINNVLSKHQFT |
| 58 | | GEELKKLDKNSNAEDNDTETDNADKKDPFA |
| | | KIRERLTKALNEESKIMFDNKLLNPKKGKN |
| | | KGECELKKWMDKAEDKYFELGNNEKIDKEA |
| | | VKADMERLEGFFTYFGGFNKNRENVYSSKK |
| | | IATAIPFRIIHDNFPIFKKNIENYKKITEK |
| | | HPELAKLLNEKGANEIFQLEHFNKCLTQDG |
| | | IDVYNNEKLGIIAKEQGKEQDKGINQLINE |
| | | YAQKKNKEIKENAKGGEKPKKIKIAVFDKL |
| | | KKQILSISKTKSFQFEVFEDTSDIINGINK |
| | | RYTFLTEAKEGMSIVDEIKKIIGSVGDEKY |
| | | SLDEIYLKEKFISTLSKKLFNYSRYIEVAL |
| | | EKWYDDRYDDKINKSGTDKRKFISAKQFSI |
| | | TSIQDAINYYLEKYEKDEELSKKYTGKNII |
| | | VDYFKNPTITIEHKQKEEVISEEKDLFKEL |
| | | EVRRNVIQHILNGDYKKDLKEEKQQDGDSE |
| | | KVKAFLDALLEFNYILNPFIIKDKNLRKEQ |
| | | EKDEEFYNEIKKLQESIFEAEILDLYNQTR |
| | | NYITKKPYKLDKFKLTFGSGYFLSGWSNDM |
| | | EEREGSILIKYNEDRSKNYYLIIMAKPLTD |
| | | DDKKQLFSDNGTHSKICIYEFQKMDMKNFP |
| | | RMFINSKGSNPAPAIEKYNLPIKTIWADYQ |
| | | KYKNLNQKGKDKFLEENPDFRHNLIGYFKI |
| | | CAEKHESLAPFKHQFSSIWKPTKEYENLAQ |
| | | FYKDTLEACYNLKFENVNFDNISQLVSSGK |
| | | LHLFKIHNKDFNPGSTGKKNLHTLYWEMLF |
| | | DEKNLQDVIFKLSGGAELFYREASILKNKI |
| | | IHKIGEKVLKKFFKLPDGKLEPVPAESIKN |
| | | LSAYFRKELPEHELTEIDRKYIDNYSIIGK |
| | | KDDKLGIMKDERFTVDKIQFHCPITINFKS |
| | | KNKNFINDDVLEYLHKRDDVHIIGLDRGER |
| | | HLIYLTMINKDGKIVDNMQFSLNELQRRYK |
| | | INGNEEIQKINYQKLLDTREVSRTEARRNW |
| | | QTIENIKNLKEGYLSLIVHQLAKLMIEKNA |
| | | IVVMENLNYGFKDSRARVEKQIYQKFESIL |
| | | IKKLQYLVMDKNNLYDSGGVLSAYQLTNQE |
| | | VPAYKYISKQNGFLFYVPPDYTSKIDPETG |
| | | FINLLDTRYYSRKNAVALLNKFDKIYYDRD |
| | | NKYFRFDFDYNSTDSNGNKNFDKLRVDISE |
| | | LTRTKWSVCSHPAKRSITVQINNKWVRQPI |
| | | NDVTDKLIKLFEDKQIGYESGKCLKDEILK |
| | | VEDAKFFEDLLRYLSVLLALRHTYTENGVE |
| | | YDLIISSVEKAPGSNEFFVSGKDNNLPANA |
| | | DANGAYNIARKGLWLLRKLDEIDNQELAIK |
| | | KFNELKHAKEIKKNGEESKEDKGDRKRKKK |
| | | WVSQWCPNKEWLAFAQSMQDVSEK |
| SEQ | Cas12 | MNNGTNNFQNFIGISSLQKTLRNALIPTET |
| ID | Variant | TQQFIVKNGIIKEDELRGENRQILKDIMDD |
| NO: | | YYRGFISETLSSIDDIDWTSLFEKMEIQLK |
| 59 | | NGDNKDTLIKEQTEYRKAIHKKFANDDRFK |
| | | NMFSAKLISDILPEFVIHNNNYSASEKEEK |
| | | TQVIKLFSRFATSFKDYFKNRANCFSADDI |
| | | SSSSCHRIVNDNAEIFFSNALVYRRIVKSL |
| | | SNDDINKISGDMKDSLKEMSLEEIYSYEKY |
| | | GEFITQEGISFYNDICGKVNSFMNLYCQKN |
| | | KENKNLYKLQKLHKQILCIADTSYEVPYKF |
| | | ESDEEVYQSVNGFLDNISSKHIVERLRKIG |
| | | DNYNGYNLDKIYIVSKFYESVSQKTYRDWE |
| | | TINTALEIHYNNILPGNGKSKADKVKKAVK |
| | | NDLQKSITEINELVSNYKLCSDDNIKAETY |
| | | IHEISHILNNFEAQELKYNPEIHLVESELK |
| | | ASELKNVLDVIMNAFHWCSVFMTEELVDKD |
| | | NNFYAELEEIYDEIYPVISLYNLVRNYVTQ |
| | | KPYSTKKIKLNFGIPTLADGWSKSKEYSNN |
| | | AIILMRDNLYYLGIFNAKNKPDKKIIEGNT |
| | | SENKGDYKKMIYNLLPGPNKMIPKVFLSSK |
| | | TGVETYKPSAYILEGYKQNKHIKSSKDFDI |
| | | TFCHDLIDYFKNCIAIHPEWKNFGFDFSDT |
| | | STYEDISGFYREVELQGYKIDWTYISEKDI |
| | | DLLQEKGQLYLFQIYNKDFSKKSTGNDNLH |
| | | TMYLKNLFSEENLKDIVLKLNGEAEIFFRK |
| | | SSIKNPIIHKKGSILVNRTYEAEEKDQFGN |
| | | IQIVRKNIPENIYQELYKYFNDKSDKELSD |
| | | EAAKLKNVVGHHEAATNIVKDYRYTYDKYF |
| | | LHMPITINFKANKTGFINDRILQYIAKEKD |
| | | LHVIGIDRGERNLIYVSVIDTCGNIVEQKS |
| | | FNIVNGYDYQIKLKQQEGARQIARKEWKEI |
| | | GKIKEIKEGYLSLVIHEISKMVIKYNAIIA |
| | | MEDLSYGFKKGRFKVERQVYQKFETMLINK |
| | | LNYLVFKDISITENGGLLKGYQLTYIPDKL |
| | | KNVGHQCGCIFYVPAAYTSKIDPTTGFVNI |
| | | FKFKDLTVDAKREFIKKFDSIRYDSEKNLF |
| | | CFTFDYNNFITQNTVMSKSSWSVYTYGVRI |
| | | KRRFVNGRFSNESDTIDITKDMEKTLEMTD |
| | | INWRDGHDLRQDIIDYEIVQHIFEIFRLTV |
| | | QMRNSLSELEDRDYDRLISPVLNENNIFYD |
| | | SAKAGDALPKDADANGAYCIALKGLYEIKQ |
| | | ITENWKEDGKFSRDKLKISNKDWFDFIQNK |
| | | RYL |
| SEQ | Cas12 | MSNLNTFISPEFTGKIKMTKSLKVSMIPIG |
| ID | Variant | ETEHWIAKHKVFEKDRELFDKNLKARPILD |
| NO: | | EFIKYTVSRALPNLLFDFEAYYLVKKDRTK |
| 60 | | ARAFEKELAKTVTDLILKEMDELKSASLID |
| | | SADFVKTTLKKFAGTHDIPGLSRIEAIESL |
| | | EAASKLTALNGKFNTSRIAIINTLIPKRII |
| | | ENFDIYLSNMEKIRNVYESGEFGFLFERYP |
| | | DTLLFMEPANYRTVCSPEAIEDYNRFISGY |
| | | GDSTESWIKGFNQELSEASNSSKSSNGGVR |
| | | RYSLIKPLHKQHLFETKKFFTFASISSDDD |
| | | VRELINSVKGSTEDACLNALAFFSSSDPKT |
| | | LFVKGSYLHTLSAFLYGSANSYILPERIKE |
| | | GEKARLTAEYDSVAKKTKAVTTRYNVAMNN |
| | | ISKKINEKIFSLADIDAYCCDISKRRSVRE |
| | | ILLGIMQEMYAAVYGENGKWSNIEAEAVLD |
| | | SKTKIWKAKNGAVAKAVNDYLTAILEIRKF |
| | | IRPFALRMEELEELGLDTSSALDAGEITNT |
| | | LFEAVRAQKLVHAYLTRNDADIALSTQVYF |
| | | GGTQKAAASWWNYETGDIQNRQIALAKKDG |
| | | MYYFIGTFDERGSYSIEPASPGEDYYEMLD |
| | | VKKGQDANKQIKKVLFSNKAIREHFADSSN |
| | | DYVITTKVNSPITVRREIFDKYQAGEFKLT |
| | | SQKIRKGDLVGEKEMTYYREYMDLLFQMAK |
| | | GYTEYSRFNMDTLLPIEEYDTENDLLDDVN |
| | | TNTIDYRWVRISAACIDDGVRNGDIFVFRA |
| | | QTSSMYGKRENKKGYTGLFLELVSDENLLV |
| | | TRGMSLNSAMSIYYRAKVHDAITVHKKGDV |
| | | LVNKFTNARERIPENSYKAICAFYNSGKSI |
| | | EELTIEDRDWLAKATTRICSGEIIKDRRYT |
| | | KNQYSISISYNINRSVNNRKRVDLATIVDD |
| | | TASAGRIISVTRGTKDLVYYTVIDDGGSVI |
| | | EARSLNVINGINYAKMLAQISEERHDSNAN |
| | | FDIPKRVETIKEAYCAFAVHEIISAALKHN |
| | | ALIVVELISDAIKDKYSLLDNQVFLKFENV |
| | | LKNCLMSVKVKGARGMEPGSISNPLQLCNA |
| | | DDKSFRNGILYQIPSSYINICPVTGYADII |
| | | DYYNIVSAGDIRNFFVRFENIVYNKEKARF |
| | | EFSFDLKNIPIKLEKCPDRTKWTVLGRGEI |
| | | TTYDPLTKSNHYVFDAAQMLAETVSKEGLD |
| | | PCANIVEHIDELSAATLKKMFNTFRNIAKG |
| | | IVSECDEVPVSYYKSPVIDEADIKNKSLDN |
| | | KSISEIKCYNLDEKARYYLALAKSSSDGEN |
| | | KNRYVSSTAIEWLNYIQEKRTHE |

Alternatively, the Type V CRISPR-Cas enzyme is a programmable Cas14 nuclease. A Cas14 enzyme of the present disclosure includes 3 partial RuvC domains (RuvC-I, RuvC-II, and RuvC-III, also referred to herein as subdomains) that are not contiguous with respect to the primary amino acid sequence of the Cas14 enzyme, but form a RuvC domain once the protein is produced and folds. A naturally occurring Cas14 enzyme functions as an endonuclease that catalyzes cleavage at a specific sequence in a target nucleic acid. A programmable Cas14 enzyme can be a Cas14a enzyme, a Cas14b enzyme, a Cas14c enzyme, a Cas14d enzyme, a Cas14e enzyme, a Cas14f enzyme, a Cas14g enzyme, a Cas14h enzyme, or a Cas14u enzyme. In some cases, a suitable Cas14 enzyme comprises an amino acid sequence having at least 80%, at least 85%, at least 90%, at least 95%, at least 98%, at least 99%, or 100%, amino acid sequence identity to any one of SEQ ID NO: 61-SEQ ID NO: 152.

TABLE 2
Cas14 Enzyme Sequences

| SEQ | |
|---|---|
| ID | |
| NO | Sequence |
| SEQ | MEVQKTVMKTLSLRILRPLYSQEIEKEIKEEKERR |
| ID | KQAGGTGELDGGFYKKLEKKHSEMFSFDRLNLLLN |
| NO: | QLQREIAKVYNHAISELYIATIAQGNKSNKHYISS |
| 61 | IVYNRAYGYFYNAYIALGICSKVEANFRSNELLTQ |
| | QSALPTAKSDNFPIVLHKQKGAEGEDGGFRISTEG |
| | SDLIFEIPIPFYEYNGENRKEPYKWVKKGGQKPVL |
| | KLILSTFRRQRNKGWAKDEGTDAEIRKVTEGKYQV |
| | SQIEINRGKKLGEHQKWFANFSIEQPIYERKPNRS |
| | IVGGLDVGIRSPLVCAINNSFSRYSVDSNDVFKFS |
| | KQVFAFRRRLLSKNSLKRKGHGAAHKLEPITEMTE |
| | KNDKFRKKIIERWAKEVTNFFVKNQVGIVQIEDLS |
| | TMKDREDHFFNQYLRGFWPYYQMQTLIENKLKEYG |
| | IEVKRVQAKYTSQLCSNPNCRYWNNYFNFEYRKVN |
| | KFPKFKCEKCNLEISADYNAARNLSTPDIEKFVAK |
| | ATKGINLPEK |
| SEQ | MEEAKTVSKTLSLRILRPLYSAEIEKEIKEEKERR |
| ID | KQGGKSGELDSGFYKKLEKKHTQMFGWDKLNLMLS |
| NO: | QLQRQIARVFNQSISELYIETVIQGKKSNKHYTSK |
| 62 | IVYNRAYSVFYNAYLALGITSKVEANFRSTELLMQ |
| | KSSLPTAKSDNFPILLHKQKGVEGEEGGFKISADG |
| | NDLIFEIPIPFYEYDSANKKEPFKWIKKGGQKPTI |
| | KLILSTFRRQRNKGWAKDEGTDAEIRKVIEGKYQV |
| | SHIEINRGKKLGDHQKWFVNFTIEQPIYERKLDKN |
| | IIGGIDVGIKSPLVCAVNNSFARYSVDSNDVLKFS |
| | KQAFAFRRRLLSKNSLKRSGHGSKNKLDPITRMTE |
| | KNDRFRKKIIERWAKEVTNFFIKNQVGTVQIEDLS |
| | TMKDRQDNFFNQYLRGFWPYYQMQNLIENKLKEYG |
| | IETKRIKARYTSQLCSNPSCRHWNSYFSFDHRKTN |
| | NFPKFKCEKCALEISADYNAARNISTPDIEKFVAK |
| | ATKGINLPDKNENVILE |
| SEQ | MAKNTITKTLKLRIVRPYNSAEVEKIVADEKNNRE |
| ID | KIALEKNKDKVKEACSKHLKVAAYCTTQVERNACL |
| NO: | FCKARKLDDKFYQKLRGQFPDAVFWQEISEIFRQL |
| 63 | QKQAAEIYNQSLIELYYEIFIKGKGIANASSVEHY |
| | LSDVCYTRAAELFKNAAIASGLRSKIKSNFRLKEL |
| | KNMKSGLPTTKSDNFPIPLVKQKGGQYTGFEISNH |
| | NSDFIIKIPFGRWQVKKEIDKYRPWEKFDFEQVQK |
| | SPKPISLLLSTQRRKRNKGWSKDEGTEAEIKKVMN |
| | GDYQTSYIEVKRGSKIGEKSAWMLNLSIDVPKIDK |
| | GVDPSIIGGIDVGVKSPLVCAINNAFSRYSISDND |
| | LFHFNKKMFARRRILLKKNRHKRAGHGAKNKLKPI |
| | TILTEKSERFRKKLIERWACEIADFFIKNKVGTVQ |
| | MENLESMKRKEDSYFNIRLRGFWPYAEMQNKIEFK |
| | LKQYGIEIRKVAPNNTSKTCSKCGHLNNYFNFEYR |
| | KKNKFPHFKCEKCNFKENADYNAALNISNPKLKST |
| | KEEP |
| SEQ | MERQKVPQIRKIVRVVPLRILRPKYSDVIENALKK |
| ID | FKEKGDDTNTNDFWRAIRDRDTEFFRKELNFSEDE |
| NO: | INQLERDTLFRVGLDNRVLFSYFDFLQEKLMKDYN |
| 64 | KIISKLFINRQSKSSFENDLTDEEVEELIEKDVTP |
| | FYGAYIGKGIKSVIKSNLGGKFIKSVKIDRETKKV |
| | TKLTAINIGLMGLPVAKSDTFPIKIIKTNPDYITF |
| | QKSTKENLQKIEDYETGIEYGDLLVQITIPWFKNE |
| | NKDFSLIKTKEAIEYYKLNGVGKKDLLNINLVLTT |
| | YHIRKKKSWQIDGSSQSLVREMANGELEEKWKSFF |
| | DTFIKKYGDEGKSALVKRRVNKKSRAKGEKGRELN |
| | LDERIKRLYDSIKAKSFPSEINLIPENYKWKLHFS |
| | IEIPPMVNDIDSNLYGGIDFGEQNIATLCVKNIEK |
| | DDYDFLTIYGNDLLKHAQASYARRRIMRVQDEYKA |
| | RGHGKSRKTKAQEDYSERMQKLRQKITERLVKQIS |
| | DFFLWRNKFHMAVCSLRYEDLNTLYKGESVKAKRM |
| | RQFINKQQLFNGIERKLKDYNSEIYVNSRYPHYTS |
| | RLCSKCGKLNLYFDFLKFRTKNIIIRKNPDGSEIK |
| | YMPFFICEFCGWKQAGDKNASANIADKDYQDKLNK |
| | EKEFCNIRKPKSKKEDIGEENEEERDYSRRFNRNS |
| | FIYNSLKKDNKLNQEKLFDEWKNQLKRKIDGRNKF |
| | EPKEYKDRFSYLFAYYQEIIKNESES |
| SEQ | MVPTELITKTLQLRVIRPLYFEEIEKELAELKEQK |
| ID | EKEFEETNSLLLESKKIDAKSLKKLKRKARSSAAV |
| NO: | EFWKIAKEKYPDILTKPEMEFIFSEMQKMMARFYN |
| 65 | KSMTNIFIEMNNDEKVNPLSLISKASTEANQVIKC |
| | SSISSGLNRKIAGSINKTKFKQVRDGLISLPTART |
| | ETFPISFYKSTANKDEIPISKINLPSEEEADLTIT |
| | LPFPFFEIKKEKKGQKAYSYFNIIEKSGRSNNKID |
| | LLLSTHRRQRRKGWKEEGGTSAEIRRLMEGEFDKE |
| | WEIYLGEAEKSEKAKNDLIKNMTRGKLSKDIKEQL |
| | EDIQVKYFSDNNVESWNDLSKEQKQELSKLRKKKV |
| | EELKDWKHVKEILKTRAKIGWVELKRGKRQRDRNK |
| | WFVNITITRPPFINKELDDTKFGGIDLGVKVPFVC |
| | AVHGSPARLIIKENEILQFNKMVSARNRQITKDSE |
| | QRKGRGKKNKFIKKEIFNERNELFRKKIIERWANQ |
| | IVKFFEDQKCATVQIENLESFDRTSYK |
| SEQ | MKSDTKDKKIIIHQTKTLSLRIVKPQSIPMEEFTD |
| ID | LVRYHQMIIFPVYNNGAIDLYKKLFKAKIQKGNEA |
| NO: | RAIKYFMNKIVYAPIANTVKNSYIALGYSTKMQSS |
| 66 | FSGKRLWDLRFGEATPPTIKADFPLPFYNQSGFKV |
| | SSENGEFIIGIPFGQYTKKTVSDIEKKTSFAWDKF |
| | TLEDTTKKTLIELLLSTKTRKMNEGWKNNEGTEAE |
| | IKRVMDGTYQVTSLEILQRDDSWFVNFNIAYDSLK |
| | KQPDRDKIAGIHMGITRPLTAVIYNNKYRALSIYP |
| | NTVMHLTQKQLARIKEQRTNSKYATGGHGRNAKVT |
| | GTDTLSEAYRQRRKKIIEDWIASIVKFAINNEIGT |
| | IYLEDISNTNSFFAAREQKLIYLEDISNTNSFLST |
| | YKYPISAISDTLQHKLEEKAIQVIRKKAYYVNQIC |
| | SLCGHYNKGFTYQFRRKNKFPKMKCQGCLEATSTE |
| | FNAAANVANPDYEKLLIKHGLLQLKK |
| SEQ | MSTITRQVRLSPTPEQSRLLMAHCQQYISTVNVLV |
| ID | AAFDSEVLTGKVSTKDFRAALPSAVKNQALRDAQS |
| NO: | VFKRSVELGCLPVLKKPHCQWNNQNWRVEGDQLIL |
| 67 | PICKDGKTQQERFRCAAVALEGKAGILRIKKKRGK |
| | WIADLTVTQEDAPESSGSAIMGVDLGIKVPAVAHI |
| | GGKGTRFFGNGRSQRSMRRRFYARRKTLQKAKKLR |
| | AVRKSKGKEARWMKTINHQLSRQIVNHAHALGVGT |
| | IKIEALQGIRKGTTRKSRGAAARKNNRMTNTWSFS |
| | QLTLFITYKAQRQGITVEQVDPAYTSQDCPACRAR |
| | NGAQDRTYVCSECGWRGHRDTVGAINISRRAGLSG |
| | HRRGATGA |
| SEQ | MIAQKTIKIKLNPTKEQIIKLNSIIEEYIKVSNFT |
| ID | AKKIAEIQESFTDSGLTQGTCSECGKEKTYRKYHL |
| NO: | LKKDNKLFCITCYKRKYSQFTLQKVEFQNKTGLRN |
| 68 | VAKLPKTYYTNAIRFASDTFSGFDEIIKKKQNRLN |
| | SIQNRLNFWKELLYNPSNRNEIKIKVVKYAPKTDT |
| | REHPHYYSEAEIKGRIKRLEKQLKKFKMPKYPEFT |
| | SETISLQRELYSWKNPDELKISSITDKNESMNYYG |
| | KEYLKRYIDLINSQTPQILLEKENNSFYLCFPITK |
| | NIEMPKIDDTFEPVGIDWGITRNIAVVSILDSKTK |
| | KPKFVKFYSAGYILGKRKHYKSLRKHFGQKKRQDK |
| | INKLGTKEDRFIDSNIHKLAFLIVKEIRNHSNKPI |
| | ILMENITDNREEAEKSMRQNILLHSVKSRLQNYIA |
| | YKALWNNIPTNLVKPEHTSQICNRCGHQDRENRPK |
| | GSKLFKCVKCNYMSNADFNASINIARKFYIGEYEP |
| SEQ | FYKDNEKMKSGVNSISM |
| ID | |
| NO: | |
| 69 | LKLSEQENITTGVKFKLKLDKETSEGLNDYFDEYG |
| | KAINFAIKVIQKELAEDRFAGKVRLDENKKPLLNE |
| | DGKKIWDFPNEFCSCGKQVNRYVNGKSLCQECYKN |
| | KFTEYGIRKRMYSAKGRKAEQDINIKNSTNKISKT |
| | HFNYAIREAFILDKSIKKQRKERFRRLREMKKKLQ |
| | EFIEIRDGNKILCPKIEKQRVERYIHPSWINKEKK |
| | LEDFRGYSMSNVLGKIKILDRNIKREEKSLKEKGQ |
| | INFKARRLMLDKSVKFLNDNKISFTISKNLPKEYE |
| | LDLPEKEKRLNWLKEKIKIIKNQKPKYAYLLRKDD |
| | NFYLQYTLETEFNLKEDYSGIVGIDRGVSHIAVYT |
| | FVHNNGKNERPLFLNSSEILRLKNLQKERDRFLRR |
| | KHNKKRKKSNMRNIEKKIQLILHNYSKQIVDFAKN |
| | KNAFIVFEKLEKPKKNRSKMSKKSQYKLSQFTFKK |
| | LSDLVDYKAKREGIKVLYISPEYTSKECSHCGEKV |
| | NTQRPFNGNSSLFKCNKCGVELNADYNASINIAKK |
| | GLNILNSTN |
| SEQ | MEESIITGVKFKLRIDKETTKKLNEYFDEYGKAIN |
| ID | FAVKIIQKELADDRFAGKAKLDQNKNPILDENGKK |
| NO: | IYEFPDEFCSCGKQVNKYVNNKPFCQECYKIRFTE |
| 70 | NGIRKRMYSAKGRKAEHKINILNSTNKISKTHFNY |
| | AIREAFILDKSIKKQRKKRNERLRESKKRLQQFID |
| | MRDGKREICPTIKGQKVDRFIHPSWITKDKKLEDF |
| | RGYTLSIINSKIKILDRNIKREEKSLKEKGQIIFK |
| | AKRLMLDKSIRFVGDRKVLFTISKTLPKEYELDLP |
| | SKEKRLNWLKEKIEIIKNQKPKYAYLLRKNIESEK |
| | KPNYEYYLQYTLEIKPELKDFYDGAIGIDRGINHI |
| | AVCTFISNDGKVTPPKFFSSGEILRLKNLQKERDR |
| | FLLRKHNKNRKKGNMRVIENKINLILHRYSKQIVD |
| | MAKKLNASIVFEELGRIGKSRTKMKKSQRYKLSLF |
| | IFKKLSDLVDYKSRREGIRVTYVPPEYTSKECSHC |
| | GEKVNTQRPFNGNYSLFKCNKCGIQLNSDYNASIN |
| | IAKKGLKIPNST |
| SEQ | LWTIVIGDFIEMPKQDLVTTGIKFKLDVDKETRKK |
| ID | LDDYFDEYGKAINFAVKIIQKNLKEDRFAGKIALG |
| NO: | EDKKPLLDKDGKKIYNYPNESCSCGNQVRRYVNAK |
| 71 | PFCVDCYKLKFTENGIRKRMYSARGRKADSDINIK |
| | NSTNKISKTHFNYAIREGFILDKSLKKQRSKRIKK |
| | LLELKRKLQEFIDIRQGQMVLCPKIKNQRVDKFIH |
| | PSWLKRDKKLEEFRGYSLSVVEGKIKIFNRNILRE |
| | EDSLRQRGHVNFKANRIMLDKSVRFLDGGKVNFNL |
| | NKGLPKEYLLDLPKKENKLSWLNEKISLIKLQKPK |
| | YAYLLRREGSFFIQYTIENVPKTFSDYLGAIGIDR |
| | GISHIAVCTFVSKNGVNKAPVFFSSGEILKLKSLQ |
| | KQRDLFLRGKHNKIRKKSNMRNIDNKINLILHKYS |
| | RNIVNLAKSEKAFIVFEKLEKIKKSRFKMSKSLQY |
| | KLSQFTFKKLSDLVEYKAKIEGIKVDYVPPEYTSK |
| | ECSHCGEKVDTQRPFNGNSSLFKCNKCRVOLNADY |
| | NASINIAKKSLNISN |
| SEQ | MSKTTISVKLKIIDLSSEKKEFLDNYFNEYAKATT |
| ID | FCQLRIRRLLRNTHWLGKKEKSSKKWIFESGICDL |
| NO: | CGENKELVNEDRNSGEPAKICKRCYNGRYGNQMIR |
| 72 | KLFVSTKKREVQENMDIRRVAKLNNTHYHRIPEEA |
| | FDMIKAADTAEKRRKKNVEYDKKRQMEFIEMENDE |
| | KKRAARPKKPNERETRYVHISKLESPSKGYTLNGI |
| | KRKIDGMGKKIERAEKGLSRKKIFGYQGNRIKLDS |
| | NWVRFDLAESEITIPSLFKEMKLRITGPTNVHSKS |
| | GQIYFAEWFERINKQPNNYCYLIRKTSSNGKYEYY |
| | LQYTYEAEVEANKEYAGCLGVDIGCSKLAAAVYYD |
| | SKNKKAQKPIEIFTNPIKKIKMRREKLIKLLSRVK |
| | VRHRRRKLMQLSKTEPIIDYTCHKTARKIVEMANT |
| | AKAFISMENLETGIKQKQQARETKKQKFYRNMFLF |
| | RKLSKLIEYKALLKGIKIVYVKPDYTSQTCSSCGA |
| | DKEKTERPSQAIFRCLNPTCRYYQRDINADFNAAV |
| | NIAKKALNNTEVVTTLL |
| SEQ | MARAKNQPYQKLTTTTGIKFKLDLSEEEGKRFDEY |
| ID | FSEYAKAVNFCAKVIYQLRKNLKFAGKKELAAKEW |
| NO: | KFEISNCDFCNKQKEIYYKNIANGQKVCKGCHRTN |
| 73 | FSDNAIRKKMIPVKGRKVESKFNIHNTTKKISGTH |
| | RHWAFEDAADIIESMDKQRKEKQKRLRREKRKLSY |
| | FFELFGDPAKRYELPKVGKQRVPRYLHKIIDKDSL |
| | TKKRGYSLSYIKNKIKISERNIERDEKSLRKASPI |
| | AFGARKIKMSKLDPKRAFDLENNVFKIPGKVIKGQ |
| | YKFFGTNVANEHGKKFYKDRISKILAGKPKYFYLL |
| | RKKVAESDGNPIFEYYVQWSIDTETPAITSYDNIL |
| | GIDAGITNLATTVLIPKNLSAEHCSHCGNNHVKPI |
| | FTKFFSGKELKAIKIKSRKQKYFLRGKHNKLVKIK |
| | RIRPIEQKVDGYCHVVSKQIVEMAKERNSCIALEK |
| | LEKPKKSKFRQRRREKYAVSMFVFKKLATFIKYKA |
| | AREGIEIIPVEPEGTSYTCSHCKNAQNNQRPYFKP |
| | NSKKSWTSMFKCGKCGIELNSDYNAAFNIAQKALN |
| | MTSA |
| SEQ | MDEKHFFCSYCNKELKISKNLINKISKGSIREDEA |
| ID | VSKAISIHNKKEHSLILGIKFKLFIENKLDKKKLN |
| NO: | EYFDNYSKAVTFAARIFDKIRSPYKFIGLKDKNTK |
| 74 | KWTFPKAKCVFCLEEKEVAYANEKDNSKICTECYL |
| | KEFGENGIRKKIYSTRGRKVEPKYNIFNSTKELSS |
| | THYNYAIRDAFOLLDALKKQRQKKLKSIFNQKLRL |
| | KEFEDIFSDPQKRIELSLKPHQREKRYIHLSKSGQ |
| | ESINRGYTLRFVRGKIKSLTRNIEREEKSLRKKTP |
| | IHFKGNRLMIFPAGIKFDFASNKVKISISKNLPNE |
| | FNFSGTNVKNEHGKSFFKSRIELIKTQKPKYAYVL |
| | RKIKREYSKLRNYEIEKIRLENPNADLCDFYLQYT |
| | IETESRNNEEINGIIGIDRGITNLACLVLLKKGDK |
| | KPSGVKFYKGNKILGMKIAYRKHLYLLKGKRNKLR |
| | KQRQIRAIEPKINLILHQISKDIVKIAKEKNFAIA |
| | LEQLEKPKKARFAQRKKEKYKLALFTFKNLSTLIE |
| | YKSKREGIPVIYVPPEKTSQMCSHCAINGDEHVDT |
| | QRPYKKPNAQKPSYSLFKCNKCGIELNADYNAAFN |
| | IAQKGLKTLMLNHSH |
| SEQ | MLQTLLVKLDPSKEQYKMLYETMERFNEACNQIAE |
| ID | TVFAIHSANKIEVQKTVYYPIREKFGLSAQLTILA |
| NO: | IRKVCEAYKRDKSIKPEFRLDGALVYDQRVLSWKG |
| 75 | LDKVSLVTLQGRQIIPIKFGDYQKARMDRIRGQAD |
| | LILVKGVFYLCVVVEVSEESPYDPKGVLGVDLGIK |
| | NLAVDSDGEVHSGEQTTNTRERLDSLKARLQSKGT |
| | KSAKRHLKKLSGRMAKFSKDVNHCISKKLVAKAKG |
| | TLMSIALEDLQGIRDRVTVRKAQRRNLHTWNFGLL |
| | RMFVDYKAKIAGVPLVFVDPRNTSRTCPSCGHVAK |
| | ANRPTRDEFRCVSCGFAGAADHIAAMNIAFRAEVS |
| | QPIVTRFFVQSQAPSFRVG |
| SEQ | MDEEPDSAEPNLAPISVKLKLVKLDGEKLAALNDY |
| ID | FNEYAKAVNFCELKMQKIRKNLVNIRGTYLKEKKA |
| NO: | WINQTGECCICKKIDELRCEDKNPDINGKICKKCY |
| 76 | NGRYGNQMIRKLFVSTNKRAVPKSLDIRKVARLHN |
| | THYHRIPPEAADIIKAIETAERKRRNRILFDERRY |
| | NELKDALENEEKRVARPKKPKEREVRYVPISKKDT |
| | PSKGYTMNALVRKVSGMAKKIERAKRNLNKRKKIE |
| | YLGRRILLDKNWVRFDFDKSEISIPTMKEFFGEMR |
| | FEITGPSNVMSPNGREYFTKWFDRIKAQPDNYCYL |
| | LRKESEDETDFYLQYTWRPDAHPKKDYTGCLGIDI |
| | GGSKLASAVYFDADKNRAKQPIQIFSNPIGKWKTK |
| | RQKVIKVLSKAAVRHKTKKLESLRNIEPRIDVHCH |
| | RIARKIVGMALAANAFISMENLEGGIREKQKAKET |
| | KKQKFSRNMFVFRKLSKLIEYKALMEGVKVVYIVP |
| | DYTSQLCSSCGTNNTKRPKQAIFMCQNTECRYFGK |
| | NINADFNAAINIAKKALNRKDIVRELS |
| SEQ | MEKNNSEQTSITTGIKFKLKLDKETKEKLNNYFDE |
| ID | YGKAINFAVRIIQMQLNDDRLAGKYKRDEKGKPIL |
| NO: | GEDGKKILEIPNDFCSCGNQVNHYVNGVSFCQECY |
| 77 | KKRFSENGIRKRMYSAKGRKAEQDINIKNSTNKIS |
| | KTHFNYAIREAFNLDKSIKKQREKRFKKLKDMKRK |
| | LQEFLEIRDGKRVICPKIEKQKVERYIHPSWINKE |
| | KKLEEFRGYSLSIVNSKIKSFDRNIQREEKSLKEK |
| | GQINFKAQRLMLDKSVKFLKDNKVSFTISKELPKT |
| | FELDLPKKEKKLNWLNEKLEIIKNQKPKYAYLLRK |
| | ENNIFLQYTLDSIPEIHSEYSGAVGIDRGVSHIAV |
| | YTFLDKDGKNERPFFLSSSGILRLKNLQKERDKFL |
| | RKKHNKIRKKGNMRNIEQKINLILHEYSKQIVNFA |
| | KDKNAFIVFELLEKPKKSRERMSKKIQYKLSQFTF |
| | KKLSDLVDYKAKREGIKVIYVEPAYTSKDCSHCGE |
| | RVNTQRPFNGNFSLFKCNKCGIVLNSDYNASLNIA |
| | RKGLNISAN |
| SEQ | MAEEKFFFCEKCNKDIKIPKNYINKQGAEEKARAK |
| ID | HEHRVHALILGIKFKIYPKKEDISKLNDYFDEYAK |
| NO: | AVTFTAKIVDKLKAPFLFAGKRDKDTSKKKWVFPV |
| 78 | DKCSFCKEKTEINYRTKQGKNICNSCYLTEFGEQG |
| | LLEKIYATKGRKVSSSFNLFNSTKKLTGTHNNYVV |
| | KESLQLLDALKKQRSKRLKKLSNTRRKLKQFEEMF |
| | EKEDKRFQLPLKEKQRELRFIHVSQKDRATEFKGY |
| | TMNKIKSKIKVLRRNIEREQRSLNRKSPVFFRGTR |
| | IRLSPSVQFDDKDNKIKLTLSKELPKEYSFSGLNV |
| | ANEHGRKFFAEKLKLIKENKSKYAYLLRRQVNKNN |
| | KKPIYDYYLQYTVEFLPNIITNYNGILGIDRGINT |
| | LACIVLLENKKEKPSFVKFFSGKGILNLKNKRRKQ |
| | LYFLKGVHNKYRKQQKIRPIEPRIDQILHDISKQI |
| | IDLAKEKRVAISLEQLEKPQKPKFRQSRKAKYKLS |
| | QFNFKTLSNYIDYKAKKEGIRVIYIAPEMTSQNCS |
| | RCAMKNDLHVNTQRPYKNTSSLFKCNKCGVELNAD |
| | YNAAFNIAQKGLKILNS |
| SEQ | MISLKLKLLPDEEQKKLLDEMFWKWASICTRVGFG |
| ID | RADKEDLKPPKDAEGVWFSLTQLNQANTDINDLRE |
| NO: | AMKHQKHRLEYEKNRLEAQRDDTQDALKNPDRREI |
| 79 | STKRKDLFRPKASVEKGFLKLKYHQERYWVRRLKE |
| | INKLIERKTKTLIKIEKGRIKFKATRITLHQGSFK |
| | IRFGDKPAFLIKALSGKNQIDAPFVVVPEQPICGS |
| | VVNSKKYLDEITTNFLAYSVNAMLFGLSRSEEMLL |
| | KAKRPEKIKKKEEKLAKKQSAFENKKKELQKLLGR |
| | ELTQQEEAIIEETRNQFFQDFEVKITKQYSELLSK |
| | IANELKQKNDFLKVNKYPILLRKPLKKAKSKKINN |
| | LSPSEWKYYLQFGVKPLLKQKSRRKSRNVLGIDRG |
| | LKHLLAVTVLEPDKKTFVWNKLYPNPITGWKWRRR |
| | KLLRSLKRLKRRIKSQKHETIHENQTRKKLKSLQG |
| | RIDDLLHNISRKIVETAKEYDAVIVVEDLQSMRQH |
| | GRSKGNRLKTLNYALSLFDYANVMQLIKYKAGIEG |
| | IQIYDVKPAGTSQNCAYCLLAQRDSHEYKRSQENS |
| | KIGVCLNPNCQNHKKQIDADLNAARVIASCYALKI |
| SEQ | NDSQPFGTRKRFKKRTTN |
| ID | |
| NO: | |
| 80 | METLSLKLKLNPSKEQLLVLDKMFWKWASICTRLG |
| | LKKAEMSDLEPPKDAEGVWFSKTQLNQANTDVNDL |
| | RKAMQHQGKRIEYELDKVENRRNEIQEMLEKPDRR |
| | DISPNRKDLFRPKAAVEKGYLKLKYHKLGYWSKEL |
| | KTANKLIERKRKTLAKIDAGKMKFKPTRISLHTNS |
| | FRIKFGEEPKIALSTTSKHEKIELPLITSLQRPLK |
| | TSCAKKSKTYLDAAILNFLAYSTNAALFGLSRSEE |
| | MLLKAKKPEKIEKRDRKLATKRESFDKKLKTLEKL |
| | LERKLSEKEKSVFKRKQTEFFDKFCITLDETYVEA |
| | LHRIAEELVSKNKYLEIKKYPVLLRKPESRLRSKK |
| | LKNLKPEDWTYYIQFGFQPLLDTPKPIKTKTVLGI |
| | DRGVRHLLAVSIFDPRTKTFTFNRLYSNPIVDWKW |
| | RRRKLLRSIKRLKRRLKSEKHVHLHENQFKAKLRS |
| | LEGRIEDHFHNLSKEIVDLAKENNSVIVVENLGGM |
| | RQHGRGRGKWLKALNYALSHFDYAKVMQLIKYKAE |
| | LAGVFVYDVAPAGTSINCAYCLLNDKDASNYTRGK |
| | VINGKKNTKIGECKTCKKEFDADLNAARVIALCYE |
| | KRLNDPQPFGTRKQFKPKKP |
| SEQ | MKALKLQLIPTRKQYKILDEMFWKWASLANRVSQK |
| ID | GESKETLAPKKDIQKIQFNATQLNQIEKDIKDLRG |
| NO: | AMKEQQKQKERLLLQIQERRSTISEMLNDDNNKER |
| 81 | DPHRPLNFRPKGWRKFHTSKHWVGELSKILRQEDR |
| | VKKTIERIVAGKISFKPKRIGIWSSNYKINFFKRK |
| | ISINPLNSKGFELTLMTEPTQDLIGKNGGKSVLNN |
| | KRYLDDSIKSLLMFALHSRFFGLNNTDTYLLGGKI |
| | NPSLVKYYKKNQDMGEFGREIVEKFERKLKQEINE |
| | QQKKIIMSQIKEQYSNRDSAFNKDYLGLINEFSEV |
| | FNQRKSERAEYLLDSFEDKIKQIKQEIGESLNISD |
| | WDFLIDEAKKAYGYEEGFTEYVYSKRYLEILNKIV |
| | KAVLITDIYFDLRKYPILLRKPLDKIKKISNLKPD |
| | EWSYYIQFGYDSINPVQLMSTDKFLGIDRGLTHLL |
| | AYSVFDKEKKEFIINQLEPNPIMGWKWKLRKVKRS |
| | LQHLERRIRAQKMVKLPENQMKKKLKSIEPKIEVH |
| | YHNISRKIVNLAKDYNASIVVESLEGGGLKQHGRK |
| | KNARNRSLNYALSLFDYGKIASLIKYKADLEGVPM |
| | YEVLPAYTSQQCAKCVLEKGSFVDPEIIGYVEDIG |
| | IKGSLLDSLFEGTELSSIQVLKKIKNKIELSARDN |
| | HNKEINLILKYNFKGLVIVRGQDKEEIAEHPIKEI |
| | NGKFAILDFVYKRGKEKVGKKGNQKVRYTGNKKVG |
| | YCSKHGQVDADLNASRVIALCKYLDINDPILFGEQ |
| | RKSFK |
| SEQ | MVTRAIKLKLDPTKNQYKLLNEMFWKWASLANRFS |
| ID | QKGASKETLAPKDGTQKIQFNATQLNQIKKDVDDL |
| NO: | RGAMEKQGKQKERLLIQIQERLLTISEILRDDSKK |
| 82 | EKDPHRPQNFRPFGWRRFHTSAYWSSEASKLTRQV |
| | DRVRRTIERIKAGKINFKPKRIGLWSSTYKINFLK |
| | KKINISPLKSKSFELDLITEPQQKIIGKEGGKSVA |
| | NSKKYLDDSIKSLLIFAIKSRLFGLNNKDKPLFEN |
| | IITPNLVRYHKKGQEQENFKKEVIKKFENKLKKEI |
| | SQKQKEIIFSQIERQYENRDATFSEDYLRAISEFS |
| | EIFNQRKKERAKELLNSFNEKIRQLKKEVNGNISE |
| | EDLKILEVEAEKAYNYENGFIEWEYSEQFLGVLEK |
| | IARAVLISDNYFDLKKYPILIRKPTNKSKKITNLK |
| | PEEWDYYIQFGYGLINSPMKIETKNFMGIDRGLTH |
| | LLAYSIFDRDSEKFTINQLELNPIKGWKWKLRKVK |
| | RSLQHLERRMRAQKGVKLPENQMKKRLKSIEPKIE |
| | SYYHNLSRKIVNLAKANNASIVVESLEGGGLKQHG |
| | RKKNSRHRALNYALSLFDYGKIASLIKYKSDLEGV |
| | PMYEVLPAYTSQQCAKCVLKKGSFVEPEIIGYIEE |
| | IGFKENLLTLLFEDTGLSSVQVLKKSKNKMTLSAR |
| | DKEGKMVDLVLKYNFKGLVISQEKKKEEIVEFPIK |
| | EIDGKFAVLDSAYKRGKERISKKGNQKLVYTGNKK |
| | VGYCSVHGQVDADLNASRVIALCKYLGINEPIVFG |
| | EQRKSFK |
| SEQ | LDLITEPIQPHKSSSLRSKEFLEYQISDFLNFSLH |
| ID | SLFFGLASNEGPLVDFKIYDKIVIPKPEERFPKKE |
| NO: | SEEGKKLDSFDKRVEEYYSDKLEKKIERKLNTEEK |
| 83 | NVIDREKTRIWGEVNKLEEIRSIIDEINEIKKQKH |
| | ISEKSKLLGEKWKKVNNIQETLLSQEYVSLISNLS |
| | DELTNKKKELLAKKYSKFDDKIKKIKEDYGLEFDE |
| | NTIKKEGEKAFLNPDKFSKYQFSSSYLKLIGEIAR |
| | SLITYKGFLDLNKYPIIFRKPINKVKKIHNLEPDE |
| | WKYYIQFGYEQINNPKLETENILGIDRGLTHILAY |
| | SVFEPRSSKFILNKLEPNPIEGWKWKLRKLRRSIQ |
| | NLERRWRAQDNVKLPENQMKKNLRSIEDKVENLYH |
| | NLSRKIVDLAKEKNACIVFEKLEGQGMKQHGRKKS |
| | DRLRGLNYKLSLFDYGKIAKLIKYKAEIEGIPIYR |
| | IDSAYTSQNCAKCVLESRRFAQPEEISCLDDFKEG |
| | DNLDKRILEGTGLVEAKIYKKLLKEKKEDFEIEED |
| | IAMFDTKKVIKENKEKTVILDYVYTRRKEIIGTNH |
| | KKNIKGIAKYTGNTKIGYCMKHGQVDADLNASRTI |
| | ALCKNFDINNPEIWK |
| SEQ | MSDESLVSSEDKLAIKIKIVPNAEQAKMLDEMFKK |
| ID | WSSICNRISRGKEDIETLRPDEGKELQFNSTQLNS |
| NO: | ATMDVSDLKKAMARQGERLEAEVSKLRGRYETIDA |
| 84 | SLRDPSRRHTNPQKPSSFYPSDWDISGRLTPRFHT |
| | ARHYSTELRKLKAKEDKMLKTINKIKNGKIVFKPK |
| | RITLWPSSVNMAFKGSRLLLKPFANGFEMELPIVI |
| | SPQKTADGKSQKASAEYMRNALLGLAGYSINQLLF |
| | GMNRSQKMLANAKKPEKVEKFLEQMKNKDANFDKK |
| | IKALEGKWLLDRKLKESEKSSIAVVRTKFFKSGKV |
| | ELNEDYLKLLKHMANEILERDGFVNLNKYPILSRK |
| | PMKRYKQKNIDNLKPNMWKYYIQFGYEPIFERKAS |
| | GKPKNIMGIDRGLTHLLAVAVFSPDQQKFLFNHLE |
| | SNPIMHWKWKLRKIRRSIQHMERRIRAEKNKHIHE |
| | AQLKKRLGSIEEKTEQHYHIVSSKIINWAIEYEAA |
| | IVLESLSHMKQRGGKKSVRTRALNYALSLFDYEKV |
| | ARLITYKARIRGIPVYDVLPGMTSKTCATCLLNGS |
| | QGAYVRGLETTKAAGKATKRKNMKIGKCMVCNSSE |
| | NSMIDADLNAARVIAICKYKNLNDPQPAGSRKVFK |
| | RF |
| SEQ | MLALKLKIMPTEKQAEILDAMFWKWASICSRIAKM |
| ID | KKKVSVKENKKELSKKIPSNSDIWFSKTQLCQAEV |
| NO: | DVGDHKKALKNFEKRQESLLDELKYKVKAINEVIN |
| 85 | DESKREIDPNNPSKFRIKDSTKKGNLNSPKFFTLK |
| | KWQKILQENEKRIKKKESTIEKLKRGNIFFNPTKI |
| | SLHEEEYSINFGSSKLLLNCFYKYNKKSGINSDQL |
| | ENKFNEFQNGLNIICSPLQPIRGSSKRSFEFIRNS |
| | IINFLMYSLYAKLFGIPRSVKALMKSNKDENKLKL |
| | EEKLKKKKSSFNKTVKEFEKMIGRKLSDNESKILN |
| | DESKKFFEIIKSNNKYIPSEEYLKLLKDISEEIYN |
| | SNIDFKPYKYSILIRKPLSKFKSKKLYNLKPTDYK |
| | YYLQLSYEPFSKQLIATKTILGIDRGLKHLLAVSV |
| | FDPSQNKFVYNKLIKNPVFKWKKRYHDLKRSIRNR |
| | ERRIRALTGVHIHENQLIKKLKSMKNKINVLYHNV |
| | SKNIVDLAKKYESTIVLERLENLKQHGRSKGKRYK |
| | KLNYVLSNFDYKKIESLISYKAKKEGVPVSNINPK |
| | YTSKTCAKCLLEVNQLSELKNEYNRDSKNSKIGIC |
| | NIHGQIDADLNAARVIALCYSKNLNEPHFK |
| SEQ | VINLFGYKFALYPNKTQEELLNKHLGECGWLYNKA |
| ID | IEQNEYYKADSNIEEAQKKFELLPDKNSDEAKVLR |
| NO: | GNISKDNYVYRTLVKKKKSEINVQIRKAVVLRPAE |
| 86 | TIRNLAKVKKKGLSVGRLKFIPIREWDVLPFKQSD |
| | QIRLEENYLILEPYGRLKFKMHRPLLGKPKTFCIK |
| | RTATDRWTISFSTEYDDSNMRKNDGGQVGIDVGLK |
| | THLRLSNENPDEDPRYPNPKIWKRYDRRLTILQRR |
| | ISKSKKLGKNRTRLRLRLSRLWEKIRNSRADLIQN |
| | ETYEILSENKLIAIEDLNVKGMQEKKDKKGRKGRT |
| | RAQEKGLHRSISDAAFSEFRRVLEYKAKRFGSEVK |
| | PVSAIDSSKECHNCGNKKGMPLESRIYECPKCGLK |
| | IDRDLNSAKVILARATGVRPGSNARADTKISATAG |
| | ASVQTEGTVSEDFRQQMETSDQKPMQGEGSKEPPM |
| | NPEHKSSGRGSKHVNIGCKNKVGLYNEDENSRSTE |
| | KQIMDENRSTTEDMVEIGALHSPVLTT |
| SEQ | MIASIDYEAVSQALIVFEFKAKGKDSQYQAIDEAI |
| ID | RSYRFIRNSCLRYWMDNKKVGKYDLNKYCKVLAKQ |
| NO: | YPFANKLNSQARQSAAECSWSAISRFYDNCKRKVS |
| 87 | GKKGFPKFKKHARSVEYKTSGWKLSENRKAITFTD |
| | KNGIGKLKLKGTYDLHFSQLEDMKRVRLVRRADGY |
| | YVQFCISVDVKVETEPTGKAIGLDVGIKYFLADSS |
| | GNTIENPQFYRKAEKKLNRANRRKSKKYIRGVKPQ |
| | SKNYHKARCRYARKHLRVSRQRKEYCKRVAYCVIH |
| | SNDVVAYEDLNVKGMVKNRHLAKSISDVAWSTFRH |
| | WLEYFAIKYGKLTIPVAPHNTSQNCSNCDKKVPKS |
| | LSTRTHICHHCGYSEDRDVNAAKNILKKALSTVGQ |
| | TGSLKLGEIEPLLVLEQSCTRKFDL |
| SEQ | LAEENTLHLTLAMSLPLNDLPENRTRSELWRRQWL |
| ID | PQKKLSLLLGVNQSVRKAAADCLRWFEPYQELLWW |
| NO: | EPTDPDGKKLLDKEGRPIKRTAGHMRVLRKLEEIA |
| 88 | PFRGYQLGSAVKNGLRHKVADLLLSYAKRKLDPQF |
| | TDKTSYPSIGDQFPIVWTGAFVCYEQSITGQLYLY |
| | LPLFPRGSHQEDITNNYDPDRGPALQVFGEKEIAR |
| | LSRSTSGLLLPLQFDKWGEATFIRGENNPPTWKAT |
| | HRRSDKKWLSEVLLREKDFQPKRVELLVRNGRIFV |
| | NVACEIPTKPLLEVENFMGVSFGLEHLVTVVVINR |
| | DGNVVHQRQEPARRYEKTYFARLERLRRRGGPFSQ |
| | ELETFHYRQVAQIVEEALRFKSVPAVEQVGNIPKG |
| | RYNPRLNLRLSYWPFGKLADLTSYKAVKEGLPKPY |
| | SVYSATAKMLCSTCGAANKEGDQPISLKGPTVYCG |
| | NCGTRHNTGFNTALNLARRAQELFVKGVVAR |
| SEQ | MSQSLLKWHDMAGRDKDASRSLQKSAVEGVLLHLT |
| ID | ASHRVALEMLEKSVSQTVAVTMEAAQQRLVIVLED |
| NO: | DPTKATSRKRVISADLQFTREEFGSLPNWAQKLAS |
| 89 | TCPEIATKYADKHINSIRIAWGVAKESTNGDAVEQ |
| | KLQWQIRLLDVTMFLQQLVLQLADKALLEQIPSSI |
| | RGGIGQEVAQQVTSHIQLLDSGTVLKAELPTISDR |
| | NSELARKQWEDAIQTVCTYALPFSRERARILDPGK |
| | YAAEDPRGDRLINIDPMWARVLKGPTVKSLPLLFV |
| | SGSSIRIVKLTLPRKHAAGHKHTFTATYLVLPVSR |
| | EWINSLPGTVQEKVQWWKKPDVLATQELLVGKGAL |
| | KKSANTLVIPISAGKKRFFNHILPALQRGFPLQWQ |
| | RIVGRSYRRPATHRKWFAQLTIGYTNPSSLPEMAL |
| | GIHFGMKDILWWALADKQGNILKDGSIPGNSILDF |
| | SLQEKGKIERQQKAGKNVAGKKYGKSLLNATYRVV |
| | NGVLEFSKGISAEHASQPIGLGLETIRFVDKASGS |
| | SPVNARHSNWNYGQLSGIFANKAGPAGFSVTEITL |
| | KKAQRDLSDAEQARVLAIEATKRFASRIKRLATKR |
| | KDDTLFV |
| SEQ | VEPVEKERFYYRTYTFRLDGQPRTQNLTTQSGWGL |
| ID | LTKAVLDNTKHYWEIVHHARIANQPIVFENPVIDE |
| NO: | QGNPKLNKLGQPRFWKRPISDIVNQLRALFENQNP |
| 90 | YQLGSSLIQGTYWDVAENLASWYALNKEYLAGTAT |
| | WGEPSFPEPHPLTEINQWMPLTFSSGKVVRLLKNA |
| | SGRYFIGLPILGENNPCYRMRTIEKLIPCDGKGRV |
| | TSGSLILFPLVGIYAQQHRRMTDICESIRTEKGKL |
| | AWAQVSIDYVREVDKRRRMRRTRKSQGWIQGPWQE |
| | VFILRLVLAHKAPKLYKPRCFAGISLGPKTLASCV |
| | ILDQDERVVEKQQWSGSELLSLIHQGEERLRSLRE |
| | QSKPTWNAAYRKQLKSLINTQVFTIVTFLRERGAA |
| | VRLESIARVRKSTPAPPVNFLLSHWAYRQITERLK |
| | DLAIRNGMPLTHSNGSYGVRFTCSQCGATNQGIKD |
| | PTKYKVDIESETFLCSICSHREIAAVNTATNLAKQ |
| | LLDE |
| SEQ | MNDTETSETLTSHRTVCAHLHVVGETGSLPRLVEA |
| ID | ALAELITLNGRATQALLSLAKNGLVLRRDKEENLI |
| NO: | AAELTLPCRKNKYADVAAKAGEPILATRINNKGKL |
| 91 | VTKKWYGEGNSYHIVRFTPETGMFTVRVFDRYAFD |
| | EELLHLHSEVVFGSDLPKGIKAKTDSLPANFLQAV |
| | FTSFLELPFQGFPDIVVKPAMKQAAEQLLSYVQLE |
| | AGENQQAEYPDTNERDPELRLVEWQKSLHELSVRT |
| | EPFEFVRARDIDYYAETDRRGNRFVNITPEWTKFA |
| | ESPFARRLPLKIPPEFCILLRRKTEGHAKIPNRIY |
| | LGLQIFDGVTPDSTLGVLATAEDGKLFWWHDHLDE |
| | FSNLEGKPEPKLKNKPQLLMVSLEYDREQRFEESV |
| | GGDRKICLVTLKETRNFRRGWNGRILGIHFQHNPV |
| | ITWALMDHDAEVLEKGFIEGNAFLGKALDKQALNE |
| | YLQKGGKWVGDRSFGNKLKGITHTLASLIVRLARE |
| | KDAWIALEEISWVQKQSADSVANHEIVEQPHHSLT |
| | R |
| SEQ | MNDTETSETLTSHRTVCAHLHVVGETGSLPRLVEA |
| ID | ALAELITLNGRATQALLSLAKNGLVLRRDKEENLI |
| NO: | AAELTLPCRKNKYADVAAKAGEPILATRINNKGKL |
| 92 | VTKKWYGEGNSYHIVRFTPETGMFTVRVFDRYAFD |
| | EELLHLHSEVVFGSDLPKGIKAKTDSLPANFLQAV |
| | FTSFLELPFQGFPDIVVKPAMKQAAEQLLSYVQLE |
| | AGENQQAEYPDTNERDPELRLVEWQKSLHELSVRT |
| | EPFEFVRARDIDYYAETDRRGNRFVNITPEWTKFA |
| | ESPFARRLPLKIPPEFCILLRRKTEGHAKIPNRIY |
| | LGLQIFDGVTPDSTLGVLATAEDGKLFWWHDHLDE |
| | FSNLEGKPEPKLKNKPQLLMVSLEYDREQRFEESV |
| | GGDRKICLVILKETRNFRRGRHGHTRTDRLPAGNT |
| | LWRADFATSAEVAAPKWNGRILGIHFQHNPVITWA |
| | LMDHDAEVLEKGFIEGNAFLGKALDKQALNEYLQK |
| | GGKWVGDRSFGNKLKGITHTLASLIVRLAREKDAW |
| | IALEEISWVQKQSADSVANRRFSMWNYSRLATLIE |
| | WLGTDIATRDCGTAAPLAHKVSDYLTHFTCPECGA |
| | CRKAGQKKEIADTVRAGDILTCRKCGFSGPIPDNF |
| | IAEFVAKKALERMLKKKPV |
| SEQ | MAKRNFGEKSEALYRAVRFEVRPSKEELSILLAVS |
| ID | EVLRMLFNSALAERQQVFTEFIASLYAELKSASVP |
| NO: | EEISEIRKKLREAYKEHSISLFDQINALTARRVED |
| 93 | EAFASVTRNWQEETLDALDGAYKSFLSLRRKGDYD |
| | AHSPRSRDSGFFQKIPGRSGFKIGEGRIALSCGAG |
| | RKLSFPIPDYQQGRLAETTKLKKFELYRDQPNLAK |
| | SGRFWISVVYELPKPEATTCQSEQVAFVALGASSI |
| | GVVSQRGEEVIALWRSDKHWVPKIEAVEERMKRRV |
| | KGSRGWLRLLNSGKRRMHMISSRQHVQDEREIVDY |
| | LVRNHGSHFVVTELVVRSKEGKLADSSKPERGGSL |
| | GLNWAAQNTGSLSRLVRQLEEKVKEHGGSVRKHKL |
| | TLTEAPPARGAENKLWMARKLRESFLKEV |
| SEQ | LAKNDEKELLYQSVKFEIYPDESKIRVLTRVSNIL |
| ID | VLVWNSALGERRARFELYIAPLYEELKKFPRKSAE |
| NO: | SNALRQKIREGYKEHIPTFFDQLKKLLTPMRKEDP |
| 94 | ALLGSVPRAYQEETLNTLNGSFVSFMTLRRNNDMD |
| | AKPPKGRAEDRFHEISGRSGFKIDGSEFVLSTKEQ |
| | KLRFPIPNYQLEKLKEAKQIKKFTLYQSRDRRFWI |
| | SIAYEIELPDQRPFNPEEVIYIAFGASSIGVISPE |
| | GEKVIDFWRPDKHWKPKIKEVENRMRSCKKGSRAW |
| | KKRAAARRKMYAMTQRQQKLNHREIVASLLRLGFH |
| | FVVTEYTVRSKPGKLADGSNPKRGGAPQGFNWSAQ |
| | NTGSFGEFILWLKQKVKEQGGTVQTFRLVLGQSER |
| | PEKRGRDNKIEMVRLLREKYLESQTIVV |
| SEQ | MAKGKKKEGKPLYRAVRFEIFPTSDQITLFLRVSK |
| ID | NLQQVWNEAWQERQSCYEQFFGSIYERIGQAKKRA |
| NO: | QEAGFSEVWENEAKKGLNKKLRQQEISMQLVSEKE |
| 95 | SLLQELSIAFQEHGVTLYDQINGLTARRIIGEFAL |
| | IPRNWQEETLDSLDGSFKSFLALRKNGDPDAKPPR |
| | QRVSENSFYKIPGRSGFKVSNGQIYLSFGKIGQTL |
| | TSVIPEFQLKRLETAIKLKKFELCRDERDMAKPGR |
| | FWISVAYEIPKPEKVPVVSKQITYLAIGASRLGVV |
| | SPKGEFCLNLPRSDYHWKPQINALQERLEGVVKGS |
| | RKWKKRMAACTRMFAKLGHQQKQHGQYEVVKKLLR |
| | HGVHFVVTELKVRSKPGALADASKSDRKGSPTGPN |
| | WSAQNTGNIARLIQKLTDKASEHGGTVIKRNPPLL |
| | SLEERQLPDAQRKIFIAKKLREEFLADQK |
| SEQ | MAKREKKDDVVLRGTKMRIYPTDRQVTLMDMWRRR |
| ID | CISLWNLLLNLETAAYGAKNTRSKLGWRSIWARVV |
| NO: | EENHAKALIVYQHGKCKKDGSFVLKRDGTVKHPPR |
| 96 | ERFPGDRKILLGLFDALRHTLDKGAKCKCNVNQPY |
| | ALTRAWLDETGHGARTADIIAWLKDFKGECDCTAI |
| | STAAKYCPAPPTAELLTKIKRAAPADDLPVDQAIL |
| | LDLFGALRGGLKQKECDHTHARTVAYFEKHELAGR |
| | AEDILAWLIAHGGTCDCKIVEEAANHCPGPRLFIW |
| | EHELAMIMARLKAEPRTEWIGDLPSHAAQTVVKDL |
| | VKALQTMLKERAKAAAGDESARKTGFPKFKKQAYA |
| | AGSVYFPNTTMFFDVAAGRVQLPNGCGSMRCEIPR |
| | QLVAELLERNLKPGLVIGAQLGLLGGRIWRQGDRW |
| | YLSCQWERPQPTLLPKTGRTAGVKIAASIVFTTYD |
| | NRGQTKEYPMPPADKKLTAVHLVAGKQNSRALEAQ |
| | KEKEKKLKARKERLRLGKLEKGHDPNALKPLKRPR |
| | VRRSKLFYKSAARLAACEAIERDRRDGFLHRVTNE |
| | IVHKFDAVSVQKMSVAPMMRRQKQKEKQIESKKNE |
| | AKKEDNGAAKKPRNLKPVRKLLRHVAMARGRQFLE |
| | YKYNDLRGPGSVLIADRLEPEVQECSRCGTKNPQM |
| | KDGRRLLRCIGVLPDGTDCDAVLPRNRNAARNAEK |
| | RLRKHREAHNA |
| SEQ | MNEVLPIPAVGEDAADTIMRGSKMRIYPSVRQAAT |
| ID | MDLWRRRCIQLWNLLLELEQAAYSGENRRTQIGWR |
| NO: | SIWATVVEDSHAEAVRVAREGKKRKDGTFRKAPSG |
| 97 | KEIPPLDPAMLAKIQRQMNGAVDVDPKTGEVTPAQ |
| | PRLFMWEHELQKIMARLKQAPRTHWIDDLPSHAAQ |
| | SVVKDLIKALQAMLRERKKRASGIGGRDTGFPKFK |
| | KNRYAAGSVYFANTQLRFEAKRGKAGDPDAVRGEF |
| | ARVKLPNGVGWMECRMPRHINAAHAYAQATLMGGR |
| | IWRQGENWYLSCQWKMPKPAPLPRAGRTAAIKIAA |
| | AIPITTVDNRGQTREYAMPPIDRERIAAHAAAGRA |
| | QSRALEARKRRAKKREAYAKKRHAKKLERGIAAKP |
| | PGRARIKLSPGFYAAAAKLAKLEAEDANAREAWLH |
| | EITTQIVRNFDVIAVPRMEVAKLMKKPEPPEEKEE |
| | QVKAPWQGKRRSLKAARVMMRRTAMALIQTTLKYK |
| | AVDLRGPQAYEEIAPLDVTAAACSGCGVLKPEWKM |
| | ARAKGREIMRCQEPLPGGKTCNTVLTYTRNSARVI |
| | GRELAVRLAERQKA |
| SEQ | MTTQKTYNFCFYDQRFFELSKEAGEVYSRSLEEFW |
| ID | KIYDETGVWLSKFDLQKHMRNKLERKLLHSDSFLG |
| NO: | AMQQVHANLASWKQAKKVVPDACPPRKPKFLQAIL |
| 98 | FKKSQIKYKNGFLRLTLGTEKEFLYLKWDINIPLP |
| | IYGSVTYSKTRGWKINLCLETEVEQKNLSENKYLS |
| | IDLGVKRVATIFDGENTITLSGKKFMGLMHYRNKL |
| | NGKTQSRLSHKKKGSNNYKKIQRAKRKTTDRLLNI |
| | QKEMLHKYSSFIVNYAIRNDIGNIIIGDNSSTHDS |
| | PNMRGKTNQKISQNPEQKLKNYIKYKFESISGRVD |
| | IVPEPYTSRKCPHCKNIKKSSPKGRTYKCKKCGFI |
| | FDRDGVGAINIYNENVSFGQIISPGRIRSLTEPIG |
| | MKFHNEIYFKSYVAA |
| SEQ | MSVRSFQARVECDKQTMEHLWRTHKVFNERLPEII |
| ID | KILFKMKRGECGQNDKQKSLYKSISQSILEANAQN |
| NO: | ADYLLNSVSIKGWKPGTAKKYRNASFTWADDAAKL |
| 99 | SSQGIHVYDKKQVLGDLPGMMSQMVCRQSVEAISG |
| | HIELTKKWEKEHNEWLKEKEKWESEDEHKKYLDLR |
| | EKFEQFEQSIGGKITKRRGRWHLYLKWLSDNPDFA |
| | AWRGNKAVINPLSEKAQIRINKAKPNKKNSVERDE |
| | FFKANPEMKALDNLHGYYERNFVRRRKTKKNPDGF |
| | DHKPTFTLPHPTIHPRWFVFNKPKTNPEGYRKLIL |
| | PKKAGDLGSLEMRLLTGEKNKGNYPDDWISVKFKA |
| | DPRLSLIRPVKGRRVVRKGKEQGQTKETDSYEFFD |
| | KHLKKWRPAKLSGVKLIFPDKTPKAAYLYFTCDIP |
| | DEPLTETAKKIQWLETGDVTKKGKKRKKKVLPHGL |
| | VSCAVDLSMRRGTTGFATLCRYENGKIHILRSRNL |
| | WVGYKEGKGCHPYRWTEGPDLGHIAKHKREIRILR |
| | SKRGKPVKGEESHIDLQKHIDYMGEDRFKKAARTI |
| | VNFALNTENAASKNGFYPRADVLLLENLEGLIPDA |
| | EKERGINRALAGWNRRHLVERVIEMAKDAGFKRRV |
| | FEIPPYGTSQVCSKCGALGRRYSIIRENNRREIRF |
| | GYVEKLFACPNCGYCANADHNASVNLNRRFLIEDS |
| | FKSYYDWKRLSEKKQKEEIETIESKLMDKLCAMHK |
| | ISRGSISK |
| SEQ | MHLWRTHCVFNQRLPALLKRLFAMRRGEVGGNEAQ |
| ID | RQVYQRVAQFVLARDAKDSVDLLNAVSLRKRSANS |
| NO: | AFKKKATISCNGQAREVTGEEVFAEAVALASKGVF |
| 100 | AYDKDDMRAGLPDSLFQPLTRDAVACMRSHEELVA |
| | TWKKEYREWRDRKSEWEAEPEHALYLNLRPKFEEG |
| | EAARGGRFRKRAERDHAYLDWLEANPQLAAWRRKA |
| | PPAVVPIDEAGKRRIARAKAWKQASVRAEEFWKRN |
| | PELHALHKIHVQYLREFVRPRRTRRNKRREGFKQR |
| | PTFTMPDPVRHPRWCLFNAPQTSPQGYRLLRLPQS |
| | RRTVGSVELRLLTGPSDGAGFPDAWVNVRFKADPR |
| | LAQLRPVKVPRTVTRGKNKGAKVEADGFRYYDDQL |
| | LIERDAQVSGVKLLFRDIRMAPFADKPIEDRLLSA |
| | TPYLVFAVEIKDEARTERAKAIRFDETSELTKSGK |
| | KRKTLPAGLVSVAVDLDTRGVGFLTRAVIGVPEIQ |
| | QTHHGVRLLQSRYVAVGQVEARASGEAEWSPGPDL |
| | AHIARHKREIRRLRQLRGKPVKGERSHVRLQAHID |
| | RMGEDRFKKAARKIVNEALRGSNPAAGDPYTRADV |
| | LLYESLETLLPDAERERGINRALLRWNRAKLIEHL |
| | KRMCDDAGIRHFPVSPFGTSQVCSKCGALGRRYSL |
| | ARENGRAVIRFGWVERLFACPNPECPGRRPDRPDR |
| | PFTCNSDHNASVNLHRVFALGDQAVAAFRALAPRD |
| | SPARTLAVKRVEDTLRPQLMRVHKLADAGVDSPF |
| SEQ | MATLVYRYGVRAHGSARQQDAVVSDPAMLEQLRLG |
| ID | HELRNALVGVQHRYEDGKRAVWSGFASVAAADHRV |
| NO: | TTGETAVAELEKQARAEHSADRTAATRQGTAESLK |
| 101 | AARAAVKQARADRKAAMAAVAEQAKPKIQALGDDR |
| | DAEIKDLYRRFCQDGVLLPRCGRCAGDLRSDGDCT |
| | DCGAAHEPRKLYWATYNAIREDHQTAVKLVEAKRK |
| | AGQPARLRFRRWTGDGTLTVQLQRMHGPACRCVTC |
| | AEKLTRRARKTDPQAPAVAADPAYPPTDPPRDPAL |
| | LASGQGKWRNVLQLGTWIPPGEWSAMSRAERRRVG |
| | RSHIGWQLGGGRQLTLPVQLHRQMPADADVAMAQL |
| | TRVRVGGRHRMSVALTAKLPDPPQVQGLPPVALHL |
| | GWRQRPDGSLRVATWACPQPLDLPPAVADVVVSHG |
| | GRWGEVIMPARWLADAEVPPRLLGRRDKAMEPVLE |
| | ALADWLEAHTEACTARMTPALVRRWRSQGRLAGLT |
| | NRWRGQPPTGSAEILTYLEAWRIQDKLLWERESHL |
| | RRRLAARRDDAWRRVASWLARHAGVLVVDDADIAE |
| | LRRRDDPADTDPTMPASAAQAARARAALAAPGRLR |
| | HLATITATRDGLGVHTVASAGLTRLHRKCGHQAQP |
| | DPRYAASAVVTCPGCGNGYDQDYNAAMLMLDRQQQ |
| | P |
| SEQ | MSRVELHRAYKFRLYPTPAQVAELAEWERQLRRLY |
| ID | NLAHSQRLAAMQRHVRPKSPGVLKSECLSCGAVAV |
| NO: | AEIGTDGKAKKTVKHAVGCSVLECRSCGGSPDAEG |
| 102 | RTAHTAACSFVDYYRQGREMTQLLEEDDQLARVVC |
| | SARQETLRDLEKAWQRWHKMPGFGKPHFKKRIDSC |
| | RIYFSTPKSWAVDLGYLSFTGVASSVGRIKIRQDR |
| | VWPGDAKFSSCHVVRDVDEWYAVFPLTFTKEIEKP |
| | KGGAVGINRGAVHAIADSTGRVVDSPKFYARSLGV |
| | IRHRARLLDRKVPFGRAVKPSPTKYHGLPKADIDA |
| | AAARVNASPGRLVYEARARGSIAAAEAHLAALVLP |
| | APRQTSQLPSEGRNRERARRFLALAHQRVRRQREW |
| | FLHNESAHYAQSYTKIAIEDWSTKEMTSSEPRDAE |
| | EMKRVTRARNRSILDVGWYELGRQIAYKSEATGAE |
| | FAKVDPGLRETETHVPEAIVRERDVDVSGMLRGEA |
| | GISGTCSRCGGLLRASASGHADAECEVCLHVEVGD |
| | VNAAVNVLKRAMFPGAAPPSKEKAKVTIGIKGRKK |
| | KRAA |
| SEQ | MSRVELHRAYKFRLYPTPVQVAELSEWERQLRRLY |
| ID | NLGHEQRLLTLTRHLRPKSPGVLKGECLSCDSTQV |
| NO: | QEVGADGRPKTTVRHAEQCPTLACRSCGALRDAEG |
| 103 | RTAHTVACAFVDYYRQGREMTELLAADDQLARVVC |
| | SARQEVLRDLDKAWQRWRKMPGFGKPRFKRRTDSC |
| | RIYFSTPKAWKLEGGHLSFTGAATTVGAIKMRQDR |
| | NWPASVQFSSCHVVRDVDEWYAVFPLTFVAEVARP |
| | KGGAVGINRGAVHAIADSTGRVVDSPRYYARALGV |
| | IRHRARLFDRKVPSGHAVKPSPTKYRGLSAIEVDR |
| | VARATGFTPGRVVTEALNRGGVAYAECALAAIAVL |
| | GHGPERPLTSDGRNREKARKFLALAHQRVRRQREW |
| | FLHNESAHYARTYSKIAIEDWSTKEMTASEPQGEE |
| | TRRVTRSRNRSILDVGWYELGRQLAYKTEATGAEF |
| | AQVDPGLKETETNVPKAIADARDVDVSGMLRGEAG |
| | ISGTCSKCGGLLRAPASGHADAECEICLNVEVGDV |
| | NAAVNVLKRAMFPGDAPPASGEKPKVSIGIKGRQK |
| | KKKAA |
| SEQ | MEAIATGMSPERRVELGILPGSVELKRAYKFRLYP |
| ID | MKVQQAELSEWERQLRRLYNLAHEQRLAALLRYRD |
| NO: | WDFQKGACPSCRVAVPGVHTAACDHVDYFRQAREM |
| 104 | TQLLEVDAQLSRVICCARQEVLRDLDKAWQRWRKK |
| | LGGRPRFKRRTDSCRIYLSTPKHWEIAGRYLRLSG |
| | LASSVGEIRIEQDRAFPEGALLSSCSIVRDVDEWY |
| | ACLPLTFTQPIERAPHRSVGLNRGVVHALADSDGR |
| | VVDSPKFFERALATVQKRSRDLARKVSGSRNAHKA |
| | RIKLAKAHQRVRRQRAAFLHQESAYYSKGFDLVAL |
| | EDMSVRKMTATAGEAPEMGRGAQRDLNRGILDVGW |
| | YELARQIDYKRLAHGGELLRVDPGQTTPLACVTEE |
| | QPARGISSACAVCGIPLARPASGNARMRCTACGSS |
| | QVGDVNAAENVLTRALSSAPSGPKSPKASIKIKGR |
| | QKRLGTPANRAGEASGGDPPVRGPVEGGTLAYVVE |
| | PVSESQSDT |
| SEQ | MTVRTYKYRAYPTPEQAEALTSWLRFASQLYNAAL |
| ID | EHRKNAWGRHDAHGRGFRFWDGDAAPRKKSDPPGR |
| NO: | WVYRGGGGAHISKNDQGKLLTEFRREHAELLPPGM |
| 105 | PALVQHEVLARLERSMAAFFQRATKGQKAGYPRWR |
| | SEHRYDSLTFGLTSPSKERFDPETGESLGRGKTVG |
| | AGTYHNGDLRLTGLGELRILEHRRIPMGAIPKSVI |
| | VRRSGKRWFVSIAMEMPSVEPAASGRPAVGLDMGV |
| | VTWGTAFTADTSAAAALVADLRRMATDPSDCRRLE |
| | ELEREAAQLSEVLAHCRARGLDPARPRRCPKELTK |
| | LYRRSLHRLGELDRACARIRRRLQAAHDIAEPVPD |
| | EAGSAVLIEGSNAGMRHARRVARTQRRVARRTRAG |
| | HAHSNRRKKAVQAYARAKERERSARGDHRHKVSRA |
| | LVRQFEEISVEALDIKQLTVAPEHNPDPQPDLPAH |
| | VQRRRNRGELDAAWGAFFAALDYKAADAGGRVARK |
| | PAPHTTQECARCGTLVPKPISLRVHRCPACGYTAP |
| | RTVNSARNVLQRPLEEPGRAGPSGANGRGVPHAVA |
| SEQ | MNCRYRYRIYPTPGQRQSLARLFGCVRVVWNDALF |
| ID | LCRQSEKLPKNSELQKLCITQAKKTEARGWLGQVS |
| NO: | AIPLQQSVADLGVAFKNFFQSRSGKRKGKKVNPPR |
| 106 | VKRRNNRQGARFTRGGFKVKTSKVYLARIGDIKIK |
| | WSRPLPSEPSSVTVIKDCAGQYFLSFVVEVKPEIK |
| | PPKNPSIGIDLGLKTFASCSNGEKIDSPDYSRLYR |
| | KLKRCQRRLAKRQRGSKRRERMRVKVAKLNAQIRD |
| | KRKDFLHKLSTKVVNENQVIALEDLNVGGMLKNRK |
| | LSRAISQAGWYEFRSLCEGKAEKHNRDFRVISRWE |
| | PTSQVCSECGYRWGKIDLSVRSIVCINCGVEHDRD |
| | DNASVNIEQAGLKVGVGHTHDSKRTGSACKTSNGA |
| | VCVEPSTHREYVQLTLFDW |
| SEQ | MKSRWTFRCYPTPEQEQHLARTFGCVRFVWNWALR |
| ID | ARTDAFRAGERIGYPATDKALTLLKQQPETVWLNE |
| NO: | VSSVCLQQALRDLQVAFSNFFDKRAAHPSFKRKEA |
| 107 | RQSANYTERGFSFDHERRILKLAKIGAIKVKWSRK |
| | AIPHPSSIRLIRTASGKYFVSLVVETQPAPMPETG |
| | ESVGVDFGVARLATLSNGERISNPKHGAKWQRRLA |
| | FYQKRLARATKGSKRRMRIKRHVARIHEKIGNSRS |
| | DTLHKLSTDLVTRFDLICVEDLNLRGMVKNHSLAR |
| | SLHDASIGSAIRMIEEKAERYGKNVVKIDRWFPSS |
| | KTCSDCGHIVEQLPLNVREWTCPECGTTHDRDANA |
| | AANILAVGQTVSAHGGTVRRSRAKASERKSQRSAN |
| | RQGVNRA |
| SEQ | KEPLNIGKTAKAVFKEIDPTSLNRAANYDASIELN |
| ID | CKECKFKPFKNVKRYEFNFYNNWYRCNPNSCLQST |
| NO: | YKAQVRKVEIGYEKLKNEILTQMQYYPWFGRLYQN |
| 108 | FFHDERDKMTSLDEIQVIGVQNKVFFNTVEKAWRE |
| | IIKKRFKDNKETMETIPELKHAAGHGKRKLSNKSL |
| | LRRRFAFVQKSFKFVDNSDVSYRSFSNNIACVLPS |
| | RIGVDLGGVISRNPKREYIPQEISFNAFWKQHEGL |
| | KKGRNIEIQSVQYKGETVKRIEADTGEDKAWGKNR |
| | QRRFTSLILKLVPKQGGKKVWKYPEKRNEGNYEYF |
| | PIPIEFILDSGETSIRFGGDEGEAGKQKHLVIPFN |
| | DSKATPLASQQTLLENSRFNAEVKSCIGLAIYANY |
| | FYGYARNYVISSIYHKNSKNGQAITAIYLESIAHN |
| | YVKAIERQLQNLLLNLRDFSFMESHKKELKKYFGG |
| | DLEGTGGAQKRREKEEKIEKEIEQSYLPRLIRLSL |
| | TKMVTKQVEM |
| SEQ | ELIVNENKDPLNIGKTAKAVFKEIDPTSINRAANY |
| ID | DASIELACKECKFKPFNNTKRHDFSFYSNWHRCSP |
| NO: | NSCLQSTYRAKIRKTEIGYEKLKNEILNQMQYYPW |
| 109 | FGRLYQNFFNDQRDKMTSLDEIQVTGVQNKIFFNT |
| | VEKAWREIIKKRFRDNKETMRTIPDLKNKSGHGSR |
| | KLSNKSLLRRRFAFAQKSFKLVDNSDVSYRAFSNN |
| | VACVLPSKIGVDIGGIINKDLKREYIPQEITFNVF |
| | WKQHDGLKKGRNIEIHSVQYKGEIVKRIEADTGED |
| | KAWGKNRQRRFTSLILKITPKQGGKKIWKFPEKKN |
| | ASDYEYFPIPIEFILDNGDASIKFGGEEGEVGKQK |
| | HLLIPFNDSKATPLSSKQMLLETSRFNAEVKSTIG |
| | LALYANYFVSYARNYVIKSTYHKNSKKGQIVTEIY |
| | LESISQNFVRAIQRQLQSLMLNLKDWGFMQTHKKE |
| | LKKYFGSDLEGSKGGQKRREKEEKIEKEIEASYLP |
| | RLIRLSLTKSVTKAEEM |
| SEQ | PEEKTSKLKPNSINLAANYDANEKFNCKECKFHPF |
| ID | KNKKRYEFNFYNNLHGCKSCTKSTNNPAVKRIEIG |
| NO: | YQKLKFEIKNQMEAYPWFGRLRINFYSDEKRKMSE |
| 110 | LNEMQVTGVKNKIFFDAIECAWREILKKRFRESKE |
| | TLITIPKLKNKAGHGARKHRNKKLLIRRRAFMKKN |
| | FHFLDNDSISYRSFANNIACVLPSKVGVDIGGIIS |
| | PDVGKDIKPVDISLNLMWASKEGIKSGRKVEIYST |
| | QYDGNMVKKIEAETGEDKSWGKNRKRRQTSLLLSI |
| | PKPSKQVQEFDFKEWPRYKDIEKKVQWRGFPIKII |
| | FDSNHNSIEFGTYQGGKQKVLPIPFNDSKTTPLGS |
| | KMNKLEKLRFNSKIKSRLGSAIAANKFLEAARTYC |
| | VDSLYHEVSSANAIGKGKIFIEYYLEILSQNYIEA |
| | AQKQLQRFIESIEQWFVADPFQGRLKQYFKDDLKR |
| | AKCFLCANREVQTTCYAAVKLHKSCAEKVKDKNKE |
| | LAIKERNNKEDAVIKEVEASNYPRVIRLKLTKTIT |
| | NKAM |
| SEQ | SESENKIIEQYYAFLYSFRDKYEKPEFKNRGDIKR |
| ID | KLONKWEDFLKEQNLKNDKKLSNYIFSNRNFRRSY |
| NO: | DREEENEEGIDEKKSKPKRINCFEKEKNLKDQYDK |
| 111 | DAINASANKDGAQKWGCFECIFFPMYKIESGDPNK |
| | RIIINKTRFKLFDFYLNLKGCKSCLRSTYHPYRSN |
| | VYIESNYDKLKREIGNFLQQKNIFQRMRKAKVSEG |
| | KYLTNLDEYRLSCVAMHFKNRWLFFDSIQKVLRET |
| | IKQRLKQMRESYDEQAKTKRSKGHGRAKYEDQVRM |
| | IRRRAYSAQAHKLLDNGYITLFDYDDKEINKVCLT |
| | AINQEGFDIGGYLNSDIDNVMPPIEISFHLKWKYN |
| | EPILNIESPFSKAKISDYLRKIREDLNLERGKEGK |
| | ARSKKNVRRKVLASKGEDGYKKIFTDFFSKWKEEL |
| | EGNAMERVLSQSSGDIQWSKKKRIHYTTLVLNINL |
| | LDKKGVGNLKYYEIAEKTKILSFDKNENKFWPITI |
| | QVLLDGYEIGTEYDEIKQLNEKTSKQFTIYDPNTK |
| | IIKIPFTDSKAVPLGMLGINIATLKTVKKTERDIK |
| | VSKIFKGGLNSKIVSKIGKGIYAGYFPTVDKEILE |
| | EVEEDTLDNEFSSKSQRNIFLKSIIKNYDKMLKEQ |
| | LFDFYSFLVRNDLGVRFLTDRELQNIEDESFNLEK |
| | RFFETDRDRIARWFDNTNTDDGKEKFKKLANEIVD |
| | SYKPRLIRLPVVRVIKRIQPVKQREM |
| SEQ | KYSTRDFSELNEIQVTACKQDEFFKVIQNAWREII |
| ID | KKRFLENRENFIEKKIFKNKKGRGKRQESDKTIQR |
| NO: | NRASVMKNFQLIENEKIILRAPSGHVACVFPVKVG |
| 112 | LDIGGFKTDDLEKNIFPPRTITINVFWKNRDRQRK |
| | GRKLEVWGIKARTKLIEKVHKWDKLEEVKKKRLKS |
| | LEQKQEKSLDNWSEVNNDSFYKVQIDELQEKIDKS |
| | LKGRTMNKILDNKAKESKEAEGLYIEWEKDFEGEM |
| | LRRIEASTGGEEKWGKRRQRRHTSLLLDIKNNSRG |
| | SKEIINFYSYAKQGKKEKKIEFFPFPLTITLDAEE |
| | ESPLNIKSIPIEDKNATSKYFSIPFTETRATPLSI |
| | LGDRVQKFKTKNISGAIKRNLGSSISSCKIVQNAE |
| | TSAKSILSLPNVKEDNNMEIFINTMSKNYFRAMMK |
| | QMESFIFEMEPKTLIDPYKEKAIKWFEVAASSRAK |
| | RKLKKLSKADIKKSELLLSNTEEFEKEKQEKLEAL |
| | EKEIEEFYLPRIVRLQLTKTILETPVM |
| SEQ | KKLQLLGHKILLKEYDPNAVNAAANFETSTAELCG |
| ID | QCKMKPFKNKRRFQYTFGKNYHGCLSCIQNVYYAK |
| NO: | KRIVQIAKEELKHQLTDSIASIPYKYTSLFSNTNS |
| 113 | IDELYILKQERAAFFSNTNSIDELYITGIENNIAF |
| | KVISAIWDEIIKKRRQRYAESLTDTGTVKANRGHG |
| | GTAYKSNTRQEKIRALQKQTLHMVTNPYISLARYK |
| | NNYIVATLPRTIGMHIGAIKDRDPQKKLSDYAINF |
| | NVFWSDDRQLIELSTVQYTGDMVRKIEAETGENNK |
| | WGENMKRTKTSLLLEILTKKTTDELTFKDWAFSTK |
| | KEIDSVTKKTYQGFPIGIIFEGNESSVKFGSQNYF |
| | PLPFDAKITPPTAEGFRLDWLRKGSFSSQMKTSYG |
| | LAIYSNKVTNAIPAYVIKNMFYKIARAENGKQIKA |
| | KFLKKYLDIAGNNYVPFIIMQHYRVLDTFEEMPIS |
| | QPKVIRLSLTKTQHIIIKKDKTDSKM |
| SEQ | NTSNLINLGKKAINISANYDANLEVGCKNCKFLSS |
| ID | NGNFPRQTNVKEGCHSCEKSTYEPSIYLVKIGERK |
| NO: | AKYDVLDSLKKFTFQSLKYQSKKSMKSRNKKPKEL |
| 114 | KEFVIFANKNKAFDVIQKSYNHLILQIKKEINRMN |
| | SKKRKKNHKRRLFRDREKQLNKLRLIESSNLFLPR |
| | ENKGNNHVFTYVAIHSVGRDIGVIGSYDEKLNFET |
| | ELTYQLYFNDDKRLLYAYKPKQNKIIKIKEKLWNL |
| | RKEKEPLDLEYEKPLNKSITFSIKNDNLFKVSKDL |
| | MLRRAKFNIQGKEKLSKEERKINRDLIKIKGLVNS |
| | MSYGRFDELKKEKNIWSPHIYREVRQKEIKPCLIK |
| | NGDRIEIFEQLKKKMERLRRFREKRQKKISKDLIF |
| | AERIAYNFHTKSIKNTSNKINIDQEAKRGKASYMR |
| | KRIGYETFKNKYCEQCLSKGNVYRNVQKGCSCFEN |
| | PFDWIKKGDENLLPKKNEDLRVKGAFRDEALEKQI |
| | VKIAFNIAKGYEDFYDNLGESTEKDLKLKFKVGTT |
| | INEQESLKL |
| SEQ | TSNPIKLGKKAINISANYDSNLQIGCKNCKFLSYN |
| ID | GNFPRQTNVKEGCHSCEKSTYEPPVYTVRIGERRS |
| NO: | KYDVLDSLKKFIFLSLKYRQSKKMKTRSKGIRGLE |
| 115 | EFVISANLKKAMDVIQKSYRHLILNIKNEIVRMNG |
| | KKRNKNHKRLLFRDREKQLNKLRLIEGSSFFKPPT |
| | VKGDNSIFTCVAIHNIGRDIGIAGDYFDKLEPKIE |
| | LTYQLYYEYNPKKESEINKRLLYAYKPKQNKIIEI |
| | KEKLWNLRKEKSPLDLEYEKPLTKSITFLVKRDGV |
| | FRISKDLMLRKAKFIIQGKEKLSKEERKINRDLIK |
| | IKSNIISLTYGRFDELKKDKTIWSPHIFRDVKQGK |
| | ITPCIERKGDRMDIFQQLRKKSERLRENRKKRQKK |
| | ISKDLIFAERIAYNFHTKSIKNTSNLINIKHEAKR |
| | GKASYMRKRIGNETFRIKYCEQCFPKNNVYKNVQK |
| | GCSCFEDPFEYIKKGNEDLIPNKNQDLKAKGAFRD |
| | DALEKQIIKVAFNIAKGYEDFYENLKKTTEKDIRL |
| | KFKVGTIISEEM |
| SEQ | NNSINLSKKAINISANYDANLQVRCKNCKFLSSNG |
| ID | NFPRQTDVKEGCHSCEKSTYEPPVYDVKIGEIKAK |
| NO: | YEVLDSLKKFTFQSLKYQLSKSMKFRSKKIKELKE |
| 116 | FVIFAKESKALNVINRSYKHLILNIKNDINRMNSK |
| | KRIKNHKGRLFLDRQKQLSKLKLIEGSSFFVPAKN |
| | VGNKSVFTCVAIHSIGRDIGIAGLYDSFTKPVNEI |
| | TYQIFFSGERRLLYAYKPKQLKILSIKENLWSLKN |
| | EKKPLDLLYEKPLGKNLNFNVKGGDLFRVSKDLMI |
| | RNAKFNVHGRQRLSDEERLINRNFIKIKGEVVSLS |
| | YGRFEELKKDRKLWSPHIFKDVRQNKIKPCLVMQG |
| | QRIDIFEQLKRKLELLKKIRKSRQKKLSKDLIFGE |
| | RIAYNFHTKSIKNTSNKINIDSDAKRGRASYMRKR |
| | IGNETFKLKYCDVCFPKANVYRRVQNGCSCSENPY |
| | NYIKKGDKDLLPKKDEGLAIKGAFRDEKLNKQIIK |
| | VAFNIAKGYEDFYDDLKKRTEKDVDLKFKIGTTVL |
| | DQKPMEIFDGIVITWL |
| SEQ | LLTTVVETNNLAKKAINVAANFDANIDRQYYRCTP |
| ID | NLCRFIAQSPRETKEKDAGCSSCTQSTYDPKVYVI |
| NO: | KIGKLLAKYEILKSLKRFLFMNRYFKQKKTERAQQ |
| 117 | KQKIGTELNEMSIFAKATNAMEVIKRATKHCTYDI |
| | IPETKSLQMLKRRRHRVKVRSLLKILKERRMKIKK |
| | IPNTFIEIPKQAKKNKSDYYVAAALKSCGIDVGLC |
| | GAYEKNAEVEAEYTYQLYYEYKGNSSTKRILYCYN |
| | NPQKNIREFWEAFYIQGSKSHVNTPGTIRLKMEKF |
| | LSPITIESEALDFRVWNSDLKIRNGQYGFIKKRSL |
| | GKEAREIKKGMGDIKRKIGNLTYGKSPSELKSIHV |
| | YRTERENPKKPRAARKKEDNFMEIFEMQRKKDYEV |
| | NKKRRKEATDAAKIMDFAEEPIRHYHTNNLKAVRR |
| | IDMNEQVERKKTSVFLKRIMQNGYRGNYCRKCIKA |
| | PEGSNRDENVLEKNEGCLDCIGSEFIWKKSSKEKK |
| | GLWHTNRLLRRIRLQCFTTAKAYENFYNDLFEKKE |
| | SSLDIIKLKVSITTKSM |
| SEQ | ASTMNLAKQAINFAANYDSNLEIGCKGCKFMSTWS |
| ID | KKSNPKFYPRQNNQANKCHSCTYSTGEPEVPIIEI |
| NO: | GERAAKYKIFTALKKFVFMSVAYKERRRQRFKSKK |
| 118 | PKELKELAICSNREKAMEVIQKSVVHCYGDVKQEI |
| | PRIRKIKVLKNHKGRLFYKQKRSKIKIAKLEKGSF |
| | FKTFIPKVHNNGCHSCHEASLNKPILVTTALNTIG |
| | ADIGLINDYSTIAPTETDISWQVYYEFIPNGDSEA |
| | VKKRLLYFYKPKGALIKSIRDKYFKKGHENAVNTG |
| | FFKYQGKIVKGPIKFVNNELDFARKPDLKSMKIKR |
| | AGFAIPSAKRLSKEDREINRESIKIKNKIYSLSYG |
| | RKKTLSDKDIIKHLYRPVRQKGVKPLEYRKAPDGF |
| | LEFFYSLKRKERRLRKQKEKRQKDMSEIIDAADEF |
| | AWHRHTGSIKKTTNHINFKSEVKRGKVPIMKKRIA |
| | NDSFNTRHCGKCVKQGNAINKYYIEKQKNCFDCNS |
| | IEFKWEKAALEKKGAFKLNKRLQYIVKACFNVAKA |
| | YESFYEDFRKGEEESLDLKFKIGTTTTLKQYPQNK |
| | ARAM |
| SEQ | HSHNLMLTKLGKQAINFAANYDANLEIGCKNCKFL |
| ID | SYSPKQANPKKYPRQTDVHEDGNIACHSCMQSTKE |
| NO: | PPVYIVPIGERKSKYEILTSLNKFTFLALKYKEKK |
| 119 | RQAFRAKKPKELQELAIAFNKEKAIKVIDKSIQHL |
| | ILNIKPEIARIQRQKRLKNRKGKLLYLHKRYAIKM |
| | GLIKNGKYFKVGSPKKDGKKLLVLCALNTIGRDIG |
| | IIGNIEENNRSETEITYQLYFDCLDANPNELRIKE |
| | IEYNRLKSYERKIKRLVYAYKPKQTKILEIRSKFF |
| | SKGHENKVNTGSFNFENPLNKSISIKVKNSAFDFK |
| | IGAPFIMLRNGKFHIPTKKRLSKEEREINRTLSKI |
| | KGRVFRLTYGRNISEQGSKSLHIYRKERQHPKLSL |
| | EIRKQPDSFIDEFEKLRLKQNFISKLKKQRQKKLA |
| | DLLQFADRIAYNYHTSSLEKTSNFINYKPEVKRGR |
| | TSYIKKRIGNEGFEKLYCETCIKSNDKENAYAVEK |
| | EELCFVCKAKPFTWKKTNKDKLGIFKYPSRIKDFI |
| | RAAFTVAKSYNDFYENLKKKDLKNEIFLKFKIGLI |
| | LSHEKKNHISIAKSVAEDERISGKSIKNILNKSIK |
| | LEKNCYSCFFHKEDM |
| SEQ | SLERVIDKRNLAKKAINIAANFDANINKGFYRCET |
| ID | NQCMFIAQKPRKTNNTGCSSCLQSTYDPVIYVVKV |
| NO: | GEMLAKYEILKSLKRFVFMNRSFKQKKTEKAKQKE |
| 120 | RIGGELNEMSIFANAALAMGVIKRAIRHCHVDIRP |
| | EINRLSELKKTKHRVAAKSLVKIVKQRKTKWKGIP |
| | NSFIQIPQKARNKDADFYVASALKSGGIDIGLCGT |
| | YDKKPHADPRWTYQLYFDTEDESEKRLLYCYNDPQ |
| | AKIRDFWKTFYERGNPSMVNSPGTIEFRMEGFFEK |
| | MTPISIESKDFDFRVWNKDLLIRRGLYEIKKRKNL |
| | NRKAREIKKAMGSVKRVLANMTYGKSPTDKKSIPV |
| | YRVEREKPKKPRAVRKEENELADKLENYRREDFLI |
| | RNRRKREATEIAKIIDAAEPPIRHYHTNHLRAVKR |
| | IDLSKPVARKNTSVFLKRIMQNGYRGNYCKKCIKG |
| | NIDPNKDECRLEDIKKCICCEGTQNIWAKKEKLYT |
| | GRINVLNKRIKQMKLECFNVAKAYENFYDNLAALK |
| | EGDLKVLKLKVSIPALNPEASDPEEDM |
| SEQ | NASINLGKRAINLSANYDSNLVIGCKNCKFLSFNG |
| ID | NFPRQTNVREGCHSCDKSTYAPEVYIVKIGERKAK |
| NO: | YDVLDSLKKFTFQSLKYQIKKSMRERSKKPKELLE |
| 121 | FVIFANKDKAFNVIQKSYEHLILNIKQEINRMNGK |
| | KRIKNHKKRLFKDREKQLNKLRLIGSSSLFFPREN |
| | KGDKDLFTYVAIHSVGRDIGVAGSYESHIEPISDL |
| | TYQLFINNEKRLLYAYKPKQNKIIELKENLWNLKK |
| | EKKPLDLEFTKPLEKSITFSVKNDKLFKVSKDLML |
| | RQAKFNIQGKEKLSKEERQINRDFSKIKSNVISLS |
| | YGRFEELKKEKNIWSPHIYREVKQKEIKPCIVRKG |
| | DRIELFEQLKRKMDKLKKFRKERQKKISKDLNFAE |
| | RIAYNFHTKSIKNTSNKINIDQEAKRGKASYMRKR |
| | IGNESFRKKYCEQCFSVGNVYHNVQNGCSCFDNPI |
| | ELIKKGDEGLIPKGKEDRKYKGALRDDNLQMQIIR |
| | VAFNIAKGYEDFYNNLKEKTEKDLKLKFKIGTTIS |
| | TQESNNKEM |
| SEQ | SNLIKLGKQAINFAANYDANLEVGCKNCKFLSSTN |
| ID | KYPRQTNVHLDNKMACRSCNQSTMEPAIYIVRIGE |
| NO: | KKAKYDIYNSLTKFNFQSLKYKAKRSQRFKPKQPK |
| 122 | ELQELSIAVRKEKALDIIQKSIDHLIQDIRPEIPR |
| | IKQQKRYKNHVGKLFYLQKRRKNKLNLIGKGSFFK |
| | VFSPKEKKNELLVICALTNIGRDIGLIGNYNTIIN |
| | PLFEVTYQLYYDYIPKKNNKNVQRRLLYAYKSKNE |
| | KILKLKEAFFKRGHENAVNLGSFSYEKPLEKSLTL |
| | KIKNDKDDFQVSPSLRIRTGRFFVPSKRNLSRQER |
| | EINRRLVKIKSKIKNMTYGKFETARDKQSVHIFRL |
| | ERQKEKLPLQFRKDEKEFMEEFQKLKRRTNSLKKL |
| | RKSRQKKLADLLQLSEKVVYNNHTGTLKKTSNFLN |
| | FSSSVKRGKTAYIKELLGQEGFETLYCSNCINKGQ |
| | KTRYNIETKEKCFSCKDVPFVWKKKSTDKDRKGAF |
| | LFPAKLKDVIKATFTVAKAYEDFYDNLKSIDEKKP |
| | YIKFKIGLILAHVRHEHKARAKEEAGQKNIYNKPI |
| | KIDKNCKECFFFKEEAM |
| SEQ | NTTRKKFRKRTGFPQSDNIKLAYCSAIVRAANLDA |
| ID | DIQKKHNQCNPNLCVGIKSNEQSRKYEHSDRQALL |
| NO: | CYACNQSTGAPKVDYIQIGEIGAKYKILQMVNAYD |
| 123 | FLSLAYNLTKLRNGKSRGHQRMSQLDEVVIVADYE |
| | KATEVIKRSINHLLDDIRGQLSKLKKRTQNEHITE |
| | HKQSKIRRKLRKLSRLLKRRRWKWGTIPNPYLKNW |
| | VFTKKDPELVTVALLHKLGRDIGLVNRSKRRSKQK |
| | LLPKVGFQLYYKWESPSLNNIKKSKAKKLPKRLLI |
| | PYKNVKLFDNKQKLENAIKSLLESYQKTIKVEFDQ |
| | FFQNRTEEIIAEEQQTLERGLLKQLEKKKNEFASQ |
| | KKALKEEKKKIKEPRKAKLLMEESRSLGFLMANVS |
| | YALFNTTIEDLYKKSNVVSGCIPQEPVVVFPADIQ |
| | NKGSLAKILFAPKDGFRIKFSGQHLTIRTAKFKIR |
| | GKEIKILTKTKREILKNIEKLRRVWYREQHYKLKL |
| | FGKEVSAKPRFLDKRKTSIERRDPNKLADQTDDRQ |
| | AELRNKEYELRHKQHKMAERLDNIDTNAQNLQTLS |
| | FWVGEADKPPKLDEKDARGFGVRTCISAWKWFMED |
| | LLKKQEEDPLLKLKLSIM |
| SEQ | PKKPKFQKRTGFPQPDNLRKEYCLAIVRAANLDAD |
| ID | FEKKCTKCEGIKTNKKGNIVKGRTYNSADKDNLLC |
| NO: | YACNISTGAPAVDYVFVGALEAKYKILQMVKAYDF |
| 124 | HSLAYNLAKLWKGRGRGHQRMGGLNEVVIVSNNEK |
| | ALDVIEKSLNHFHDEIRGELSRLKAKFQNEHLHVH |
| | KESKLRRKLRKISRLLKRRRWKWDVIPNSYLRNFT |
| | FTKTRPDFISVALLHRVGRDIGLVTKTKIPKPTDL |
| | LPQFGFQIYYTWDEPKLNKLKKSRLRSEPKRLLVP |
| | YKKIELYKNKSVLEEAIRHLAEVYTEDLTICFKDF |
| | FETQKRKFVSKEKESLKRELLKELTKLKKDFSERK |
| | TALKRDRKEIKEPKKAKLLMEESRSLGFLAANTSY |
| | ALFNLIAADLYTKSKKACSTKLPRQLSTILPLEIK |
| | EHKSTTSLAIKPEEGFKIRFSNTHLSIRTPKFKMK |
| | GADIKALTKRKREILKNATKLEKSWYGLKHYKLKL |
| | YGKEVAAKPRFLDKRNPSIDRRDPKELMEQIENRR |
| | NEVKDLEYEIRKGQHQMAKRLDNVDTNAQNLQTKS |
| | FWVGEADKPPELDSMEAKKLGLRTCISAWKWFMKD |
| | LVLLQEKSPNLKLKLSLTEM |
| SEQ | KFSKRQEGFLIPDNIDLYKCLAIVRSANLDADVQG |
| ID | HKSCYGVKKNGTYRVKQNGKKGVKEKGRKYVFDLI |
| NO: | AFKGNIEKIPHEAIEEKDQGRVIVLGKFNYKLILN |
| 125 | IEKNHNDRASLEIKNKIKKLVQISSLETGEFLSDL |
| | LSGKIGIDEVYGIIEPDVFSGKELVCKACQQSTYA |
| | PLVEYMPVGELDAKYKILSAIKGYDFLSLAYNLSR |
| | NRANKKRGHQKLGGGELSEVVISANYDKALNVIKR |
| | SINHYHVEIKPEISKLKKKMQNEPLKVMKQARIRR |
| | ELHQLSRKVKRLKWKWGMIPNPELQNIIFEKKEKD |
| | FVSYALLHTLGRDIGLFKDTSMLQVPNISDYGFQI |
| | YYSWEDPKLNSIKKIKDLPKRLLIPYKRLDFYIDT |
| | ILVAKVIKNLIELYRKSYVYETFGEEYGYAKKAED |
| | ILFDWDSINLSEGIEQKIQKIKDEFSDLLYEARES |
| | KRQNFVESFENILGLYDKNFASDRNSYQEKIQSMI |
| | IKKQQENIEQKLKREFKEVIERGFEGMDQNKKYYK |
| | VLSPNIKGGLLYTDTNNLGFFRSHLAFMLLSKISD |
| | DLYRKNNLVSKGGNKGILDQTPETMLTLEFGKSNL |
| | PNISIKRKFFNIKYNSSWIGIRKPKFSIKGAVIRE |
| | ITKKVRDEQRLIKSLEGVWHKSTHFKRWGKPRFNL |
| | PRHPDREKNNDDNLMESITSRREQIQLLLREKQKQ |
| | QEKMAGRLDKIDKEIQNLQTANFQIKQIDKKPALT |
| | EKSEGKQSVRNALSAWKWFMEDLIKYQKRTPILQL |
| | KLAKM |
| SEQ | KFSKRQEGFVIPENIGLYKCLAIVRSANLDADVQG |
| ID | HVSCYGVKKNGTYVLKQNGKKSIREKGRKYASDLV |
| NO: | AFKGDIEKIPFEVIEEKKKEQSIVLGKFNYKLVLD |
| 126 | VMKGEKDRASLTMKNKSKKLVQVSSLGTDEFLLTL |
| | LNEKFGIEEIYGIIEPEVFSGKKLVCKACQQSTYA |
| | PLVEYMPVGELDSKYKILSAIKGYDFLSLAYNLAR |
| | HRSNKKRGHQKLGGGELSEVVISANNAKALNVIKR |
| | SLNHYYSEIKPEISKLRKKMQNEPLKVGKQARMRR |
| | ELHQLSRKVKRLKWKWGKIPNLELQNITFKESDRD |
| | FISYALLHTLGRDIGMFNKTEIKMPSNILGYGFQI |
| | YYDWEEPKLNTIKKSKNTPKRILIPYKKLDFYNDS |
| | ILVARAIKELVGLFQESYEWEIFGNEYNYAKEAEV |
| | ELIKLDEESINGNVEKKLQRIKENFSNLLEKAREK |
| | KRQNFIESFESIARLYDESFTADRNEYQREIQSFI |
| | IEKQKQSIEKKLKNEFKKIVEKKFNEQEQGKKHYR |
| | VLNPTIINEFLPKDKNNLGFLRSKIAFILLSKISD |
| | DLYKKSNAVSKGGEKGIIKQQPETILDLEFSKSKL |
| | PSINIKKKLFNIKYTSSWLGIRKPKFNIKGAKIRE |
| | ITRRVRDVQRTLKSAESSWYASTHFRRWGFPRFNQ |
| | PRHPDKEKKSDDRLIESITLLREQIQILLREKQKG |
| | QKEMAGRLDDVDKKIQNLQTANFQIKQTGDKPALT |
| | EKSAGKQSFRNALSAWKWFMENLLKYQNKTPDLKL |
| | KIARTVM |
| SEQ | KWIEPNNIDFNKCLAITRSANLDADVQGHKMCYGI |
| ID | KTNGTYKAIGKINKKHNTGIIEKRRTYVYDLIVTK |
| NO: | EKNEKIVKKTDFMAIDEEIEFDEKKEKLLKKYIKA |
| 127 | EVLGTGELIRKDLNDGEKFDDLCSIEEPQAFRRSE |
| | LVCKACNQSTYASDIRYIPIGEIEAKYKILKAIKG |
| | YDFLSLKYNLGRLRDSKKRGHQKMGQGELKEFVIC |
| | ANKEKALDVIKRSLNHYLNEVKDEISRLNKKMQNE |
| | PLKVNDQARWRRELNQISRRLKRLKWKWGEIPNPE |
| | LKNLIFKSSRPEFVSYALIHTLGRDIGLINETELK |
| | PNNIQEYGFQIYYKWEDPELNHIKKVKNIPKRFII |
| | PYKNLDLFGKYTILSRAIEGILKLYSSSFQYKSFK |
| | DPNLFAKEGEKKITNEDFELGYDEKIKKIKDDFKS |
| | YKKALLEKKKNTLEDSLNSILSVYEQSLLTEQINN |
| | VKKWKEGLLKSKESIHKQKKIENIEDIISRIEELK |
| | NVEGWIRTKERDIVNKEETNLKREIKKELKDSYYE |
| | EVRKDFSDLKKGEESEKKPFREEPKPIVIKDYIKF |
| | DVLPGENSALGFFLSHLSFNLFDSIQYELFEKSRL |
| | SSSKHPQIPETILDL |
| SEQ | FRKFVKRSGAPQPDNLNKYKCIAIVRAANLDADIM |
| ID | SNESSNCVMCKGIKMNKRKTAKGAAKTTELGRVYA |
| NO: | GQSGNLLCTACTKSTMGPLVDYVPIGRIRAKYTIL |
| 128 | RAVKEYDFLSLAYNLARTRVSKKGGRQKMHSLSEL |
| | VIAAEYEIAWNIIKSSVIHYHQETKEEISGLRKKL |
| | QAEHIHKNKEARIRREMHQISRRIKRLKWKWHMIP |
| | NSELHNFLFKQQDPSFVAVALLHTLGRDIGMINKP |
| | KGSAKREFIPEYGFQIYYKWMNPKLNDINKQKYRK |
| | MPKRSLIPYKNLNVFGDRELIENAMHKLLKLYDEN |
| | LEVKGSKFFKTRVVAISSKESEKLKRDLLWKGELA |
| | KIKKDFNADKNKMQELFKEVKEPKKANALMKQSRN |
| | MGFLLQNISYGALGLLANRMYEASAKQSKGDATKQ |
| | PSIVIPLEMEFGNAFPKLLLRSGKFAMNVSSPWLT |
| | IRKPKFVIKGNKIKNITKLMKDEKAKLKRLETSYH |
| | RATHFRPTLRGSIDWDSPYFSSPKQPNTHRRSPDR |
| | LSADITEYRGRLKSVEAELREGQRAMAKKLDSVDM |
| | TASNLQTSNFQLEKGEDPRLTEIDEKGRSIRNCIS |
| | SWKKFMEDLMKAQEANPVIKIKIALKDESSVLSED |
| | SM |
| SEQ | KFHPENLNKSYCLAIVRAANLDADIQGHINCIGIK |
| ID | SNKSDRNYENKLESLQNVELLCKACTKSTYKPNIN |
| NO: | SVPVGEKKAKYSILSEIKKYDFNSLVYNLKKYRKG |
| 129 | KSRGHQKLNELRELVITSEYKKALDVINKSVNHYL |
| | VNIKNKMSKLKKILQNEHIHVGTLARIRRERNRIS |
| | RKLDHYRKKWKFVPNKILKNYVFKNQSPDFVSVAL |
| | LHKLGRDIGLITKTAILQKSFPEYSLQLYYKYDTP |
| | KLNYLKKSKFKSLPKRILISYKYPKFDINSNYIEE |
| | SIDKLLKLYEESPIYKNNSKIIEFFKKSEDNLIKS |
| | ENDSLKRGIMKEFEKVTKNFSSKKKKLKEELKLKN |
| | EDKNSKMLAKVSRPIGFLKAYLSYMLFNIISNRIF |
| | EFSRKSSGRIPQLPSCIINLGNQFENFKNELQDSN |
| | IGSKKNYKYFCNLLLKSSGFNISYEEEHLSIKTPN |
| | FFINGRKLKEITSEKKKIRKENEQLIKQWKKLTFF |
| | KPSNLNGKKTSDKIRFKSPNNPDIERKSEDNIVEN |
| | IAKVKYKLEDLLSEQRKEFNKLAKKHDGVDVEAQC |
| | LQTKSFWIDSNSPIKKSLEKKNEKVSVKKKMKAIR |
| | SCISAWKWFMADLIEAQKETPMIKLKLALM |
| SEQ | TTLVPSHLAGIEVMDETTSRNEDMIQKETSRSNED |
| ID | ENYLGVKNKCGINVHKSGRGSSKHEPNMPPEKSGE |
| NO: | GQMPKQDSTEMQQRFDESVTGETQVSAGATASIKT |
| 130 | DARANSGPRVGTARALIVKASNLDRDIKLGCKPCE |
| | YIRSELPMGKKNGCNHCEKSSDIASVPKVESGFRK |
| | AKYELVRRFESFAADSISRHLGKEQARTRGKRGKK |
| | DKKEQMGKVNLDEIAILKNESLIEYTENQILDARS |
| | NRIKEWLRSLRLRLRTRNKGLKKSKSIRRQLITLR |
| | RDYRKWIKPNPYRPDEDPNENSLRLHTKLGVDIGV |
| | QGGDNKRMNSDDYETSFSITWRDTATRKICFTKPK |
| | GLLPRHMKFKLRGYPELILYNEELRIQDSQKFPLV |
| | DWERIPIFKLRGVSLGKKKVKALNRITEAPRLVVA |
| | KRIQVNIESKKKKVLTRYVYNDKSINGRLVKAEDS |
| | NKDPLLEFKKQAEEINSDAKYYENQEIAKNYLWGC |
| | EGLHKNLLEEQTKNPYLAFKYGFLNIV |
| SEQ | LDFKRTCSQELVLLPEIEGLKLSGTQGVTSLAKKL |
| ID | INKAANVDRDESYGCHHCIHTRTSLSKPVKKDCNS |
| NO: | CNQSTNHPAVPITLKGYKIAFYELWHRFTSWAVDS |
| 131 | ISKALHRNKVMGKVNLDEYAVVDNSHIVCYAVRKC |
| | YEKRQRSVRLHKRAYRCRAKHYNKSQPKVGRIYKK |
| | SKRRNARNLKKEAKRYFQPNEITNGSSDALFYKIG |
| | VDLGIAKGTPETEVKVDVSICFQVYYGDARRVLRV |
| | RKMDELQSFHLDYTGKLKLKGIGNKDTFTIAKRNE |
| | SLKWGSTKYEVSRAHKKFKPFGKKGSVKRKCNDYF |
| | RSIASWSCEAASQRAQSNLKNAFPYQKALVKCYKN |
| | LDYKGVKKNDMWYRLCSNRIFRYSRIAEDIAQYQS |
| | DKGKAKFEFVILAQSVAEYDISAIM |
| SEQ | VFLTDDKRKTALRKIRSAFRKTAEIALVRAQEADS |
| ID | LDRQAKKLTIETVSFGAPGAKNAFIGSLQGYNWNS |
| NO: | HRANVPSSGSAKDVFRITELGLGIPQSAHEASIGK |
| 132 | SFELVGNVVRYTANLLSKGYKKGAVNKGAKQQREI |
| | KGKEQLSFDLISNGPISGDKLINGQKDALAWWLID |
| | KMGFHIGLAMEPLSSPNTYGITLQAFWKRHTAPRR |
| | YSRGVIRQWQLPFGRQLAPLIHNFFRKKGASIPIV |
| | LTNASKKLAGKGVLLEQTALVDPKKWWQVKEQVTG |
| | PLSNIWERSVPLVLYTATFTHKHGAAHKRPLTLKV |
| | IRISSGSVFLLPLSKVTPGKLVRAWMPDINILRDG |
| | RPDEAAYKGPDLIRARERSFPLAYTCVTQIADEWQ |
| | KRALESNRDSITPLEAKLVTGSDLLQIHSTVQQAV |
| | EQGIGGRISSPIQELLAKDALQLVLQQLFMTVDLL |
| | RIQWQLKQEVADGNTSEKAVGWAIRISNIHKDAYK |
| | TAIEPCTSALKQAWNPLSGFEERTFQLDASIVRKR |
| | STAKTPDDELVIVLRQQAAEMTVAVTQSVSKELME |
| | LAVRHSATLHLLVGEVASKQLSRSADKDRGAMDHW |
| | KLLSQSM |
| SEQ | EDLLQKALNTATNVAAIERHSCISCLFTESEIDVK |
| ID | YKTPDKIGQNTAGCQSCTFRVGYSGNSHTLPMGNR |
| NO: | IALDKLRETIQRYAWHSLLFNVPPAPTSKRVRAIS |
| 133 | ELRVAAGRERLFTVITFVQTNILSKLQKRYAANWT |
| | PKSQERLSRLREEGQHILSLLESGSWQQKEVVRED |
| | QDLIVCSALTKPGLSIGAFCRPKYLKPAKHALVLR |
| | LIFVEQWPGQIWGQSKRTRRMRRRKDVERVYDISV |
| | QAWALKGKETRISECIDTMRRHQQAYIGVLPFLIL |
| | SGSTVRGKGDCPILKEITRMRYCPNNEGLIPLGIF |
| | YRGSANKLLRVVKGSSFTLPMWQNIETLPHPEPFS |
| | PEGWTATGALYEKNLAYWSALNEAVDWYTGQILSS |
| | GLQYPNQNEFLARLQNVIDSIPRKWFRPQGLKNLK |
| | PNGQEDIVPNEFVIPQNAIRAHHVIEWYHKTNDLV |
| | AKTLLGWGSQTTLNQTRPQGDLRFTYTRYYFREKE |
| | VPEV |
| SEQ | VPKKKLMRELAKKAVFEAIFNDPIPGSFGCKRCTL |
| ID | IDGARVTDAIEKKQGAKRCAGCEPCTFHTLYDSVK |
| NO: | HALPAATGCDRTAIDTGLWEILTALRSYNWMSFRR |
| 134 | NAVSDASQKQVWSIEELAIWADKERALRVILSALT |
| | HTIGKLKNGFSRDGVWKGGKQLYENLAQKDLAKGL |
| | FANGEIFGKELVEADHDMLAWTIVPNHQFHIGLIR |
| | GNWKPAAVEASTAFDARWLTNGAPLRDTRTHGHRG |
| | RRFNRTEKLTVLCIKRDGGVSEEFRQERDYELSVM |
| | LLQPKNKLKPEPKGELNSFEDLHDHWWFLKGDEAT |
| | ALVGLTSDPTVGDFIQLGLYIRNPIKAHGETKRRL |
| | LICFEPPIKLPLRRAFPSEAFKTWEPTINVFRNGR |
| | RDTEAYYDIDRARVFEFPETRVSLEHLSKQWEVLR |
| | LEPDRENTDPYEAQQNEGAELQVYSLLQEAAQKMA |
| | PKVVIDPFGQFPLELFSTFVAQLFNAPLSDTKAKI |
| | GKPLDSGFVVESHLHLLEEDFAYRDFVRVTFMGTE |
| | PTFRVIHYSNGEGYWKKTVLKGKNNIRTALIPEGA |
| | KAAVDAYKNKRCPLTLEAAILNEEKDRRLVLGNKA |
| | LSLLAQTARGNLTILEALAAEVLRPLSGTEGVVHL |
| | HACVTRHSTLTESTETDNM |
| SEQ | VEKLFSERLKRAMWLKNEAGRAPPAETLTLKHKRV |
| ID | SGGHEKVKEELQRVLRSLSGTNQAAWNLGLSGGRE |
| NO: | PKSSDALKGEKSRVVLETVVFHSGHNRVLYDVIER |
| 135 | EDQVHQRSSIMHMRRKGSNLLRLWGRSGKVRRKMR |
| | EEVAEIKPVWHKDSRWLAIVEEGRQSVVGISSAGL |
| | AVFAVQESQCTTAEPKPLEYVVSIWFRGSKALNPQ |
| | DRYLEFKKLKTTEALRGQQYDPIPFSLKRGAGCSL |
| | AIRGEGIKFGSRGPIKQFFGSDRSRPSHADYDGKR |
| | RLSLFSKYAGDLADLTEEQWNRTVSAFAEDEVRRA |
| | TLANIQDFLSISHEKYAERLKKRIESIEEPVSASK |
| | LEAYLSAIFETFVQQREALASNFLMRLVESVALLI |
| | SLEEKSPRVEFRVARYLAESKEGFNRKAM |
| SEQ | VVITQSELYKERLLRVMEIKNDRGRKEPRESQGLV |
| ID | LRFTQVTGGQEKVKQKLWLIFEGFSGTNQASWNFG |
| NO: | QPAGGRKPNSGDALKGPKSRVTYETVVFHFGLRLL |
| 136 | SAVIERHNLKQQRQTMAYMKRRAAARKKWARSGKK |
| | CSRMRNEVEKIKPKWHKDPRWFDIVKEGEPSIVGI |
| | SSAGFAIYIVEEPNFPRQDPLEIEYAISIWFRRDR |
| | SQYLTFKKIQKAEKLKELQYNPIPFRLKQEKTSLV |
| | FESGDIKFGSRGSIEHFRDEARGKPPKADMDNNRR |
| | LTMFSVFSGNLTNLTEEQYARPVSGLLAPDEKRMP |
| | TLLKKLQDFFTPIHEKYGERIKQRLANSEASKRPF |
| | KKLEEYLPAIYLEFRARREGLASNWVLVLINSVRT |
| | LVRIKSEDPYIEFKVSQYLLEKEDNKAL |
| SEQ | KQDALFEERLKKAIFIKRQADPLQREELSLLPPNR |
| ID | KIVTGGHESAKDTLKQILRAINGTNQASWNPGTPS |
| NO: | GKRDSKSADALAGPKSRVKLETVVFHVGHRLLKKV |
| 137 | VEYQGHQKQQHGLKAFMRTCAAMRKKWKRSGKVVG |
| | ELREQLANIQPKWHYDSRPLNLCFEGKPSVVGLRS |
| | AGIALYTIQKSVVPVKEPKPIEYAVSIWFRGPKAM |
| | DREDRCLEFKKLKIATELRKLQFEPIVSTLTQGIK |
| | GFSLYIQGNSVKFGSRGPIKYFSNESVRQRPPKAD |
| | PDGNKRLALFSKFSGDLSDLTEEQWNRPILAFEGI |
| | IRRATLGNIQDYLTVGHEQFAISLEQLLSEKESVL |
| | QMSIEQQRLKKNLGKKAENEWVESFGAEQARKKAQ |
| | GIREYISGFFQEYCSQREQWAENWVQQLNKSVRLF |
| | LTIQDSTPFIEFRVARYLPKGEKKKGKAM |
| SEQ | ANHAERHKRLRKEANRAANRNRPLVADCDTGDPLV |
| ID | GICRLLRRGDKMQPNKTGCRSCEQVEPELRDAILV |
| NO: | SGPGRLDNYKYELFQRGRAMAVHRLLKRVPKLNRP |
| 138 | KKAAGNDEKKAENKKSEIQKEKQKQRRMMPAVSMK |
| | QVSVADFKHVIENTVRHLFGDRRDREIAECAALRA |
| | ASKYFLKSRRVRPRKLPKLANPDHGKELKGLRLRE |
| | KRAKLKKEKEKQAELARSNQKGAVLHVATLKKDAP |
| | PMPYEKTQGRNDYTTFVISAAIKVGATRGTKPLLT |
| | PQPREWQCSLYWRDGQRWIRGGLLGLQAGIVLGPK |
| | LNRELLEAVLQRPIECRMSGCGNPLQVRGAAVDFF |
| | MTTNPFYVSGAAYAQKKFKPFGTKRASEDGAAAKA |
| | REKLMTQLAKVLDKVVTQAAHSPLDGIWETRPEAK |
| | LRAMIMALEHEWIFLRPGPCHNAAEEVIKCDCTGG |
| | HAILWALIDEARGALEHKEFYAVTRAHTHDCEKQK |
| | LGGRLAGFLDLLIAQDVPLDDAPAARKIKTLLEAT |
| | PPAPCYKAATSIATCDCEGKFDKLWAIIDATRAGH |
| | GTEDLWARTLAYPQNVNCKCKAGKDLTHRLADFLG |
| | LLIKRDGPFRERPPHKVTGDRKLVFSGDKKCKGHQ |
| | YVILAKAHNEEVVRAWISRWGLKSRTNKAGYAATE |
| | LNLLLNWLSICRRRWMDMLTVQRDTPYIRMKTGRL |
| | VVDDKKERKAM |
| SEQ | AKQREALRVALERGIVRASNRTYTLVTNCTKGGPL |
| ID | PEQCRMIERGKARAMKWEPKLVGCGSCAAATVDLP |
| NO: | AIEEYAQPGRLDVAKYKLTTQILAMATRRMMVRAA |
| 139 | KLSRRKGQWPAKVQEEKEEPPEPKKMLKAVEMRPV |
| | AIVDFNRVIQTTIEHLWAERANADEAELKALKAAA |
| | AYFGPSLKIRARGPPKAAIGRELKKAHRKKAYAER |
| | KKARRKRAELARSQARGAAAHAAIRERDIPPMAYE |
| | RTQGRNDVTTIPIAAAIKIAATRGARPLPAPKPMK |
| | WQCSLYWNEGQRWIRGGMLTAQAYAHAANIHRPMR |
| | CEMWGVGNPLKVRAFEGRVADPDGAKGRKAEFRLQ |
| | TNAFYVSGAAYRNKKFKPFGTDRGGIGSARKKRER |
| | LMAQLAKILDKVVSQAAHSPLDDIWHTRPAQKLRA |
| | MIKQLEHEWMFLRPQAPTVEGTKPDVDVAGNMQRQ |
| | IKALMAPDLPPIEKGSPAKRFTGDKRKKGERAVRV |
| | AEAHSDEVVTAWISRWGIQTRRNEGSYAAQELELL |
| | LNWLQICRRRWLDMTAAQRVSPYIRMKSGRMITDA |
| | ADEGVAPIPLVENM |
| SEQ | KSISGRSIKHMACLKDMLKSEITEIEEKQKKESLR |
| ID | KWDYYSKFSDEILFRRNLNVSANHDANACYGCNPC |
| NO: | AFLKEVYGFRIERRNNERIISYRRGLAGCKSCVQS |
| 140 | TGYPPIEFVRRKFGADKAMEIVREVLHRRNWGALA |
| | RNIGREKEADPILGELNELLLVDARPYFGNKSAAN |
| | ETNLAFNVITRAAKKFRDEGMYDIHKQLDIHSEEG |
| | KVPKGRKSRLIRIERKHKAIHGLDPGETWRYPHCG |
| | KGEKYGVWLNRSRLIHIKGNEYRCLTAFGTTGRRM |
| | SLDVACSVLGHPLVKKKRKKGKKTVDGTELWQIKK |
| | ATETLPEDPIDCTFYLYAAKPTKDPFILKVGSLKA |
| | PRWKKLHKDFFEYSDTEKTQGQEKGKRVVRRGKVP |
| | RILSLRPDAKFKVSIWDDPYNGKNKEGTLLRMELS |
| | GLDGAKKPLILKRYGEPNTKPKNFVFWRPHITPHP |
| | LTFTPKHDFGDPNKKTKRRRVFNREYYGHLNDLAK |
| | MEPNAKFFEDREVSNKKNPKAKNIRIQAKESLPNI |
| | VAKNGRWAAFDPNDSLWKLYLHWRGRRKTIKGGIS |
| | QEFQEFKERLDLYKKHEDESEWKEKEKLWENHEKE |
| | WKKTLEIHGSIAEVSQRCVMQSMMGPLDGLVQKKD |
| | YVHIGQSSLKAADDAWTFSANRYKKATGPKWGKIS |
| | VSNLLYDANQANAELISQSISKYLSKQKDNQGCEG |
| | RKMKFLIKIIEPLRENFVKHTRWLHEMTQKDCEVR |
| | AQFSRVSM |
| SEQ | FPSDVGADALKHVRMLQPRLTDEVRKVALTRAPSD |
| ID | RPALARFAAVAQDGLAFVRHLNVSANHDSNCTFPR |
| NO: | DPRDPRRGPCEPNPCAFLREVWGFRIVARGNERAL |
| 141 | SYRRGLAGCKSCVQSTGFPSVPFHRIGADDCMRKL |
| | HEILKARNWRLLARNIGREREADPLLTELSEYLLV |
| | DARTYPDGAAPNSGRLAENVIKRAAKKFRDEGMRD |
| | IHAQLRVHSREGKVPKGRLQRLRRIERKHRAIHAL |
| | DPGPSWEAEGSARAEVQGVAVYRSQLLRVGHHTQQ |
| | IEPVGIVARTLFGVGRTDLDVAVSVLGAPLTKRKK |
| | GSKTLESTEDFRIAKARETRAEDKIEVAFVLYPTA |
| | SLLRDEIPKDAFPAMRIDRFLLKVGSVQADREILL |
| | QDDYYRFGDAEVKAGKNKGRTVTRPVKVPRLQALR |
| | PDAKFRVNVWADPFGAGDSPGTLLRLEVSGVTRRS |
| | QPLRLLRYGQPSTQPANFLCWRPHRVPDPMTFTPR |
| | QKFGERRKNRRTRRPRVFERLYQVHIKHLAHLEPN |
| | RKWFEEARVSAQKWAKARAIRRKGAEDIPVVAPPA |
| | KRRWAALQPNAELWDLYAHDREARKRFRGGRAAEG |
| | EEFKPRLNLYLAHEPEAEWESKRDRWERYEKKWTA |
| | VLEEHSRMCAVADRTLPQFLSDPLGARMDDKDYAF |
| | VGKSALAVAEAFVEEGTVERAQGNCSITAKKKFAS |
| | NASRKRLSVANLLDVSDKADRALVFQAVRQYVQRQ |
| | AENGGVEGRRMAFLRKLLAPLRQNFVCHTRWLHM |
| SEQ | AARKKKRGKIGITVKAKEKSPPAAGPFMARKLVNV |
| ID | AANVDGVEVHLCVECEADAHGSASARLLGGCRSCT |
| NO: | GSIGAEGRLMGSVDVDRERVIAEPVHTETERLGPD |
| 142 | VKAFEAGTAESKYAIQRGLEYWGVDLISRNRARTV |
| | RKMEEADRPESSTMEKTSWDEIAIKTYSQAYHASE |
| | NHLFWERQRRVRQHALALFRRARERNRGESPLQST |
| | QRPAPLVLAALHAEAAAISGRARAEYVLRGPSANV |
| | RAAAADIDAKPLGHYKTPSPKVARGFPVKRDLLRA |
| | RHRIVGLSRAYFKPSDVVRGTSDAIAHVAGRNIGV |
| | AGGKPKEIEKTFTLPFVAYWEDVDRVVHCSSFKAD |
| | GPWVRDQRIKIRGVSSAVGTFSLYGLDVAWSKPTS |
| | FYIRCSDIRKKFHPKGFGPMKHWRQWAKELDRLTE |
| | QRASCVVRALQDDEELLQTMERGQRYYDVFSCAAT |
| | HATRGEADPSGGCSRCELVSCGVAHKVTKKAKGDT |
| | GIEAVAVAGCSLCESKLVGPSKPRVHRQMAALRQS |
| | HALNYLRRLQREWEALEAVQAPTPYLRFKYARHLE |
| | VRSM |
| SEQ | AAKKKKQRGKIGISVKPKEGSAPPADGPFMARKLV |
| ID | NVAANVDGVEVNLCIECEADAHGSAPARLLGGCKS |
| NO: | CTGSIGAEGRLMGSVDVDRADAIAKPVNTETEKLG |
| 143 | PDVQAFEAGTAETKYALQRGLEYWGVDLISRNRSR |
| | TVRRTEEGQPESATMEKTSWDEIAIKSYTRAYHAS |
| | ENHLFWERQRRVRQHALALFKRAKERNRGDSTLPR |
| | EPGHGLVAIAALACEAYAVGGRNLAETVVRGPTFG |
| | TARAVRDVEIASLGRYKTPSPKVAHGSPVKRDFLR |
| | ARHRIVGLARAYYRPSDVVRGTSDAIAHVAGRNIG |
| | VAGGKPRAVEAVFTLPFVAYWEDVDRVVHCSSFQV |
| | SAPWNRDQRMKIAGVTTAAGTFSLHGGELKWAKPT |
| | SFYIRCSDTRRKFRPKGFGPMKRWRQWAKDLDRLV |
| | EQRASCVVRALQDDAALLETMERGQRYYDVFACAV |
| | THATRGEADRLAGCSRCALTPCQEAHRVTTKPRGD |
| | AGVEQVQTSDCSLCEGKLVGPSKPRLHRTLTLLRQ |
| | EHGLNYLRRLQREWESLEAVQVPTPYLRFKYARHL |
| | EVRSM |
| SEQ | TDSQSESVPEVVYALTGGEVPGRVPPDGGSAEGAR |
| ID | NAPTGLRKQRGKIKISAKPSKPGSPASSLARTLVN |
| NO: | EAANVDGVQSSGCATCRMRANGSAPRALPIGCVAC |
| 144 | ASSIGRAPQEETVCALPTTQGPDVRLLEGGHALRK |
| | YDIQRALEYWGVDLIGRNLDRQAGRGMEPAEGATA |
| | TMKRVSMDELAVLDFGKSYYASEQHLFAARQRRVR |
| | QHAKALKIRAKHANRSGSVKRALDRSRKQVTALAR |
| | EFFKPSDVVRGDSDALAHVVGRNLGVSRHPAREIP |
| | QTFTLPLCAYWEDVDRVISCSSLLAGEPFARDQEI |
| | RIEGVSSALGSLRLYRGAIEWHKPTSLYIRCSDTR |
| | RKFRPRGGLKKRWRQWAKDLDRLVEQRACCIVRSL |
| | QADVELLQTMERAQRFYDVHDCAATHVGPVAVRCS |
| | PCAGKQFDWDRYRLLAALRQEHALNYLRRLQREWE |
| | SLEAQQVKMPYLRFKYARKLEVSGPLIGLEVRREP |
| | SMGTAIAEM |
| SEQ | AGTAGRRHGSLGARRSINIAGVTDRHGRWGCESCV |
| ID | YTRDQAGNRARCAPCDQSTYAPDVQEVTIGQRQAK |
| NO: | YTIFLTLQSFSWTNTMRNNKRAAAGRSKRTTGKRI |
| 145 | GQLAEIKITGVGLAHAHNVIQRSLQHNITKMWRAE |
| | KGKSKRVARLKKAKQLTKRRAYFRRRMSRQSRGNG |
| | FFRTGKGGIHAVAPVKIGLDVGMIASGSSEPADEQ |
| | TVTLDAIWKGRKKKIRLIGAKGELAVAACRFREQQ |
| | TKGDKCIPLILQDGEVRWNQNNWQCHPKKLVPLCG |
| | LEVSRKFVSQADRLAQNKVASPLAARFDKTSVKGT |
| | LVESDFAAVLVNVTSIYQQCHAMLLRSQEPTPSLR |
| | VQRTITSM |
| SEQ | GVRFSPAQSQVFFRTVIPQSVEARFAINMAAIHDA |
| ID | AGAFGCSVCRFEDRTPRNAKAVHGCSPCTRSTNRP |
| NO: | DVFVLPVGAIKAKYDVFMRLLGFNWTHLNRRQAKR |
| 146 | VTVRDRIGQLDELAISMLTGKAKAVLKKSICHNVD |
| | KSFKAMRGSLKKLHRKASKTGKSQLRAKLSDLRER |
| | TNTTQEGSHVEGDSDVALNKIGLDVGLVGKPDYPS |
| | EESVEVVVCLYFVGKVLILDAQGRIRDMRAKQYDG |
| | FKIPIIQRGQLTVLSVKDLGKWSLVRQDYVLAGDL |
| | RFEPKISKDRKYAECVKRIALITLQASLGFKERIP |
| | YYVTKQVEIKNASHIAFVTEAIQNCAENFREMTEY |
| | LMKYQEKSPDLKVLLTQLM |
| SEQ | RAVVGKVFLEQARRALNLATNFGTNHRTGCNGCYV |
| ID | TPGKLSIPQDGEKNAAGCTSCLMKATASYVSYPKP |
| NO: | LGEKVAKYSTLDALKGFPWYSLRLNLRPNYRGKPI |
| 147 | NGVQEVAPVSKFRLAEEVIQAVQRYHFTELEQSFP |
| | GGRRRLRELRAFYTKEYRRAPEQRQHVVNGDRNIV |
| | VVTVLHELGFSVGMFNEVELLPKTPIECAVNVFIR |
| | GNRVLLEVRKPQFDKERLLVESLWKKDSRRHTAKW |
| | TPPNNEGRIFTAEGWKDFQLPLLLGSTSRSLRAIE |
| | KEGFVQLAPGRDPDYNNTIDEQHSGRPFLPLYLYL |
| | QGTISQEYCVFAGTWVIPFQDGISPYSTKDTFQPD |
| | LKRKAYSLLLDAVKHRLGNKVASGLQYGRFPAIEE |
| | LKRLVRMHGATRKIPRGEKDLLKKGDPDTPEWWLL |
| | EQYPEFWRLCDAAAKRVSQNVGLLLSLKKQPLWQR |
| | RWLESRTRNEPLDNLPLSMALTLHLTNEEAL |
| SEQ | AAVYSKFYIENHFKMGIPETLSRIRGPSIIQGFSV |
| ID | NENYINIAGVGDRDFIFGCKKCKYTRGKPSSKKIN |
| NO: | KCHPCKRSTYPEPVIDVRGSISEFKYKIYNKLKQE |
| 148 | PNQSIKQNTKGRMNPSDHTSSNDGIIINGIDNRIA |
| | YNVIFSSYKHLMEKQINLLRDTTKRKARQIKKYNN |
| | SGKKKHSLRSQTKGNLKNRYHMLGMFKKGSLTITN |
| | EGDFITAVRKVGLDISLYKNESLNKQEVETELCLN |
| | IKWGRTKSYTVSGYIPLPINIDWKLYLFEKETGLT |
| | LRLFGNKYKIQSKKFLIAQLFKPKRPPCADPVVKK |
| | AQKWSALNAHVQQMAGLFSDSHLLKRELKNRMHKQ |
| | LDFKSLWVGTEDYIKWFEELSRSYVEGAEKSLEFF |
| | RQDYFCFNYTKQTTM |
| SEQ | PQQQRDLMLMAANYDQDYGNGCGPCTVVASAAYRP |
| ID | DPQAQHGCKRHLRTLGASAVTHVGLGDRTATITAL |
| NO: | HRLRGPAALAARARAAQAASAPMTPDTDAPDDRRR |
| 149 | LEAIDADDVVLVGAHRALWSAVRRWADDRRAALRR |
| | RLHSEREWLLKDQIRWAELYTLIEASGTPPQGRWR |
| | NTLGALRGQSRWRRVLAPTMRATCAETHAELWDAL |
| | AELVPEMAKDRRGLLRPPVEADALWRAPMIVEGWR |
| | GGHSVVVDAVAPPLDLPQPCAWTAVRLSGDPRQRW |
| | GLHLAVPPLGQVQPPDPLKATLAVSMRHRGGVRVR |
| | TLQAMAVDADAPMQRHLQVPLTLQRGGGLQWGIHS |
| | RGVRRREARSMASWEGPPIWTGLQLVNRWKGQGSA |
| | LLAPDRPPDTPPYAPDAAVAPAQPDTKRARRTLKE |
| | ACTVCRCAPGHMRQLQVTLTGDGTWRRFRLRAPQG |
| | AKRKAEVLKVATQHDERIANYTAWYLKRPEHAAGC |
| | DTCDGDSRLDGACRGCRPLLVGDQCFRRYLDKIEA |
| | DRDDGLAQIKPKAQEAVAAMAAKRDARAQKVAARA |
| | AKLSEATGQRTAATRDASHEARAQKELEAVATEGT |
| | TVRHDAAAVSAFGSWVARKGDEYRHQVGVLANRLE |
| | HGLRLQELMAPDSVVADQQRASGHARVGYRYVLTA |
| | M |
| SEQ | AVAHPVGRGNAGSPGARGPEELPRQLVNRASNVTR |
| ID | PATYGCAPCRHVRLSIPKPVLTGCRACEQTTHPAP |
| NO: | KRAVRGGADAAKYDLAAFFAGWAADLEGRNRRRQV |
| 150 | HAPLDPQPDPNHEPAVTLQKIDLAEVSIEEFQRVL |
| | ARSVKHRHDGRASREREKARAYAQVAKKRRNSHAH |
| | GARTRRAVRRQTRAVRRAHRMGANSGEILVASGAE |
| | DPVPEAIDHAAQLRRRIRACARDLEGLRHLSRRYL |
| | KTLEKPCRRPRAPDLGRARCHALVESLQAAERELE |
| | ELRRCDSPDTAMRRLDAVLAAAASTDATFATGWTV |
| | VGMDLGVAPRGSAAPEVSPMEMAISVFWRKGSRRV |
| | IVSKPIAGMPIRRHELIRLEGLGTLRLDGNHYTGA |
| | GVTKGRGLSEGTEPDFREKSPSTLGFTLSDYRHES |
| | RWRPYGAKQGKTARQFFAAMSRELRALVEHQVLAP |
| | MGPPLLEAHERRFETLLKGQDNKSIHAGGGGRYVW |
| | RGPPDSKKRPAADGDWFRFGRGHADHRGWANKRHE |
| | LAANYLQSAFRLWSTLAEAQEPTPYARYKYTRVTM |
| SEQ | WDFLTLQVYERHTSPEVCVAGNSTKCASGTRKSDH |
| ID | THGVGVKLGAQEINVSANDDRDHEVGCNICVISRV |
| NO: | SLDIKGWRYGCESCVQSTPEWRSIVRFDRNHKEAK |
| 151 | GECLSRFEYWGAQSIARSLKRNKLMGGVNLDELAI |
| | VQNENVVKTSLKHLFDKRKDRIQANLKAVKVRMRE |
| | RRKSGRQRKALRRQCRKLKRYLRSYDPSDIKEGNS |
| | CSAFTKLGLDIGISPNKPPKIEPKVEVVFSLFYQG |
| | ACDKIVTVSSPESPLPRSWKIKIDGIRALYVKSTK |
| | VKFGGRTFRAGQRNNRRKVRPPNVKKGKRKGSRSQ |
| | FFNKFAVGLDAVSQQLPIASVQGLWGRAETKKAQT |
| | ICLKQLESNKPLKESQRCLFLADNWVVRVCGFLRA |
| | LSQRQGPTPYIRYRYRCNM |
| SEQ | ARNVGQRNASRQSKRESAKARSRRVTGGHASVTQG |
| ID | VALINAAANADRDHTTGCEPCTWERVNLPLQEVIH |
| NO: | GCDSCTKSSPFWRDIKVVNKGYREAKEEIMRIASG |
| 152 | ISADHLSRALSHNKVMGRLNLDEVCILDFRTVLDT |
| | SLKHLTDSRSNGIKEHIRAVHRKIRMRRKSGKTAR |
| | ALRKQYFALRRQWKAGHKPNSIREGNSLTALRAVG |
| | FDVGVSEGTEPMPAPQTEVVLSVFYKGSATRILRI |
| | SSPHPIAKRSWKVKIAGIKALKLIRREHDFSFGRE |
| | TYNASQRAEKRKFSPHAARKDFFNSFAVQLDRLAQ |
| | QLCVSSVENLWVTEPQQKLLTLAKDTAPYGIREGA |
| | RFADTRARLAWNWVFRVCGFTRALHQEQEPTPYCR |
| | FTWRSKM |

In some embodiments, the Type VI CRISPR-Cas enzyme is a programmable Cas13 nuclease. The general architecture of a Cas13 enzyme includes an N-terminal domain and two HEPN (higher eukaryotes and prokaryotes nucleotide-binding) domains separated by two helical domains (Liu et al., Cell 2017 Jan. 12; 168(1-2):121-134.e12). The HEPN domains each comprise aR-X₄-H motif Shared features across Cas13 enzymes include that upon binding of crRNA to a target nucleic acid, the enzyme undergoes a conformational change to bring together the HEPN domains and form a catalytically active RNase. (Tambe et al., Cell Rep. 2018 Jul. 24; 24(4): 1025-1036.). Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 enzymes cleavage efficiency or mutations that catalytically inactivate the HEPN domains. Programmable Cas13 nucleases consistent with the present disclosure also Cas13 nucleases comprising catalytic

A programmable Cas13 nuclease can be a Cas13a enzyme (also referred to as “c2c2”), a Cas13b enzyme, a Cas13c enzyme, a Cas13d enzyme, or a Cas13e enzyme. Example C2c2 enzymes are set forth as SEQ ID NO: 153-SEQ ID NO: 160. In some cases, a subject C2c2 enzymes includes an amino acid sequence having 80% or more (e.g., 85% or more, 90% or more, 95% or more, 98% or more, 99% or more, 99.5% or more, or 100%) amino acid sequence identity with the amino acid sequence set forth in any one of SEQ ID NO: 153-SEQ ID NO: 160. In some cases, a suitable C2c2 polypeptide comprises an amino acid sequence having at least 80%, at least 85%, at least 90%, at least 95%, at least 98%, at least 99%, or 100%, amino acid sequence identity to the Listeria seeligeri C2c2 amino acid sequence set forth in SEQ ID NO: 153. In some cases, a suitable C2c2 polypeptide comprises an amino acid sequence having at least 80%, at least 85%, at least 90%, at least 95%, at least 98%, at least 99%, or 100%, amino acid sequence identity to the Leptotrichia buccalis C2c2 amino acid sequence set forth in SEQ ID NO: 154. In some cases, a suitable C2c2 polypeptide comprises an amino acid sequence having at least 80%, at least 85%, at least 90%, at least 95%, at least 98%, at least 99%, or 100%, amino acid sequence identity to the Rhodobacter capsulatus C2c2 amino acid sequence set forth in SEQ ID NO: 156. In some cases, a suitable C2c2 polypeptide comprises an amino acid sequence having at least 80%, at least 85%, at least 90%, at least 95%, at least 98%, at least 99%, or 100%, amino acid sequence identity to the Carnobacterium gallinarum C2c2 amino acid sequence set forth in SEQ ID NO: 157. In some cases, a suitable C2c2 polypeptide comprises an amino acid sequence having at least 80%, at least 85%, at least 90%, at least 95%, at least 98%, at least 99%, or 100%, amino acid sequence identity to the Herbinix hemicellulosilytica C2c2 amino acid sequence set forth in SEQ ID NO: 158. In some cases, the C2c2 enzyme includes an amino acid sequence having 80% or more amino acid sequence identity with the Leptotrichia buccalis (Lbu) C2c2 amino acid sequence set forth in SEQ ID NO: 154. In some cases, the C2c2 enzyme is a Leptotrichia buccalis (Lbu) C2c2 enzyme (e.g., see SEQ ID NO: 154). In some cases, the C2c2 enzyme includes the amino acid sequence set forth in any one of SEQ ID NO: 153, SEQ ID NO: 154 and SEQ ID NO: 156-SEQ ID NO: 160. In some cases, a C2c2 enzyme used in a method of the present disclosure is not a Leptotrichia shahii (Lsh) C2c2 enzyme. In some cases, a C2c2 enzyme used in a method of the present disclosure is not a C2c2 polypeptide having at least 80%, at least 85%, at least 90%, at least 95%, at least 98%, at least 99%, or 100%, amino acid sequence identity to the Lsh C2c2 polypeptide set forth in SEQ ID NO: 155. Other Cas13 enzyme sequences are set forth in SEQ ID NO: 153-SEQ ID NO: 170.

TABLE 3
Cas13 Enzyme Sequences

| SEQ | | |
|---|---|---|
| ID | | |
| NO | Description | Sequence |
| SEQ | Listeria | MWISIKTLIHHLGVLFFCDYMYNRR |
| ID | seeligeri | EKKIIEVKTMRITKVEVDRKKVLIS |
| NO: | C2c2 amino | RDKNGGKLVYENEMQDNTEQIMHHK |
| 153 | acid | KSSFYKSVVNKTICRPEQKQMKKLV |
| | sequence | HGLLQENSQEKIKVSDVTKLNISNF |
| | | LNHRFKKSLYYFPENSPDKSEEYRI |
| | | EINLSQLLEDSLKKQQGTFICWESF |
| | | SKDMELYINWAENYISSKTKLIKKS |
| | | IRNNRIQSTESRSGQLMDRYMKDIL |
| | | NKNKPFDIQSVSEKYQLEKLTSALK |
| | | ATFKEAKKNDKEINYKLKSTLQNHE |
| | | RQIIEELKENSELNQFNIEIRKHLE |
| | | TYFPIKKTNRKVGDIRNLEIGEIQK |
| | | IVNHRLKNKIVQRILQEGKLASYEI |
| | | ESTVNSNSLQKIKIEEAFALKFINA |
| | | CLFASNNLRNMVYPVCKKDILMIGE |
| | | FKNSFKEIKHKKFIRQWSQFFSQEI |
| | | TVDDIELASWGLRGAIAPIRNEIIH |
| | | LKKHSWKKFFNNPTFKVKKSKIING |
| | | KTKDVTSEFLYKETLFKDYFYSELD |
| | | SVPELIINKMESSKILDYYSSDQLN |
| | | QVFTIPNFELSLLTSAVPFAPSFKR |
| | | VYLKGFDYQNQDEAQPDYNLKLNIY |
| | | NEKAFNSEAFQAQYSLFKMVYYQVF |
| | | LPQFTTNNDLFKSSVDFILTLNKER |
| | | KGYAKAFQDIRKMNKDEKPSEYMSY |
| | | IQSQLMLYQKKQEEKEKINHFEKFI |
| | | NQVFIKGFNSFIEKNRLTYICHPTK |
| | | NTVPENDNIEIPFHTDMDDSNIAFW |
| | | LMCKLLDAKQLSELRNEMIKFSCSL |
| | | QSTEEISTFTKAREVIGLALLNGEK |
| | | GCNDWKELFDDKEAWKKNMSLYVSE |
| | | ELLQSLPYTQEDGQTPVINRSIDLV |
| | | KKYGTETILEKLFSSSDDYKVSAKD |
| | | IAKLHEYDVTEKIAQQESLHKQWIE |
| | | KPGLARDSAWTKKYQNVINDISNYQ |
| | | WAKTKVELTQVRHLHQLTIDLLSRL |
| | | AGYMSIADRDFQFSSNYILERENSE |
| | | YRVTSWILLSENKNKNKYNDYELYN |
| | | LKNASIKVSSKNDPQLKVDLKQLRL |
| | | TLEYLELFDNRLKEKRNNISHFNYL |
| | | NGQLGNSILELFDDARDVLSYDRKL |
| | | KNAVSKSLKEILSSHGMEVTFKPLY |
| | | QTNHHLKIDKLQPKKIHHLGEKSTV |
| | | SSNQVSNEYCQLVRTLLTMK |
| SEQ | Leptotrichia | MKVTKVGGISHKKYTSEGRLVKSES |
| ID | buccalis | EENRTDERLSALLNMRLDMYIKNPS |
| NO: | (Lbu) C2c2 | STETKENQKRIGKLKKFFSNKMVYL |
| 154 | amino acid | KDNTLSLKNGKKENIDREYSETDIL |
| | sequence | ESDVRDKKNFAVLKKIYLNENVNSE |
| | | ELEVFRNDIKKKLNKINSLKYSFEK |
| | | NKANYQKINENNIEKVEGKSKRNII |
| | | YDYYRESAKRDAYVSNVKEAFDKLY |
| | | KEEDIAKLVLEIENLTKLEKYKIRE |
| | | FYHEIIGRKNDKENFAKIIYEEIQN |
| | | VNNMKELIEKVPDMSELKKSQVFYK |
| | | YYLDKEELNDKNIKYAFCHFVEIEM |
| | | SQLLKNYVYKRLSNISNDKIKRIFE |
| | | YQNLKKLIENKLLNKLDTYVRNCGK |
| | | YNYYLQDGEIATSDFIARNRQNEAF |
| | | LRNIIGVSSVAYFSLRNILETENEN |
| | | DITGRMRGKTVKNNKGEEKYVSGEV |
| | | DKIYNENKKNEVKENLKMFYSYDFN |
| | | MDNKNEIEDFFANIDEAISSIRHGI |
| | | VHFNLELEGKDIFAFKNIAPSEISK |
| | | KMFQNEINEKKLKLKIFRQLNSANV |
| | | FRYLEKYKILNYLKRTRFEFVNKNI |
| | | PFVPSFTKLYSRIDDLKNSLGIYWK |
| | | TPKTNDDNKTKEIIDAQIYLLKNIY |
| | | YGEFLNYFMSNNGNFFEISKEIIEL |
| | | NKNDKRNLKTGFYKLQKFEDIQEKI |
| | | PKEYLANIQSLYMINAGNQDEEEKD |
| | | TYIDFIQKIFLKGFMTYLANNGRLS |
| | | LIYIGSDEETNTSLAEKKQEFDKFL |
| | | KKYEQNNNIKIPYEINEFLREIKLG |
| | | NILKYTERLNMFYLILKLLNHKELT |
| | | NLKGSLEKYQSANKEEAFSDQLELI |
| | | NLLNLDNNRVTEDFELEADEIGKFL |
| | | DFNGNKVKDNKELKKFDTNKIYFDG |
| | | ENIIKHRAFYNIKKYGMLNLLEKIA |
| | | DKAGYKISIEELKKYSNKKNEIEKN |
| | | HKMQENLHRKYARPRKDEKFTDEDY |
| | | ESYKQAIENIEEYTHLKNKVEFNEL |
| | | NLLQGLLLRILHRLVGYTSIWERDL |
| | | RFRLKGEFPENQYIEEIFNFENKKN |
| | | VKYKGGQIVEKYIKFYKELHQNDEV |
| | | KINKYSSANIKVLKQEKKDLYIRNY |
| | | IAHFNYIPHAEISLLEVLENLRKLL |
| | | SYDRKLKNAVMKSVVDILKEYGFVA |
| | | TFKIGADKKIGIQTLESEKIVHLKN |
| | | LKKKKLMTDRNSEELCKLVKIMFEY |
| | | KMEEKKSEN |
| SEQ | Leptotrichia | MGNLFGHKRWYEVRDKKDFKIKRKV |
| ID | shahii (Lsh) | KVKRNYDGNKYILNINENNNKEKID |
| NO: | C2c2 amino | NNKFIRKYINYKKNDNILKEFTRKF |
| 155 | acid | HAGNILFKLKGKEGIIRIENNDDFL |
| | sequence | ETEEVVLYIEAYGKSEKLKALGITK |
| | | KKIIDEAIRQGITKDDKKIEIKRQE |
| | | NEEEIEIDIRDEYTNKTLNDCSIIL |
| | | RIIENDELETKKSIYEIFKNINMSL |
| | | YKIIEKIIENETEKVFENRYYEEHL |
| | | REKLLKDDKIDVILTNFMEIREKIK |
| | | SNLEILGFVKFYLNVGGDKKKSKNK |
| | | KMLVEKILNINVDLTVEDIADFVIK |
| | | ELEFWNITKRIEKVKKVNNEFLEKR |
| | | RNRTYIKSYVLLDKHEKFKIERENK |
| | | KDKIVKFFVENIKNNSIKEKIEKIL |
| | | AEFKIDELIKKLEKELKKGNCDTEI |
| | | FGIFKKHYKVNFDSKKFSKKSDEEK |
| | | ELYKIIYRYLKGRIEKILVNEQKVR |
| | | LKKMEKIEIEKILNESILSEKILKR |
| | | VKQYTLEHIMYLGKLRHNDIDMTTV |
| | | NTDDFSRLHAKEELDLELITFFAST |
| | | NMELNKIFSRENINNDENIDFFGGD |
| | | REKNYVLDKKILNSKIKIIRDLDFI |
| | | DNKNNITNNFIRKFTKIGTNERNRI |
| | | LHAISKERDLQGTQDDYNKVINIIQ |
| | | NLKISDEEVSKALNLDVVFKDKKNI |
| | | ITKINDIKISEENNNDIKYLPSFSK |
| | | VLPEILNLYRNNPKNEPFDTIETEK |
| | | IVLNALIYVNKELYKKLILEDDLEE |
| | | NESKNIFLQELKKTLGNIDEIDENI |
| | | IENYYKNAQISASKGNNKAIKKYQK |
| | | KVIECYIGYLRKNYEELFDFSDFKM |
| | | NIQEIKKQIKDINDNKTYERITVKT |
| | | SDKTIVINDDFEYIISIFALLNSNA |
| | | VINKIRNRFFATSVWLNTSEYQNII |
| | | DILDEIMQLNTLRNECITENWNLNL |
| | | EEFIQKMKEIEKDFDDFKIQTKKEI |
| | | FNNYYEDIKNNILTEFKDDINGCDV |
| | | LEKKLEKIVIFDDETKFEIDKKSNI |
| | | LQDEQRKLSNINKKDLKKKVDQYIK |
| | | DKDQEIKSKILCRIIFNSDFLKKYK |
| | | KEIDNLIEDMESENENKFQEIYYPK |
| | | ERKNELYIYKKNLFLNIGNPNFDKI |
| | | YGLISNDIKMADAKFLFNIDGKNIR |
| | | KNKISEIDAILKNLNDKLNGYSKEY |
| | | KEKYIKKLKENDDFFAKNIQNKNYK |
| | | SFEKDYNRVSEYKKIRDLVEFNYLN |
| | | KIESYLIDINWKLAIQMARFERDMH |
| | | YIVNGLRELGIIKLSGYNTGISRAY |
| | | PKRNGSDGFYTTTAYYKFFDEESYK |
| | | KFEKICYGFGIDLSENSEINKPENE |
| | | SIRNYISHFYIVRNPFADYSIAEQI |
| | | DRVSNLLSYSTRYNNSTYASVFEVF |
| | | KKDVNLDYDELKKKFKLIGNNDILE |
| | | RLMKPKKVSVLELESYNSDYIKNLI |
| | | IELLTKIENTNDTL |
| SEQ | Rhodobacter | MQIGKVQGRTISEFGDPAGGLKRKI |
| ID | capsulatus | STDGKNRKELPAHLSSDPKALIGQW |
| NO: | C2c2 amino | ISGIDKIYRKPDSRKSDGKAIHSPT |
| 156 | acid | PSKMQFDARDDLGEAFWKLVSEAGL |
| | sequence | AQDSDYDQFKRRLHPYGDKFQPADS |
| | | GAKLKFEADPPEPQAFHGRWYGAMS |
| | | KRGNDAKELAAALYEHLHVDEKRID |
| | | GQPKRNPKTDKFAPGLVVARALGIE |
| | | SSVLPRGMARLARNWGEEEIQTYFV |
| | | VDVAASVKEVAKAAVSAAQAFDPPR |
| | | QVSGRSLSPKVGFALAEHLERVTGS |
| | | KRCSFDPAAGPSVLALHDEVKKTYK |
| | | RLCARGKNAARAFPADKTELLALMR |
| | | HTHENRVRNQMVRMGRVSEYRGQQA |
| | | GDLAQSHYWTSAGQTEIKESEIFVR |
| | | LWVGAFALAGRSMKAWIDPMGKIVN |
| | | TEKNDRDLTAAVNIRQVISNKEMVA |
| | | EAMARRGIYFGETPELDRLGAEGNE |
| | | GFVFALLRYLRGCRNQTFHLGARAG |
| | | FLKEIRKELEKTRWGKAKEAEHVVL |
| | | TDKTVAAIRAIIDNDAKALGARLLA |
| | | DLSGAFVAHYASKEHFSTLYSEIVK |
| | | AVKDAPEVSSGLPRLKLLLKRADGV |
| | | RGYVHGLRDTRKHAFATKLPPPPAP |
| | | RELDDPATKARYIALLRLYDGPFRA |
| | | YASGITGTALAGPAARAKEAATALA |
| | | QSVNVTKAYSDVMEGRSSRLRPPND |
| | | GETLREYLSALTGETATEFRVQIGY |
| | | ESDSENARKQAEFIENYRRDMLAFM |
| | | FEDYIRAKGFDWILKIEPGATAMTR |
| | | APVLPEPIDTRGQYEHWQAALYLVM |
| | | HFVPASDVSNLLHQLRKWEALQGKY |
| | | ELVQDGDATDQADARREALDLVKRF |
| | | RDVLVLFLKTGEARFEGRAAPFDLK |
| | | PFRALFANPATFDRLFMATPTTARP |
| | | AEDDPEGDGASEPELRVARTLRGLR |
| | | QIARYNHMAVLSDLFAKHKVRDEEV |
| | | ARLAEIEDETQEKSQIVAAQELRTD |
| | | LHDKVMKCHPKTISPEERQSYAAAI |
| | | KTIEEHRFLVGRVYLGDHLRLHRLM |
| | | MDVIGRLIDYAGAYERDTGTFLINA |
| | | SKQLGAGADWAVTIAGAANTDARTQ |
| | | TRKDLAHFNVLDRADGTPDLTALVN |
| | | RAREMMAYDRKRKNAVPRSILDMLA |
| | | RLGLTLKWQMKDHLLQDATITQAAI |
| | | KHLDKVRLTVGGPAAVTEARFSQDY |
| | | LQMVAAVFNGSVQNPKPRRRDDGDA |
| | | WHKPPKPATAQSQPDQKPPNKAPSA |
| | | GSRLPPPQVGEVYEGVVVKVIDTGS |
| | | LGFLAVEGVAGNIGLHISRLRRIRE |
| | | DAIIVGRRYRFRVEIYVPPKSNTSK |
| | | LNAADLVRID |
| SEQ | Carnobacterium | MRITKVKIKLDNKLYQVTMQKEEKY |
| ID | gallinarum | GTLKLNEESRKSTAEILRLKKASFN |
| NO: | C2c2 amino | KSFHSKTINSQKENKNATIKKNGDY |
| 157 | acid | ISQIFEKLVGVDTNKNIRKPKMSLT |
| | sequence | DLKDLPKKDLALFIKRKFKNDDIVE |
| | | IKNLDLISLFYNALQKVPGEHFTDE |
| | | SWADFCQEMMPYREYKNKFIERKII |
| | | LLANSIEQNKGFSINPETFSKRKRV |
| | | LHQWAIEVQERGDFSILDEKLSKLA |
| | | EIYNFKKMCKRVQDELNDLEKSMKK |
| | | GKNPEKEKEAYKKQKNFKIKTIWKD |
| | | YPYKTHIGLIEKIKENEELNQFNIE |
| | | IGKYFEHYFPIKKERCTEDEPYYLN |
| | | SETIATTVNYQLKNALISYLMQIGK |
| | | YKQFGLENQVLDSKKLQEIGIYEGF |
| | | QTKFMDACVFATSSLKNIIEPMRSG |
| | | DILGKREFKEAIATSSFVNYHHFFP |
| | | YFPFELKGMKDRESELIPFGEQTEA |
| | | KQMQNIWALRGSVQQIRNEIFHSFD |
| | | KNQKFNLPQLDKSNFEFDASENSTG |
| | | KSQSYIETDYKFLFEAEKNQLEQFF |
| | | IERIKSSGALEYYPLKSLEKLFAKK |
| | | EMKFSLGSQVVAFAPSYKKLVKKGH |
| | | SYQTATEGTANYLGLSYYNRYELKE |
| | | ESFQAQYYLLKLIYQYVFLPNFSQG |
| | | NSPAFRETVKAILRINKDEARKKMK |
| | | KNKKFLRKYAFEQVREMEFKETPDQ |
| | | YMSYLQSEMREEKVRKAEKNDKGFE |
| | | KNITMNFEKLLMQIFVKGFDVFLTT |
| | | FAGKELLLSSEEKVIKETEISLSKK |
| | | INEREKTLKASIQVEHQLVATNSAI |
| | | SYWLFCKLLDSRHLNELRNEMIKFK |
| | | QSRIKFNHTQHAELIQNLLPIVELT |
| | | ILSNDYDEKNDSQNVDVSAYFEDKS |
| | | LYETAPYVQTDDRTRVSFRPILKLE |
| | | KYHTKSLIEALLKDNPQFRVAATDI |
| | | QEWMHKREEIGELVEKRKNLHTEWA |
| | | EGQQTLGAEKREEYRDYCKKIDRFN |
| | | WKANKVTLTYLSQLHYLITDLLGRM |
| | | VGFSALFERDLVYFSRSFSELGGET |
| | | YHISDYKNLSGVLRLNAEVKPIKIK |
| | | NIKVIDNEENPYKGNEPEVKPFLDR |
| | | LHAYLENVIGIKAVHGKIRNQTAHL |
| | | SVLQLELSMIESMNNLRDLMAYDRK |
| | | LKNAVTKSMIKILDKHGMILKLKID |
| | | ENHKNFEIESLIPKEIIHLKDKAIK |
| | | TNQVSEEYCQLVLALLTTNPGNQLN |
| SEQ | Herbinix | MKLTRRRISGNSVDQKITAAFYRDM |
| ID | hemicellulo | SQGLLYYDSEDNDCTDKVIESMDFE |
| NO: | silytica | RSWRGRILKNGEDDKNPFYMFVKGL |
| 158 | C2c2 amino | VGSNDKIVCEPIDVDSDPDNLDILI |
| | acid | NKNLTGFGRNLKAPDSNDTLENLIR |
| | sequence | KIQAGIPEEEVLPELKKIKEMIQKD |
| | | IVNRKEQLLKSIKNNRIPFSLEGSK |
| | | LVPSTKKMKWLFKLIDVPNKTFNEK |
| | | MLEKYWEIYDYDKLKANITNRLDKT |
| | | DKKARSISRAVSEELREYHKNLRTN |
| | | YNRFVSGDRPAAGLDNGGSAKYNPD |
| | | KEEFLLFLKEVEQYFKKYFPVKSKH |
| | | SNKSKDKSLVDKYKNYCSYKVVKKE |
| | | VNRSIINQLVAGLIQQGKLLYYFYY |
| | | NDTWQEDFLNSYGLSYIQVEEAFKK |
| | | SVMTSLSWGINRLTSFFIDDSNTVK |
| | | FDDITTKKAKEAIESNYFNKLRTCS |
| | | RMQDHFKEKLAFFYPVYVKDKKDRP |
| | | DDDIENLIVLVKNAIESVSYLRNRT |
| | | FHFKESSLLELLKELDDKNSGQNKI |
| | | DYSVAAEFIKRDIENLYDVFREQIR |
| | | SLGIAEYYKADMISDCFKTCGLEFA |
| | | LYSPKNSLMPAFKNVYKRGANLNKA |
| | | YIRDKGPKETGDQGQNSYKALEEYR |
| | | ELTWYIEVKNNDQSYNAYKNLLQLI |
| | | YYHAFLPEVRENEALITDFINRTKE |
| | | WNRKETEERLNTKNNKKHKNFDEND |
| | | DITVNTYRYESIPDYQGESLDDYLK |
| | | VLQRKQMARAKEVNEKEEGNNNYIQ |
| | | FIRDVVVWAFGAYLENKLKNYKNEL |
| | | QPPLSKENIGLNDTLKELFPEEKVK |
| | | SPFNIKCRFSISTFIDNKGKSTDNT |
| | | SAEAVKTDGKEDEKDKKNIKRKDLL |
| | | CFYLFLRLLDENEICKLQHQFIKYR |
| | | CSLKERRFPGNRTKLEKETELLAEL |
| | | EELMELVRFTMPSIPEISAKAESGY |
| | | DTMIKKYFKDFIEKKVFKNPKTSNL |
| | | YYHSDSKTPVTRKYMALLMRSAPLH |
| | | LYKDIFKGYYLITKKECLEYIKLSN |
| | | IIKDYQNSLNELHEQLERIKLKSEK |
| | | QNGKDSLYLDKKDFYKVKEYVENLE |
| | | QVARYKHLQHKINFESLYRIFRIHV |
| | | DIAARMVGYTQDWERDMHFLFKALV |
| | | YNGVLEERRFEAIFNNNDDNNDGRI |
| | | VKKIQNNLNNKNRELVSMLCWNKKL |
| | | NKNEFGAIIWKRNPIAHLNHFTQTE |
| | | QNSKSSLESLINSLRILLAYDRKRQ |
| | | NAVTKTINDLLLNDYHIRIKWEGRV |
| | | DEGQIYFNIKEKEDIENEPIIHLKH |
| | | LHKKDCYIYKNSYMFDKQKEWICNG |
| | | IKEEVYDKSILKCIGNLFKFDYEDK |
| | | NKSSANPKHT |
| SEQ | Paludibacter | MRVSKVKVKDGGKDKMVLVHRKTTG |
| ID | propionicigenes | AQLVYSGQPVSNETSNILPEKKRQS |
| NO: | C2c2 | FDLSTLNKTIIKFDTAKKQKLNVDQ |
| 159 | amino acid | YKIVEKIFKYPKQELPKQIKAEEIL |
| | sequence | PFLNHKFQEPVKYWKNGKEESFNLT |
| | | LLIVEAVQAQDKRKLQPYYDWKTWY |
| | | IQTKSDLLKKSIENNRIDLTENLSK |
| | | RKKALLAWETEFTASGSIDLTHYHK |
| | | VYMTDVLCKMLQDVKPLTDDKGKIN |
| | | TNAYHRGLKKALQNHQPAIFGTREV |
| | | PNEANRADNQLSIYHLEVVKYLEHY |
| | | FPIKTSKRRNTADDIAHYLKAQTLK |
| | | TTIEKQLVNAIRANIIQQGKTNHHE |
| | | LKADTTSNDLIRIKTNEAFVLNLTG |
| | | TCAFAANNIRNMVDNEQTNDILGKG |
| | | DFIKSLLKDNTNSQLYSFFFGEGLS |
| | | TNKAEKETQLWGIRGAVQQIRNNVN |
| | | HYKKDALKTVFNISNFENPTITDPK |
| | | QQTNYADTIYKARFINELEKIPEAF |
| | | AQQLKTGGAVSYYTIENLKSLLTTF |
| | | QFSLCRSTIPFAPGFKKVFNGGINY |
| | | QNAKQDESFYELMLEQYLRKENFAE |
| | | ESYNARYFMLKLIYNNLFLPGFTTD |
| | | RKAFADSVGFVQMQNKKQAEKVNPR |
| | | KKEAYAFEAVRPMTAADSIADYMAY |
| | | VQSELMQEQNKKEEKVAEETRINFE |
| | | KFVLQVFIKGFDSFLRAKEFDFVQM |
| | | PQPQLTATASNQQKADKLNQLEASI |
| | | TADCKLTPQYAKADDATHIAFYVFC |
| | | KLLDAAHLSNLRNELIKFRESVNEF |
| | | KFHHLLEIIEICLLSADVVPTDYRD |
| | | LYSSEADCLARLRPFIEQGADITNW |
| | | SDLFVQSDKHSPVIHANIELSVKYG |
| | | TTKLLEQIINKDTQFKTTEANFTAW |
| | | NTAQKSIEQLIKQREDHHEQWVKAK |
| | | NADDKEKQERKREKSNFAQKFIEKH |
| | | GDDYLDICDYINTYNWLDNKMHFVH |
| | | LNRLHGLTIELLGRMAGFVALFDRD |
| | | FQFFDEQQIADEFKLHGFVNLHSID |
| | | KKLNEVPTKKIKEIYDIRNKIIQIN |
| | | GNKINESVRANLIQFISSKRNYYNN |
| | | AFLHVSNDEIKEKQMYDIRNHIAHF |
| | | NYLTKDAADFSLIDLINELRELLHY |
| | | DRKLKNAVSKAFIDLFDKHGMILKL |
| | | KLNADHKLKVESLEPKKIYHLGSSA |
| | | KDKPEYQYCTNQVMMAYCNMCRSLL |
| | | EMKK |
| SEQ | Leptotrichia | MYMKITKIDGVSHYKKQDKGILKKK |
| ID | wadei (Lwa) | WKDLDERKQREKIEARYNKQIESKI |
| NO: | C2c2 amino | YKEFFRLKNKKRIEKEEDQNIKSLY |
| 160 | acid | FFIKELYLNEKNEEWELKNINLEIL |
| | sequence | DDKERVIKGYKFKEDVYFFKEGYKE |
| | | YYLRILFNNLIEKVQNENREKVRKN |
| | | KEFLDLKEIFKKYKNRKIDLLLKSI |
| | | NNNKINLEYKKENVNEEIYGINPTN |
| | | DREMTFYELLKEIIEKKDEQKSILE |
| | | EKLDNFDITNFLENIEKIFNEETEI |
| | | NIIKGKVLNELREYIKEKEENNSDN |
| | | KLKQIYNLELKKYIENNFSYKKQKS |
| | | KSKNGKNDYLYLNFLKKIMFIEEVD |
| | | EKKEINKEKFKNKINSNFKNLFVQH |
| | | ILDYGKLLYYKENDEYIKNTGQLET |
| | | KDLEYIKTKETLIRKMAVLVSFAAN |
| | | SYYNLFGRVSGDILGTEVVKSSKTN |
| | | VIKVGSHIFKEKMLNYFFDFEIFDA |
| | | NKIVEILESISYSIYNVRNGVGHFN |
| | | KLILGKYKKKDINTNKRIEEDLNNN |
| | | EEIKGYFIKKRGEIERKVKEKFLSN |
| | | NLQYYYSKEKIENYFEVYEFEILKR |
| | | KIPFAPNFKRIIKKGEDLFNNKNNK |
| | | KYEYFKNFDKNSAEEKKEFLKTRNF |
| | | LLKELYYNNFYKEFLSKKEEFEKIV |
| | | LEVKEEKKSRGNINNKKSGVSFQSI |
| | | DDYDTKINISDYIASIHKKEMERVE |
| | | KYNEEKQKDTAKYIRDFVEEIFLTG |
| | | FINYLEKDKRLHFLKEEFSILCNNN |
| | | NNVVDFNININEEKIKEFLKENDSK |
| | | TLNLYLFFNMIDSKRISEFRNELVK |
| | | YKQFTKKRLDEEKEFLGIKIELYET |
| | | LIEFVILTREKLDTKKSEEIDAWLV |
| | | DKLYVKDSNEYKEYEEILKLFVDEK |
| | | ILSSKEAPYYATDNKTPILLSNFEK |
| | | TRKYGTQSFLSEIQSNYKYSKVEKE |
| | | NIEDYNKKEEIEQKKKSNIEKLQDL |
| | | KVELHKKWEQNKITEKEIEKYNNTT |
| | | RKINEYNYLKNKEELQNVYLLHEML |
| | | SDLLARNVAFFNKWERDFKFIVIAI |
| | | KQFLRENDKEKVNEFLNPPDNSKGK |
| | | KVYFSVSKYKNTVENIDGIHKNFMN |
| | | LIFLNNKFMNRKIDKMNCAIWVYFR |
| | | NYIAHFLHLHTKNEKISLISQMNLL |
| | | IKLFSYDKKVQNHILKSTKTLLEKY |
| | | NIQINFEISNDKNEVFKYKIKNRLY |
| | | SKKGKMLGKNNKFEILENEFLENVK |
| | | AMLEYSE |
| SEQ | Bergeyella | MENKTSLGNNIYYNPFKPQDKSYFA |
| ID | zoohelcum | GYFNAAMENTDSVFRELGKRLKGKE |
| NO: | Cas13b | YTSENFFDAIFKENISLVEYERYVK |
| 161 | | LLSDYFPMARLLDKKEVPIKERKEN |
| | | FKKNFKGIIKAVRDLRNFYTHKEHG |
| | | EVEITDEIFGVLDEMLKSTVLTVKK |
| | | KKVKTDKTKEILKKSIEKQLDILCQ |
| | | KKLEYLRDTARKIEEKRRNQRERGE |
| | | KELVAPFKYSDKRDDLIAAIYNDAF |
| | | DVYIDKKKDSLKESSKAKYNTKSDP |
| | | QQEEGDLKIPISKNGVVFLLSLFLT |
| | | KQEIHAFKSKIAGFKATVIDEATVS |
| | | EATVSHGKNSICFMATHEIFSHLAY |
| | | KKLKRKVRTAEINYGEAENAEQLSV |
| | | YAKETLMMQMLDELSKVPDVVYQNL |
| | | SEDVQKTFIEDWNEYLKENNGDVGT |
| | | MEEEQVIHPVIRKRYEDKFNYFAIR |
| | | FLDEFAQFPTLRFQVHLGNYLHDSR |
| | | PKENLISDRRIKEKITVFGRLSELE |
| | | HKKALFIKNTETNEDREHYWEIFPN |
| | | PNYDFPKENISVNDKDFPIAGSILD |
| | | REKQPVAGKIGIKVKLLNQQYVSEV |
| | | DKAVKAHQLKQRKASKPSIQNIIEE |
| | | IVPINESNPKEAIVFGGQPTAYLSM |
| | | NDIHSILYEFFDKWEKKKEKLEKKG |
| | | EKELRKEIGKELEKKIVGKIQAQIQ |
| | | QIIDKDTNAKILKPYQDGNSTAIDK |
| | | EKLIKDLKQEQNILQKLKDEQTVRE |
| | | KEYNDFIAYQDKNREINKVRDRNHK |
| | | QYLKDNLKRKYPEAPARKEVLYYRE |
| | | KGKVAVWLANDIKRFMPTDFKNEWK |
| | | GEQHSLLQKSLAYYEQCKEELKNLL |
| | | PEKVFQHLPFKLGGYFQQKYLYQFY |
| | | TCYLDKRLEYISGLVQQAENFKSEN |
| | | KVFKKVENECFKFLKKQNYTHKELD |
| | | ARVQSILGYPIFLERGFMDEKPTII |
| | | KGKTFKGNEALFADWFRYYKEYQNF |
| | | QTFYDTENYPLVELEKKQADRKRKT |
| | | KIYQQKKNDVFTLLMAKHIFKSVFK |
| | | QDSIDQFSLEDLYQSREERLGNQER |
| | | ARQTGERNTNYIWNKTVDLKLCDGK |
| | | ITVENVKLKNVGDFIKYEYDQRVQA |
| | | FLKYEENIEWQAFLIKESKEEENYP |
| | | YVVEREIEQYEKVRREELLKEVHLI |
| | | EEYILEKVKDKEILKKGDNQNFKYY |
| | | ILNGLLKQLKNEDVESYKVFNLNTE |
| | | PEDVNINQLKQEATDLEQKAFVLTY |
| | | IRNKFAHNQLPKKEFWDYCQEKYGK |
| | | IEKEKTYAEYFAEVFKKEKEALIK |
| SEQ | Prevotella | MEDDKKTTDSIRYELKDKHFWAAFL |
| ID | intermedia | NLARHNVYITVNHINKILEEGEINR |
| NO: | Cas13b | DGYETTLKNTWNEIKDINKKDRLSK |
| 162 | | LIIKHFPFLEAATYRLNPTDTTKQK |
| | | EEKQAEAQSLESLRKSFFVFIYKLR |
| | | DLRNHYSHYKHSKSLERPKFEEGLL |
| | | EKMYNIFNASIRLVKEDYQYNKDIN |
| | | PDEDFKHLDRTEEEFNYYFTKDNEG |
| | | NITESGLLFFVSLFLEKKDAIWMQQ |
| | | KLRGFKDNRENKKKMTNEVFCRSRM |
| | | LLPKLRLQSTQTQDWILLDMLNELI |
| | | RCPKSLYERLREEDREKFRVPIEIA |
| | | DEDYDAEQEPFKNTLVRHQDRFPYF |
| | | ALRYFDYNEIFTNLRFQIDLGTYHF |
| | | SIYKKQIGDYKESHHLTHKLYGFER |
| | | IQEFTKQNRPDEWRKFVKTFNSFET |
| | | SKEPYIPETTPHYHLENQKIGIRFR |
| | | NDNDKIWPSLKTNSEKNEKSKYKLD |
| | | KSFQAEAFLSVHELLPMMFYYLLLK |
| | | TENTDNDNEIETKKKENKNDKQEKH |
| | | KIEEIIENKITEIYALYDTFANGEI |
| | | KSIDELEEYCKGKDIEIGHLPKQMI |
| | | AILKDEHKVMATEAERKQEEMLVDV |
| | | QKSLESLDNQINEEIENVERKNSSL |
| | | KSGKIASWLVNDMMRFQPVQKDNEG |
| | | KPLNNSKANSTEYQLLQRTLAFFGS |
| | | EHERLAPYFKQTKLIESSNPHPFLK |
| | | DTEWEKCNNILSFYRSYLEAKKNFL |
| | | ESLKPEDWEKNQYFLKLKEPKTKPK |
| | | TLVQGWKNGFNLPRGIFTEPIRKWF |
| | | MKHRENITVAELKRVGLVAKVIPLF |
| | | FSEEYKDSVQPFYNYHFNVGNINKP |
| | | DEKNFLNCEERRELLRKKKDEFKKM |
| | | TDKEKEENPSYLEFKSWNKFERELR |
| | | LVRNQDIVTWLLCMELFNKKKIKEL |
| | | NVEKIYLKNINTNTTKKEKNTEEKN |
| | | GEEKNIKEKNNILNRIMPMRLPIKV |
| | | YGRENFSKNKKKKIRRNTFFTVYIE |
| | | EKGTKLLKQGNFKALERDRRLGGLF |
| | | SFVKTPSKAESKSNTISKLRVEYEL |
| | | GEYQKARIEIIKDMLALEKTLIDKY |
| | | NSLDTDNFNKMLTDWLELKGEPDKA |
| | | SFQNDVDLLIAVRNAFSHNQYPMRN |
| | | RIAFANINPFSLSSANTSEEKGLGI |
| | | ANQLKDKTHKTIEKIIEIEKPIETK |
| | | E |
| SEQ | Prevotella | MQKQDKLFVDRKKNAIFAFPKYITI |
| ID | buccae | MENKEKPEPIYYELTDKHFWAAFLN |
| NO: | Cas13b | LARHNVYTTINHINRRLEIAELKDD |
| 163 | | GYMMGIKGSWNEQAKKLDKKVRLRD |
| | | LIMKHFPFLEAAAYEMTNSKSPNNK |
| | | EQREKEQSEALSLNNLKNVLFIFLE |
| | | KLQVLRNYYSHYKYSEESPKPIFET |
| | | SLLKNMYKVFDANVRLVKRDYMHHE |
| | | NIDMQRDFTHLNRKKQVGRTKNIID |
| | | SPNFHYHFADKEGNMTIAGLLFFVS |
| | | LFLDKKDAIWMQKKLKGFKDGRNLR |
| | | EQMTNEVFCRSRISLPKLKLENVQT |
| | | KDWMQLDMLNELVRCPKSLYERLRE |
| | | KDRESFKVPFDIFSDDYNAEEEPFK |
| | | NTLVRHQDRFPYFVLRYFDLNEIFE |
| | | QLRFQIDLGTYHFSIYNKRIGDEDE |
| | | VRHLTHHLYGFARIQDFAPQNQPEE |
| | | WRKLVKDLDHFETSQEPYISKTAPH |
| | | YHLENEKIGIKFCSAHNNLFPSLQT |
| | | DKTCNGRSKFNLGTQFTAEAFLSVH |
| | | ELLPMMFYYLLLTKDYSRKESADKV |
| | | EGIIRKEISNIYAIYDAFANNEINS |
| | | IADLTRRLQNTNILQGHLPKQMISI |
| | | LKGRQKDMGKEAERKIGEMIDDTQR |
| | | RLDLLCKQTNQKIRIGKRNAGLLKS |
| | | GKIADWLVNDMMRFQPVQKDQNNIP |
| | | INNSKANSTEYRMLQRALALFGSEN |
| | | FRLKAYFNQMNLVGNDNPHPFLAET |
| | | QWEHQTNILSFYRNYLEARKKYLKG |
| | | LKPQNWKQYQHFLILKVQKTNRNTL |
| | | VTGWKNSFNLPRGIFTQPIREWFEK |
| | | HNNSKRIYDQILSFDRVGFVAKAIP |
| | | LYFAEEYKDNVQPFYDYPFNIGNRL |
| | | KPKKRQFLDKKERVELWQKNKELFK |
| | | NYPSEKKKTDLAYLDFLSWKKFERE |
| | | LRLIKNQDIVTWLMFKELFNMATVE |
| | | GLKIGEIHLRDIDTNTANEESNNIL |
| | | NRIMPMKLPVKTYETDNKGNILKER |
| | | PLATFYIEETETKVLKQGNFKALVK |
| | | DRRLNGLFSFAETTDLNLEEHPISK |
| | | LSVDLELIKYQTTRISIFEMTLGLE |
| | | KKLIDKYSTLPTDSFRNMLERWLQC |
| | | KANRPELKNYVNSLIAVRNAFSHNQ |
| | | YPMYDATLFAEVKKFTLFPSVDTKK |
| | | IELNIAPQLLEIVGKAIKEIEKSEN |
| | | KN |
| SEQ | Porphyromonas | MNTVPASENKGQSRTVEDDPQYFGL |
| ID | gingivalis | YLNLARENLIEVESHVRIKFGKKKL |
| NO: | Cas13b | NEESLKQSLLCDHLLSVDRWTKVYG |
| 164 | | HSRRYLPFLHYFDPDSQIEKDHDSK |
| | | TGVDPDSAQRLIRELYSLLDFLRND |
| | | FSHNRLDGTTFEHLEVSPDISSFIT |
| | | GTYSLACGRAQSRFAVFFKPDDFVL |
| | | AKNRKEQLISVADGKECLTVSGFAF |
| | | FICLFLDREQASGMLSRIRGFKRTD |
| | | ENWARAVHETFCDLCIRHPHDRLES |
| | | SNTKEALLLDMLNELNRCPRILYDM |
| | | LPEEERAQFLPALDENSMNNLSENS |
| | | LDEESRLLWDGSSDWAEALTKRIRH |
| | | QDRFPYLMLRFIEEMDLLKGIRFRV |
| | | DLGEIELDSYSKKVGRNGEYDRTIT |
| | | DHALAFGKLSDFQNEEEVSRMISGE |
| | | ASYPVRFSLFAPRYAIYDNKIGYCH |
| | | TSDPVYPKSKTGEKRALSNPQSMGF |
| | | ISVHDLRKLLLMELLCEGSFSRMQS |
| | | DFLRKANRILDETAEGKLQFSALFP |
| | | EMRHRFIPPQNPKSKDRREKAETTL |
| | | EKYKQEIKGRKDKLNSQLLSAFDMD |
| | | QRQLPSRLLDEWMNIRPASHSVKLR |
| | | TYVKQLNEDCRLRLRKFRKDGDGKA |
| | | RAIPLVGEMATFLSQDIVRMIISEE |
| | | TKKLITSAYYNEMQRSLAQYAGEEN |
| | | RRQFRAIVAELRLLDPSSGHPFLSA |
| | | TMETAHRYTEGFYKCYLEKKREWLA |
| | | KIFYRPEQDENTKRRISVFFVPDGE |
| | | ARKLLPLLIRRRMKEQNDLQDWIRN |
| | | KQAHPIDLPSHLFDSKVMELLKVKD |
| | | GKKKWNEAFKDWWSTKYPDGMQPFY |
| | | GLRRELNIHGKSVSYIPSDGKKFAD |
| | | CYTHLMEKTVRDKKRELRTAGKPVP |
| | | PDLAADIKRSFHRAVNEREFMLRLV |
| | | QEDDRLMLMAINKMMTDREEDILPG |
| | | LKNIDSILDEENQFSLAVHAKVLEK |
| | | EGEGGDNSLSLVPATIEIKSKRKDW |
| | | SKYIRYRYDRRVPGLMSHFPEHKAT |
| | | LDEVKTLLGEYDRCRIKIFDWAFAL |
| | | EGAIMSDRDLKPYLHESSSREGKSG |
| | | EHSTLVKMLVEKKGCLTPDESQYLI |
| | | LIRNKAAHNQFPCAAEMPLIYRDVS |
| | | AKVGSIEGSSAKDLPEGSSLVDSLW |
| | | KKYEMIIRKILPILDPENRFFGKLL |
| | | NNMSQPINDL |
| SEQ | Bacteroides | MESIKNSQKSTGKTLQKDPPYFGLY |
| ID | pyogenes | LNMALLNVRKVENHIRKWLGDVALL |
| NO: | Cas13b | PEKSGFHSLLTTDNLSSAKWTRFYY |
| 165 | | KSRKFLPFLEMFDSDKKSYENRRET |
| | | AECLDTIDRQKISSLLKEVYGKLQD |
| | | IRNAFSHYHIDDQSVKHTALIISSE |
| | | MHRFIENAYSFALQKTRARFTGVFV |
| | | ETDFLQAEEKGDNKKFFAIGGNEGI |
| | | KLKDNALIFLICLFLDREEAFKFLS |
| | | RATGFKSTKEKGFLAVRETFCALCC |
| | | RQPHERLLSVNPREALLMDMLNELN |
| | | RCPDILFEMLDEKDQKSFLPLLGEE |
| | | EQAHILENSLNDELCEAIDDPFEMI |
| | | ASLSKRVRYKNRFPYLMLRYIEEKN |
| | | LLPFIRFRIDLGCLELASYPKKMGE |
| | | ENNYERSVTDHAMAFGRLTDFHNED |
| | | AVLQQITKGITDEVRFSLYAPRYAI |
| | | YNNKIGFVRTSGSDKISFPTLKKKG |
| | | GEGHCVAYTLQNTKSFGFISIYDLR |
| | | KILLLSFLDKDKAKNIVSGLLEQCE |
| | | KHWKDLSENLFDAIRTELQKEFPVP |
| | | LIRYTLPRSKGGKLVSSKLADKQEK |
| | | YESEFERRKEKLTEILSEKDFDLSQ |
| | | IPRRMIDEWLNVLPTSREKKLKGYV |
| | | ETLKLDCRERLRVFEKREKGEHPLP |
| | | PRIGEMATDLAKDIIRMVIDQGVKQ |
| | | RITSAYYSEIQRCLAQYAGDDNRRH |
| | | LDSIIRELRLKDTKNGHPFLGKVLR |
| | | PGLGHTEKLYQRYFEEKKEWLEATF |
| | | YPAASPKRVPRFVNPPTGKQKELPL |
| | | IIRNLMKERPEWRDWKQRKNSHPID |
| | | LPSQLFENEICRLLKDKIGKEPSGK |
| | | LKWNEMFKLYWDKEFPNGMQRFYRC |
| | | KRRVEVFDKVVEYEYSEEGGNYKKY |
| | | YEALIDEVVRQKISSSKEKSKLQVE |
| | | DLTLSVRRVFKRAINEKEYQLRLLC |
| | | EDDRLLFMAVRDLYDWKEAQLDLDK |
| | | IDNMLGEPVSVSQVIQLEGGQPDAV |
| | | IKAECKLKDVSKLMRYCYDGRVKGL |
| | | MPYFANHEATQEQVEMELRHYEDHR |
| | | RRVFNWVFALEKSVLKNEKLRRFYE |
| | | ESQGGCEHRRCIDALRKASLVSEEE |
| | | YEFLVHIRNKSAHNQFPDLEIGKLP |
| | | PNVTSGFCECIWSKYKAIICRIIPF |
| | | IDPERRFFGKLLEQK |
| SEQ | Cas13c | MTEKKSIIFKNKSSVEIVKKDIFSQ |
| ID | | TPDNMIRNYKITLKISEKNPRVVEA |
| NO: | | EIEDLMNSTILKDGRRSARREKSMT |
| 166 | | ERKLIEEKVAENYSLLANCPMEEVD |
| | | SIKIYKIKRFLTYRSNMLLYFASIN |
| | | SFLCEGIKGKDNETEEIWHLKDNDV |
| | | RKEKVKENFKNKLIQSTENYNSSLK |
| | | NQIEEKEKLLRKESKKGAFYRTIIK |
| | | KLQQERIKELSEKSLTEDCEKIIKL |
| | | YSELRHPLMHYDYQYFENLFENKEN |
| | | SELTKNLNLDIFKSLPLVRKMKLNN |
| | | KVNYLEDNDTLFVLQKTKKAKTLYQ |
| | | IYDALCEQKNGFNKFINDFFVSDGE |
| | | ENTVFKQIINEKFQSEMEFLEKRIS |
| | | ESEKKNEKLKKKFDSMKAHFHNINS |
| | | EDTKEAYFWDIHSSSNYKTKYNERK |
| | | NLVNEYTELLGSSKEKKLLREEITQ |
| | | INRKLLKLKQEMEEITKKNSLFRLE |
| | | YKMKIAFGFLFCEFDGNISKFKDEF |
| | | DASNQEKIIQYHKNGEKYLTYFLKE |
| | | EEKEKFNLEKMQKIIQKTEEEDWLL |
| | | PETKNNLFKFYLLTYLLLPYELKGD |
| | | FLGFVKKHYYDIKNVDFMDENQNNI |
| | | QVSQTVEKQEDYFYHKIRLFEKNTK |
| | | KYEIVKYSIVPNEKLKQYFEDLGID |
| | | IKYLTGSVESGEKWLGENLGIDIKY |
| | | LTVEQKSEVSEEKIKKFL |
| SEQ | Cas13c | MEKDKKGEKIDISQEMIEEDLRKIL |
| ID | | ILFSRLRHSMVHYDYEFYQALYSGK |
| NO: | | DFVISDKNNLENRMISQLLDLNIFK |
| 167 | | ELSKVKLIKDKAISNYLDKNTTIHV |
| | | LGQDIKAIRLLDIYRDICGSKNGFN |
| | | KFINTMITISGEEDREYKEKVIEHF |
| | | NKKMENLSTYLEKLEKQDNAKRNNK |
| | | RVYNLLKQKLIEQQKLKEWFGGPYV |
| | | YDIHSSKRYKELYIERKKLVDRHSK |
| | | LFEEGLDEKNKKELTKINDELSKLN |
| | | SEMKEMTKLNSKYRLQYKLQLAFGF |
| | | ILEEFDLNIDTFINNFDKDKDLIIS |
| | | NFMKKRDIYLNRVLDRGDNRLKNII |
| | | KEYKFRDTEDIFCNDRDNNLVKLYI |
| | | LMYILLPVEIRGDFLGFVKKNYYDM |
| | | KHVDFIDKKDKEDKDTFFHDLRLFE |
| | | KNIRKLEITDYSLSSGFLSKEHKVD |
| | | IEKKINDFINRNGAMKLPEDITIEE |
| | | FNKSLILPIMKNYQINFKLLNDIEI |
| | | SALFKIAKDRSITFKQAIDEIKNED |
| | | IKKNSKKNDKNNHKDKNINFTQLMK |
| | | RALHEKIPYKAGMYQIRNNISHIDM |
| | | EQLYIDPLNSYMNSNKNNITISEQI |
| | | EKIIDVCVTGGVTGKELNNNIINDY |
| | | YMKKEKLVFNLKLRKQNDIVSIESQ |
| | | EKNKREEFVFKKYGLDYKDGEINII |
| | | EVIQKVNSLQEELRNIKETSKEKLK |
| | | NKETLFRDISLINGTIRKNINFKIK |
| | | EMVLDIVRMDEIRHINIHIYYKGEN |
| | | YTRSNIIKFKYAIDGENKKYYLKQH |
| | | EINDINLELKDKFVTLICNMDKHPN |
| | | KNKQTINLESNYIQNVKFIIP |
| SEQ | Cas13c | MENKGNNKKIDFDENYNILVAQIKE |
| ID | | YFTKEIENYNNRIDNIIDKKELLKY |
| NO: | | SEKKEESEKNKKLEELNKLKSQKLK |
| 168 | | ILTDEEIKADVIKIIKIFSDLRHSL |
| | | MHYEYKYFENLFENKKNEELAELLN |
| | | LNLFKNLTLLRQMKIENKTNYLEGR |
| | | EEFNIIGKNIKAKEVLGHYNLLAEQ |
| | | KNGFNNFINSFFVQDGTENLEFKKL |
| | | IDEHFVNAKKRLERNIKKSKKLEKE |
| | | LEKMEQHYQRLNCAYVWDIHTSTTY |
| | | KKLYNKRKSLIEEYNKQINEIKDKE |
| | | VITAINVELLRIKKEMEEITKSNSL |
| | | FRLKYKMQIAYAFLEIEFGGNIAKF |
| | | KDEFDCSKMEEVQKYLKKGVKYLKY |
| | | YKDKEAQKNYEFPFEEIFENKDTHN |
| | | EEWLENTSENNLFKFYILTYLLLPM |
| | | EFKGDFLGVVKKHYYDIKNVDFTDE |
| | | SEKELSQVQLDKMIGDSFFHKIRLF |
| | | EKNTKRYEIIKYSILTSDEIKRYFR |
| | | LLELDVPYFEYEKGTDEIGIFNKNI |
| | | ILTIFKYYQIIFRLYNDLEIHGLFN |
| | | ISSDLDKILRDLKSYGNKNINFREF |
| | | LYVIKQNNNSSTEEEYRKIWENLEA |
| | | KYLRLHLLTPEKEEIKTKTKEELEK |
| | | LNEISNLRNGICHLNYKEIIEEILK |
| | | TEISEKNKEATLNEKIRKVINFIKE |
| | | NELDKVELGFNFINDFFMKKEQFMF |
| | | GQIKQVKEGNSDSITTERERKEKNN |
| | | KKLKETYELNCDNLSEFYETSNNLR |
| | | ERANSSSLLEDSAFLKKIGLYKVKN |
| | | NKVNSKVKDEEKRIENIKRKLLKDS |
| | | SDIMGMYKAEVVKKLKEKLILIFKH |
| | | DEEKRIYVTVYDTSKAVPENISKEI |
| | | LVKRNNSKEEYFFEDNNKKYVTEYY |
| | | TLEITETNELKVIPAKKLEGKEFKT |
| | | EKNKENKLMLNNHYCFNVKIIY |
| SEQ | Cas13c | MEEIKHKKNKSSIIRVIVSNYDMTG |
| ID | | IKEIKVLYQKQGGVDTFNLKTIINL |
| NO: | | ESGNLEIISCKPKEREKYRYEFNCK |
| 169 | | TEINTISITKKDKVLKKEIRKYSLE |
| | | LYFKNEKKDTVVAKVTDLLKAPDKI |
| | | EGERNHLRKLSSSTERKLLSKTLCK |
| | | NYSEISKTPIEEIDSIKIYKIKRFL |
| | | NYRSNFLIYFALINDFLCAGVKEDD |
| | | INEVWLIQDKEHTAFLENRIEKITD |
| | | YIFDKLSKDIENKKNQFEKRIKKYK |
| | | TSLEELKTETLEKNKTFYIDSIKTK |
| | | ITNLENKITELSLYNSKESLKEDLI |
| | | KIISIFTNLRHSLMHYDYKSFENLF |
| | | ENIENEELKNLLDLNLFKSIRMSDE |
| | | FKTKNRTNYLDGTESFTIVKKHQNL |
| | | KKLYTYYNNLCDKKNGFNTFINSFF |
| | | VTDGIENTDFKNLIILHFEKEMEEY |
| | | KKSIEYYKIKISNEKNKSKKEKLKE |
| | | KIDLLQSELINMREHKNLLKQIYFF |
| | | DIHNSIKYKELYSERKNLIEQYNLQ |
| | | INGVKDVTAINHINTKLLSLKNKMD |
| | | KITKQNSLYRLKYKLKIAYSFLMIE |
| | | FDGDVSKFKNNFDPTNLEKRVEYLD |
| | | KKEEYLNYTAPKNKFNFAKLEEELQ |
| | | KIQSTSEMGADYLNVSPENNLFKFY |
| | | ILTYIMLPVEFKGDFLGFVKNHYYN |
| | | IKNVDFMDESLLDENEVDSNKLNEK |
| | | IENLKDSSFFNKIRLFEKNIKKYEI |
| | | VKYSVSTQENMKEYFKQLNLDIPYL |
| | | DYKSTDEIGIFNKNMILPIFKYYQN |
| | | VFKLCNDIEIHALLALANKKQQNLE |
| | | YAIYCCSKKNSLNYNELLKTFNRKT |
| | | YQNLSFIRNKIAHLNYKELFSDLFN |
| | | NELDLNTKVRCLIEFSQNNKFDQID |
| | | LGMNFINDYYMKKTRFIFNQRRLRD |
| | | LNVPSKEKIIDGKRKQQNDSNNELL |
| | | KKYGLSRTNIKDIFNKAWY |
| SEQ | Cas13c | MKVRYRKQAQLDTFIIKTEIVNNDI |
| ID | | FIKSIIEKAREKYRYSFLFDGEEKY |
| NO: | | HFKNKSSVEIVKNDIFSQTPDNMIR |
| 170 | | NYKITLKISEKNPRVVEAEIEDLMN |
| | | STILKDGRRSARREKSMTERKLIEE |
| | | KVAENYSLLANCPIEEVDSIKIYKI |
| | | KRFLTYRSNMLLYFASINSFLCEGI |
| | | KGKDNETEEIWHLKDNDVRKEKVKE |
| | | NFKNKLIQSTENYNSSLKNQIEEKE |
| | | KLSSKEFKKGAFYRTIIKKLQQERI |
| | | KELSEKSLTEDCEKIIKLYSELRHP |
| | | LMHYDYQYFENLFENKENSELTKNL |
| | | NLDIFKSLPLVRKMKLNNKVNYLED |
| | | NDTLFVLQKTKKAKTLYQIYDALCE |
| | | QKNGFNKFINDFFVSDGEENTVFKQ |
| | | IINEKFQSEMEFLEKRISESEKKNE |
| | | KLKKKLDSMKAHFRNINSEDTKEAY |
| | | FWDIHSSRNYKTKYNERKNLVNEYT |
| | | KLLGSSKEKKLLREEITKINRQLLK |
| | | LKQEMEEITKKNSLFRLEYKMKIAF |
| | | GFLFCEFDGNISKFKDEFDASNQEK |
| | | IIQYHKNGEKYLTSFLKEEEKEKFN |
| | | LEKMQKIIQKTEEEDWLLPETKNNL |
| | | FKFYLLTYLLLPYELKGDFLGFVKK |
| | | HYYDIKNVDFMDENQNNIQVSQTVE |
| | | KQEDYFYHKIRLFEKNTKKYEIVKY |
| | | SIVPNEKLKQYFEDLGIDIKYLTGS |
| | | VESGEKWLGENLGIDIKYLTVEQKS |
| | | EVSEEKNKKVSLKNNGMFNKTILLF |
| | | VFKYYQIAFKLFNDIELYSLFFLRE |
| | | KSEKPFEVFLEELKDKMIGKQLNFG |
| | | QLLYVVYEVLVKNKDLDKILSKKID |
| | | YRKDKSFSPEIAYLRNFLSHLNYSK |
| | | FLDNFMKINTNKSDENKEVLIPSIK |
| | | IQKMIQFIEKCNLQNQIDFDFNFVN |
| | | DFYMRKEKMFFIQLKQIFPDINSTE |
| | | KQKKSEKEEILRKRYHLINKKNEQI |
| | | KDEHEAQSQLYEKILSLQKIFSCDK |
| | | NNFYRRLKEEKLLFLEKQGKKKISM |
| | | KEIKDKIASDISDLLGILKKEITRD |
| | | IKDKLTEKFRYCEEKLLNISFYNHQ |
| | | DKKKEEGIRVFLIRDKNSDNFKFES |
| | | ILDDGSNKIFISKNGKEITIQCCDK |
| | | VLETLMIEKNTLKISSNGKIISLIP |
| | | HYSYSIDVKY |

The programmable nuclease can be Cas13 Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a Type V CRISPR-Cas enzyme. In some cases, the programmable nuclease can be a Type VI CRISPR-Cas enzyme. Sometimes the programmable nuclease can be a Type III CRISPR-Cas enzyme. In some cases, the programmable nuclease can be from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the Cas enzyme can be activated when the crRNA is complexed with the target nucleic acid. The trans cleavage activity of the Cas enzyme can be activated when the guide nucleic acid comprising a tracrRNA and crRNA are complexed with the target nucleic acid. The target nucleic acid can be RNA or DNA.

In some embodiments, a programmable nuclease as disclosed herein is an RNA-activated programmable RNA nuclease. In some embodiments, a programmable nuclease as disclosed herein is a DNA-activated programmable RNA nuclease. In some embodiments, a programmable nuclease is capable of being activated by a target RNA to initiate trans cleavage of an RNA detector nucleic acid and is capable of being activated by a target DNA to initiate trans cleavage of an RNA detector nucleic acid, such as a Type VI CRISPR-Cas enzyme (e.g., Cas13). For example, Cas13a of the present disclosure can be activated by a target RNA to initiate trans cleavage activity of the Cas13a for the cleavage of an RNA detector nucleic acid and can be activated by a target DNA to initiate trans cleavage activity of the Cas13a for trans cleavage of an RNA detector nucleic acid. An RNA detector nucleic acid can be an RNA-based detector nucleic acid molecule. In some embodiments, the Cas13a recognizes and detects ssDNA to initiate transcleavage of RNA detector nucleic acids. Multiple Cas13a isolates can recognize, be activated by, and detect target DNA, including ssDNA, upon hybridization of a guide nucleic acid with the target DNA. For example, Lbu-Cas13a and Lwa-Cas13a can both be activated to transcollaterally cleave RNA detector nucleic acids by target DNA. Thus, Type VI CRISPR-Cas enzyme (e.g., Cas13, such as Cas13a) can be DNA-activated programmable RNA nucleases, and therefore, can be used to detect a target DNA using the methods as described herein. DNA-activated programmable RNA nuclease detection of ssDNA can be robust at multiple pH values. For example, target ssDNA detection by Cas13 can exhibit consistent cleavage across a wide range of pH conditions, such as from a pH of 6.8 to a pH of 8.2. In contrast, target RNA detection by Cas13 may exhibit high cleavage activity of pH values from 7.9 to 8.2. In some embodiments, a DNA-activated programmable RNA nuclease that also is capable of being an RNA-activated programmable RNA nuclease, can have DNA targeting preferences that are distinct from its RNA targeting preferences. For example, the optimal ssDNA targets for Cas13a have different properties than optimal RNA targets for Cas13a. As one example, gRNA performance on ssDNA may not necessarily correlate with the performance of the same gRNAs on RNA. As another example, gRNAs can perform at a high level regardless of target nucleotide identity at a 3′ position on a target RNA sequence. In some embodiments, gRNAs can perform at a high level in the absence of a G at a 3′ position on a target ssDNA sequence. Furthermore, target DNA detected by Cas13 disclosed herein can be directly from organisms, or can be indirectly generated by nucleic acid amplification methods, such as PCR and LAMP or any amplification method described herein. Key steps for the sensitive detection of a target DNA, such as a target ssDNA, by a DNA-activated programmable RNA nuclease, such as Cas13a, can include: (1) production or isolation of DNA to concentrations above about 0.1 nM per reaction for in vitro diagnostics, (2) selection of a target sequence with the appropriate sequence features to enable DNA detection as these features are distinct from those required for RNA detection, and (3) buffer composition that enhances DNA detection. The detection of a target DNA by a DNA-activated programmable RNA nuclease can be connected to a variety of readouts including fluorescence, lateral flow, electrochemistry, or any other readouts described herein. Multiplexing of programmable DNA nuclease, such as a Type V CRISPR-Cas enzyme, with a DNA-activated programmable RNA nuclease, such as a Type VI CRISPR-Cas enzyme, with a DNA detector nucleic acid and an RNA detector nucleic acid, can enable multiplexed detection of target ssDNAs or a combination of a target dsDNA and a target ssDNA, respectively. Multiplexing of different RNA-activated programmable RNA nucleases that have distinct RNA detector nucleic acid cleavage preferences can enable additional multiplexing. Methods for the generation of ssDNA for DNA-activated programmable RNA nuclease-based diagnostics can include (1) asymmetric PCR, (2) asymmetric isothermal amplification, such as RPA, LAMP, SDA, etc. (3) NEAR for the production of short ssDNA molecules, and (4) conversion of RNA targets into ssDNA by a reverse transcriptase followed by RNase H digestion. Thus, DNA-activated programmable RNA nuclease detection of target DNA is compatible with the various systems, kits, compositions, reagents, and methods disclosed herein. For example target ssDNA detection by Cas13a can be employed in a DETECTR assay disclosed herein (e.g., a multiplexed DETECTR reaction or a high-plex DETECTR reaction).

Described herein are reagents comprising a single-stranded detector nucleic acid comprising a detection moiety, wherein the detector nucleic acid is capable of being cleaved by the activated nuclease, thereby generating a first detectable signal. In some cases, the detector nucleic acid is a single-stranded nucleic acid comprising deoxyribonucleotides. In other cases, the detector nucleic acid is a single-stranded nucleic acid comprising ribonucleotides. The detector nucleic acid can be a single-stranded nucleic acid comprising at least one deoxyribonucleotide and at least one ribonucleotide. In some cases, the detector nucleic acid is a single-stranded nucleic acid comprising at least one ribonucleotide residue at an internal position that functions as a cleavage site. In some cases, the detector nucleic acid comprises at least 2, 3, 4, 5, 6, 7, 8, 9, or 10 ribonucleotide residues at an internal position. Sometimes the ribonucleotide residues are continuous. Alternatively, the ribonucleotide residues are interspersed in between non-ribonucleotide residues. In some cases, the detector nucleic acid has only ribonucleotide residues. In some cases, the detector nucleic acid has only deoxyribonucleotide residues. In some cases, the detector nucleic acid comprises nucleotides resistant to cleavage by the programmable nuclease described herein. In some cases, the detector nucleic acid comprises synthetic nucleotides. In some cases, the detector nucleic acid comprises at least one ribonucleotide residue and at least one non-ribonucleotide residue. In some cases, detector nucleic acid is 5-20, 5-15, 5-10, 7-20, 7-15, or 7-10 nucleotides in length. In some cases, the detector nucleic acid comprises at least one uracil ribonucleotide. In some cases, the detector nucleic acid comprises at least two uracil ribonucleotides. Sometimes the detector nucleic acid has only uracil ribonucleotides. In some cases, the detector nucleic acid comprises at least one adenine ribonucleotide. In some cases, the detector nucleic acid comprises at least two adenine ribonucleotide. In some cases, the detector nucleic acid has only adenine ribonucleotides. In some cases, the detector nucleic acid comprises at least one cytosine ribonucleotide. In some cases, the detector nucleic acid comprises at least two cytosine ribonucleotide. In some cases, the detector nucleic acid comprises at least one guanine ribonucleotide. In some cases, the detector nucleic acid comprises at least two guanine ribonucleotide. A detector nucleic acid can comprise only unmodified ribonucleotides, only unmodified deoxyribonucleotides, or a combination thereof. In some cases, the detector nucleic acid is from 5 to 12 nucleotides in length. In some cases, the detector nucleic acid is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the detector nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a detector nucleic acid can be 5, 8, or 10 nucleotides in length. For cleavage by a programmable nuclease comprising Cas12, a detector nucleic acid can be 10 nucleotides in length.

The single-stranded detector nucleic acid comprises a detection moiety capable of generating a first detectable signal. Sometimes the detector nucleic acid comprises a protein capable of generating a signal. A signal can be a calorimetric, potentiometric, amperometric, optical (e.g., fluorescent, colorometric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the detector nucleic acid. Sometimes the detection moiety is at the 3′ terminus of the detector nucleic acid. In some cases, the detection moiety is at the 5′ terminus of the detector nucleic acid. In some cases, the quenching moiety is at the 3′ terminus of the detector nucleic acid. In some cases, the single-stranded detector nucleic acid is at least one population of the single-stranded nucleic acid capable of generating a first detectable signal. In some cases, the single-stranded detector nucleic acid is a population of the single-stranded nucleic acid capable of generating a first detectable signal. Optionally, there is more than one population of single-stranded detector nucleic acid. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded detector nucleic acids capable of generating a detectable signal.

TABLE 4
Exemplary Single Stranded Detector Nucleic Acid

| 5′ Detection Moiety* | Sequence (SEQ ID NO:) | 3′ Quencher* |
|---|---|---|
| /56-FAM/ | rUrUrUrUrU (SEQ ID NO: 1) | /3IABkFQ/ |
| /5IRD700/ | rUrUrUrUrU (SEQ ID NO: 1) | /3IRQCIN/ |
| /5TYE665/ | rUrUrUrUrU (SEQ ID NO: 1) | /3IAbRQSp/ |
| /5Alex594N/ | rUrUrUrUrU (SEQ ID NO: 1) | /3IAbRQSp/ |
| /5ATTO633N/ | rUrUrUrUrU (SEQ ID NO: 1) | /3IAbRQSp/ |
| /56-FAM/ | rUrUrUrUrUrUrUrU(SEQ ID NO: 2) | /3IABkFQ/ |
| /5IRD700/ | rUrUrUrUrUrUrUrU(SEQ ID NO: 2) | /3IRQCIN/ |
| /5TYE665/ | rUrUrUrUrUrUrUrU(SEQ ID NO: 2) | /3IAbRQSp/ |
| /5Alex594N/ | rUrUrUrUrUrUrUrU(SEQ ID NO: 2) | /3IAbRQSp/ |
| /5ATTO633N/ | rUrUrUrUrUrUrUrU(SEQ ID NO: 2) | /3IAbRQSp/ |
| /56-FAM/ | rUrUrUrUrUrUrUrUrUrU(SEQ ID NO: 3) | /3IABkFQ/ |
| /5IRD700/ | rUrUrUrUrUrUrUrUrUrU(SEQ ID NO: 3) | /3IRQCIN/ |
| /5TYE665/ | rUrUrUrUrUrUrUrUrUrU(SEQ ID NO: 3) | /3IAbRQSp/ |
| /5Alex594N/ | rUrUrUrUrUrUrUrUrUrU(SEQ ID NO: 3) | /3IAbRQSp/ |
| /5ATTO633N/ | rUrUrUrUrUrUrUrUrUrU(SEQ ID NO: 3) | /3IAbRQSp/ |
| /56-FAM/ | TTTTrUrUTTTT(SEQ ID NO: 4) | /3IABkFQ/ |
| /5IRD700/ | TTTTrUrUTTTT(SEQ ID NO: 4) | /3IRQCIN/ |
| /5TYE665/ | TTTTrUrUTTTT(SEQ ID NO: 4) | /3IAbRQSp/ |
| /5Alex594N/ | TTTTrUrUTTTT(SEQ ID NO: 4) | /31AbRQSp/ |
| /5ATTO633N/ | TTTTrUrUTTTT(SEQ ID NO: 4) | /3IAbRQSp/ |
| /56-FAM/ | TTrUrUTT(SEQ ID NO: 5) | /3IABkFQ/ |
| /5IRD700/ | TTrUrUTT(SEQ ID NO: 5) | /3IRQCIN/ |
| /5TYE665/ | TTrUrUTT(SEQ ID NO: 5) | /3IAbRQSp/ |
| /5Alex594N/ | TTrUrUTT(SEQ ID NO: 5) | /3IAbRQSp/ |
| /5ATTO633N/ | TTrUrUTT(SEQ ID NO: 5) | /3IAbRQSp/ |
| /56-FAM/ | TArArUGC(SEQ ID NO: 6) | /3IABkFQ/ |
| /5IRD700/ | TArArUGC(SEQ ID NO: 6) | /3IRQCIN/ |
| /5TYE665/ | TArArUGC(SEQ ID NO: 6) | /3IAbRQSp/ |
| /5Alex594N/ | TArArUGC(SEQ ID NO: 6) | /3IAbRQSp/ |
| /5ATTO633N/ | TArArUGC(SEQ ID NO: 6) | /3IAbRQSp/ |
| /56-FAM/ | TArUrGGC(SEQ ID NO: 7) | /3IABkFQ/ |
| /5IRD700/ | TArUrGGC(SEQ ID NO: 7) | /3IRQCIN/ |
| /5TYE665/ | TArUrGGC(SEQ ID NO: 7) | /3IAbRQSp/ |
| /5Alex594N/ | TArUrGGC(SEQ ID NO: 7) | /3IAbRQSp/ |
| /5ATTO633N/ | TArUrGGC(SEQ ID NO: 7) | /3IAbRQSp/ |
| /56-FAM/ | rUrUrUrUrU(SEQ ID NO: 8) | /3IABkFQ/ |
| /5IRD700/ | rUrUrUrUrU(SEQ ID NO: 8) | /3IRQCIN/ |
| /5TYE665/ | rUrUrUrUrU(SEQ ID NO: 8) | /3IAbRQSp/ |
| /5Alex594N/ | rUrUrUrUrU(SEQ ID NO: 8) | /3IAbRQSp/ |
| /5ATTO633N/ | rUrUrUrUrU(SEQ ID NO: 8) | /3IAbRQSp/ |
| /56-FAM/ | TTATTATT (SEQ ID NO: 9) | /3IABkFQ/ |
| /56-FAM/ | TTATTATT (SEQ ID NO: 9) | /3IABkFQ/ |
| /5IRD700/ | TTATTATT (SEQ ID NO: 9) | /3IRQCIN/ |
| /5TYE665/ | TTATTATT (SEQ ID NO: 9) | /3IAbRQSp/ |
| /5Alex594N/ | TTATTATT (SEQ ID NO: 9) | /3IAbRQSp/ |
| /5ATTO633N/ | TTATTATT (SEQ ID NO: 9) | /3IAbRQSp/ |
| /56-FAM/ | TTTTTT (SEQ ID NO: 10) | /3IABkFQ/ |
| /56-FAM/ | TTTTTTTT (SEQ ID NO: 11) | /3IABkFQ/ |
| /56-FAM/ | TTTTTTTTTT (SEQ ID NO: 12) | /3IABkFQ/ |
| /56-FAM/ | TTTTTTTTTTTT (SEQ ID NO: 13) | /3IABkFQ/ |
| /56-FAM/ | TTTTTTTTTTTTTT (SEQ ID NO: 14) | /3IABkFQ/ |
| /56-FAM/ | AAAAAA (SEQ ID NO: 15) | /3IABkFQ/ |
| /56-FAM/ | CCCCCC (SEQ ID NO: 16) | /3IABkFQ/ |
| /56-FAM/ | GGGGGG (SEQ ID NO: 17) | /3IABkFQ/ |
| /56-FAM/ | TTATTATT (SEQ ID NO: 9) | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5 Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQCIN/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 6890 to 690, 690 to 700, 700 to 710, 710 to 720, or 720 to 730 nm. A detection moiety can be a fluorophore that emits a fluorescence in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 1 with a fluorophore that emits around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 6890 to 690, 690 to 700, 700 to 710, 710 to 720, or 720 to 730 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A detector nucleic acid, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorimetric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose and DNS reagent.

Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the detector nucleic acid comprises a nucleic acid conjugated to an affinity molecule and the affinity molecule conjugated to the fluorophore (e.g., nucleic acid—affinity molecule—fluorophore) or the nucleic acid conjugated to the fluorophore and the fluorophore conjugated to the affinity molecule (e.g., nucleic acid—fluorophore—affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the detector nucleic acid can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicated that no intervening molecules, polypeptides, proteins, or other moieties are present between the two moieties directly conjugated to each other. For example, if a detector nucleic acid comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule.

In some cases, the detector nucleic acid comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A major advantage of the devices and methods disclosed herein is the design of excess detector nucleic acids to total nucleic acids in an unamplified or an amplified sample, not including the nucleic acid of the detector nucleic acid. Total nucleic acids can include the target nucleic acids and non-target nucleic acids, not including the nucleic acid of the detector nucleic acid. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nuclease may be inhibited in its ability to bind and cleave the detector nucleic acid sequences. This is because the activated programmable nucleases collaterally cleaves any nucleic acids. If total nucleic acids are in present in large amounts, they may outcompete detector nucleic acids for the programmable nucleases. The devices and methods disclosed herein are designed to have an excess of detector nucleic acid to total nucleic acids, such that the detectable signals from cleavage reactions (e.g., DETECTR reactions) are particularly superior. In some embodiments, the detector nucleic acid can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

A second significant advantage of the devices and methods disclosed herein is the design of an excess volume comprising the guide nucleic acid (e.g., guide RNA), the programmable nuclease, and the detector nucleic acid, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nuclease to find and cleave the nucleic acid of the detector nucleic acid. This may be due to nucleic acids that are not the detector nucleic acid, which outcompete the nucleic acid of the detector nucleic acid, for the programmable nuclease. Alternatively, various reagents in the sample may simply inhibit the activity of the programmable nuclease. Thus, the devices and methods provided herein for contacting an excess volume comprising the guide nucleic acid (e.g., guide RNA), the programmable nuclease, and the detector nucleic acid to a smaller volume comprising the sample with the target nucleic acid of interest provides for superior detection of the target nucleic acid by ensuring that the programmable nuclease is able to find and cleaves the nucleic acid of the detector nucleic acid. In some embodiments, the volume comprising the guide nucleic acid, the programmable nuclease, and the detector nucleic acid (can be referred to as “a second volume”) is 4-fold greater than a volume comprising the sample (can be referred to as “a first volume”). In some embodiments, the volume comprising the guide nucleic acid, the programmable nuclease, and the detector nucleic acid (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 ul, at least 1 ul, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, from 0.5 μL to 5 ul μL, from 5 μL to 10 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 10 μL to 20 μL, from 5 μL to 20 μL, from 1 μL to 40 μL, from 2 μL to 10 μL, or from 1 μL to 10 μL. In some embodiments, the volume comprising the programmable nuclease, the guide nucleic acid (e.g., guide RNA), and the detector nucleic acid is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, from 10 μL to 15 ul μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 50 μL to 55 μL, from 55 μL to 60 μL, from 60 μL to 65 μL, from 65 μL to 70 μL, from 70 μL to 75 μL, from 75 μL to 80 μL, from 80 μL to 85 μL, from 85 μL to 90 μL, from 90 μL to 95 μL, from 95 μL to 100 μL, from 100 μL to 150 μL, from 150 μL to 200 μL, from 200 μL to 250 μL, from 250 μL to 300 μL, from 300 μL to 350 μL, from 350 μL to 400 μL, from 400 μL to 450 μL, from 450 μL to 500 μL, from 10 μL to 20 μL, from 10 μL to 30 μL, from 25 μL to 35 μL, from 10 μL to 40 μL, from 20 μL to 50 μL, from 18 μL to 28 μL, or from 17 μL to 22 μL.

A detector nucleic acid may be a hybrid nucleic acid detector nucleic acid. A hybrid nucleic acid detector nucleic acid comprises a nucleic acid with at least one deoxyribonucleotide and at least one ribonucleotide. In some embodiments, the nucleic acid of the hybrid nucleic acid detector nucleic acid can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid detector nucleic acid is increased stability as compared to a pure RNA nucleic acid detector nucleic acid. For example, a hybrid nucleic acid detector nucleic acid can be more stable in solution, lyophilized, or vitrified as compared to a pure DNA or pure RNA detector nucleic acid.

The detector nucleic acid can be lyophilized or vitrified. The detector nucleic acid can be suspended in solution or immobilized on a surface. For example, the detector nucleic acid can be immobilized on the surface of a chamber in a device as disclosed herein. In some cases, the detector nucleic acid is immobilized on beads, such as magnetic beads, in a chamber of a device as disclosed herein where they are held in position by a magnet placed below the chamber.

Additionally, target nucleic acid can be amplified before binding to the crRNA of the CRISPR-Cas nucleoprotein complex. This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

Disclosed herein are methods of assaying for a target nucleic acid as described herein wherein a signal is detected. For example, a method of assaying for a target nucleic acid in a sample comprises contacting the sample to a complex comprising a guide nucleic acid (e.g., guide RNA) comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid (e.g., guide RNA) and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby nucleic acids by the activated programmable nuclease, such as trans cleavage of detector nucleic acids with a detection moiety. Once the detector nucleic acid is cleaved by the activated programmable nuclease, the detection moiety can be released from the detector nucleic acid and can generate a signal. The signal can be immobilized on a support medium for detection. The signal can be visualized to assess whether a target nucleic acid comprises a modification.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorometric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid (e.g., guide RNA) and more than one type of detector nucleic acid. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single-stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, fom 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single-stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single-stranded target nucleic acid can be detected in a sample is in a range of from 1 aM to 100 pM. In some cases, the minimum concentration at which a single-stranded target nucleic acid can be detected in a sample is in a range of from 1 fM to 100 pM. In some cases, the minimum concentration at which a single-stranded target nucleic acid can be detected in a sample is in a range of from 10 fM to 100 pM. In some cases, the minimum concentration at which a single-stranded target nucleic acid can be detected in a sample is in a range of from 800 fM to 100 pM. In some cases, the minimum concentration at which a single-stranded target nucleic acid can be detected in a sample is in a range of from 1 pM to 10 pM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some cases, the devices, systems, fluidic devices, kits, and methods described herein detect a target single-stranded nucleic acid in a sample where the sample is contacted with the reagents for a predetermined length of time sufficient for the trans cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes.

When a guide nucleic acid (e.g., guide RNA) binds to a target nucleic acid, the programmable nuclease's trans cleavage activity can be initiated, and detector nucleic acids can be cleaved, resulting in the detection of fluorescence. Some methods as described herein can a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the detector nucleic acid using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single-stranded detector nucleic acid comprising a detection moiety, wherein the detector nucleic acid is capable of being cleaved by the activated programmable nuclease, thereby generating a first detectable signal, cleaving the single-stranded detector nucleic acid using the programmable nuclease that cleaves as measured by a change in color, and measuring the first detectable signal on the support medium. The cleaving of the single-stranded detector nucleic acid using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single-stranded detector nucleic acid comprising a detection moiety, wherein the detector nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

In some cases, the methods, reagents, and devices described herein detect a plurality of target nucleic acids with a programmable nuclease and a single-stranded detector nucleic acid in a sample where the sample is contacted with the reagents for a predetermined length of time sufficient for trans cleavage of the detector nucleic acid. The reagents may comprise a pool of different guide nucleic acid sequences (e.g., guide RNA sequences) directed to different segments of target nucleic acids. Each guide nucleic acid may be capable of forming a complex comprising the guide nucleic acid, a programmable nuclease, and the target nucleic acid to which the guide nucleic acid is directed. In some embodiments, a programmable nuclease is a Cas12 programmable nuclease that detects a target nucleic acid and a detector nucleic acid (e.g., a single-stranded DNA or double-stranded DNA). In some embodiments, a programmable nuclease is a Cas14 programmable nuclease that detects a target nucleic acid and a single-stranded detector nucleic acid (e.g., single-stranded DNA). In some embodiments, a programmable nuclease is a Cas13 programmable nuclease that detects a target nucleic acid and a single-stranded detector nucleic acid (e.g., a single-stranded RNA). The target nucleic acid may be a single-stranded nucleic acid (e.g., a single-stranded DNA (ssDNA) or a single-stranded RNA), or the target nucleic acid may be a double-stranded nucleic acid (e.g., a double-stranded DNA (dsDNA) or a double-stranded RNA). The detector nucleic acid may be a single-stranded nucleic acid (e.g., a ssDNA or a single-stranded RNA), or the detector nucleic acid may be a double-stranded nucleic acid (e.g., a dsDNA or a double-stranded RNA).

In some cases, the methods, reagents, and devices described herein detect a target nucleic acid with a programmable nuclease and a single-stranded detector nucleic acid in a sample where the sample is contacted with the reagents for a predetermined length of time sufficient for trans cleavage of the single-stranded detector nucleic acid. For example, a programmable nuclease is LbuCas13a that detects a target nucleic acid and a single-stranded detector nucleic acid comprises two adjacent uracil nucleotides with a green detectable moiety that is detected upon cleavage. As another example, a programmable nuclease is LbaCas13a that detects a target nucleic acid and a single-stranded detector nucleic acid comprises two adjacent adenine nucleotides with a red detectable moiety that is detected upon cleavage. The target nucleic acid may be a single-stranded nucleic acid (e.g., a single-stranded DNA (ssDNA) or a single-stranded RNA), or the target nucleic acid may be a double-stranded nucleic acid (e.g., a double-stranded DNA (dsDNA) or a double-stranded RNA).

The reagents described herein can also include buffers, which are compatible with the devices, systems, fluidic devices, kits, and methods disclosed herein. These buffers are compatible with the other reagents, samples, and support mediums as described herein for detection of an ailment, such as a disease, including those caused by viruses such as influenza. The methods described herein can also include the use of buffers, which are compatible with the methods disclosed herein. For example, a buffer comprises 20 mM HEPES pH 6.8, 50 mM KCl, 5 mM MgCl₂, and 5% glycerol. In some instances the buffer comprises from 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM HEPES pH 6.8. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer can comprise 0 to 25, 0 to 20, 0 to 10, 0 to 5, 5 to 10, 5 to 15, 5 to 20, 5 to 25, 5 to 30% glycerol.

As another example, a buffer comprises 100 mM Imidazole pH 7.5; 250 mM KCl, 25 mM MgCl₂, 50 ug/mL BSA, 0.05% Igepal Ca-630, and 25% Glycerol. In some instances the buffer comprises 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM Imidazole pH 7.5. The buffer can comprise to 0 to 500, 0 to 400, 0 to 300, 0 to 250, 0 to 200, 0 to 150, 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 5 to 150, 5 to 200, 5 to 250, 5 to 300, 5 to 400, 5 to 500, 25 to 50, 25 to 75, 25 to 100, 50 to 100, 50 150, 50 to 200, 50 to 250, 50 to 300, 100 to 200, 100 to 250, 100 to 300, or 150 to 250 mM KCl. In other instances the buffer comprises 0 to 100, 0 to 75, 0 to 50, 0 to 25, 0 to 20, 0 to 10, 0 to 5, 5 to 10, 5 to 15, 5 to 20, 5 to 25, to 30, 5 to 40, 5 to 50, 5 to 75, 5 to 100, 10 to 20, 10 to 30, 10 to 40, 10 to 50, 15 to 20, 15 to 25, 15 to 30, 15 to 4, 15 to 50, 20 to 25, 20 to 30, 20 to 40, or 20 to 50 mM MgCl₂. The buffer, in some instances, comprises 0 to 100, 0 to 75, 0 to 50, 0 to 25, 0 to 20, 0 to 10, 0 to 5, 5 to 50, 5 to 75, 5 to 100, 10 to 20, 10 to 50, 10 to 75, 10 to 100, 25 to 50, 25 to 75 25 to 100, 50 to 75, or 50 to 100 ug/mL BSA. In some instances, the buffer comprises 0 to 1, 0 to 0.5, 0 to 0.25, 0 to 0.01, 0 to 0.05, 0 to 0.025, 0 to 0.01, 0.01 to 0.025, 0.01 to 0.05, 0.01 to 0.1, 0.01 to 0.25, 0.01, to 0.5, 0.01 to 1, 0.025 to 0.05, 0.025 to 0.1, 0.025, to 0.5, 0.025 to 1, 0.05 to 0.1, 0.05 to 0.25, 0.05 to 0.5, 0.05 to 0.75, 0.05 to 1, 0.1 to 0.25, 0.1 to 0.5, or 0.1 to 1% Igepal Ca-630. The buffer can comprise 0 to 25, 0 to 20, 0 to 10, 0 to 5, 5 to 10, 5 to 15, 5 to 20, 5 to 25, 5 to 30% glycerol.

A number of detection devices and methods are consistent with methods disclosed herein. For example, any device that can measure or detect a calorimetric, potentiometric, amperometric, optical (e.g., fluorescent, colorometric, etc.), or piezo-electric signal. Often a calorimetric signal is heat produced after cleavage of the detector nucleic acids. Sometimes, a calorimetric signal is heat absorbed after cleavage of the detector nucleic acids. A potentiometric signal, for example, is electrical potential produced after cleavage of the detector nucleic acids. An amperometric signal can be movement of electrons produced after the cleavage of detector nucleic acid. Often, the signal is an optical signal, such as a colorometric signal or a fluorescence signal. An optical signal is, for example, a light output produced after the cleavage of the detector nucleic acids. Sometimes, an optical signal is a change in light absorbance between before and after the cleavage of detector nucleic acids. Often, a piezo-electric signal is a change in mass between before and after the cleavage of the detector nucleic acid. Sometimes, the detector nucleic acid is protein-nucleic acid. Often, the protein-nucleic acid is an enzyme-nucleic acid.

The results from the detection region from a completed assay can be detected and analyzed in various ways, for example, by a glucometer. In some cases, the positive control spot and the detection spot in the detection region is visible by eye, and the results can be read by the user. In some cases, the positive control spot and the detection spot in the detection region is visualized by an imaging device or other device depending on the type of signal. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the housing, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, cleanup of an environment.

### Disease Detection

Disclosed herein are methods of assaying for a plurality of target nucleic acid as described herein that can be used for disease detection. These methods are consistent for use with a pool of guide nucleic acids (e.g., guide RNAs), wherein at least two guide nucleic acid sequences of the pool of guide nucleic acids hybridizes to different segments of the same target nucleic acid or hybridizes to different segments of different target nucleic acids. method of assaying for a plurality of target nucleic acid (e.g., one or more target nucleic acid populations associated with a disease) in a sample comprises contacting the sample to a plurality of complexes comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of a target nucleic acid of the plurality of target nucleic acids; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The plurality of complexes may comprise complexes with distinct guide nucleic acids directed to different target nucleic acids. The detection of the signal can indicate the presence of the target nucleic acid. Sometimes, a target nucleic acid of the plurality of target nucleic acids comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Methods described herein can be used to identify multiple target nucleic acids from a bacteria, virus, or microbe, or any combination thereof. The multiple target nucleic acids may comprise sequence variations (e.g., mutations). The multiple target nucleic acids may be from a single target nucleic acid population associated with a disease (e.g., a single chromosome, plasmid, bacterial genome, viral genome, fungal genome, or amoeboid genome). The multiple target nucleic acids may be from multiple target nucleic acid populations (e.g., one or more of a chromosome, a plasmid, a bacterial genome, a viral genome, a fungal genome, or an amoeboid genome, or any combination thereof). The methods can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Sometimes, a status of a target nucleic acid mutation is used to determine a pathogenicity of a bacteria, virus, or microbe or treatment resistance, such as resistance to antibiotic treatment. Often, a status of a mutation is used to diagnose or identify diseases associated with the mutation of target nucleic acids in the bacteria, virus, or microbe. Often, the mutation is a single nucleotide mutation.

### Detection as a Research Tool, Point-of-Care, or Over-the-Counter

Disclosed herein are methods of assaying for a plurality of target nucleic acid (e.g., from a target population associated with a disease) as described herein that can be used as a research tool, and can be provided as reagent kits. For example, a method of assaying for a plurality of target nucleic acid in a sample comprises contacting the sample to a plurality of complexes comprising a guide nucleic acid (e.g., guide RNA) comprising a segment that is reverse complementary to a segment of a target nucleic acid of the plurality of target nucleic acids and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The plurality of complexes may comprise complexes with distinct guide nucleic acids directed to different target nucleic acids. The detection of the signal can indicate the presence of the target nucleic acid. Sometimes, a target nucleic acid of the plurality of target nucleic acids comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

The methods as described herein can be used to identify multiple target nucleic acids. The multiple target nucleic acids may comprise sequence variations (e.g., mutations). The multiple target nucleic acids may be from a single target nucleic acid populations associated with a disease (e.g., a single chromosome, plasmid, bacterial genome, viral genome, fungal genome, or amoeboid genome). The multiple target nucleic acids may be from multiple target nucleic acid populations (e.g., one or more of a chromosome, a plasmid, a bacterial genome, a viral genome, a fungal genome, or an amoeboid genome, or any combination thereof). The methods can be used to identify mutation of a target nucleic acid that affects the expression of a gene. A mutation that affects the expression of gene can be a single nucleotide mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation.

The reagent kits or research tools can be used to detect any number of target nucleic acids, mutations, or other indications disclosed herein in a laboratory setting. Reagent kits can be provided as reagent packs for open box instrumentation.

In other embodiments, any of the systems, assay formats, guide nucleic acids (e.g., guide RNAs), detector nucleic acids, programmable nucleases, or other reagents can be used in a point-of-care (POC) test, which can be carried out at a decentralized location such as a hospital, POL, or clinic. These point-of-care tests can be used to diagnose any of the indications disclosed herein, such as influenza or streptococcal infections, or can be used to measure the presence or absence of a particular mutation in a target nucleic acid (e.g., EGFR). POC tests can be provided as small instruments with a consumable test card, wherein the test card is any of the assay formats (e.g., a lateral flow assay) disclosed herein.

In still other embodiments, any of the systems, assay formats, detector nucleic acids, programmable nucleases, or other reagents can be used in an over-the-counter (OTC), readerless format, which can be used at remote sites or at home to diagnose a range of indications, such as influenza. These indications can include influenza A, influenza B, streptococcal infections, or CT/NG infections. OTC products can include a consumable test card, wherein the test card is any of the assay formats (e.g., a lateral flow assay) disclosed herein. In an OTC product, the test card can be interpreted visually or using a mobile phone.

### Multiplexing

The devices, systems, fluidic devices, kits, and methods described herein can be multiplexed in a number of ways. These methods of multiplexing are, for example, consistent with methods, reagents, and devices disclosed herein for detection of a target nucleic acid within the sample. A fluidic device may comprise multiple pumps, valves, reservoirs, and chambers for sample preparation, amplification of one or more than one sequences of target nucleic acids within the sample, mixing with a programmable nuclease, and detection of a detectable signal arising from cleavage of detector nucleic acids by the programmable nuclease within the fluidic system itself.

Methods consistent with the present disclosure include a multiplexing method of assaying for a target nucleic acid in a sample. A multiplexing method comprises contacting the sample to a complex comprising a guide nucleic acid (e.g., guide RNA) comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, multiplexing method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Multiplexing can be either spatial multiplexing wherein multiple different target nucleic acids are detected at the same time, but the reactions are spatially separated. Often, the multiple target nucleic acids are detected using the same programmable nuclease, but different guide nucleic acids (e.g., guide RNAs). The multiple target nucleic acids sometimes are detected using the different programmable nucleases. Sometimes, multiplexing can be single reaction multiplexing wherein multiple different target acids are detected in a single reaction volume. Often, a single population of programmable nucleases is used in single reaction multiplexing. Sometimes, at least two different programmable nucleases are used in single reaction multiplexing. For example, multiplexing can be enabled by immobilization of multiple categories of detector nucleic acids within a fluidic system, to enable detection of multiple target nucleic acids within a single sample. In another example, multiplexing can be enabled using a single category of detector nucleic acids in a single high-plex reaction (e.g., a reaction with a pool of guide nucleic acids (e.g., guide RNAs), wherein at least 21 guide nucleic acid sequences (e.g., guide RNA sequences) of the pool are distinct), to enable detection of the presence or absence of multiple target nucleic acids within a single sample. Multiplexing allows for detection of multiple target nucleic acids in one kit or system. In some cases, the multiple target nucleic acids comprise different target nucleic acids associated with a disease. In some cases, the multiple target nucleic acids comprise different target nucleic acids associated with a disease (e.g., a tick-borne pathogen, a healthcare-associated infection, sepsis, or a respiratory infection, such as an upper respiratory tract virus). The multiple target nucleic acids may be from the same target nucleic population associated with a single disease. The multiple target nucleic acids may be from multiple target nucleic acid populations associated with one or more diseases. Multiplexing for one disease increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. For example, multiplexing comprises method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different influenza strains, for example, influenza A and influenza B. Often, multiplexing allows for discrimination between multiple target nucleic acids, such as target nucleic acids that comprise different genotypes, for example, for a wild-type genotype and for SNP genotype. Multiplexing for multiple viral infections provides the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Multiplexing may comprise the detecting the presence or absence of any number of target nucleic acids. For example, multiplexing may comprise detecting the presence or absence of at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, or more target nucleic acids. The target nucleic acid populations may be from at least 1, at least 2, at least 3, at least 4, at least, 5, at least 6, at least 7, at least 8, at least 9, at least 10, at least 20, at least 30, at least 40, at least 50, at least 60, at least 70, at least 80, at least 90, at least 100, or more target nucleic acid populations. The target nucleic acids may be detected with at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, or more guide nucleic acids (e.g., guide RNAs). Each guide nucleic acid sequence in the plurality of guide nucleic acids may be directed to a distinct segment of a target nucleic acid or distinct segments of distinct target nucleic acids. The distinct target nucleic acids may be from a single target nucleic acid population. The distinct target nucleic acids may be from multiple target nucleic acid populations. The distinct target nucleic acids may be different variants of a target sequence from a single target nucleic acid population or multiple target nucleic acid populations. Each guide nucleic acid sequence of the pool of guide nucleic acids may be complexed with a programmable nuclease.

Furthermore, signals from multiplexing can be quantified. For example, a method of quantification for a disease panel comprises assaying for a plurality of unique target nucleic acids in a plurality of aliquots from a sample, assaying for a control nucleic acid control in a second aliquot of the sample, and quantifying a plurality of signals of the plurality of unique target nucleic acids by measuring signals produced by cleavage of detector nucleic acids compared to the signal produced in the second aliquot. Often the plurality of unique target nucleic acids are from a plurality of viruses in the sample. Sometimes the quantification of a signal of the plurality correlates with a concentration of a unique target nucleic acid of the plurality for the unique target nucleic acid of the plurality that produced the signal of the plurality. Sometimes the quantification comprises assaying for a plurality of unique target nucleic acids in a single sample and quantifying a single signal indicative of a total amount of the plurality of unique target nucleic acids.

The methods, reagents, and devices described herein can be multiplexed by various configurations of the reagents and the support medium. In some cases, the kit or system is designed to have multiple support mediums encased in a single housing. Sometimes, the multiple support mediums housed in a single housing share a single sample pad. The single sample pad may be connected to the support mediums in various designs such as a branching or a radial formation. Alternatively, each of the multiple support mediums has its own sample pad. In some cases, the kit or system is designed to have a single support medium encased in a housing, where the support medium comprises multiple detection spots for detecting multiple target nucleic acids. Sometimes, the reagents for multiplexed assays comprise multiple guide nucleic acids, multiple programmable nucleases, and multiple single stranded detector nucleic acids, where a combination of one of the guide nucleic acids, one of the programmable nucleases, and one of the single stranded detector nucleic acids detects one target nucleic acid and can provide a detection spot on the detection region. In some cases, the combination of a guide nucleic acid, a programmable nuclease, and a single stranded detector nucleic acid configured to detect one target nucleic acid is mixed with at least one other combination in a single reagent chamber. In some cases, the combination of a guide nucleic acid, a programmable nuclease, and a single stranded detector nucleic acid configured to detect one target nucleic acid is mixed with at least one other combination on a single support medium. When these combinations of reagents are contacted with the sample, the reaction for the multiple target nucleic acids occurs simultaneously in the same medium or reagent chamber. Sometimes, this reacted sample is applied to the multiplexed support medium described herein. In some cases, the methods, reagents, and devices described herein can be multiplexed in a configuration lacking a support medium.

In some cases, the combination of a guide nucleic acid, a programmable nuclease, and a single stranded detector nucleic acid configured to detect one target nucleic acid is provided in its own reagent chamber or its own support medium. In this case, multiple reagent chambers or support mediums are provided in the device, kit, or system, where one reagent chamber is designed to detect one target nucleic acid. In this case, multiple support mediums are used to detect the panel of viral infections, or other diseases of interest.

In some instances, the multiplexed methods, reagents, and devices detect at least 21 different target nucleic acids in a single reaction. In some instances, the multiplexed methods, reagents, and devices detect at least 30 different target nucleic acids in a single reaction. In some instances, the multiplexed methods, reagents, and devices detect at least 40 different target nucleic acids in a single reaction. In some instances, the multiplexed methods, reagents, and devices detect at least 50 different target nucleic acids in a single reaction. In some cases, the multiplexed methods, reagents, and devices detect at least 60, 70, 80, 90, or 100 different target nucleic acids in a single reaction. In some cases, the multiplexed methods, reagents, and devices detect at least 200, 300, 400, 500, 600, 700, 800, 900, or 1000 different target nucleic acids in a single reaction. In some instances, the multiplexed kits detect at least 20 different target nucleic acids in a single kit. In some instances, the multiplexed kits detect at least 30 different target nucleic acids in a single kit. In some instances, the multiplexed kits detect at least 40 different target nucleic acids in a single kit. In some instances, the multiplexed kits detect at least 50 different target nucleic acids in a single kit. In some instances, the multiplexed kits detect at least 60, 70, 80, 90, or 100 different target nucleic acids in a single kit. In some instances, the multiplexed kits detect at least 200, 300, 400, 500, 600, 700, 800, 900, or 1000 different target nucleic acids in a single kit.

### Detection of a Target Nucleic Acid in a Fluidic Device

Disclosed herein are various fluidic devices for detection of a target nucleic acid of interest in a biological sample. The fluidic devices described in detail below can be used to monitor the reaction of target nucleic acids in samples with a programmable nuclease, thereby allowing for the detection of said target nucleic acid. All samples and reagents disclosed herein are compatible for use with a fluidic device disclosed below. Any programmable nuclease, such as any Cas nuclease described herein, are compatible for use with a fluidic device disclosed below. Support mediums and housing disclosed herein are also compatible for use in conjunction with the fluidic devices disclosed below. Multiplexing detection, as described throughout the present disclosure, can be carried out within the fluidic devices disclosed herein. Compositions and methods for detection and visualization disclosed herein are also compatible for use within the below described fluidic systems.

In the below described fluidic systems, any programmable nuclease (e.g., a Cas enzyme) reaction can be monitored. For example, any programmable nuclease disclosed herein can be used to cleave the detector nucleic acids to generate a detection signal. In some cases, the programmable nuclease is Cas13. Sometimes the Cas13 is Cas13a, Cas13b, Cas13c, Cas13d, or Cas13e. In some cases, the programmable nuclease is Mad7 or Mad2. In some cases, the programmable nuclease is Cas12. Sometimes the Cas12 is Cas12a, Cas12b, Cas12c, Cas12d, or Cas12e. In some cases, the programmable nuclease is Csm1, Cas9, C2c4, C2c8, C2c5, C2c10, C2c9, or CasZ. Sometimes, the Csm1 is also called smCms1, miCms1, obCms1, or suCms1. Sometimes Cas13a is also called C2c2. Sometimes CasZ is also called Cas14a, Cas14b, Cas14c, Cas14d, Cas14e, Cas14f, Cas14g, or Cas14h. Sometimes, the programmable nuclease is a Type V CRISPR-Cas enzyme. In some cases, the programmable nuclease is a Type VI CRISPR-Cas enzyme. Sometimes the programmable nuclease is a Type III CRISPR-Cas enzyme. In some cases, the programmable nuclease is from at least one of Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

A workflow of a method for detecting a target nucleic acid in a sample within a fluidic device can include sample preparation, nucleic acid amplification, incubation with a programmable nuclease, and/or detection (readout). An exemplary workflow of a programmable nuclease reaction includes: Step 1—sample preparation; Step 2—nucleic acid amplification; Step 3—programmable nuclease incubation; and Step 4—detection (readout). Steps 1 and 2 are optional, and steps 3 and 4 can occur concurrently, if incubation and detection of programmable nuclease activity are within the same chamber. Sample preparation and amplification can be carried out within a fluidic device described herein or, alternatively, can be carried out prior to introduction into the fluidic device. As mentioned above, sample preparation of any nucleic acid amplification are optional, and can be excluded. In further cases, programmable nuclease reaction incubation and detection (readout) can be performed sequentially (one after another) or concurrently (at the same time). In some embodiments, sample preparation and/or amplification can be performed within a first fluidic device and then the sample can be transferred to a second fluidic device to carry out Steps 3 and 4 and, optionally, Step 2.

Workflows and systems compatible with the compositions and methods provided herein include one-pot reactions and two-pot reactions. In a one-pot reaction, amplification, reverse transcription, amplification and reverse transcription, or amplification and in vitro transcription, and detection can be carried out simultaneously in one chamber. In other words, in a one-pot reaction, any combination of reverse transcription, amplification, and in vitro transcription can be performed in the same reaction as detection. In a two-pot reaction, any combination of reverse transcription, amplification, and in vitro transcription can be performed in a first reaction, followed by detection in a second reaction. The one-pot or two-pot reactions can be carried out in any of the chambers of the devices disclosed herein.

A fluidic device for sample preparation can be referred to as a filtration device. In some embodiments, the filtration device for sample preparation resembles a syringe or, comprises, similar functional elements to a syringe. For example, a functional element of the filtration device for sample preparation includes a narrow tip for collection of liquid samples. Liquid samples can include blood, saliva, urine, or any other biological fluid. Liquid samples can also include liquid tissue homogenates. The tip, for collection of liquid samples, can be manufactured from glass, metal, plastic, or other biocompatible materials. The tip may be replaced with a glass capillary that may serve as a metering apparatus for the amount of biological sample added downstream to the fluidic device. For some samples, e.g., blood, the capillary may be the only fluidic device required for sample preparation. Another functional element of the filtration device for sample preparation may include a channel that can carry volumes from nL to mL, containing lysis buffers compatible with the programmable nuclease reaction downstream of this process. The channel may be manufactured from metal, plastic, or other biocompatible materials. The channel may be large enough to hold an entire fecal, buccal, or other biological sample collection swab. The filtration device may further contain a solution of reagents that will lyse the cells in each type of samples and release the nucleic acids so that they are accessible to the programmable nuclease. Active ingredients of the solution may be chaotropic agents, detergents, salts, and can be of high osmolality, ionic strength and pH. Chaotropic agents or chaotropes are substances that disrupt the three-dimensional structure in macromolecules such as proteins, DNA, or RNA. One example protocol comprises a 4 M guanidinium isothiocyanate, 25 mM sodium citrate·2H₂O, 0.5% (w/v) sodium lauryl sarcosinate, and 0.1 M β-mercaptoethanol), but numerous commercial buffers for different cellular targets may also be used. Alkaline buffers may also be used for cells with hard shells, particularly for environmental samples. Detergents such as sodium dodecyl sulphate (SDS) and cetyl trimethylammonium bromide (CTAB) may also be implemented to chemical lysis buffers. Cell lysis may also be performed by physical, mechanical, thermal or enzymatic means, in addition to chemically-induced cell lysis mentioned previously. The device may include more complex architecture depending on the type of sample, such as nanoscale barbs, nanowires, sonication capability in a separate chamber of the device, integrated laser, integrated heater, for example, a Peltier-type heater, or a thin-film planar heater, and/or microcapillary probes for electrical lysis. Any samples described herein can be used in this workflow. For example samples may include liquid samples collected from a subject being tested for a condition of interest. A fluidic, or filtration, device for sample preparation may be used for Step 1 of a workflow, as described above. A sample preparation fluidic device can process different types of biological sample: finger-prick blood, urine or swabs with fecal, cheek or other collection.

A fluidic device may be used to carry out any one of, or any combination of, Steps 2-4 (nucleic acid amplification, programmable nuclease reaction incubation, detection (readout)), as described above. Several variations of the fluidic device are consistent with the present disclosure. For example, fluidic devices can be compatible with a fluorescence or electrochemical readout that may be used in Step 2 to Step 4 of the workflow. Devices can perform three iterations of Steps 2 through 4 of the workflow. In one variation a this fluidic device, the programmable nuclease reaction, incubation, and detection (readout) steps are carried out, but not amplification. In another variation of said fluidic device, the device comprises a one-chamber reaction with amplification. In yet another variation of the fluidic device, the device comprises a two-chamber reaction with amplification.

In some embodiments, the fluidic device may be a pneumatic device. The pneumatic device may comprise one or more sample chambers connected to one or more detection chambers by one or more pneumatic valves. Optionally, the pneumatic device may further comprise one or more amplification chamber between the one or more sample chambers and the one or more detection chambers. The one or more amplification chambers may be connected to the one or more sample chambers and the one or more detection chambers by one or more pneumatic valves. A pneumatic valve may be made from PDMS, or any other suitable material. A pneumatic valve may comprise a channel perpendicular to a microfluidic channel connecting the chambers and allowing fluid to pass between chambers when the valve is open. In some embodiments, the channel deflects downward upon application of air pressure through the channel perpendicular to the microfluidic channel.

In some embodiments, the fluidic device may be a sliding valve device. The sliding valve device may comprise a sliding layer with one or more channels and a fixed layer with one or more sample chambers and one or more detection chambers. Optionally, the fixed layer may further comprise one or more amplification chambers. In some embodiments, the sliding layer is the upper layer and the fixed layer is the lower layer. In other embodiments, the sliding layer is the lower layer and the fixed layer is the upper layer. The sliding valve device may further comprise one or more of a side channel with an opening aligned with an opening in the sample chamber, a side channel with an opening aligned with an opening in the amplification chamber, or a side channel with an opening aligned with the opening in the detection chamber. In some embodiments the side channels are connected to a mixing chamber to allow transfer of fluid between the chambers. In some embodiments, the sliding valve device comprises a pneumatic pump for mixing, aspirating, and dispensing fluid in the device.

The chip (also referred to as fluidic device) may be manufactured from a variety of different materials. Exemplary materials that may be used include plastic polymers, such as poly-methacrylate (PMMA), cyclic olefin polymer (COP), cyclic olefin copolymer (COC), polyethylene (PE), high-density polyethylene (HDPE), polypropylene (PP); glass; and silicon. Features of the chip may be manufactured by various processes. For example, features may be (1) embossed using injection molding, (2) micro-milled or micro-engraved using computer numerical control (CNC) micromachining, or non-contact laser drilling (by means of a C02 laser source); (3) additive manufacturing, and/or (4) photolithographic methods.

The design may include up to three (3) input ports operated by three (3) pumps. The pumps may be operated by external syringe pumps using low pressure or high pressure. The pumps may be passive, and/or active (pneumatic, piezoelectric, Braille pin, electroosmotic, acoustic, gas permeation, or other).

The ports may be connected to pneumatic pressure pumps, air or gas may be pumped into the microfluidic channels to control the injection of fluids into the fluidic device. At least three reservoirs may be connected to the device, each containing buffered solutions of: (1) sample, which may be a solution containing purified nucleic acids processed in a separate fluidic device, or neat sample (blood, saliva, urine, stool, and/or sputum); (2) amplification mastermix, which varies depending on the method used, wherein the method may include any of loop-mediated isothermal amplification (LAMP), strand displacement amplification (SDA), recombinase polymerase amplification (RPA), helicase dependent amplification (HDA), multiple displacement amplification (MDA), rolling circle amplification (RCA), and nucleic acid sequence-based amplification (NASBA), transcription mediated amplification (TMA), circular helicase dependent amplification (cHDA), exponential amplification reaction (EXPAR), ligase chain reaction (LCR), simple method amplifying RNA targets (SMART), single primer isothermal amplification (SPIA), hinge-initiated primer-dependent amplification of nucleic acids (HIP), nicking enzyme amplification reaction (NEAR), or improved multiple displacement amplification (IMDA); and (3) pre-complexed programmable nuclease mix, which includes one or more programmable nuclease and guide oligonucleotides. The method of nucleic acid amplification may also be polymerase chain reaction (PCR), which includes cycling of the incubation temperature at different levels, hence is not defined as isothermal. Often, the reagents for nucleic acid amplification comprise a recombinase, a oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively or in combination, the nucleic acid amplification is performed in a reagent chamber, and the resulting sample is applied to the support medium. Sometimes, the nucleic acid amplification is isothermal nucleic acid amplification. Complex formation of a programmable nuclease with guides and detector nucleic acids may occur off the chip. An additional port for output of the final reaction products is depicted at the end of the fluidic path, and is operated by a similar pump, as the ones described for P1-P3. The reactions product can be, thus, collected for additional processing and/or characterization, e.g., sequencing.

The flow of liquid in this fluidic device may be controlled using up to four (4) microvalves. These valves can be electro-kinetic microvalves, pneumatic microvalves, vacuum microvalves, capillary microvalves, pinch microvalves, phase-change microvalves, burst microvalves.

The flow to and from the fluidic channel from each of P1-P4 is controlled by valves, labelled as V1-V4. The volume of liquids pumped into the ports can vary from nL to mL depending in the overall size of the device.

In some fluidic devices, no amplification is needed. After addition of sample and pre-complexed programmable nuclease mix in P1 and P2, respectively, the reagents may be mixed in the serpentine channel, S1, which then leads to chamber C1 where the mixture may be incubated at the required temperature and time. The readout can be done simultaneously in C1. Thermoregulation in C1 may be carried out using a thin-film planar heater manufactured, from e.g. Kapton, or other similar materials, and controlled by a proportional integral derivative (PID).

In some fluidic devices, after addition of sample, amplification mix, and pre-complexed programmable nuclease mix in P1, P2 and P3, respectively, the reagents can be mixed in the serpentine channel, S1, which then leads to chamber C1 where the mixture is incubated at the required temperature and time needed to efficient amplification, as per the conditions of the method used. The readout may be done simultaneously in C1. Thermoregulation may be achieved as previously described.

In some fluidic devices, amplification and programmable nuclease reactions occur in separate chambers. The pre-complexed programmable nuclease mix is pumped into the amplified mixture from C1 using pump P3. The liquid flow is controlled by valve V3, and directed into serpentine mixer S2, and subsequently in chamber C2 for incubation the required temperature, for example at 37° C. for 90 minutes.

During the detection step, the Cas-gRNA complex can bind to its matching nucleic acid target from the amplified sample and is activated into a non-specific nuclease, which cleaves a detector nucleic acid to generate a signal readout. In the absence of a matching nucleic acid target, the Cas-gRNA complex does not cleave the detector nucleic acid. Real-time detection of the Cas reaction can be achieved by three methods: (1) fluorescence, (2) electrochemical detection, and (3) electrochemiluminescence. Detection of the signal can be achieved by multiple methods, which can detect a signal that is calorimetric, potentiometric, amperometric, optical (e.g., fluorescent, colorometric, etc.), or piezo-electric, as non-limiting examples.

Readout processes that can be used in conjunction with a fluidic device of the present disclosure includes (a) fluorescence readout and (b) electrochemical readout. The emitted fluorescence of cleaved detector nucleic acids may be monitored using a fluorimeter positioned directly above the detection and incubation chamber. The fluorimeter may be a commercially available instrument, the optical sensor of a mobile phone or smart phone, or a custom-made optical array comprising of fluorescence excitation means, e.g. CO2, other, laser and/or light emitting diodes (LEDs), and fluorescence detection means e.g. photodiode array, phototransistor, or others.

The fluorescence detection and excitation may be multiplexed, wherein, for example, fluorescence detection involves exciting and detecting more than one fluorophore in the incubation and detection chamber (C1 or C2). The fluorimeter itself may be multichannel, in which detecting and exciting light at different wavelengths, or more than one fluorimeter may be used in tandem, and their position above the incubation and detection chamber (C1 and C2) be modified by mechanical means, such as a motorized mechanism using micro or macro controllers and actuators (electric, electronic, and/or piezo-electric).

Two electrochemical detection variations are described herein, using integrated working, counter and reference electrodes in the incubation and detection chamber (C1 or C2):

### Increase in Signal

The progress of the cleavage reaction catalyzed by the programmable nuclease may be detected using a streptavidin-biotin coupled reaction. The top surface of the detection and incubation chamber may be functionalized with nucleic acid molecules (ssRNA, ssDNA or ssRNA/DNA hybrid molecules) conjugated with a biotin moiety. The bottom surface of the detection and incubation chamber operates as an electrode, comprising of working, reference, and counter areas, manufactured (or screen-printed) from carbon, graphene, silver, gold, platinum, boron-doped diamond, copper, bismuth, titanium, antimony, chromium, nickel, tin, aluminum, molybdenum, lead, tantalum, tungsten, steel, carbon steel, cobalt, indium tin oxide (ITO), ruthenium oxide, palladium, silver-coated copper, carbon nano-tubes, or other metals. The bottom surface of the detection and incubation chamber may be coated with streptavidin molecules. In the absence of any biotin molecules, the current measured by a connected electrochemical analyzer (commercial, or custom-made) is low. When the pre-complexed programmable nuclease mix with amplified target flows in the detection and incubation chamber, and is activated at a higher temperature, for example at 37° C., cleavage of the single-stranded nucleic acid (ssNA) linker releases biotin molecules that can diffuse onto the streptavidin-coated bottom surface of the detection and incubation chamber. Because of the interaction of biotin and streptavidin molecules, an increase in the current is read by a coupled electrochemical analyzer.

Other types of signal amplification that use enrichment may also be used apart from biotin-streptavidin excitation. Non-limiting examples are: (1) glutathione, glutathione S-transferase, (2) maltose, maltose-binding protein, (3) chitin, chitin-binding protein.

### Decrease in Signal

The progress of the programmable nuclease cleavage reaction may be monitored by recording the decrease in the current produced by a ferrocene (Fc), or other electroactive mediator moieties, conjugated to the individual nucleotides of nucleic acid molecules (ssRNA, ssDNA or ssRNA/DNA hybrid molecules) immobilized on the bottom surface of the detection and incubation chamber. In the absence of the amplified target, the programmable nuclease complex remains inactive, and a high current caused by the electroactive moieties is recorded. When the programmable nuclease complex with guides flows in the detection and incubation chamber and is activated by the matching nucleic acid target at 37° C., the programmable nuclease complex non-specifically degrades the immobilized Fc-conjugated nucleic acid molecules. This cleavage reaction decreases the number of electroactive molecules and, thus, leads to a decrease in recorded current.

The electrochemical detection may also be multiplexed. This is achieved by the addition of one or more working electrodes in the incubation and detection chamber (C1 or C2). The electrodes can be plain, or modified, as described above for the single electrochemical detection method.

### Electrochemiluminescence in a Combined Optical and Electrochemical Readout Method

The optical signal may be produced by luminescence of a compound, such as tri-propyl amine (TPA) generated as an oxidation product of an electroactive product, such as ruthenium bipyridine, [Ru (py)3]2+.

A number of different programmable nucleases may be multiplexed by: (1) separate fluidic paths (parallelization of channels), mixed with the same sample, for each of the nucleases, or (2) switching to digital (two-phase) microfluidics, where each individual droplet contains a separate reaction mix. The droplets could be generated from single or double emulsions of water and oil. The emulsions are compatible with programmable nuclease reaction, and optically inert.

In another example of a fluidic device consistent with the present disclosure, the device can be configured for coupled invertase/Cas reactions with colorimetric or electrochemical/glucometer readout. This diagram illustrates a fluidic device for miniaturizing a Cas reaction coupled with the enzyme invertase. Surface modification and readout processes are depicted in exploded view schemes at the bottom including (a) optical readout using DNS, or other compound and (b) electrochemical readout (electrochemical analyzer or glucometer). Described herein is the coupling of the Cas reaction with the enzyme invertase (EC 3.2.1.26), or sucrase or β-fructofuranosidase. This enzyme catalyzes the breakdown of sucrose to fructose and glucose.

The following methods may be used to couple the readout of the Cas reaction to invertase activity:

### Colorimetry Using a Camera, Standalone, or an Integrated Mobile Phone Optical Sensor

The amount of fructose and glucose is linked to a colorimetric reaction. Two examples are: (a) 3,5-Dinitrosalicylic acid (DNS), and (b) formazan dye thiazolyl blue. The color change can be monitored using a CCD camera, or the image sensor of a mobile phone. For this method, a variation of the device configured for coupled invertase/Cas reactions with colorimetric or electrochemical/glucometer readout can be used. The modification is the use of a camera, instead of a fluorimeter above C3.

### Amperometry Using a Conventional Glucometer, or an Electrochemical Analyzer

In another example variation of a fluidic device, the device comprises the addition of one more incubation chamber C3. An additional step is added to the reaction scheme, which takes place in chamber C2. The top of the chamber surface is coated with single stranded nucleic acid that is conjugated to the enzyme invertase (Inv). The target-activated programmable nuclease complex cleaves the invertase enzyme from the oligo (ssRNA, ssDNA or ssRNA/DNA hybrid molecule), in C2, and invertase is then available to catalyze the hydrolysis of sucrose injected by pump P4, and controlled by valve V4. The mixture is mixed in serpentine mixer S3, and at chamber C3, the glucose produced may be detected colorimetrically, as previously described, electrochemically. The enzyme glucose oxidase is dried on the surface on C3, and catalyzes the oxidation of glucose to hydrogen peroxide and D-glucono-6-lactone.

A number of different devices are compatible with detection of target nucleic acids using the methods and compositions disclosed herein. In some embodiments, the device is any of the microfluidic devices disclosed herein. In other embodiments, the device is a lateral flow test strip connected to a reaction chamber. In further embodiments, the lateral flow strip may be connected to a sample preparation device.

In some embodiments, the fluidic device may be a pneumatic device. The pneumatic device may comprise one or more sample chambers connected to one or more detection chambers by one or more pneumatic valves. Optionally, the pneumatic device may further comprise one or more amplification chamber between the one or more sample chambers and the one or more detection chambers. The one or more amplification chambers may be connected to the one or more sample chambers and the one or more detection chambers by one or more pneumatic valves. A pneumatic valve may be made from PDMS, or any other suitable material. A pneumatic valve may comprise a channel perpendicular to a microfluidic channel connecting the chambers and allowing fluid to pass between chambers when the valve is open. In some embodiments, the channel deflects downward upon application of air pressure through the channel perpendicular to the microfluidic channel.

In some embodiments, the fluidic device may be a sliding valve device. The sliding valve device may comprise a sliding layer with one or more channels and a fixed layer with one or more sample chambers and one or more detection chambers. Optionally, the fixed layer may further comprise one or more amplification chambers. In some embodiments, the sliding layer is the upper layer and the fixed layer is the lower layer. In other embodiments, the sliding layer is the lower layer and the fixed layer is the upper layer. In some embodiments, the upper layer is made of a plastic polymer comprising poly-methacrylate (PMMA), cyclic olefin polymer (COP), cyclic olefin copolymer (COC), polyethylene (PE), high-density polyethylene (HDPE), polypropylene (PP); a glass; or a silicon. In some embodiments, the lower layer is made of a plastic polymer comprising poly-methacrylate (PMMA), cyclic olefin polymer (COP), cyclic olefin copolymer (COC), polyethylene (PE), high-density polyethylene (HDPE), polypropylene (PP); a glass; or a silicon. The sliding valve device may further comprise one or more of a side channel with an opening aligned with an opening in the sample chamber, a side channel with an opening aligned with an opening in the amplification chamber, or a side channel with an opening aligned with the opening in the detection chamber. In some embodiments the side channels are connected to a mixing chamber to allow transfer of fluid between the chambers. In some embodiments, the sliding valve device comprises a pneumatic pump for mixing, aspirating, and dispensing fluid in the device.

### Pneumatic Valve Device

A microfluidic device particularly well suited for carrying out the DETECTR reactions described herein (e.g., multiplexed DETECTR reactions or high-plex DETECTR reactions) is one comprising a pneumatic valve, also referred to as a “quake valve”. The pneumatic valve can be closed and opened by the flow of air from, for an example, an air manifold. The opening of the pneumatic valve can lead to a downward deflection of the channel comprising the pneumatic valve, which can subsequently deflect downwards and seal off a microfluidic channel beneath the channel comprising the pneumatic valve. This can lead to stoppage of fluid flow in the microfluidic channel. When the air manifold is turned off, the flow of air through the channel comprising the quake valve ceases and the microfluidic channel beneath the channel comprising the quake valve is “open”, and fluid can flow through. In some embodiments, the channel comprising the pneumatic valve may be above or below the microfluidic channel carrying the fluid of interest. In some embodiments, the channel comprising the pneumatic valve can be parallel or perpendicular to the microfluidic channel carrying the fluid of interest. Pneumatic valves can be made of a two hard thermoplastic layers sandwiching a soft silicone layer.

One example layout is as follows. In some embodiments, the device comprises a sample chamber and a detection chamber, wherein the detection chamber is fluidically connected to the sample chamber by a pneumatic valve and wherein the detection chamber comprises any programmable nuclease of the present disclosure. Optionally, the device can also include an amplification chamber that is between the fluidic path from the sample chamber to the detection chamber, is connected to the sample chamber by a pneumatic valve, and is additionally connected to the detection chamber by a pneumatic valve. In some embodiments, the pneumatic valve is made of PDMS, or any other material for forming microfluidic valves. In some embodiments, the sample chamber has a port for inserting a sample. The sample can be inserted using a swab. The sample chamber can have a buffer for lysing the sample. The sample chamber can have a filter between the chamber and the fluidic channel to the amplification or detection chambers. The sample chamber may have an opening for insertion of a sample. A sample can be incubated in the sample chamber for from 30 seconds to 10 minutes. The air manifold may until this point be on, pushing air through the pneumatic valve and keeping the fluidic channel between the sample chamber and the amplification or detection chambers closed. At this stage, the air manifold can be turned off, such that no air is passing through the pneumatic valve, and allowing the microfluidic channel to open up and allow for fluid flow from the sample chamber to the next chamber (e.g., the amplification or detection chambers). In devices where there is an amplification chamber, the lysed sample flows from the sample chamber into the amplification chamber. Otherwise, the lysed sample flows from the sample chamber into the detection chamber. At this stage, the air manifold is turned back on, to push air through the pneumatic valve and seal the microfluidic channel. The amplification chamber holds various reagents for amplification and, optionally, reverse transcription of a target nucleic acid in the sample. These reagents may include forward and reverse primers, a deoxynucleotide triphosphate, a reverse transcriptase, a T7 promoter, a T7 polymerase, or any combination thereof. The sample is allowed to incubate in the amplification chamber for from 5 minutes to 40 minutes. The amplified and, optionally reverse transcribed, sample is moved into the detection chamber as described above: the air manifold is turned off, ceasing air flow through the pneumatic valve and opening the microfluidic channel. The detection chamber can include any programmable nuclease disclosed herein, a guide nucleic acid (e.g., a guide RNA) with a portion reverse complementary to a portion of the target nucleic acid, and any detector nucleic acid disclosed herein. In some embodiments, the detection chamber may comprise a plurality of guide RNAs. The plurality of guide RNAs may have the same sequence, or one or more of the plurality of guide RNAs may have different sequences. In some embodiments, the plurality of guide RNAs has a portion reverse complementary to a portion of a target nucleic acid different than a second RNA of the plurality of guide RNAs. The plurality of guide RNAs may comprise at least 5, at least 10, at least 15, at least 20, or at least 50 guide RNAs. Once the sample is moved into the detection chamber, the DETECTR reaction can be carried out for 1 minute to 20 minutes. Upon hybridization of the guide RNA to the target nucleic acid, the programmable nuclease is activated and begins to collaterally cleave the detector nucleic acid, which as described elsewhere in this disclosure has a nucleic acid and one or more molecules that enable detection of cleavage. The detection chamber can interface with a device for reading out for the signal. For example, in the case of a colorimetric or fluorescence signal generated upon cleavage, the detection chamber may be coupled to a spectrophotometer or fluorescence reader. In the case where an electrochemical signal is generated, the detection chamber may have one to 10 metal leads connected to a readout device (e.g., a glucometer). The top layer of a cartridge of a pneumatic valve device of the present disclosure can have dimensions of 2 inches by 1.5 inches. In a modification of a top layer of a cartridge of a pneumatic valve device of the present disclosure, the cartridge can be adapted for electrochemical detection. In this device, wiring (or “metal leads”, which are co-molded, 3D-printed, or manually assembled in the disposable cartridge to form a three-electrode system can in the detection chambers Electrodes are termed as working, counter, and reference. Electrodes can also be screen printed on the cartridges. Metals used can be carbon, gold, platinum, or silver. A major advantage of the pneumatic valve device is that the pneumatic valves connecting the various chambers of the device prevent backflow from chamber to chamber, which reduces contamination. Prevention of backflow and preventing sample contamination is especially important for the applications described herein. Sample contamination can result in false positives or can generally confound the limit of detection for a target nucleic acid. As another example, the pneumatic valves disclosed herein are particularly advantageous for devices and methods for multiplex detection. In multiplexed assays, where two or more target nucleic acids are assayed for, it is particularly important that backflow and contamination is avoided. Backflow between chambers in a multiplexed assay can lead to cross-contamination of different guide nucleic acids or different programmable nuclease and can result in false results. Thus, the pneumatic valve device, which is designed to minimize or entirely avoid backflow, is particularly superior, in comparison to other device layouts, for carrying out the detection methods disclosed herein.

In one variation, a device consistent with the compositions and methods disclosed herein can have a layout comprising a quake valve pneumatic pump configured for a DETECTR assay. A pipette pump can aspirate and dispense samples. An air manifold can be connected to a pneumatic pump to open and close the normally closed valve. The pneumatic device can move fluid from one position to the next. The pneumatic design can have reduced channel cross talk compared to other device designs. A cartridge can be adapted for use in the pneumatic valve device. The normally closed valves can comprise an elastomeric seal on top of the channel to isolate each chamber from the rest of the system when the chamber is not in use. The pneumatic pump uses air to open and close the valve as needed to move fluid to the necessary chambers within the cartridge. A sample can be placed in the sample well while all valves are closed. The sample can be lysed in the sample well. The lysed sample can be moved from the sample chamber to a second chamber by opening the first quake valve, and the sample can be aspirated using the pipette pump. The sample can then be moved to the first amplification chamber by closing the first quake valve and opening a second quake valve where it is mixed with the amplification mixture. After the sample is mixed with the amplification mixture, it can be moved to a subsequent chamber by closing the second quake valve and opening a third quake valve. The sample can be moved to the DETECTR chamber by closing the third quake valve and opening a fourth quake valve. The sample can be moved through a different series of chambers by opening and closing a different series of quake valves. Actuation of individual valves in the desired chamber series prevents cross contamination between channels. In some embodiments the sliding valve device has a surface area of 5 cm by 5 cm, 5 by 6 cm, 6 by 7 cm, 7 by 8 cm, 8 by 9 cm, 9 by 10 cm, 10 by 11 cm, 11 by 12 cm, 6 by 9 cm, 7 by 10 cm, 8 by 11 cm, 9 by 12 cm, 10 by 13 cm, 11 by 14 cm, 12 by 11 cm, about 30 sq cm, about 35 sq cm, about 40 sq cm, about 45 sq cm, about 50 sq cm, about 55 sq cm, about 60 sq cm, about 65 sq cm, about 70 sq cm, about 75 sq cm, about 25 sq cm, about 20 sq cm, about 15 sq cm, about 10 sq cm, about 5 sq cm, from 1 to 100 sq cm, from 5 to 10 sq cm, from 10 to 15 sq cm, from 15 to 20 sq cm, from 20 to 25 sq cm, from 25 to 30 sq cm, from 30 to 35 sq cm, from 35 to 40 sq cm, from 40 to 45 sq cm, from 45 to 50 sq cm, from 5 to 90 sq cm, from 10 to 0 sq cm, from 15 to 5 sq cm, from 20 to 10 sq cm, or from 25 to 15 sq cm.

### Sliding Valve Device

A microfluidic device particularly well suited for carrying out the DETECTR reactions described herein (e.g., multiplexed DETECTR reactions or high-plex DETECTR reactions) is a sliding valve device. The sliding valve device can have a sliding layer and a fixed layer. The sliding layer may be on top and the fixed layer may be on bottom. Alternatively, the sliding layer may be on bottom and the fixed layer may be on top. In some embodiments, the sliding valve has a channel. The channel can have an opening at one end that interacts with an opening in a chamber and the channel can also have an opening at the other end that interacts with an opening in a side channel. In some embodiments, the sliding layer has more than one opening. In some embodiments, the fixed layer comprises a sample chamber, an amplification chamber, and a detection chamber. The sample chamber, the amplification chamber, and the detection layer can all have an opening at the bottom of the chambers. For example, the sample chamber may have an opening for insertion of a sample. When the opening in a chamber is aligned with the opening in a channel, fluid can flow from the chamber into the channel. Further, when the opening in the channel is subsequently aligned with an opening in a side channel, fluid can flow from the channel into the side channel. The side channel can be further fluidically connected to a mixing chamber, or a port in which an instrument (e.g., a pipette pump) for mixing fluid is inserted. Alignment of openings can be enabled by physically moving or automatically actuating the sliding layer to slide along the length of the fixed layer. In some embodiment, the above described pneumatic valves can be added at any position to the sliding valve device in order to control the flow of fluid from one chamber into the next. The sliding valve device can also have multiple layers. For example, the sliding valve can have 2, 3, 4, 5, 6, 7, 8, 9, 10, or more layers.

In one layout of a device for a DETECTR assay, at top is a pneumatic pump, which interfaces with a cartridge. At middle, if observing from a top down view of the cartridge, is a layer with reservoirs. At bottom is a sliding valve containing the sample, which can flow into a a lysis chamber positioned to the left of the sample, following by amplification chambers to the right, and detection chambers further to the right. Offset pitches of the channels can allow for aspirating and dispensing into each well separately and helps to mitigate cross talk between the amplification chambers and corresponding chambers. In an initial closed position, the sample can be loaded into the sample well and lysed. The sliding valve can then be actuated by the instrument, and samples can be loaded into each of the channels using the pipette pump, which dispenses the appropriate volume into the channel. The sample can be delivered to the amplification chambers by actuating the sliding valve and mixed with the pipette pump. Samples from the amplification chamber can be aspirated into each channel and then dispensed and mixed into each DETECTR chamber by actuating the sliding valve and pipette pump. In some embodiments the sliding valve device can have a surface area of 5 cm by 8 cm, 5 by 6 cm, 6 by 7 cm, 7 by 8 cm, 8 by 9 cm, 9 by 10 cm, 10 by 11 cm, 11 by 12 cm, 6 by 9 cm, 7 by 10 cm, 8 by 11 cm, 9 by 12 cm, 10 by 13 cm, 11 by 14 cm, 12 by 11 cm, about 30 sq cm, about 35 sq cm, about 40 sq cm, about 45 sq cm, about 50 sq cm, about 55 sq cm, about 60 sq cm, about 65 sq cm, about 70 sq cm, about 75 sq cm, about 25 sq cm, about 20 sq cm, about 15 sq cm, about 10 sq cm, about 5 sq cm, from 1 to 100 sq cm, from 5 to 10 sq cm, from 10 to 15 sq cm, from 15 to 20 sq cm, from 20 to 25 sq cm, from 25 to 30 sq cm, from 30 to 35 sq cm, from 35 to 40 sq cm, from 40 to 45 sq cm, from 45 to 50 sq cm, from 5 to 90 sq cm, from 10 to 0 sq cm, from 15 to 5 sq cm, from 20 to 10 sq cm, or from 25 to 15 sq cm.

### Lateral Flow Devices

In some embodiments, a device of the present disclosure comprises a chamber and a lateral flow strip. Lateral flow strips can be used in the DETECTR assay methods disclosed herein. Detector nucleic acids of the present disclosure can comprise a DNA linker linked to a biotin-dT bound to a FAM molecule. Milenia HybridDetect lateral flow strips can be used with the modified detector nucleic acids disclosed herein. This particular layout improves the test result by generating higher signal in the case of a positive result, while also minimizing false positives. In this assay layout, the detector nucleic acid comprises a biotin and a fluorophore attached at one of a nucleic acid. The nucleic acid can be conjugated directly to the biotin molecule and then the fluorophore or directly to the fluorophore and then to the biotin. Other affinity molecules, including those described herein can be used instead of biotin. Any of the fluorophores disclosed herein can also be used in the detector nucleic acid. The detector nucleic acid can be suspended in solution or immobilized on the surface of the Cas chamber. Alternatively, the detector nucleic acid can be immobilized on beads, such as magnetic beads, in the reaction chamber where they are held in position by a magnet placed below the chamber. When the detector nucleic acid is cleaved by an activated programmable nuclease, the cleaved biotin-fluorophore accumulates at the first line, which comprises a streptavidin (or another capture molecule). Gold nanoparticles, which are on the sample pad and flown onto the strip using a chase buffer, are coated with an anti-fluorophore antibody allowing binding and accumulation of the gold nanoparticle at the first line. The nanoparticles additionally accumulate at a second line which is coated with an antibody (e.g., anti-rabbit) against the antibody coated on the gold nanoparticles (e.g., rabbit, anti-FAM). In the case of a negative result, the detector nucleic acid is not cleaved and does not flow on the lateral flow strip. Thus, the nanoparticles only bind and accumulate at the second line Multiplexing on the lateral flow strip can be performed by having two detector nucleic acids (e.g., a biotin-FAM detector nucleic acid and a biotin-DIG detector nucleic acid). Anti-FAM and anti-DIG antibodies are coated onto the lateral flow strip at two different regions. Anti-biotin antibodies are coated on gold nanoparticles. Fluorophores are conjugated directly to the affinity molecules (e.g., biotin) by first generating a biotin-dNTP following from the nucleic acids of the detector nucleic acid and then conjugating the fluorophore. In some embodiments, the lateral flow strip comprises multiple layers.

In some embodiments, the above lateral flow strip can be additionally interfaced with a sample preparation device. Individual parts of sample preparation devices of the present disclosure can include the following: a single chamber sample extraction device comprising: (a) an insert holds the sample collection device and regulates the step between sample extraction and dispensing the sample into another reaction or detection device, (b) the single chamber contains extraction buffer. The dispensing chamber can be filled with material that further purifies the nucleic acid as it is dispensed: (a) the insert holds the sample collection device and regulates the “stages” of sample extraction and nucleic acid amplification. Each set of notches in a sample preparation device between the multiple chambers can be offset 90° from the preceding set, (b) the reaction module can contain multiple chambers separated by substrates that allow for independent reactions to occur. (e.g., i. a nucleic acid separation chamber, ii. a nucleic acid amplification chamber and iii. a DETECTR reaction chamber or dispensing chamber). Each chamber has notches that prevent the insert from progressing into the next chamber without a deliberate 90° turn. The first two chambers may be separated by material that removes inhibitors between the extraction and amplification reactions. Options for the reaction/dispensing chamber can include: (a) a single dispensing chamber may release only extracted sample or extraction/amplification or extraction/amplification/DETECTR reactions, (b) a duel dispensing chamber may release extraction/multiplex amplification products, and (c) a quadruple dispensing chamber would allow for multiplexing amplification and single DETECTR or four single amplification reactions. A sample work flow using a sample processing device can be as follows. The sample collection device is attached to the insert portion of the sample processing device. The insert is placed into the device chamber and pressed until the first stop (lower tabs on top portion meet upper tabs on bottom portion). This step allows the sample to come into contact with the nucleic acid extraction reagents. After the appropriate amount of time, the insert is turned 90° and depressed to the next set of notches. These actions transfer the sample into the amplification chamber. The sample collection device is no longer in contact with the sample or amplification products. After the appropriate incubation, the insert is rotated 90° and depressed to the next set of notches. These actions release the sample into the DETECTR (green reaction). The insert is again turned 90° and depressed to dispense the reaction.

### General Features of Devices

In some embodiments, a device of the present disclosure can hold 2 or more amplification chambers. In some embodiments, a device of the present disclosure can hold 10 or more detection chambers. In some embodiments, a device of the present disclosure comprises a single chamber in which sample lysis, target nucleic acid amplification, reverse transcription, and detection are all carried out. In some cases, different buffers are present in the different chambers. In some embodiments, all the chambers of a device of the present disclosure have the same buffer. In some embodiments, the sample chamber comprises the lysis buffer and all of the materials in the amplification and detection chambers are lyophilized or vitrified. In some embodiments, the sample chamber includes any buffer for lysing a sample disclosed herein. The amplification chamber can include any buffer disclosed herein compatible with amplification and/or reverse transcription of target nucleic acids. The detection chamber can include any DETECTR or CRISPR buffer (e.g., an MBuffer) disclosed herein or otherwise capable of allowing DETECTR reactions to be carried out. In this case, once sample lysing has occurred, volume is moved from the sample chamber to the other chambers in an amount enough to rehydrate the materials in the other chambers. In some embodiments, the device further comprises a pipette pump at one end for aspirating, mixing, and dispensing liquids. In some embodiments, an automated instrument is used to control aspirating, mixing, and dispensing liquids. In some embodiments, no other instrument is needed for the fluids in the device to move from chamber to chamber or for sample mixing to occur. A device of the present disclosure may be made of any suitable thermoplastic, such as COC, polymer COP, teflon, or another thermoplastic material. Alternatively, the device may be made of glass. In some embodiments, the detection chamber may include beads, such as nanoparticles (e.g., a gold nanoparticle). In some embodiments, the detector nucleic acids are immobilized on the beads. In some embodiments, after cleavage from the bead, the liberated detector nucleic acids flow into a secondary detection chamber, where detection of a generated signal occurs by any one of the instruments disclosed herein. In some embodiments, the detection chamber is shallow, but has a large surface area that is optimized for optical detection. A device of the present disclosure may also be coupled to a thermoregulator. For example, the device may be on top of or adjacent to a planar heater that can heat the device up to high temperatures. Alternatively, a metal rod conducting heat is inserted inside the device and presses upon a soft polymer. The heat is transferred to the sample by dissipating through the polymer and into the sample. This allows for sample heating with direct contact between the metal rod and the sample. In some embodiments, in addition to or in place of a buffer for lysing a sample, the sample chamber may include an ultrasonicator for sample lysis. A swab carrying the sample may be inserted directly into the sample chamber. Commonly, a buccal swab may be used, which can carry blood, urine, or a saliva sample. A filter may be included in any of the chambers in the devices disclosed herein to filter the sample prior to carrying it to the next step of the method. Any of the devices disclosed herein can be couple to an additional sample preparation module for further manipulation of the sample before the various steps of the DETECTR reaction. In some embodiments the detector nucleic acid can be in solution in the detection chamber. In other embodiments, the detector nucleic acid can be immobilized directly on the surface of the detection chamber. The surface can be the top or the bottom of the chamber. In still other embodiments, the detector nucleic acid can be immobilized to the surface of a bead. In the case of a bead, after cleavage, the detectable signal may be washed into a subsequent chamber while the bead remains trapped—thus allowing for separation of the detectable signal from the bead. Alternatively, cleavage of the detector nucleic acid off of the surface of the bead is enough to generate a strong enough detectable signal to be measured. By sequestering or immobilizing the above described detector nucleic acids, the stability of the detector nucleic acids in the devices disclosed herein carrying out DETECTR reactions may be improved. Any of the above devices can be compatible for colorimetric, fluorescence, amperometric, potentiometric, or another electrochemical signal. In some embodiments, the colorimetric, fluorescence, amperometric, potentiometric, or another electrochemical sign may be detected using a measurement device connected to the detection chamber (e.g., a fluorescence measurement device, a spectrophotometer, or an oscilloscope).

In some embodiments, signals themselves can be amplified, for example via use of an enzyme such as horse radish peroxidase (HRP). In some embodiments, biotin and avidin reactions, which bind at a 4:1 ratio can be used to immobilize multiple enzymes or secondary signal molecules (e.g., 4 enzymes of secondary signal molecules, each on a biotin) to a single protein (e.g., avidin). In some embodiments, an electrochemical signal may be produced by an electrochemical molecule (e.g., biotin, ferrocene, digoxigenin, or invertase). In some embodiments, the above devices could be couple with an additional concentration step. For example, silica membranes may be used to capture nucleic acids off a column and directly apply the Cas reaction mixture on top of said filter. In some embodiments, the sample chamber of any one of the devices disclosed herein can hold from 20 ul to 1000 ul of volume. In some embodiments, the sample chamber holds from 20 to 500, from 40 to 400, from 30 to 300, from 20 to 200 or from 10 to 100 ul of volume. In preferred embodiments, the sample chamber holds 200 ul of volume. The amplification and detection chambers can hold a lower volume than the sample chamber. For example, the amplification and detection chambers may hold from 1 to 50, 10 to 40, 20 to 30, 10 to 40, 5 to 35, 40 to 50, or 1 to 30 ul of volume. Preferably, the amplification and detection chambers may hold about 200 ul of volume. In some embodiments, an exonuclease is present in the amplification chamber or may be added to the amplification chamber. The exonuclease can clean up single stranded nucleic acids that are not the target. In some embodiments, primers for the target nucleic acid can be phosophorothioated in order to prevent degradation of the target nucleic acid in the presence of the exonuclease. In some embodiments, any of the devices disclosed herein can have a pH balancing well for balancing the pH of a sample. In some embodiments, in each of the above devices, the detector nucleic acid is present in at least four-fold excess of total nucleic acids (target nucleic acids+non-target nucleic acids). Preferably the detector nucleic acid is present in at least 10-fold excess of total nucleic acids. In some embodiments, the detector nucleic acid is present in at least 4-fold, at least 5-fold at least 6-fold, at least 7-fold, at least 8-fold, at least 9-fold, at least 10-fold, at least 15-fold, at least 20-fold, at least 50-fold, at least 100-fold, from 1.5 to 100-fold, from 4 to 80-fold, from 4 to 10-fold, from 5 to 20-fold or from 4 to 15-fold excess of total nucleic acids. In some embodiments, any of the devices disclosed herein can carry out a DETECTR reaction (e.g., a multiplexed DETECTR reaction or a high-plex DETECTR reaction) with a limit of detection of at least 0.1 aM, at least 0.1 nM, at least 1 nM or from 0.1 aM to 1 nM. In some embodiments, the devices disclosed herein can carry out a DETECTR reaction with a positive predictive value of at least 75%, at least 80%, at least 85%, at least 90%, at least 92%, at least 95%, at least 97%, at least 99%, or 100%. In some embodiments, the devices disclosed herein can carry out a DETECTR reaction with a negative predictive value of at least 75%, at least 80%, at least 85%, at least 90%, at least 92%, at least 95%, at least 97%, at least 99%, or 100%. In some embodiments, spatial multiplexing in the above devices is carried out by having at least one, more than one, or every detection chamber in the device comprise a unique guide nucleic acid (e.g., guide RNA).

### Kit

Disclosed herein are kits, reagents, methods, and systems for use to detect a target nucleic acid. In some embodiments, the kit comprises the reagents and a support medium. The reagent may be provided in a reagent chamber or on the support medium. Alternatively, the reagent may be placed into the reagent chamber or the support medium by the individual using the kit. Optionally, the kit further comprises a buffer and a dropper. The reagent chamber be a test well or container. The opening of the reagent chamber may be large enough to accommodate the support medium. The buffer may be provided in a dropper bottle for ease of dispensing. The dropper can be disposable and transfer a fixed volume. The dropper can be used to place a sample into the reagent chamber or on the support medium.

In some embodiments, a kit for detecting a plurality of target nucleic acids comprising a support medium; a plurality of guide nucleic acids (e.g., guide RNAs) targeting the plurality of target nucleic acids; a programmable nuclease capable of being activated when complexed with a guide nucleic acid from the plurality of guide nucleic acids and a target nucleic acid from the plurality of target nucleic acid populations; and a single-stranded detector nucleic acid comprising a detection moiety, wherein the detector nucleic acid is capable of being cleaved by the activated nuclease, thereby generating a first detectable signal.

In some embodiments, a kit for detecting a target nucleic acid comprising a PCR plate; a plurality of guide nucleic acids targeting a plurality of target nucleic acids; a programmable nuclease capable of being activated when complexed with a guide nucleic acid of the plurality of guide nucleic acids and a target nucleic acid of the plurality of target nucleic acids; and a single stranded detector nucleic acid comprising a detection moiety, wherein the detector nucleic acid is capable of being cleaved by the activated nuclease, thereby generating a first detectable signal. The wells of the PCR plate can be pre-aliquoted with one or more guide nucleic acids of the plurality of guide nucleic acids targeting one or more target nucleic acids of the plurality of target nucleic acids, a programmable nuclease capable of being activated when complexed with the guide nucleic acid and the target sequence, and at least one population of a single stranded detector nucleic acid comprising a detection moiety. In some embodiments, one or more wells of the PCR plate may be pre-aliquoted with the plurality of guide nucleic acids. In some embodiments, one or more wells of the PCR plate may be pre-aliquoted with a subset of the plurality of guide nucleic acids, wherein the subset comprises one or more guide nucleic acids of the plurality of guide nucleic acids. A user can thus add the biological sample of interest to a well of the pre-aliquoted PCR plate and measure for the detectable signal with a fluorescent light reader or a visible light reader.

In some instances, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are illustrative and non-limiting to the scope of the devices, methods, reagents, systems, and kits described herein.

### Example 1

### DETECTR Reactions Simulating a 20-Plex Guide Pool, a 50-Plex Guide Pool, and a 100-Plex Guide Pool DETECTR Reactions

This example describes DETECTR reactions simulating a 20-plex guide pool, a 50-plex guide pool, and a 100-plex guide pool DETECTR reactions using an LbCas12a programmable nuclease (SEQ ID NO: 18). To demonstrate the viability of high-plex guide pooling for use in high-plex DETECTR reactions, experiments using two guide nucleic acid sequences were performed. In each experiment two guide nucleic acid sequences were combined at different concentration ratios. The first guide nucleic acid was directed to a segment of a target nucleic acid and the second guide nucleic acid was a segment of an off-target nucleic acid. The lower concentration guide nucleic acid sequence was held constant at 20 nM in each reaction while the higher concentration guide nucleic acid sequence was varied at 380 nM, 980 nM, or 1980 nM in the simulated 20-plex, 50-plex, and 100-plex DETECTR reactions, respectively. The total guide nucleic acid concentration in the simulated 20-plex, 50-plex, and 100-plex DETECTR reactions was 400 nM, 1000 nM (1 μM), and 2000 nM (2 μM), respectively. The concentration of the LbCas12a in each reaction was proportional to the total guide nucleic acid concentration. The concentration of LbCas12a in the simulated 20-plex, 50-plex, and 100-plex DETECTR reactions was 400 nM, 1000 nM (1 μM), and 2000 nM (2 μM), respectively. The sequences of the guide nucleic acids and target nucleic acids used in this assay are provided in TABLE 5.

TABLE 5
Guide Nucleic Acid and Target
Nucleic Acid Sequences

| SEQ ID | | |
|---|---|---|
| NO: | Type | Sequence |
| SEQ ID | gRNA to human | UAAUUUCUACUAAGUGUAGAU |
| NO: 171 | RNase P | CCAGAACACAUAGCGACAUG |
| SEQ ID | gRNA to human | UAAUUUCUACUAAGUGUAGAU |
| NO: 172 | ß-globin | UAUUGGUCUCCUUAAACCUG |
| SEQ ID | Human RNase | CGTGGCCCCACTGATGAGCTT |
| NO: 173 | P target | CCCTCCGCCCTATGGGAAAAA |
| | | GTGGTCTCATACAGAACTTAT |
| | | AAGATTCCCAAATCCAAAGAC |
| | | ATTTCACGTTTATGGTGATTT |
| | | CCCAGAACACATAGCGACATG |
| | | CAAATA |
| SEQ ID | Human | CCTATCAGAAACCCAAGAGTC |
| NO: 174 | ß-globin | TTCTCTGTCTCCACATGCCCA |
| | target | GTTTCTATTGGTCTCCTTAAA |
| | | CCTGTCTTGTAACCTTGATAC |
| | | CAACCTGCCCAGGGCCTCACC |
| | | ACCAACTTCATCCACGTTCAC |

For each reaction, guide nucleic acids were complexed 1:1 with the LbCas12a programmable nuclease at 4-fold the final concentration in Tris, pH 8.0 buffer (20 mM Tris HCl, pH 8.0, 100 mM NaCl, 5 mM MgCl₂, 1 mM DTT, 5% glycerol, 50 μg/mL Heparin). The concentration of each of the pooled guide nucleic acid and the programmable nuclease in the complexing reaction was 1.6 μM, 4 μM, and 8 μM for the simulated 20-plex, 50-plex, and 100-plex DETECTR reactions, respectively. Complexing reactions were incubated for 30 minutes at 37° C. to form complexes. Each complexing reaction was then combined in equal volumes with 400 nM single-stranded DNA detector nucleic acid (SEQ ID NO: 9 labeled at the 5′ end with FAM and labeled at the 3′ end with Iowa Black FQ) in Tris, pH 8.0 buffer with an additional 16% glycerol. The combined complexing reaction and detector nucleic acid were then combined in equal with a sample containing a target nucleic such that the final target nucleic acid concentration was 10 pM, 100 pM, or 1000 pM.

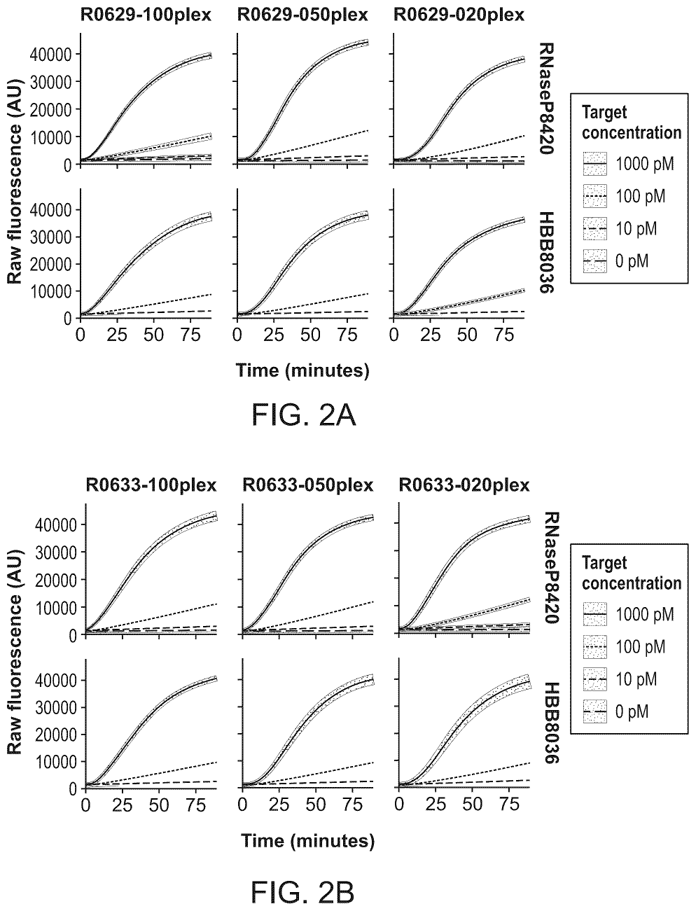

FIG. 2 shows raw fluorescence over time of multiplexed DETECTR reactions using an LbCas12a programmable nuclease (SEQ ID NO: 18). Each multiplexed DETECTR reaction was performed with two guide RNA sequences. In each reaction, a first guide nucleic acid sequence was present at either 19-fold, 49-fold, or 99-fold higher concentration than the second guide nucleic acid sequence to simulate 20-plex, 50-plex, or 100-plex high-plex DETECTR reactions, respectively. An 8-nucleotide single-stranded DNA detector nucleic acid labeled at the 5′ end with FAM and labeled at the 3′ end with Iowa Black FQ with a sequence of SEQ ID NO: 9 was used in each reaction.

FIG. 2A shows a first set of DETECTR reactions in which a guide RNA sequence targeting a human β-globin gene (SEQ ID NO: 172) was present in 19-fold (“20plex”), 49-fold (“50plex”), or 99-fold (“100plex”) higher concentration than a guide RNA sequence targeting a human RNAase P gene (SEQ ID NO: 171). The pooled guide RNAs were used to detect the presence or absence of a double-stranded DNA target nucleic acid corresponding to an amplified segment of the human RNase P gene (SEQ ID NO: 173, top row) or an amplified segment of the human β-globin gene (SEQ ID NO: 174, bottom row). Each DETECTR reaction was performed in the presence of 0 pM, 10 pM, 100 pM, or 1000 pM of the target nucleic acid.

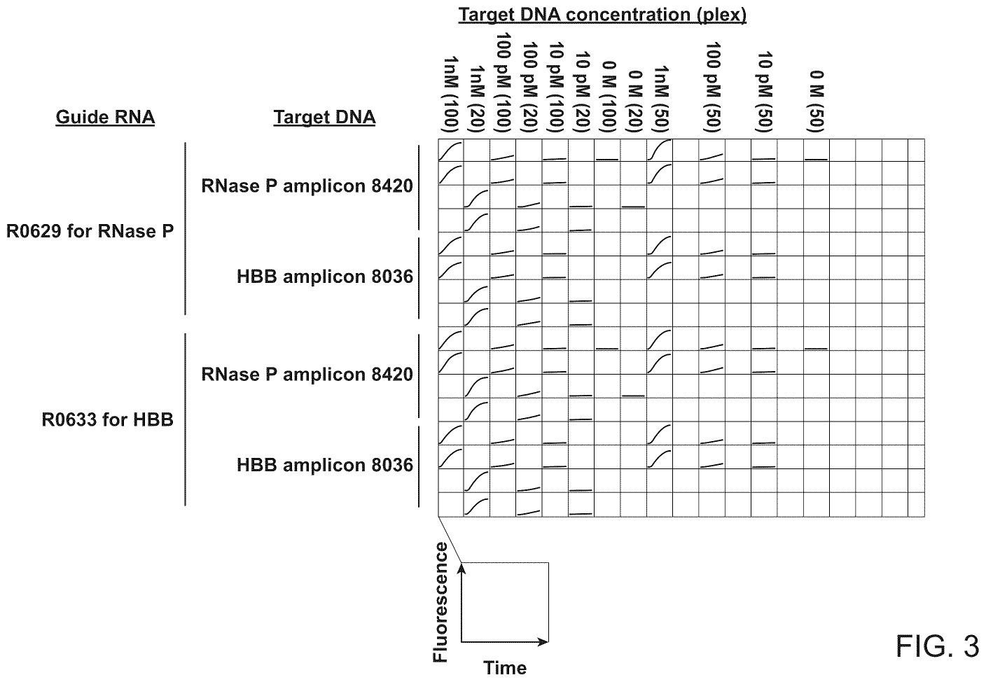

FIG. 2B shows a second set of multiplexed DETECTR reactions in which a guide RNA sequence targeting a human RNAase P gene (SEQ ID NO: 171) was present in 19-fold (“20plex”), 49-fold (“50plex”), or 99-fold (“100plex”) higher concentration than a guide RNA sequence targeting a human β-globin gene (SEQ ID NO: 172). The pooled guide RNAs were used to detect the presence or absence of a double-stranded DNA target nucleic acid corresponding an amplified segment of the human RNase P gene (SEQ ID NO: 173, top row) or an amplified segment of the human β-globin gene (SEQ ID NO: 174, bottom row). Each multiplexed DETECTR reaction was performed in the presence of 0 pM, 10 pM, 100 pM, or 1000 pM of the target nucleic acid. The aggregate results from FIGS. 2A and 2B are shown in FIG. 3. The maximum rates of fluorescence detected in this assay are provided in TABLE 6.

TABLE 6
Maximum Rate of Fluorescence Detected
in Multiplexed DETECTR Reactions

| | | Target Concentration | | |
|---|---|---|---|---|
| | | 10 | 100 | 1000 |
| Target | Pool | pM | pM | pM |
| SEQ ID NO: 173 | SEQ ID NO: 171 20-Plex | 40 | 159 | 877 |
| | SEQ ID NO: 171 50-Plex | 40 | 187 | 1070 |
| | SEQ ID NO: 171 100-Plex | 40 | 145 | 824 |
| SEQ ID NO: 173 | SEQ ID NO: 172 20-Plex | 39 | 184 | 953 |
| | SEQ ID NO: 172 50-Plex | 46 | 170 | 910 |
| | SEQ ID NO: 172 100-Plex | 40 | 164 | 844 |
| SEQ ID NO: 174 | SEQ ID NO: 171 20-Plex | 37 | 164 | 775 |
| | SEQ ID NO: 171 50-Plex | 40 | 146 | 862 |
| | SEQ ID NO: 171 100-Plex | 38 | 138 | 734 |
| SEQ ID NO: 174 | SEQ ID NO: 172 20-Plex | 44 | 150 | 857 |
| | SEQ ID NO: 172 50-Plex | 35 | 145 | 879 |
| | SEQ ID NO: 172 100-Plex | 40 | 147 | 812 |

As shown in FIG. 2, FIG. 3 and TABLE 6, signals resulting from the lower concentration guide nucleic acid sequence complexing with a target nucleic acid are not affected by high concentrations of off-target guides nucleic acid sequences in the mixture. This assay demonstrates that up to 100 individual guide nucleic acid sequences may be pooled without adversely impacting the performance of the guide nucleic acid sequence directed to the target nucleic acid present in the sample.

### Example 2

### High-Plex DETECTR Reaction for Detection of Borrelia Species

This example describes a high-plex DETECTR reaction for detection of Borrelia species using an LbCas12a programmable nuclease (SEQ ID NO: 18) or a Cas12 variant programmable nuclease (SEQ ID NO: 28). Twenty guide nucleic acid sequences directed to 20 distinct target nucleic acids were pooled and used to detect samples containing varying amounts of amplified Borrelia DNA (isolated from Borrelia burgdorferi strain B3, obtained from American Type Culture Collection). Nineteen of the 20 guide nucleic acid sequences (corresponding to SEQ ID NO: 175-SEQ ID NO: 193 in Table 7) were directed toward the 16S ribosomal RNA gene of Borrelia burgdorferi, Borrelia miyamotoi, or both. The remaining guide nucleic acid sequence was directed toward the RNase P RNA component H1 gene.

TABLE 7

| SEQ ID NO | Guide | 5′ to 3′ sequence |
|---|---|---|
| SEQ ID NO: | R0643 | UAAUUUCUACUAAGUGUAGAUAAGCUUCGCUUGU |
| 175 | | AGAUGAG |
| SEQ ID NO: | R0644 | UAAUUUCUACUAAGUGUAGAUACUUGCAUGCUUA |
| 176 | | AGACGCA |
| SEQ ID NO: | R0645 | UAAUUUCUACUAAGUGUAGAUAUCCUGGCUUAGA |
| 177 | | ACUAACG |
| SEQ ID NO: | R0646 | UAAUUUCUACUAAGUGUAGAUAUUCGAUGAUACG |
| 178 | | CGAGGAA |
| SEQ ID NO: | R0647 | UAAUUUCUACUAAGUGUAGAUCAACAUAGGUCCAC |
| 179 | | AGUUGA |
| SEQ ID NO: | R0648 | UAAUUUCUACUAAGUGUAGAUCAACAUAGUUCCAC |
| 180 | | AGUUGA |
| SEQ ID NO: | R0649 | UAAUUUCUACUAAGUGUAGAUCAGCAUAGUUCCAC |
| 181 | | AGUUGA |
| SEQ ID NO: | R0650 | UAAUUUCUACUAAGUGUAGAUCAGCGUACACUACC |
| 182 | | AGGGUA |
| SEQ ID NO: | R0651 | UAAUUUCUACUAAGUGUAGAUCCCUACCAACUAGC |
| 183 | | UAAUAA |
| SEQ ID NO: | R0652 | UAAUUUCUACUAAGUGUAGAUCUACAAAGCUUAU |
| 184 | | UCCUCAU |
| SEQ ID NO: | R0653 | UAAUUUCUACUAAGUGUAGAUGGGUCUAUAUACA |
| 185 | | GGUGCUG |
| SEQ ID NO: | R0654 | UAAUUUCUACUAAGUGUAGAUGGGUCUGUAUACA |
| 186 | | GGUGCUG |
| SEQ ID NO: | R0655 | UAAUUUCUACUAAGUGUAGAUGUGACUCAGCGUC |
| 187 | | AGUCUUG |
| SEQ ID NO: | R0656 | UAAUUUCUACUAAGUGUAGAUGUUAACACCAAGU |
| 188 | | GUGCAUC |
| SEQ ID NO: | R0657 | UAAUUUCUACUAAGUGUAGAUUAGGAAAUGACAA |
| 189 | | AGCGAUG |
| SEQ ID NO: | R0658 | UAAUUUCUACUAAGUGUAGAUUCAUUUCCUACAA |
| 190 | | AGCUUAU |
| SEQ ID NO: | R0659 | UAAUUUCUACUAAGUGUAGAUUGCAUAGACUUAU |
| 191 | | AUAUCCG |
| SEQ ID NO: | R0660 | UAAUUUCUACUAAGUGUAGAUAGGUAUGUUUAGU |
| 192 | | GAGGGGG |
| SEQ ID NO: | R0661 | UAAUUUCUACUAAGUGUAGAUGUGAGGGGGGUGA |
| 193 | | AGUCGUA |

For the Cas112 variant reactions, each of the 20 guide nucleic acid sequences were complexed individually at high concentration (1.6 μM) with 1.6 μM of the Cas112 variant programmable nuclease (SEQ ID NO: 28) in HEPES, pH 7.5 buffer (20 mM HEPES, pH 7.5, 2 mM potassium acetate, 5 mM magnesium acetate, 100 glycerol, and 0.00016% Triton X-100). The complexing reactions were incubated at 37° C. for 30 minutes. Complexing reactions for each of the 20 guide nucleic acid sequences were combined in equal volumes. The pooled complexed guide nucleic acid sequences were combined with a mixture containing a single-stranded DNA detector nucleic acid in 3×HEPES, pH 7.5 buffer.

For the LbCas12a reactions, each of the 20 guide nucleic acid sequences were complexed individually at high concentration (3.2 μM) with 3.2 μM of LbCas12a programmable nuclease (SEQ ID NO: 18) in Tris, pH 8.0 buffer (20 mM Tris HCl, pH 8.0, 100 mM NaCl, 5 mM MgCl₂, 1 mM DTT, 5% glycerol, 50 μg/mL Heparin). The complexing reactions were incubated at 37° C. for 30 minutes. Complexing reactions for each of the 20 guide nucleic acid sequences were combined in equal volumes. The pooled complexed guide nucleic acid sequences were combined with a mixture containing a single-stranded DNA detector nucleic acid in 3× Tris, pH 8.0 buffer.

Separately, Borrelia culture diluted into negative matrix at different dilution factors was PCR amplified to amplify the 16S rRNA gene. Guide nucleic acid pools complexed with either the Cas12 variant or LbCas12a were combined with the diluted and PCR-amplified Borrelia samples.

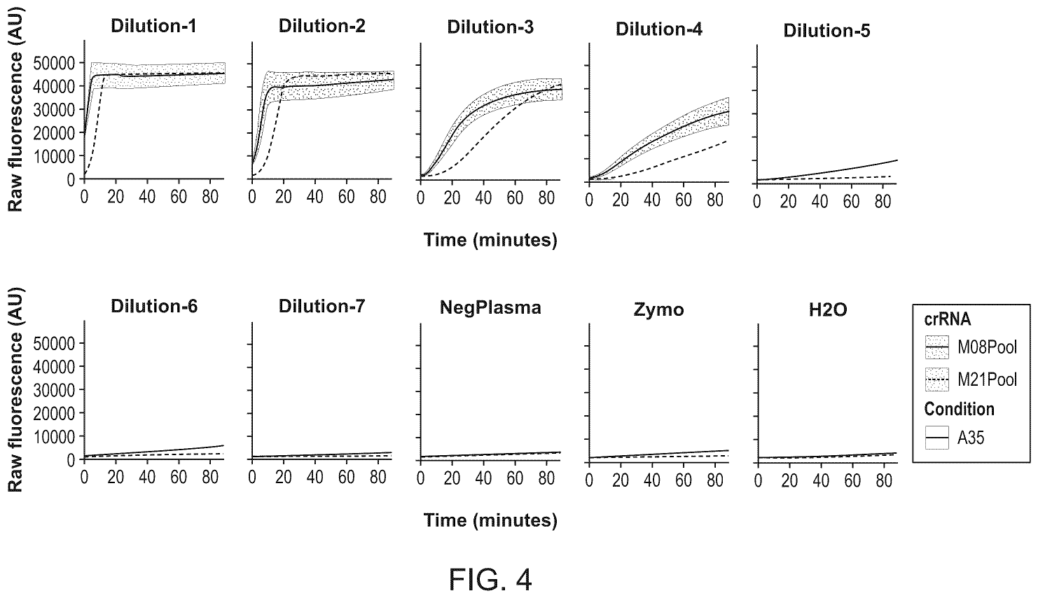

FIG. 4 shows raw fluorescence over time of high-plex DETECTR reactions using an LbCas12a programmable nuclease (SEQ ID NO: 18, dashed lines) and a Cas12 variant programmable nuclease (SEQ ID NO: 28, solid lines). A guide RNA pool of 20 distinct guide nucleic acid sequences was used to detect the presence or absence of target nucleic acids in Borrelia culture diluted 10-fold (“Dilution-1”), 10²-fold (“Dilution-2”), 10³-fold (“Dilution-3”), 10⁴-fold (“Dilution-4”), 10⁵-fold (“Dilution-5”), 10⁶-fold (“Dilution-6”), or 10⁷-fold (“Dilution-7”) in a negative matrix and PCR-amplified. Diluted Borrelia cultures were PCR-amplified prior to detection to amplify the 16S gene. Negative plasma (“NegPlasma”), Zymo standard with Pseudomonas aeruginosa, Escherichia coli, Salmonella enterica, Lactobacillus subtilis, Saccharomyces cerevisiae, and Cryptococcus neoformans (“Zymo”), and water (“H2O’) were tested as negative controls.

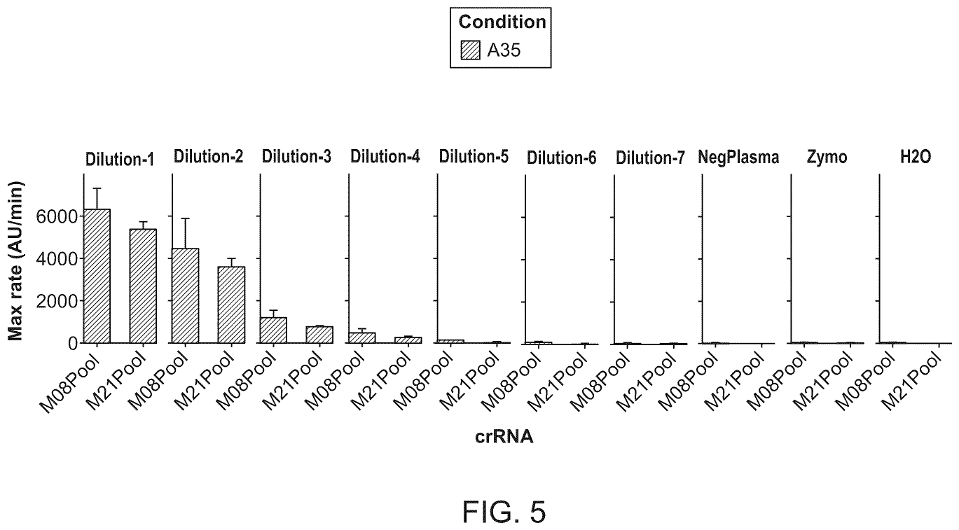

FIG. 5 shows the maximum fluorescence rate of the high-plex DETECTR reactions shown in FIG. 4. Left columns in each condition correspond to reactions using the Cas12 variant programmable nuclease (SEQ ID NO: 28), and right columns correspond to reactions using the LbCas12a programmable nuclease (SEQ ID NO: 18).

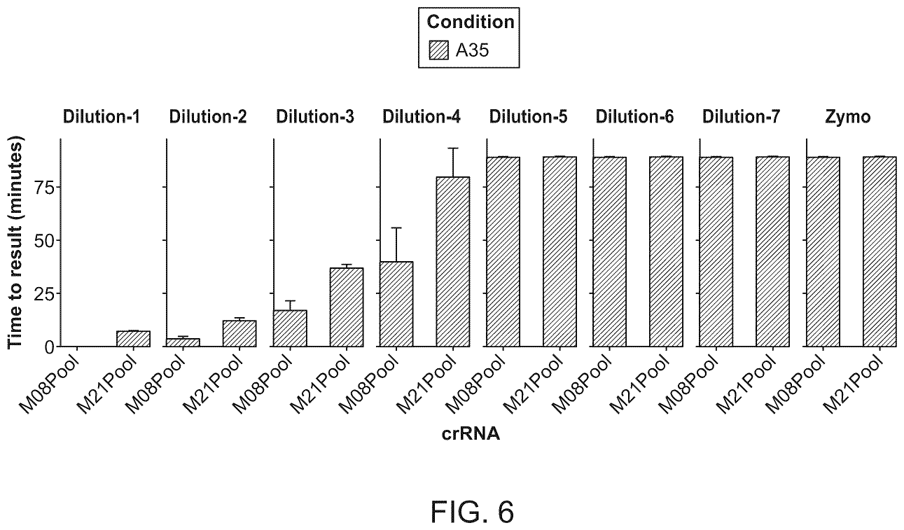

FIG. 6 shows the time to result of the high-plex DETECTR reactions shown in FIG. 4 and FIG. 5. Left columns in each condition correspond to reactions using the Cas12 variant programmable nuclease (SEQ ID NO: 28), and right columns correspond to reactions using the LbCas12a programmable nuclease (SEQ ID NO: 18). A low time to result is indicative of a positive DETECTR reaction.

As illustrated by FIG. 4-FIG. 6, the Cas12 variant (SEQ ID NO: 28) shows higher sensitivity for the target nucleic acids in a high-plex guide pooling assay than LbCas12a (SEQ ID NO: 18). Additionally, the Cas12 variant shows higher sensitivity and a faster time to result than LbCas12a. This assay demonstrates that high-plex DETECTR reactions (for example the 20-plex DETECTR reaction shown here) may be used to detect the presence of multiple species of target nucleic acids associated with a disease.

### Example 3

### High-Plex DETECTR Reaction for Detection of Healthcare-Associated Infections Using a Cas12 Programmable Nuclease

This example describes a high-plex DETECTR reaction for detection of health-care associated infections. One thousand guide nucleic acids sequences directed to target nucleic acids corresponding to distinct segments within each of Staphylococcus aureus, methicillin resistant Staphylococcus aureus, Candida albicans, Pseudomonas aeruginosa, Acinetobacter baumannii, Stenotrophomonas maltophilia, Clostridium difficile, Escherichia coli, Mycobacterium tuberculosis, and Legionella sp. are pooled and complexed at a 1:1 ratio with a Cas12 programmable nuclease. The complexed guide nucleic acids and Cas12 programmable nucleases are then combined with a detector nucleic acid and a biological sample from a patient suspected of having a healthcare-associated infection. If the biological sample is positive for a hospital-associated infection, one or more of the guide nucleic acids and Cas12 programmable nucleases binds a target nucleic acid in the sample, activating the Cas12 programmable nuclease, and initiating transcollateral cleavage of the detector nucleic acid. The cleaved detector nucleic acid produces a detectable signal.

### Example 4

### High-Plex DETECTR Reaction for Detection of Healthcare-Associated Infections Using a Cas13 Programmable Nuclease

This example describes a high-plex DETECTR reaction for detection of health-care associated infections using a Cas13 programmable nuclease. One thousand guide nucleic acid sequences directed to target nucleic acids corresponding to distinct regions within each of Staphylococcus aureus, methicillin resistant Staphylococcus aureus, Candida albicans, Pseudomonas aeruginosa, Acinetobacter baumannii, Stenotrophomonas maltophilia, Clostridium difficile, Escherichia coli, Mycobacterium tuberculosis, and Legionella sp. are pooled and complexed at a 1:1 ratio with a Cas13 programmable nuclease. The complexed guide nucleic acids and Cas13 programmable nucleases are then combined with a detector nucleic acid and a biological sample from a patient suspected of having a healthcare-associated infection. If the biological sample is positive for a hospital-associated infection, one or more of the guide nucleic acids and Cas13 programmable nucleases binds a target nucleic acid in the sample, activating the Cas13 programmable nuclease, and initiating transcollateral cleavage of the detector nucleic acid. The cleaved detector nucleic acid produces a detectable signal. Optionally, target RNA in the sample are reverse transcribed, amplified, and in vitro transcribed prior to contacting the sample with the pool of guide nucleic acids complexed with the Cas13 programmable nuclease, and the detector nucleic acid.

### Example 5

### High-Plex DETECTR Reaction for Detection of Healthcare-Associated Infections Using a Cas14 Programmable Nuclease

This example describes a high-plex DETECTR reaction for detection of health-care associated infections using a Cas14 programmable nuclease. One thousand guide nucleic acid sequences directed to target nucleic acids corresponding to distinct segments within each of Staphylococcus aureus, methicillin resistant Staphylococcus aureus, Candida albicans, Pseudomonas aeruginosa, Acinetobacter baumannii, Stenotrophomonas maltophilia, Clostridium difficile, Escherichia coli, Mycobacterium tuberculosis, and Legionella sp. are pooled and complexed at a 1:1 ratio with a Cas14 programmable nuclease. The complexed guide nucleic acids and Cas14 programmable nucleases are then combined with a detector nucleic acid and a biological sample from a patient suspected of having a healthcare-associated infection. If the biological sample is positive for a hospital-associated infection, one or more of the guide nucleic acids and Cas14 programmable nucleases binds a target nucleic acid in the sample, activating the Cas14 programmable nuclease, and initiating transcollateral cleavage of the detector nucleic acid. The cleaved detector nucleic acid produces a detectable signal.

### Example 6

### DETECTR Reactions Simulating a 20-Plex Guide Pool, a 50-Plex Guide Pool, and a 100-Plex Guide Pool DETECTR Reactions

This example describes a set of DETECTR reactions for a single plex (single sequence of a guide nucleic acid) assay and simulated 500-plex and simulated 1000-plex guide pool assays using a Cas12 programmable nuclease (SEQ ID NO: 18). Reaction components included a first guide nucleic acid directed to a segment of a target nucleic acid comprising a human RNase P gene (SEQ ID NO: 172). The multiplex reactions further comprised three guide nucleic acids directed toward segments of off-target nucleic acids. The guide nucleic acid directed toward human RNase P was held constant at 20 μM in each reaction while the aggregate concentrations of off-target guide nucleic acid sequences were provided at 9.8 μM and 19.8 μM for the simulated 500-plex and 1000-plex DETECTR reactions, respectively. The concentration of SEQ ID NO: 18 in each reaction was proportional to the total guide nucleic acid concentration, at 20 nM, 10 μM and 20 μM for the single plex, 500-plex and 1000-plex DETECTR reactions, respectively.

Guide nucleic acids were complexed 1:1 with SEQ ID NO: 18 at 37° C. for 30 minutes to form complexes, yielding a first sample comprising 4 μM complex with RNase P gene guide nucleic acid and a second sample comprising 40 μM complex with off-target guide nucleic. The two samples were mixed with an 8-nucleotide single-stranded DNA detector nucleic acid labeled at the 5′ end with FAM and labeled at the 3′ end with Iowa Black FQ with a sequence of SEQ ID NO: 9, and then combined at volume ratios of 1:49 and 1:99 ratios (RNase P gene to off-target guide nucleic acid) to simulate 500-plex and 1000-plex DETECTR reactions, respectively. A further portion of the RNase P gene guide nucleic acid complex left unmixed with the off-target guide nucleic pool was used for the single plex DETECTR reactions. The resulting mixtures were combined with sample containing target nucleic acid to achieve final target nucleic acid concentrations of 1000 pM (1 nM), 100 pM, 10 pM or 0 pM.

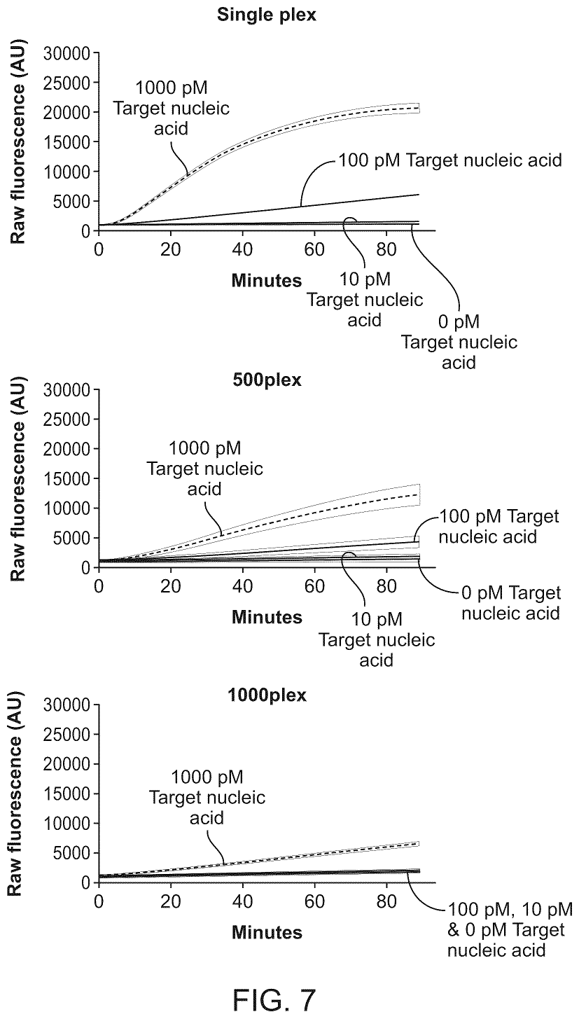

FIG. 7 shows raw fluorescence data for the single plex, 500-plex, and 1000-plex samples with 1000 pM (1 nM), 100 pM, 10 pM or 0 pM target nucleic acid present.

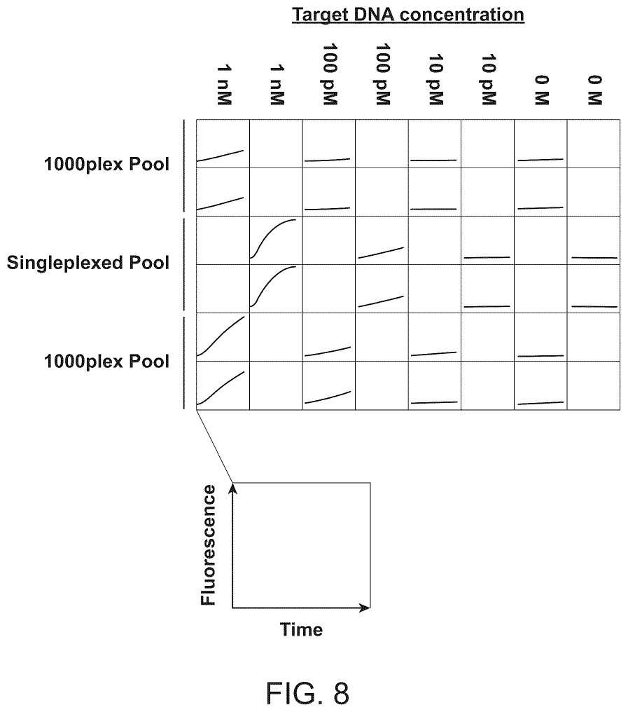

FIG. 8 Shows raw fluorescence data for the single plex and simulated 500-plex and 1000-plex reactions. In assays with 100 pM or 1000 pM (1 nM) target nucleic acid present, the maximum rate of fluorescence signal increase was inversely correlated with simulated plex, such that the single plex reactions had the highest maximum rates of fluorescence signal increase and the simulated 1000-plex reactions had the lowest maximum rates of fluorescence signal increase. The maximum rates of fluorescence signal increase detected in this assay are provided in TABLE 8.

TABLE 8
Maximum Rate of Fluorescence Detected in DETECTR Reactions

| | Target Nucleic Acid Concentration | | | |
|---|---|---|---|---|
| Plex | 0 pM | 10 pM | 100 pM | 1000 pM |
| Human RNase P Gene Single | 34 | 23 | 89 | 467 |
| Plex | | | | |
| Human RNase P Gene 500-plex | 21 | 32 | 60 | 194 |
| Human RNase P Gene 1000-plex | 30 | 43 | 29 | 93 |

### Example 7

### DETECTR Reactions Simulating a 20-Plex Guide Pool, a 50-Plex Guide Pool, and a 100-Plex Guide Pool DETECTR Reactions

This example describes a set of DETECTR reactions simulating a 20-plex guide pool, a 50-plex guide pool, a 100-plex and a 200-plex guide pool. Experiments were performed using a programmable nuclease of SEQ ID NO: 28, and two guide nucleic acids to mimic high-plex DETECTR reactions. The first guide nucleic acid was directed to a segment of a target nucleic acid and the second guide nucleic acid was directed toward a segment of an off-target nucleic acid. The lower concentration guide nucleic acid sequence was held constant at 10 nM in each reaction while the higher concentration guide nucleic acid sequence was varied at 190 nM, 490 nM, 990 nM or 1990 nM in the simulated 20-plex, 50-plex, 100-plex and 200-plex DETECTR reactions, respectively. The total guide nucleic acid concentrations in the simulated 20-plex, 50-plex, 100-plex and 200-plex DETECTR reactions were 200 nM, 500 nM, 1000 nM (1 μM) and 2000 nM (2 μM), respectively. The concentration of SEQ ID NO: 28 in each reaction was proportional to the total guide nucleic acid concentration, at 200 nM, 500 nM, 1000 nM (1 μM) and 2000 nM (2 μM) for the 20-plex, 50-plex, 100-plex and 200-plex DETECTR reactions, respectively. An 8-nucleotide single-stranded DNA detector nucleic acid labeled at the 5′ end with FAM and labeled at the 3′ end with Iowa Black FQ with a sequence of SEQ ID NO: 9 was used in each reaction The sequences of the guide nucleic acids and target nucleic acids used in this assay are provided in TABLE 9.

TABLE 9
Guide Nucleic Acid and Target
Nucleic Acid Sequences

| SEQ ID NO: | Type | Sequence |
|---|---|---|
| SEQ ID | gRNA to human | UAAUUUCUACUAAGUGUAG |
| NO: 194 | RNase P | AUGAUUUGGGAAUCUUAUA |
| | 8644 | AGU |
| SEQ ID | gRNA to human | UAAUUUCUACUAAGUGUA |
| NO: 172 | ß-globin | GAUUAUUGGUCUCCUUAA |
| | | ACCUG |
| SEQ ID | Human RNase | ATGGGAAAAAGTGGTCTCA |
| NO: 195 | P 8644 | TACAGAACTTATAAGATTC |
| | target | CCAAATCCAAAGACATTTC |
| | | ACGTTTATGGTGATTTCCC |
| | | AGAACACATAGCGACATGC |
| | | AAATATTGCAGGGCGCCAC |
| | | TCCCCTGTCCCTCACAG |
| SEQ ID | Human | CCTATCAGAAACCCAAGAG |
| NO: 174 | ß-globin | TCTTCTCTGTCTCCACATG |
| | target | CCCAGTTTCTATTGGTCTC |
| | | CTTAAACCTGTCTTGTAAC |
| | | CTTGATACCAACCTGCCCA |
| | | GGGCCTCACCACCAACTTC |
| | | ATCCACGTTCAC |

Guide nucleic acids were complexed 1:1 with SEQ ID NO: 28 at 4-fold the final concentration in HEPES, pH 7.5 buffer (100 mM HEPES, 10 mM potassium Acetate, 25 mM magnesium acetate, 5% glycerol, 0.0008% Triton X-100) and incubated for 30 minutes at 37° C. to form complexes with concentrations of 400 nM, 1000 nM (1 μM), 2000 nM (2 μM) and 4000 nM (4 μM) for the simulated 20-plex, 50-plex, 100-plex and 200-plex DETECTR reactions, respectively. The complexing reactions were then combined in equal volumes with 200 nM single-stranded DNA detector nucleic acid (SEQ ID NO: 9 labeled at the 5′ end with FAM and labeled at the 3′ end with Iowa Black FQ) in Tris, pH 8.0 buffer with an additional 16% glycerol. The resulting mixtures were then combined with equal volumes of sample containing target nucleic acid to achieve final target nucleic acid concentrations of 1000 pM (1 nM), 100 pM, or 0 pM.

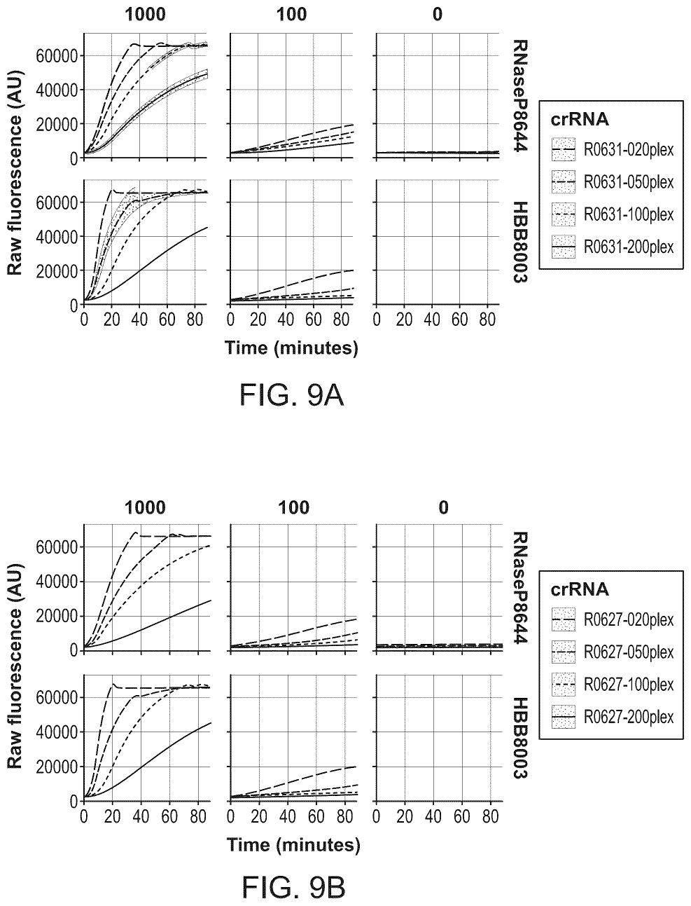

FIG. 9A shows raw fluorescence data for simulated 20-plex, 50-plex, 100-plex, and 200-plex DETECTR reactions with target nucleic acid corresponding to an amplified segment of the human RNase P gene (top, SEQ ID NO: 173) and with an amplified segment of the human B-globin gene (bottom, SEQ ID NO: 174) at concentrations of 1 nM, 100 pM, or 0 pM (left, middle, and right columns, respectively). Guide RNA targeting a human RNase P gene (SEQ ID NO: 171) was present in 19-fold (“20plex”), 49-fold (“50plex”) 99-fold (“100plex”), or 199-fold (“200plex) higher concentration than a guide RNA sequence targeting a human RNase P gene (SEQ ID NO: 171).

FIG. 9B shows raw fluorescence data for simulated 20-plex, 50-plex, 100-plex, and 200-plex DETECTR reactions with target nucleic acid corresponding to an amplified segment of the human RNase P gene (top, SEQ ID NO: 173) and with an amplified segment of the human B-globin gene (bottom, SEQ ID NO: 174) at concentrations of 1 nM, 100 pM, or 0 pM (left, middle, and right columns, respectively). Guide RNA targeting a human β-globin gene (SEQ ID NO: 172) was present in 19-fold (“20plex”), 49-fold (“50plex”) 99-fold (“100plex”), or 199-fold (“200plex) higher concentration than a guide RNA sequence targeting a β-globin gene (SEQ ID NO: 171).

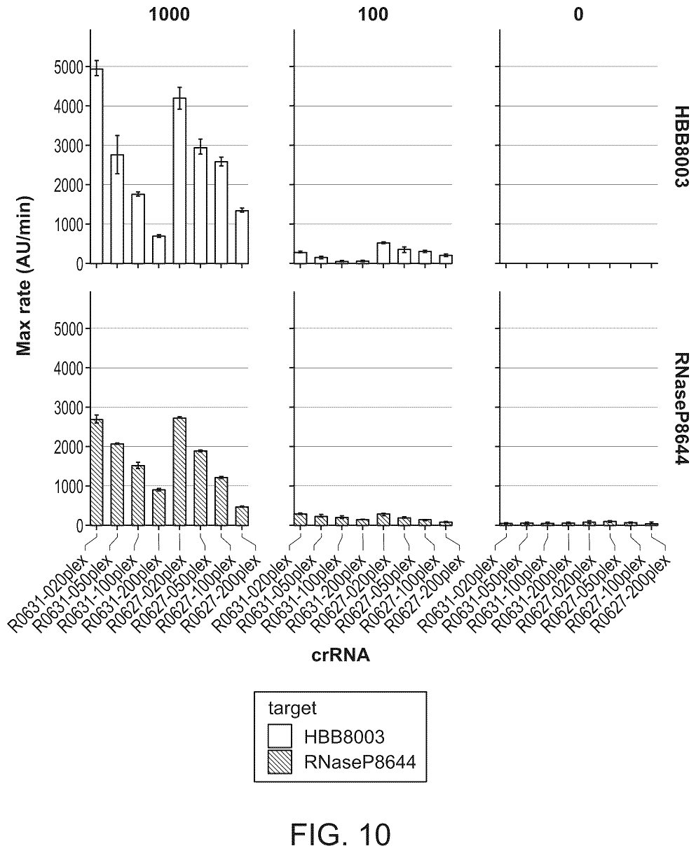

FIG. 10 provides the maximum rates of fluorescence from the above assays. Low fluorescence signal was detected in assays with 0 pM target nucleic acid present. In assays with 100 pM or 1 nM target nucleic acid present, the maximum rate of fluorescence signal was inversely correlated with simulated plex, such that the simulated 20-plex reactions had the highest maximum rate of fluorescence and the simulated 200-plex reactions had the lowest maximum rate of fluorescence.

### Example 8

### DETECTR Reaction Enabling Bacterial Community Profiling at the Species-Level

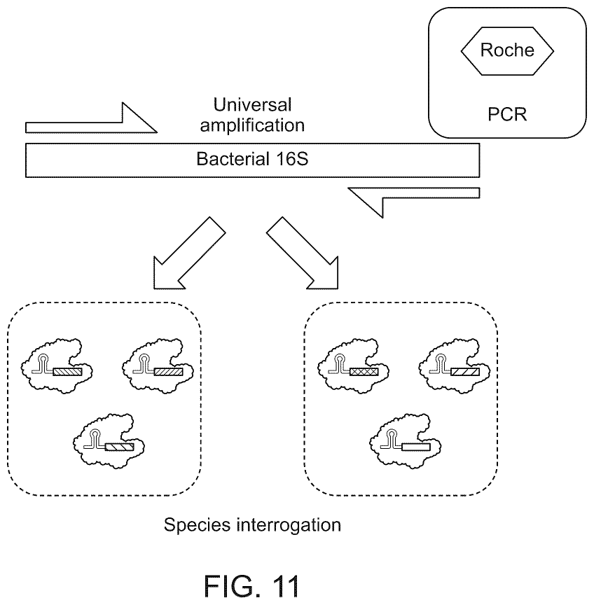

This example describes a multi-plex DETECTR reaction for profiling a bacterial population. DNA extraction is performed on a community of bacteria using techniques standard to the field. PCR amplification is performed using a set of universal primers targeting DNA encoding the 16S ribosomal subunit. In parallel, a set of guide nucleic acids targeting loci encoding 16S ribosomal subunits from species of interest are pooled and complexed at a 1:1 ratio with a programmable nuclease e.g., a Cas12 programmable nuclease. The complexed guide nucleic acids and programmable nuclease are then combined with a pool of detector nucleic acids and the PCR product (FIG. 11). If the biological sample is positive for a species of interest, one or more of the guide nucleic acids and programmable nucleases binds a target nucleic acid in the sample, activating the Cas14 programmable nuclease, and initiating transcollateral cleavage of the detector nucleic acid. The cleaved detector nucleic acid produces a detectable signal. In some cases, the rate of fluorescence increase upon initiation of the DETECTR reaction correlates with the number of target species present. In some cases, the rate of fluorescence increase upon initiation of the DETECTR reaction is proportional to the number of target species present.

### Example 9

### Assaying for SNPs in a Bacterial Population with a Single DETECTR Reaction

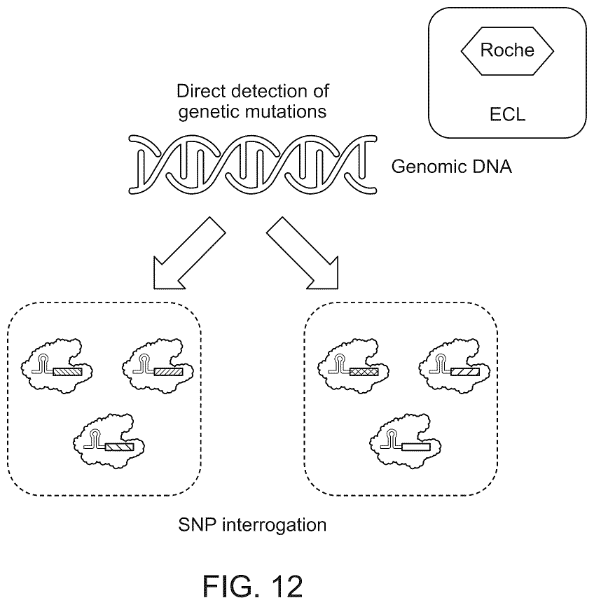

This example describes a multi-plex DETECTR reaction for determining the presence of an SNP in a bacterial population. A set of guide nucleic acids targeting potential SNPs of interest from a bacteria of interest are pooled and complexed at a 1:1 ratio with a programmable nuclease, e.g., a Cas12 programmable nuclease. DNA is extracted and amplified from a bacterial population suspected of harboring an SNP. The amplicons are then mixed with the complexed guide nucleic acids and programmable nuclease (FIG. 12). After an optional incubation period, a pool of detector nucleic acids is added to the mixture. If the biological sample is positive for an SNP targeted by a guide nucleic acid, a guide nucleic acid and Cas14 programmable nucleases binds a target nucleic acid in the sample, activating the Cas14 programmable nuclease, and initiating transcollateral cleavage of the detector nucleic acid. The cleaved detector nucleic acid produces a detectable signal, indicating that an SNP is present in the bacterial population.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Example 10

### Guide Pooling for Enhanced Target Detection Signal in DETECTR Assays

Guide RNAs that were designed to bind to a different region within a single target molecule were pooled as a strategy for enhancing the target detection signal from DETECTR assays. For examples, in this strategy, each DETECTR™ reaction contained a pool of CRISPR-Cas RNP complexes each of which targeted a different region within a single molecule. As discussed in the paragraphs below, this strategy resulted in increased sensitivity to target detection by using increased number of complexes/single target such that the signal is strong enough to detect within a Poisson distribution (sub-one copy/droplet) and provide a quantitative evaluation of target numbers within a sample.

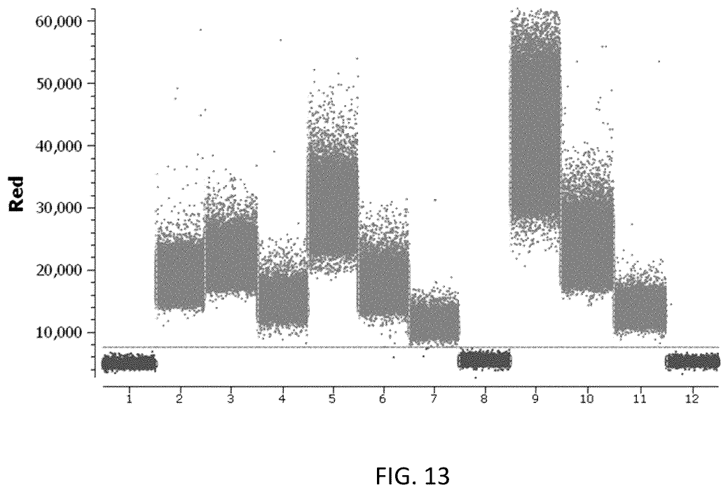

To test the effect of guide pooling on target detection using the Cas12a nuclease, first, a Cas12a complexing mix was prepared wherein the R1965 (off-target guide), R1767, R3164, R3178 guides were present in either a pooled-gRNA format (a pool of two or more of the three guides selected from R1767, R3164, or R3178) or in a single-gRNA format (wherein R1767, R3164, R3178 were present individually) and the mix was incubated for 20 minutes at 37° C. A 2-fold dilution series for the template RNA (GF184) was created from a starting dilution concentration (wherein 5.4 μl of GF184 at 0.1 ng/μL was added to 44.6 μl of nuclease-free water). DETECTR master mixes which included the Cas12 complex, Reporter substrate, Fluorescein, Buffer, and diluted template (GF184 or off-target template GF577) were then assembled as shown in Table 10. The DETECTR mixes were then loaded into a Stilla Sapphire chip and placed into the Naica Geode. Crystals were created from thousands of droplets from each samples. No amplification step was performed The signal from the Sapphire chips was measured in the Red channel. The results of the DETECTR assay showed enhanced Cas12a-based detection of the GF184 target using a pooled-guide format compared to DETECTR Cas12a-based assay using an individual guide format. For example, the DETECTR assays showed an enhanced signal from chamber 5 containing a pool of two guides R1767 and R3178, compared to the signal from chamber 2 or chamber 4 which contained the R1767 and R3178 in individual guide format respectively (FIG. 13). Similarly, the DETECTR assays showed an enhanced signal from chamber 9 containing a pool of three guides (R1767, R3164, and R3178), compared to the signal from chamber 5 which contained a pool of two guides (R1767 and R3178) and compared to the signal from chamber 2, chamber 3, or chamber 4 which contained the R1767, R3164, and R3178 in individual guide format respectively (FIG. 13).

TABLE 10

| | | Copies/ | | copies/ |
|---|---|---|---|---|
| Chamber | Condition | Chamber | # Droplets | droplet |
| 1 | Off Target | 2.5 × 10⁷ | 29336 | 852 |
| | Guide (1965) | | | |
| 2 | Single R1767 | 2.5 × 10⁷ | 26838 | 931 |
| 3 | Single R3164 | 2.5 × 10⁷ | 29590 | 845 |
| 4 | Single R3178 | 2.5 × 10⁷ | 27769 | 900 |
| 5 | 2 × pool | 2.5 × 10⁷ | 27929 | 895 |
| | (R1767, | | | |
| | R3178) | | | |
| 6 | 2 × pool | 1.25 × 10⁷ | 28787 | 434 |
| | (R1767, | | | |
| | R3178) | | | |
| 7 | 2 × pool | 6.125 × 10⁶ | 27503 | 223 |
| | (R1767, | | | |
| | R3178) | | | |
| 8 | 2 × pool | 0 | 28814 | 0 |
| | (R1767, | | | |
| | R3178) | | | |
| 9 | 3 × Pool | 2.5 × 10⁷ | 27881 | 897 |
| | (R1767, | | | |
| | R3164, | | | |
| | R3178) | | | |
| 10 | 3 × Pool | 1.25 × 10⁷ | 29523 | 423 |
| | (R1767, | | | |
| | R3164, | | | |
| | R3178) | | | |
| 11 | 3 × Pool | 6.125 × 10⁶ | 28957 | 211 |
| | (R1767, | | | |
| | R3164, | | | |
| | R3178) | | | |
| 12 | 3 × Pool | 0 | 29087 | 0 |
| | (R1767, | | | |
| | R3164, | | | |
| | R3178) | | | |

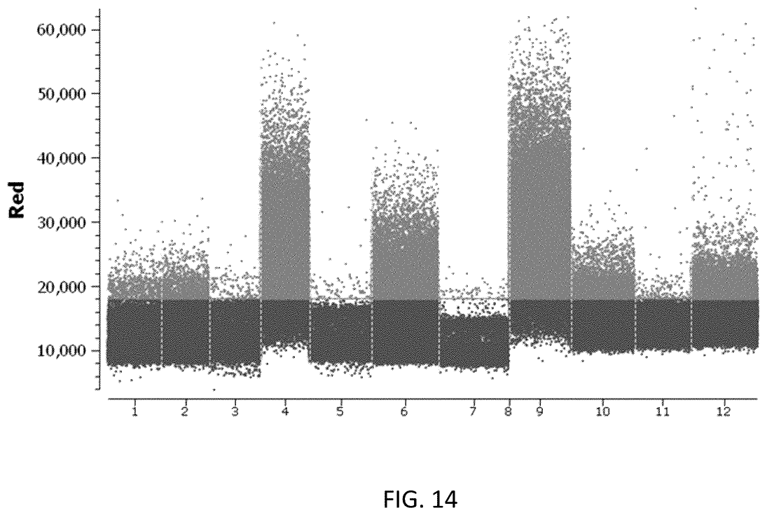

Enhanced sensitivity to target detection with guide-pooling was observed in the case of Cas13a nuclease also. In these assays, a Cas13a complexing mix was prepared wherein the R002(off-target guide), R4517, R4519, R4530 guides were present in either a pooled-gRNA format (a pool of two or more of the three guides R4517, R4519, and R4530) or single-gRNA format (wherein R4517, R4519, and R4530 were present individually) and the mix was incubated for 20 minutes at 37 C. DETECTR master mixes which included the Cas13a complex, FAM-U5 Reporter substrate, Buffer, and diluted template SC2 RNA (or off-target template 5S-87) was then assembled as shown in Table 11. The DETECTR mixes were then loaded into a Stilla Sapphire chip and placed into the Naica Geode. Crystals were created from thousands of droplets from each samples and incubated at 37 C. No amplification step was performed. The signal from the Sapphire chips was measured in the Red channel. The results of the DETECTR assay showed enhanced Cas13a-based detection of the SC2 target RNA using a pooled-guide format compared to a Cas13a-based detection of the SC2 target RNA using a single-guide format. For example, the DETECTR assays showed an enhanced signal from chamber 8, containing the template at a concentration of 1×10⁶ copies, and a pool of the three guides R4517, R4519, and R4530, compared to the signal from chamber 2, chamber 4, or chamber 6 which contained the template at a concentration of 1×10⁶ copies, and the guides R4517, R4519, and R4530 in individual guide format respectively (FIG. 14). Similarly, the DETECTR assays showed an enhanced signal from chamber 9 which contained the template at a concentration of 1×10⁵ copies and a pool of three guides (R1767, R3164, and R3178), compared to the signal from chamber 2, chamber 6, or chamber 4, which contained the template at a concentration of 1×10⁶ copies, and which contained the R1767, R3164, and R3178 in individual guide format respectively (FIG. 14).

TABLE 11

| | | Copies/ | | copies/ |
|---|---|---|---|---|
| Chamber | Condition | Chamber | # Droplets | droplet |
| 1 | Off Target | 1 × 10⁶ | 19960 | 50 |
| | Guide (R002) | | | |
| 2 | Single R4517 | 1 × 10⁶ | 18102 | 55 |
| 3 | Single R4517 | 0 | 19146 | 0 |
| 4 | Single R4519 | 1 × 10⁶ | 18289 | 55 |
| 5 | Single R4519 | 0 | 23324 | 0 |
| 6 | Single R4530 | 1 × 10⁶ | 25402 | 39 |
| 7 | Single R4530 | 0 | 26285 | 0 |
| 8 | 3 pool | 1 × 10⁶ | saturated | ~40 |
| 9 | 3 pool | 1 × 10⁵ | 23209 | 4.3 |
| 10 | 3 pool | 1 × 10⁴ | 24064 | 0.41 |
| 11 | 3 pool | 0 | 21137 | 0 |
| 12 | 3 pool | 1 × 10⁶ | 24885 | 40 |

Next, the sensitivity of a target detection in Cas13a digital droplet DETECTR assays containing guide RNA in either a pooled-guide format versus a single guide format was assayed. DETECTR reaction master-mixes was prepared for each gRNA (R4637, R4638, R4667, R4676, R4684, R4689, R4691, or R4785 (RNaseP)) and included, in addition to the gRNA, the Cas13a nuclease, and the reporter substrate. After complexing, 2 μL of each RNP was combined in either a pooled-gRNA format (a pool of the seven gRNAs, i.e., R4637, R4638, R4676, R4689, R4691, R4667, and R4684) or remained in the single-gRNA format (wherein R4667, R4684, and R4785 (RNAse P were present individually). The template RNAs (Twist SC2, ATCC SC2, and 5s-87 off-target) were diluted to obtain a series of template concentrations. DETECTR reactions directed to the detection of the template RNAs (Twist SC2, ATCC SC2, and 5s-87 off-target template RNAs) were assembled by combining the Cas13a-gRNA RNPs with the diluted template RNA from the previous step as shown in Table 12. The assembled DETECTR reactions were loaded into chambers on a Stilla Sapphire Chip. The Chips were placed into the Naica Geode and crystals were generated using the droplet generation program. The chips were incubated and the crystals generated were imaged to reveal droplets that contain detected targets.

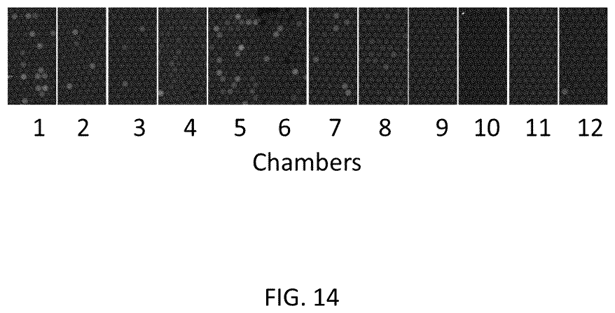

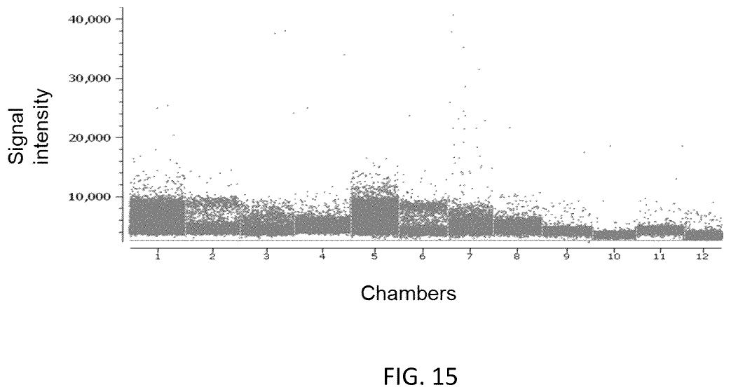

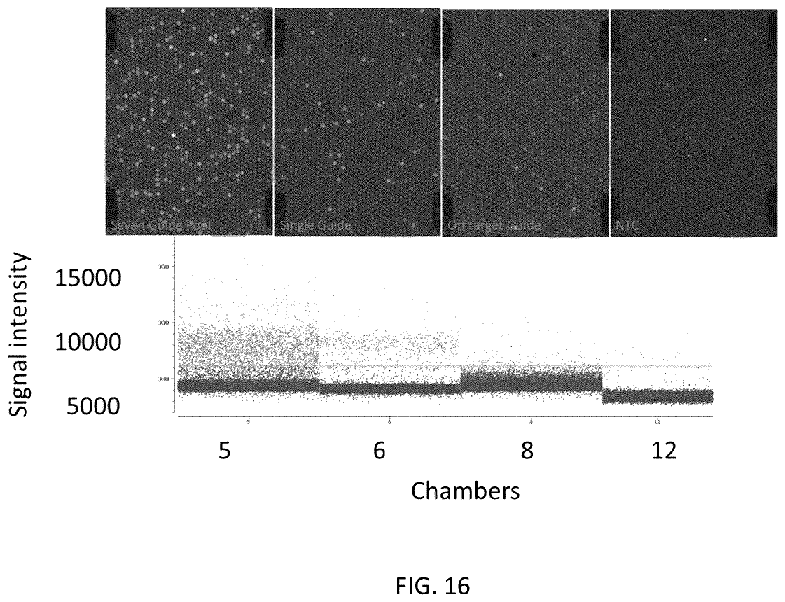

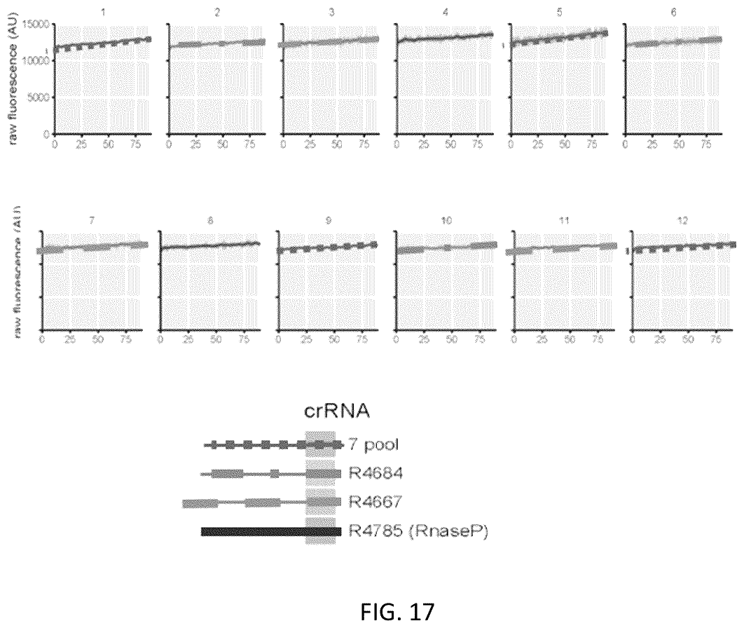

The sensitivity of target detection by the DETECTR assays containing the pooled guides (R4637, R4638, R4667, R4676, R4684, R4689, R4691) was compared with the sensitivity of target detection by the DETECTR assays containing the single guides R4684, R4667, R4785 (RNAseP guide) in individual format. Relative quantification performed by counting the number of these positive droplets showed that the samples containing the pooled guide RNAs generated more crystals containing the amplified products per copy of starting target RNA than the samples containing the guide RNAs in individual format (FIG. 15). For example, the number of droplets from chamber 1 is higher than the number of droplets in chamber 2 and 3; and the number of droplets from chamber 5 is higher than the number of droplets in chambers 6 and 7 (FIG. 15 and FIG. 17). Measurement of the target detection signal intensity from the chips also confirmed that the sensitivity of target detection per copy of starting target RNA by the DETECTR assays containing the pooled guides (R4637, R4638, R4667, R4676, R4684, R4689, R4691) was higher than the sensitivity of target detection by the DETECTR assays containing the single guides R4684, R4667, R4785 (RNAseP guide) in individual format (FIG. 16). For example, signal intensity from chamber 1 (containing the seven-guide pool and the Twist SC2 template RNA is higher than the signal intensity in chamber 2 and 3 (containing the R4684, and the R4667 gRNAs in individual format respectively in the presence of the Twist SC2 RNA); and the signal intensity from chamber 5 (containing the seven-guide pool and the ATCC SC2 template RNA) is higher than the signal intensity in chambers 6 and 7 (containing the R4684, and the R4667 gRNAs in individual format respectively, in the presence of the ATCC SC2 RNA) (FIG. 16). Similarly, the signal intensity from chamber 5 (containing the seven-guide pool and the ATCC SC2 template RNA) is higher than the signal intensity in chamber 6 (containing the gRNA R4684 in individual format and the ATCC SC2 RNA), the signal intensity from chamber 8 (containing the control RNaseP gRNA in individual format with the ATCC SC2 template RNA) and the signal intensity from chamber 12 (containing the seven pooled gRNAs with no template RNA) (FIG. 16). Similarly, the relative quantification of the number of droplets containing amplified target (per copy of starting target RNA) observed in chamber 5 (containing the seven-guide pool and the ATCC SC2 template RNA) is higher than the number of droplets observed in chamber 6 (containing the gRNA R4684 in individual format and the ATCC SC2 RNA), the number of droplets observed in chamber 8 (containing the control RNaseP gRNA in individual format with the ATCC SC2 template RNA) and the number of droplets observed in chamber 12 (containing the seven pooled gRNAs with no template RNA) (FIG. 17) The sensitivity of target detection by the assays containing the pooled guides (R4637, R4638, R4667, R4676, R4684, R4689, R4691) was compared with the sensitivity of target detection by the assays containing the single guides R4684, R4667, R4785 (RNAseP guide) in individual format, when the assays were conducted in a benchtop assay format (FIG. 18). Results from the bench top assay showed that the samples containing the pooled guides (R4637, R4638, R4667, R4676, R4684, R4689, R4691) was not higher than the sensitivity of target detection by the in the samples containing the single guides R4684, R4667, or R4785 (RNAseP guide) in individual format (FIG. 18).

TABLE 12

| Chamber | Guide | Template |
|---|---|---|
| 1 | 7 pool | 5000 copies Twist SC2 |
| 2 | R4684 | 5000 copies Twist SC2 |
| 3 | R4667 | 5000 copies Twist SC2 |
| 4 | R4785(RNaseP) | 5000 copies Twist SC2 |
| 5 | 7 pool | 5000 copies ATCC SC2 |
| 6 | R4684 | 5000 copies ATCC SC2 |
| 7 | R4667 | 5000 copies ATCC SC2 |
| 8 | R4785(RNaseP) | 5000 copies ATCC SC2 |
| 9 | 7 pool | 5000 copies 5s-87 |
| 10 | R4684 | 5000 copies 5s-87 |
| 11 | R4667 | 5000 copies 5s-87 |
| 12 | 7 pool | NTC |

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition comprising a programmable nuclease and a pool of guide nucleic acids comprising greater than 20 distinct guide nucleic acid sequences, wherein at least one guide nucleic acid of the pool hybridizes to a segment of a target nucleic acid.

2. The composition of claim 1, wherein the pool of guide nucleic acids comprises at least 50 distinct guide nucleic acid sequences, at least 100 distinct guide nucleic acid sequences, at least 500 distinct guide nucleic acid sequences, or at least 1000 distinct guide nucleic acid sequences.

3. The composition of claim 1, wherein the pool of guide nucleic acids comprises at least two guide nucleic acids that hybridize to a different segment of the target nucleic acid.

4. The composition of claim 1, wherein a guide nucleic acid of the pool of guide nucleic acids has a sequence selected from a group of tiled guide nucleic acids that correspond to nucleic acids of the target nucleic acid.

5. The composition of claim 4, wherein:
a) the tiled guide nucleic acids are sequential along the target nucleic acid upon hybridization to the target nucleic acid;
b) the tiled guide nucleic acids are non-sequential along the target nucleic acid upon hybridization to the target nucleic acid;
c) the tiled guide nucleic acids are overlapping along the target nucleic acid upon hybridization to the target nucleic acid; or
d) any combination thereof.

6. The composition of claim 1, wherein the target nucleic acid is from a pathogen.

7. The composition of claim 1, wherein at least two guide nucleic acids of the pool of guide nucleic acids hybridize to segments of distinct target nucleic acids.

8. The composition of claim 7, wherein at least two target nucleic acids of the distinct target nucleic acids are from different pathogens.

9. The composition of claim 6, wherein the pathogen is a virus, a bacterium, a fungus, a protozoan, or a worm.

10. (canceled)

11. The composition of claim 1, wherein at least two guide nucleic acids of the pool of guide nucleic acids differs from one another by at least one base.

12. The composition of claim 1, wherein a total concentration of the pool of guide nucleic acids is about 400 nM, about 1000 nM (1 μM), or about 2000 nM (2 μM).

13. The composition of claim 1, wherein each guide nucleic acid of the pool of guide nucleic acids comprises from 20 to 50 bases.

14. (canceled)

15. The composition of claim 1, wherein the programmable nuclease is a Type V CRISPR-Cas enzyme or a Type VI CRISPR-Cas enzyme.

16.-26. (canceled)

27. The composition of claim 1, wherein the target nucleic acid is DNA or RNA.

28. (canceled)

29. The composition of claim 1, wherein the composition further comprises the target nucleic acid.

30. The composition of claim 29, wherein the target nucleic acid comprises distinct target nucleic acids.

31. A method of assaying for a segment of a target nucleic acid in a sample, the method comprising:
contacting the sample to the composition of claim 1; and
assaying for a signal produce by cleavage of a detector nucleic acid.

32. The method of claim 31, further comprising reverse transcribing the target nucleic acid, amplifying the target nucleic acid, in vitro transcribing the target nucleic acid, or any combination thereof.

33. The method of claim 32, wherein the amplifying is isothermal amplification.
